(19) United States
(12) Patent Application Publication
(10) Pub. No.: US 2003/0134281 A1
(43) Pub. Date: Jul. 17, 2003

(54) Nanomachine compositions and methods of use

(76) Inventor: Glen A. Evans, San Marcos, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/960,870

(22) Filed: Sep. 20, 2001

(51) Int. Cl. C12Q 1/68; C12N 5/00

(52) U.S. Cl. 435/6; 435/325; 435/455

(57) ABSTRACT

The invention provides a basic genetic operating system for an autonomous prototrophic nanomachine having a nanomachine genome encoding a minimal gene set sufficient for viability. Also provided is a basic genetic operating system for an autonomous auxotrophic nanomachine having a nanomachine genome encoding a minimal gene set sufficient for viability in the presence of an auxotrophic biomolecule. The minimal gene set encoded by the basic genetic operating system can contain the functional categories of transcription, translation, aerobic metabolism, glycolysis/pyruvate dehydrogenase/pentose phosphate pathways, carbohydrate metabolism, central intermediary metabolism, nucleotide metabolism, transport and binding proteins, and housekeeping functions. Functional categories can be arranged in a predetermined physical or temporal order. A prototrophic basic genetic operating system sufficient for autonomous viability can contain a minimal gene set of about 152 or less fundamental genes, orthologs or nonothorologous displacements thereof. An auxotrophic basic genetic operating system sufficient for autonomous viability in the presence of an auxotrophic biomolecule can contain about 151 or less fundamental genes, orthologs or nonothorologous displacements thereof. Also provided is a basic genetic operating system sufficient for autonomous prototrophic or auxotrophic viability which can have an expression control region for the production of a biomolecule. Viable autonomous prototrophic and auxotrophic nanomachines are also provided.

### BACKGROUND OF THE INVENTION

This invention relates generally to organismic biology and, more specifically to construction and operation of DNA-based nanomachines.

The diagnosis and treatment of human diseases continues to be a major area of social concern. The importance of improving health care is self-evident, so long as there continues to be diseases that affect individuals, there will be an effort to understand the cause of such diseases as well as efforts to diagnose and treat such diseases. Preservation of life is an inherent force motivating the vast amount of time and expenditure continually invested into scientific discovery and development processes. The application of results from these scientific process to the medical field has led to surprising advancements in diagnosis and treatment over the last century, and especially over the last quarter century. Such advancements have improved both the quality of life and life-span of affected individuals.

However significant in both scientific and medical contribution to their respective fields, the progression of advancements have been slow and painstaking, generally resulting from step-wise trial and error hypothesis-driven research. Moreover, with each advancement there can be cumulative progression in the overall scientific understanding of a problem but there is no guarantee that the threshold needed to translate a discovery into a practical medical application has been achieved. Additionally, with the achievement of all too many advancements comes the sobering realization that the perceived final answer for a complete understanding of a particular physiological or biochemical process is, instead, just a beginning to a more complex process still needed to be dissected and understood.

Further complicating the progression of scientific advancements and their practical application can result from technical limitations in available methodology or materials. Each discovery or advancement can push the frontiers of science to new extremes. Many times, continued progress can be stalled due to the unavailability or insufficiency in technological sophistication needed to continue studies at the new extremes. Therefore, further advancements in the scientific discovery and medical fields necessarily have to await progress in other fields for the advent and development of more capable technologies and materials. As a result, the progression of scientific advancements having practical diagnostic and therapeutic applications can occur relatively slowly because it results from the accumulation of many smaller discoveries, contributions and advancements in technologies.

Nanotechnology has been one such scientific advancement purported to open new avenues into the discovery and development processes and achieve new dimensions in the medical diagnostic and therapeutic fields. Nanotechnology has been described as the production of systems on the order of one to one hundred nanometers in size or the manipulation of matter at the atomic level. Futuristic speculation of nanotechnology for medical applications has been directed to the production of miniature devices and machines that in effect mimic or control biochemical process through hybrid biomechanical and bioelectrical assemblies. Similarly, the construction of nanostructures also has been purported as an advancement that will revolutionize diagnostic applications because of their precise physical characteristics and comparable size to their molecular targets.

The construction of atomic level substances through molecular manipulation is a technology imagined five decades ago. Similarly, the idea of merging biological and nonbiological materials also is not new. With the expanding availability of a variety of materials and with advancements in physical and chemical methods for manipulation of matter at the nanoscale level, the construction of structures with highly controlled and unique properties can be accomplished. A fledgling industry has now emerged which is attempting to exploit these properties of nanostructures. However, except for physical and chemical approaches for manipulating matter, the application of nanotechnology to biology is still in the conception stage.

Therefore, while spectacular in its potential ramifications, nanotechnology as initially imagined has not yet come to fruition. Despite the numerous descriptions of miniature devices and machines probing and surveying the body, the only commercial applications to result from nanotechnology have been dirt-repelling surface coatings and paint additives. One drawback hindering the application and development of nanotechnology to biology is due to its bottom-up synthesis approach from single atoms or molecules for precise miniaturization. Such an approach requires sophisticated and advanced technology derived from the combination of numerous disciplines. However, for many assembly steps, the envisioned technology required for precise synthesis of complicated nanodevices and biomechanical machines is not yet available or fully developed.

Thus, there exists a need for nanoscale compositions with defined characteristics that can probe and mimic physiological and biochemical processes without hindrance by limitations in technology development. The present invention satisfies this need and provides related advantages as well.

### SUMMARY OF THE INVENTION

The invention provides a basic genetic operating system for an autonomous prototrophic nanomachine having a nanomachine genome encoding a minimal gene set sufficient for viability. Also provided is a basic genetic operating system for an autonomous auxotrophic nanomachine having a nanomachine genome encoding a minimal gene set sufficient for viability in the presence of an auxotrophic biomolecule. The minimal gene set encoded by the basic genetic operating system can contain the functional categories of transcription, translation, aerobic metabolism, glycolysis/pyruvate dehydrogenase/pentose phosphate pathways, carbohydrate metabolism, central intermediary metabolism, nucleotide metabolism, transport and binding proteins, and housekeeping functions. Functional categories can be arranged in a predetermined physical or temporal order. A prototrophic basic genetic operating system sufficient for autonomous viability can contain a minimal gene set of about 152 or less fundamental genes, orthologs or nonothorologous displacements thereof. An auxotrophic basic genetic operating system sufficient for autonomous viability in the presence of an auxotrophic biomolecule can contain about 151 or less fundamental genes, orthologs or nonothorologous displacements thereof. Also provided is a basic genetic operating system sufficient for autonomous prototrophic or auxotrophic viability which can have an expression control region for the production of a biomolecule. Viable autonomous prototrophic and auxotrophic nanomachines are also provided.

Further provided is a basic genetic operating system for an autonomous prototrophic nanomachine having a nanomachine genome encoding a minimal gene set sufficient for autonomous prototrophic replication. Also provided is a basic genetic operating system for an autonomous auxotrophic nanomachine having a nanomachine genome encoding a minimal gene set sufficient for autonomous replication in the presence of an auxotrophic biological molecule. The minimal gene set encoded by the basic genetic operating system can direct synthesis of the minimal gene set in a relative order of functional categories corresponding to replication, transcription, translation, aerobic metabolism and glycolysis/pyruvate dehydrogenase/pentose phosphate pathways. Additional functional categories can be for carbohydrate metabolism, central intermediary metabolism, nucleotide metabolism, signal transduction regulation, transport and binding proteins, particle division, chaperone system, fatty acid/lipid metabolism, particle envelope and housekeeping functions. The functional categories can be arranged in a predetermined physical or temporal order. A prototrophic basic genetic operating system sufficient for autonomous replication can contain about 247 or less fundamental genes, orthologs or nonorthologous displacements thereof. An auxotrophic basic genetic operating system sufficient for autonomous replication in the presence of an auxotrophic biomolecule can contain about 246 or less fundamental genes, orthologs or nonothorologous displacements thereof. Also provided is a basic genetic operating system sufficient for autonomous prototrophic or auxotrophic replication which can have an expression control region for the production of a biomolecule. Replication competent autonomous prototrophic and auxotrophic nanomachines are also provided.

### BRIEF DESCRIPTION OF THE DRAWINGS

FIG. 1 shows fundamental genes and functional categories of a basic genetic operating system for a viable prototrophic nanomachine.

FIG. 2 shows fundamental genes and functional categories of a basic genetic operating system for a replication competent prototrophic nanomachine.

### DETAILED DESCRIPTION OF THE INVENTION

This invention is directed to biological nanomachines programed and self-produced by nucleic acid-based information. Nanomachine genomes can be created that encode all essential information for autonomous existence and operation. Additionally, nanomachines can be programmed to perform essentially any activity exhibited by cellular life. Nanomachine programming is implemented through nucleic acid-based information. Genetic instructions can be created, such as a genetic operating system, that encodes all functions sufficient for a biological nanomachine of the invention to self-produce required components and perform cellular life functions. The biological nanomachines of the invention can be further programmed to perform a wide variety of activities by modification of their genome to incorporate or modify a predetermined function. Therefore, additional genes can be added to the genetic operating system which encode further instructions sufficient to self-produce and maintain supplemental cellular functions and activities. Versatility is one advantage of the nanomachines of the invention because they can be programmed for minimal functions, basic cellular life functions or to additionally include a wide variety of complicated activities.

The genetic instructions, or nucleic acid material, are read using ordinary cellular machinery and converted into other nucleic acids, polypeptides, macromolecules or other organic compounds that perform the work of the encoded cellular functions. The nanomachines of the invention are therefore produced through biosynthesis of constituent components and self-assembly into functional biological structures. Using nucleic acid-based information, biochemical rules and complex mechanisms of manipulating matter can be reliably harnessed without the need for sophisticated or advanced nanotechnology. Therefore, another advantage of the biological nanomachines of the invention is that they can be produced and maintained by bottom-up synthesis using rules and self-assembly processes of nature that have been evolutionary selected and are well understood. Moreover, the use of nucleic acid encoded information is a further advantage of the invention because it can be maintained through biological replication processes and can be continually employed to direct the production of constituent nanomachine components through reliable biosynthetic processes.

In one embodiment, the invention is directed to a basic genetic operating system that is sufficient to sustain viability for an autonomous nanomachine. A basic genetic operating system is a nanomachine genome which contains the genetic programming required to direct the synthesis and operation of an autonomous nanomachine. Such genetic programming consists of a minimal gene set sufficient to carry out component synthesis required for fundamental functions of an autonomous nanomachine. A minimal compilation of genes with sufficient information to support viability will contain, for example, genes required to effect basic cellular and biochemical process such as transcription, translation and energy production as well as other basic cellular homeostasis processes such as nucleotide metabolism, carbohydrate metabolism, central intermediate metabolism and housekeeping functions. In a specific embodiment, such a basic genetic operating system specifying nanomachine viability contains about 152 genes. Additional genes or gene sets, such as for the production of a therapeutic polypeptide or diagnostic indicator, can be incorporated into the basic genetic operating system to generate a genome further programmed to execute and carry out activities and operations additional to those specified by the basic operating system. The basic genetic operating systems of the invention also can be harbored in a lipid vesical or other biologically compatible materials to produce an autonomous nanomachine of the invention.

In another embodiment, the invention is directed to a basic genetic operating system for autonomous nanomachines that are replication competent. A minimal gene set sufficient to carry out component synthesis for fundamental functions of replication competent nanomachines can contain in addition to those required for viability, genes required for replication, particle division, fatty acid/lipid metabolism and particle envelope components, for example. In a specific embodiment, such a basic genetic operating system specifying a replication competent nanomachine contains about 247 genes. Additional genetic programming can be overlaid onto a basic genetic operating system directing autonomous replication by incorporating instructions for a wide variety of activities and operations into the nanomachine genome. Therefore, replication competent nanomachines can be advantageously used for persistent performance of useful activities such as the production of therapeutic polypeptides or diagnostic indicators. Basic genetic operating systems specifying replication competence can be harbored in lipid bilayer membranes directed and synthesized from the nanomachine's basic genetic operating system as well as a lipid vesical or other biologically compatible material to produce an replication competent autonomous nanomachine of the invention.

In another embodiment, autonomous nanomachines of the invention can be programmed with prototrophic or auxotrophic basic genetic operating systems. A nanomachine harboring a prototrophic basic genetic operating system is a genotypically complete genome so as to encode all mandatory gene products for nanomachine autonomy. For example, a prototrophic nanomachine programmed with a basic genetic operating system conferring replication competence will encode the requisite gene products sufficient to sustain replication similar to cellular life forms. A nanomachine harboring an auxotrophic basic genetic operating system is an incomplete genome for at least one gene product required for nanomachine autonomy. Autonomy can be conferred on such auxotrophic nanomachines programmed with a basic genetic operating system by exogenously supplying the gene product or biosynthetic intermediate to the nanomachine.

As used herein, the term “basic” when used in reference to a genetic operating system, is intended to mean a elementary or foundational set of genetic instructions that can direct an autonomous function of a nanomachine. An elementary or foundational set of genetic instructions will contain, for example, a substantially non-redundant set of genes that encode a minimal number of gene products required to effect one or more autonomous functions of a nanomachine. Substantially non-redundant genetic instructions are genes or gene sets that are non-coextensive in structure or function and include similar but functionally distinguishable genes or gene sets and their respective gene products. The term basic therefore refers to an underlying set of genes that encode products required for fundamental activities of a nanomachine. A basic genetic system therefore provides the essential genetic program which directs autonomy of a nanomachine. A basic system also allows for the integration of additional genetic programs that, when executed, can perform a variety of other activities, including for example, preforming useful work or directing the production of useful molecules and biological processes.

As used herein, the term “genetic operating system” is intended to mean a genetic program or set of instructions encoded in a nucleic acid that controls the operation of one or more autonomous functions of a nanomachine. A genetic operating system therefore specifies nanomachine gene products that provide fundamental activities and direct the regulation of such activities to achieve functional autonomy. A genetic operating system also controls integration and directs the regulation and execution of additional genetic programs that can perform numerous general or specialized functions of a nanomachine. Such overlying or operating system-dependent genetic programs specify, for example, non-autonomous functions of a nanomachine as they are dependent on the underlying basic genetic operating system to supply components or activities essential for initiation, execution or completion of the encoded task. A genetic operating system can encode genes sufficient for the control and operation of a single autonomous nanomachine function as well as for the control, integration and operation of multiple autonomous functions, including for example, nanomachine viability, replication and proliferation.

The structure of a genetic operating system can be arranged in a variety of different formats so long as it encodes sufficient genetic information for the control and operation of one or more autonomous functions of a nanomachine. For example, a genetic operating system can be composed of a single nucleic acid genome containing a complete integrated set of genes that specify the functionality of the basic operating system. Alternatively, it can be composed of two or more nucleic acid genomes that together specify the functionality of the basic operating system. Similarly, genes which make up a genetic operating system can be integrated into a nanomachine genome in any arrangement so long as they direct the control and operation of an encoded autonomous function. For example, constituent genes can be organized linearly, functionally or randomly within the genetic operating system. Similarly, constituent genes can be composed of subsets, defined for example, by various structural or functional criteria known to those skilled in the art, and such subsets or modules can be organized linearly, functionally or randomly within the genetic operating system. Therefore, so long as the genetic operating system sufficiently encodes and produces gene products that execute the control and operation of an autonomous nanomachine function, the structure of a genetic operating system can be arranged, for example, as a single or multiple component genome, with fundamental genes individually or modularly integrated, or in a linear, functional or random organization.

As used herein, the term “autonomous” is intended to mean independent operation. Independence is used to characterize an autonomous operation in relation to an engineered activity of a referenced nanomachine or process thereof. Therefore, an autonomous operation or activity can function on its own resources given a particular environment consistent with the engineered activity or function. Similarly, an autonomous operation or activity can be performed without the need for external sources of nucleic acid-encodable molecules for production, activity, regulation or homeostasis, for example, with respect to the referenced nanomachine operation or activity. Autonomous operations or activities of a nanomachine include, for example, viability, replication, proliferation or protein synthesis. The term “autonomous” is intended to include, for example, dependence on external sources of essential nutritional requirements for survival. Such essential nutritional requirements include, for example, a carbon source, an oxygen source for aerobic conditions, a nitrogen source, and inorganic compounds. Autonomous operation also can include, for example, dependence on a sulphur source.

For example, a protrotrophic nanomachine capable of autonomous replication harbors sufficient nucleic acid-encodable information to synthesize the required molecules necessary to generate and perform obligatory processes for replication. Therefore, a autonomous prototrophic nanomachine that is replication competent can carry out transcription, translation and nucleic acid replication functions without dependence on external sources for encodable factors such as macromolecules. Self-contained replication would be one phenotype of such a replication competent prototrophic nanomachine. The genotype of such a prototrophic nanomachine will consist of requisite genes necessary to initiate and execute the biological functions of transcription, translation, replication and energy production.

Similarly, an auxotrophic nanomachine capable of autonomous replication will harbor sufficient nucleic acid-encodable information to synthesize the required molecules necessary to generate and perform obligatory processes for replication with the inclusion of one or more auxotrophic biological molecules. Therefore, a autonomous auxotrophic nanomachine that is replication competent can carry out transcription, translation and nucleic acid replication functions without dependence on external sources for encodable factors other than an auxotrophic molecule. Self-contained replication in the presence of an auxotrophic molecule would be one phenotype of such a replication competent auxotrophic nanomachine. The genotype of such a auxotrophic nanomachine will consist at least one defective gene corresponding to an auxotrophic molecule as well as all other requisite genes necessary to initiate and execute the biological functions of transcription, translation, replication and energy production.

As used herein, the term “prototroph” or “prototrophic” is intended to mean a nanomachine, or operation thereof, having the nutritional requirements corresponding to a referenced phenotype of a genotypically complete nanomachine. A nanomachine, or operation thereof, is genetypically complete when it encodes the requisite obligatory gene products to synthesize required biological components and autonomously perform the engineered activity or activities in the referenced phenotype. A referenced phenotype of a nanomachine, or operation thereof, is also referred to as a wild type phenotype when used to describe an operation or activity of a genotypically complete nanomachine. Therefore, a prototrophic nanomachine references the designed nutritional requirements corresponding to the engineered activity or activities of a genotypically complete nanomachine.

For example, where an engineered activity is amino acid synthesis through salvage pathways, obligatory encoded gene products of a genotypically complete nanomachine would consist of the required salvage pathway enzymes for amino acid synthesis. Similarly, where de novo amino acid synthesis is an engineered activity, a genotypically complete nanomachine would consist of the required set of encoded gene products sufficient to biochemically synthesize all twenty naturally occurring amino acids. In both of the above specific examples, the reference phenotype can be replication competent. The former having an engineered activity of salvage synthesis of amino acids whereas the latter having an engineered activity of de novo amino acid synthesis.

As used herein, the term “auxotroph” or “auxotrophic” is intended to mean a nanomachine, or operation thereof, having the nutritional requirements corresponding to a referenced phenotype of a genotypically incomplete nanomachine. A nanomachine, or operation thereof, is genetypically incomplete when it is deficient in encoding at least one obligatory gene product for synthesis of required biological components sufficient for autonomous performance of the engineered activity or activities of the referenced phenotype. Therefore, an auxotrophic nanomachine references the requirement of the deficient gene product, or a downstream product, that can restore autonomous performance of the engineered activity or activities in addition to referencing the designed nutritional requirements corresponding to the engineered activity of an otherwise genotypically complete nanomachine.

For example, where an engineered activity is nucleotide synthesis through salvage pathways and the nanomachine is auxotrophic for purines, nutritional requirements would include a supply of purines or precursors of purines. The obligatory encoded gene products of an otherwise genotypically complete nanomachine would consist of the required salvage pathway enzymes for complete nucleotide synthesis except for one or more gene products in the purine salvage pathway. Similarly, where de novo nucleotide synthesis is an engineered activity, nutritional requirements would include a supply of substrates or precursors, or a downstream product within the pathway. An otherwise genotypically complete nanomachine would consist of the required set of encoded gene products sufficient to biochemically synthesize all naturally occurring nucleotides. In both of the above specific examples, the reference phenotype can be replication competent. The former having an engineered activity of salvage synthesis of nucleotides whereas the latter having an engineered activity of de novo nucleotide synthesis.

An “auxotrophic biological molecule” or “auxotrophic biomolecule” as it is used herein, is a molecule that restores autonomy to an auxotrophic nanomachine, or operation thereof, when supplied in the growth medium or living environment of the nanomachine. Similarly, the gene or genes responsible for the referenced biosynthetic defect is referred to herein as an “auxotrophic gene” or “auxotrophic genes.”

As used here, the term “nanomachine” is intended to mean a biochemically-based particle that can be genetically programed to perform biochemical or physiological work. Biochemically-based particles are those bodies that can synthesize components required for autonomous function from molecules found in nature, including for example, those molecules in physiological systems. Therefore, a biochemically-based particle also can be considered a nucleic acid-based particle where the instructions required for component synthesis are encoded in a nucleic acid. Generally, a nanomachine will contain at least a basic genetic operation system and a particle envelope. A particle envelope can be, for example, a physical partition or other physical or chemical means which can control a microenvironment. The basic genetic operating system directs, for example, the control and operation of autonomous nanomachine functions whereas the particle envelope partitions, for example, nanomachine components from non-nanomachine components. A nanomachine also can contain, for example, additional genetic programs that perform numerous general or specialized biochemical activities of a nanomachine. Biochemical or physiological work of a nanomachine can include, for example, particle viability, proliferation, replication, transcription and translation. Moreover, a nanomachine can be loaded with various additional components either pre- or post-operational start-up and still be included within the meaning of the term. The actual shape or size of a nanomachine can vary so long as it is a biochemically-based particle and is, or can be made to be, genetically programed to perform biochemical or physiological work.

As used herein, the term “minimal” when used in reference to a gene set is intended to mean a substantially non-redundant threshold number of genes that are sufficient or adequate to perform a referenced activity. Therefore, a minimal set of genes are those genes that are required to competently perform a referenced nanomachine activity. For example, a minimal gene set can be specific to a referenced functional category such as replication or aerobic metabolism. Alternatively, a minimal gene set can be directed to combined functions of a referenced activity such as replication competency or viability. A threshold number of genes can be, for example, at least those genes that are indispensable to the performance of a nanomachine operation or activity encoded by the referenced gene set. A threshold number of genes also can include, for example, other genes able to increase the competency of the process without substantial overlap in gene product function. Therefore, a minimal gene set can be, or will include for example, the least possible number of genes sufficient to perform a referenced operation or activity.

It is understood that a minimal gene set is not restricted to genes derived from one species or even from a few different species. Instead, minimal gene sets can be composed of all genes derived from the same species, different related species, different divergent species or from various combinations thereof. Such species can include, for example, procaryotes such as Mycoplasma genitalium, Haemophilus influenzae and Escherichia coli, and eucaryotes such as yeast, nematodes, insects, other invertebrates, vertebrates, mammalian, including rodent, primate and human. Minimal gene sets include, for example, those for M. genitalium, H. influenzae, and E. coli described by Fraser et al., Science, 270:397-403 (1995); Mushegian and Koonin, Proc. Natl. Acad. Sci. U.S.A., 93:10268-73 (1996); Koonin et al., Trends Genet., 12, 334-336 (1996); Hutchison et al., Science, 286:2165-69 (1999), or at NCBI URL ncbi.nlm.nih.gov/cgi-bin/Complete_Genomes/mglist, all of which are incorporated herein by reference. A set of fundamental genes is a further specific example of a minimal gene set.

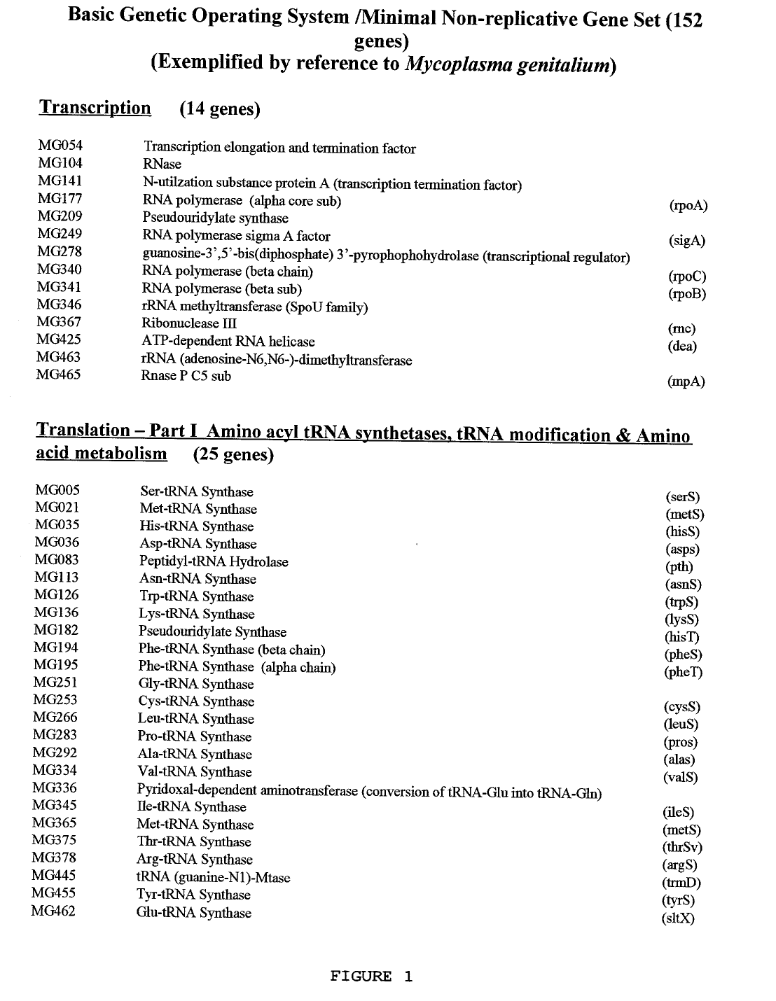

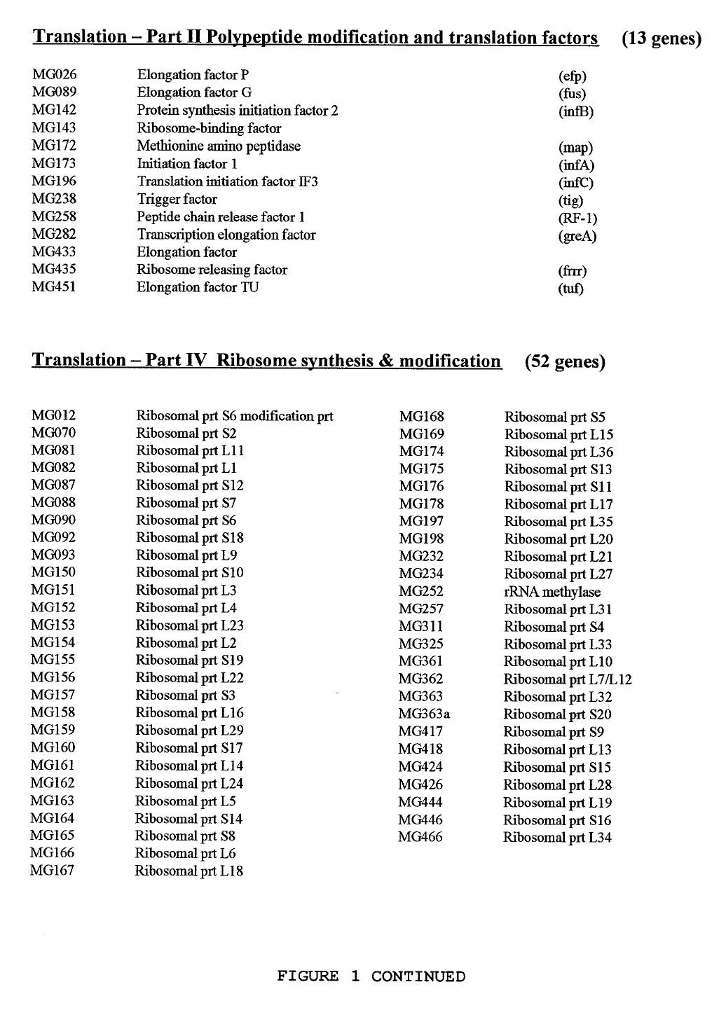

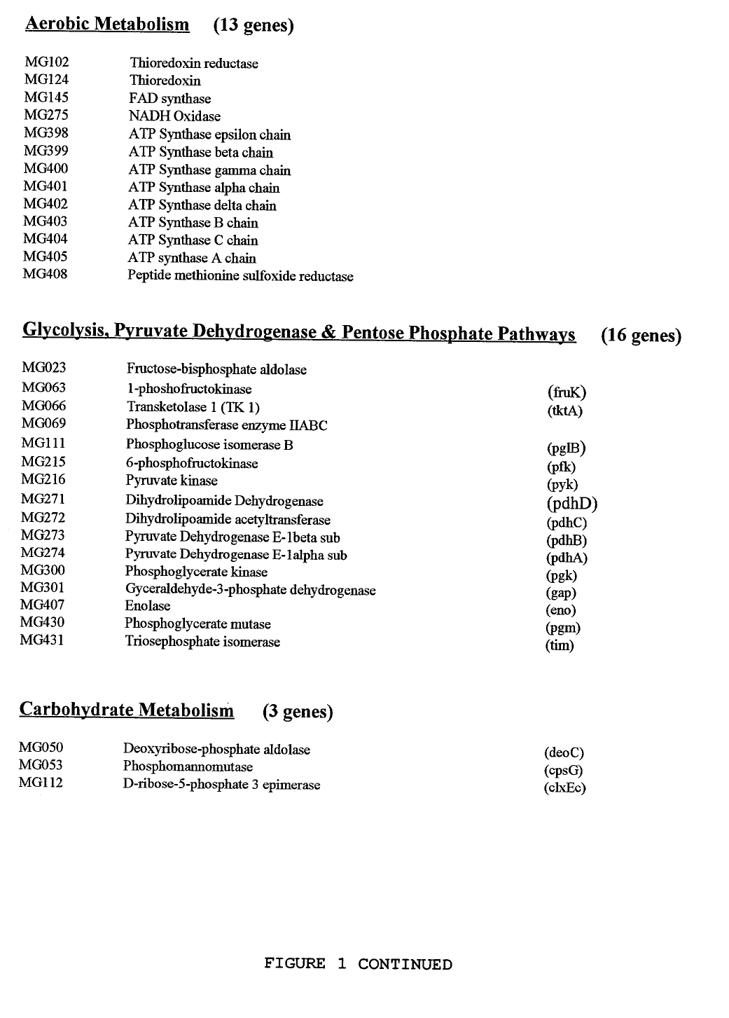

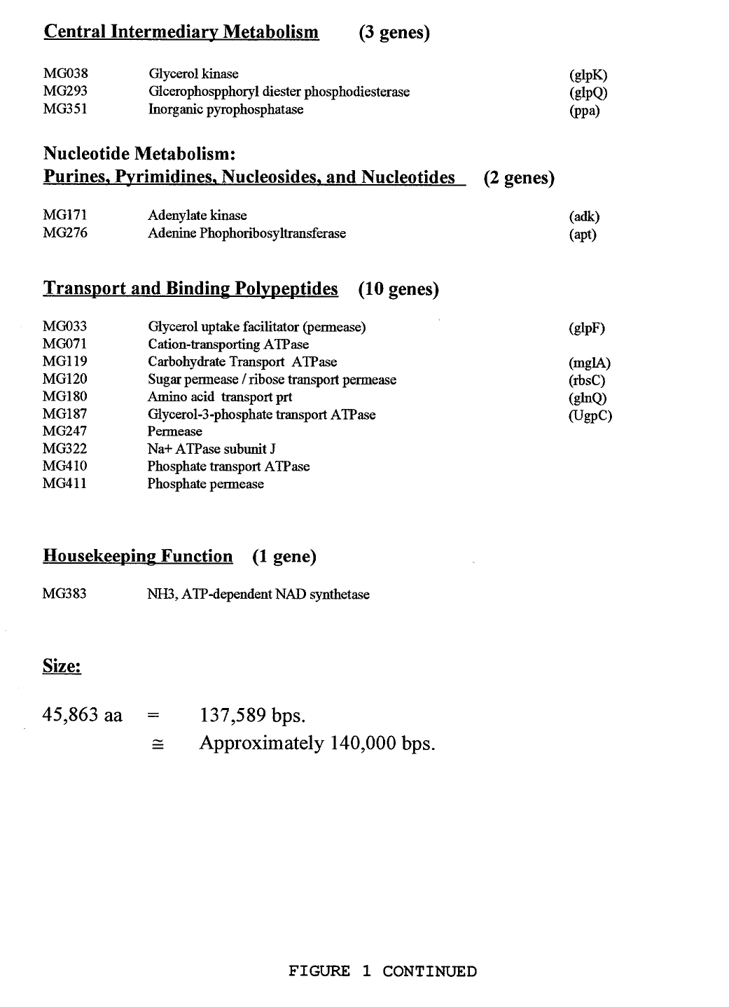

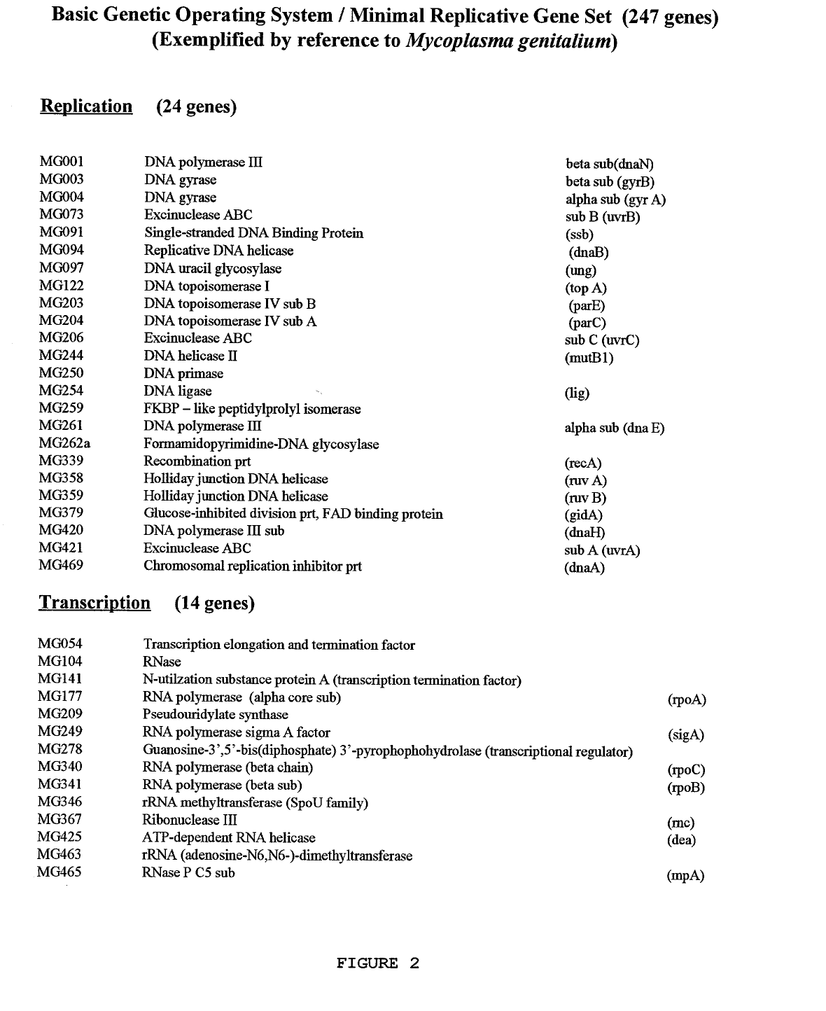

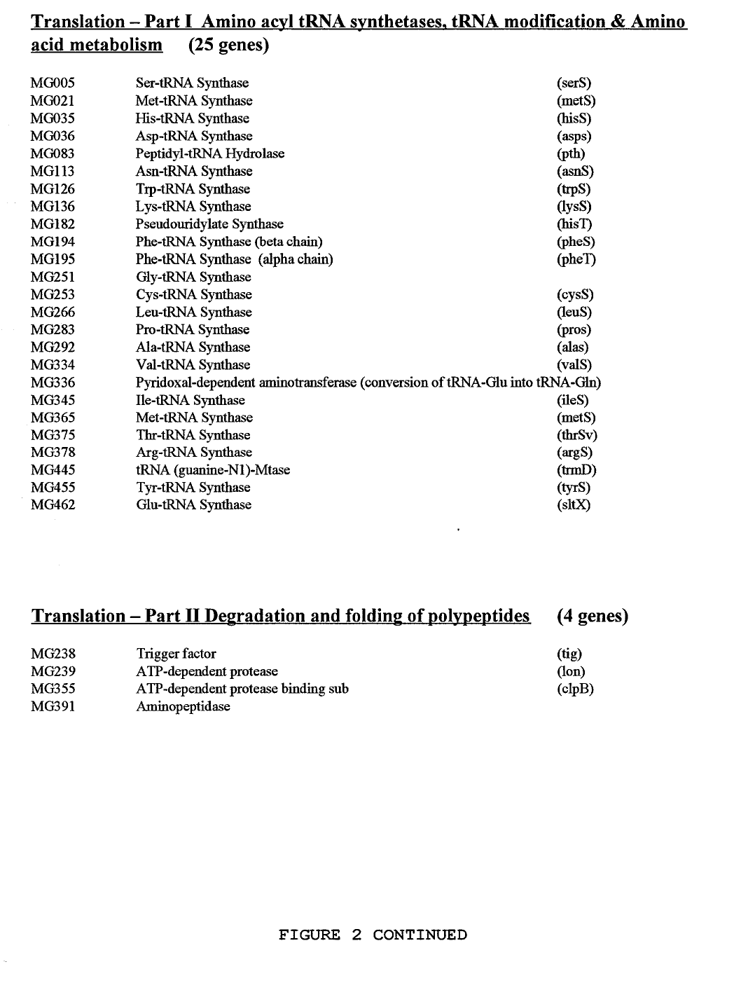

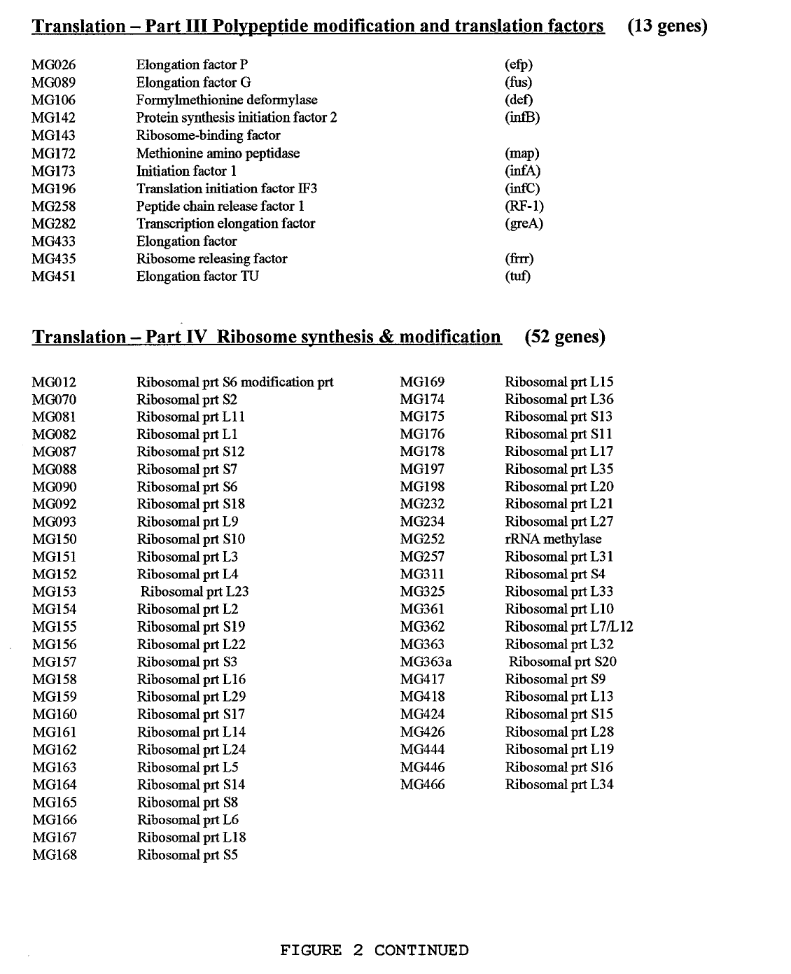

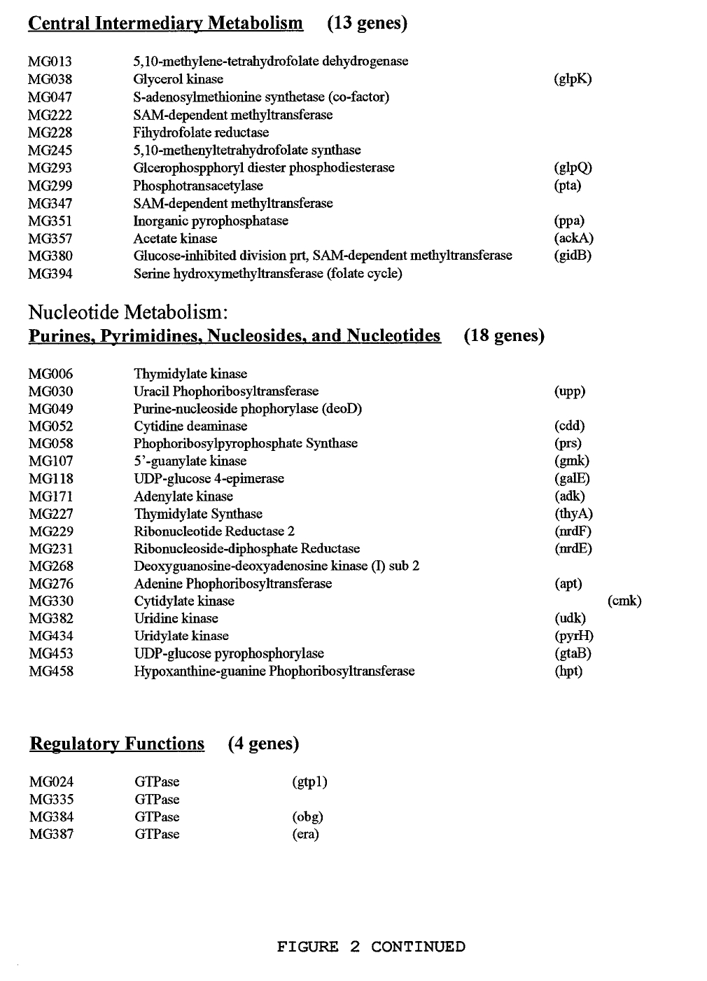

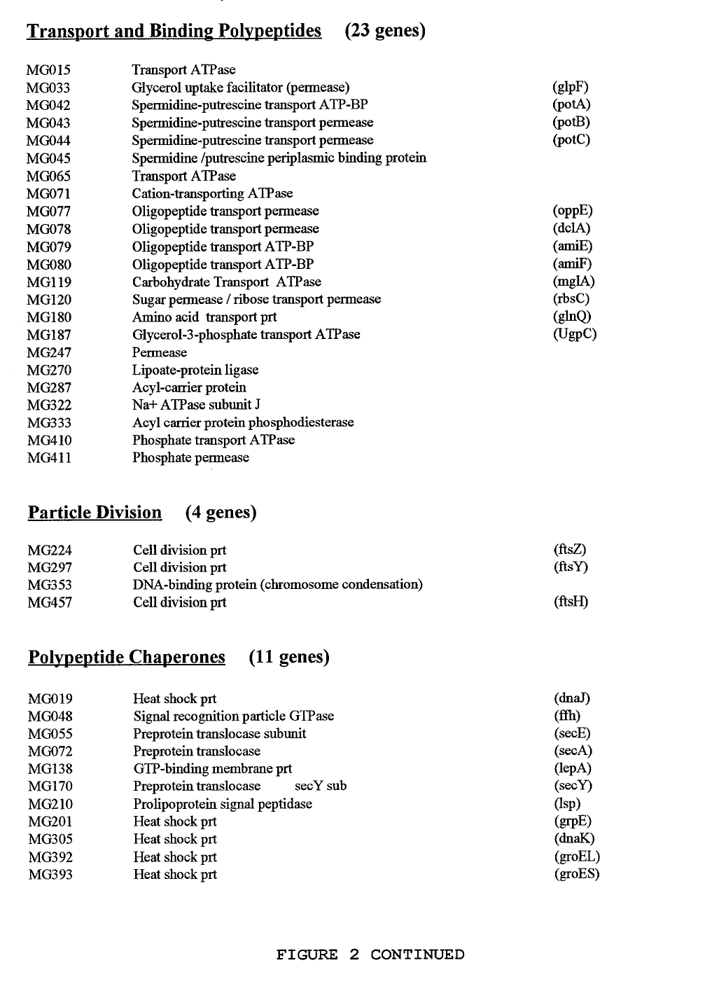

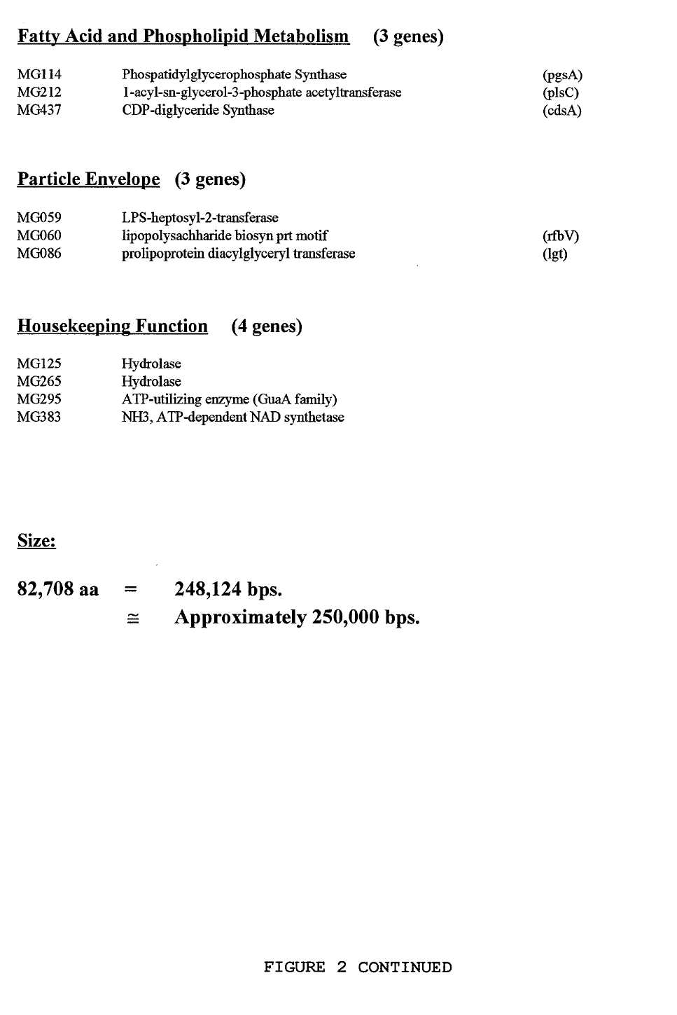

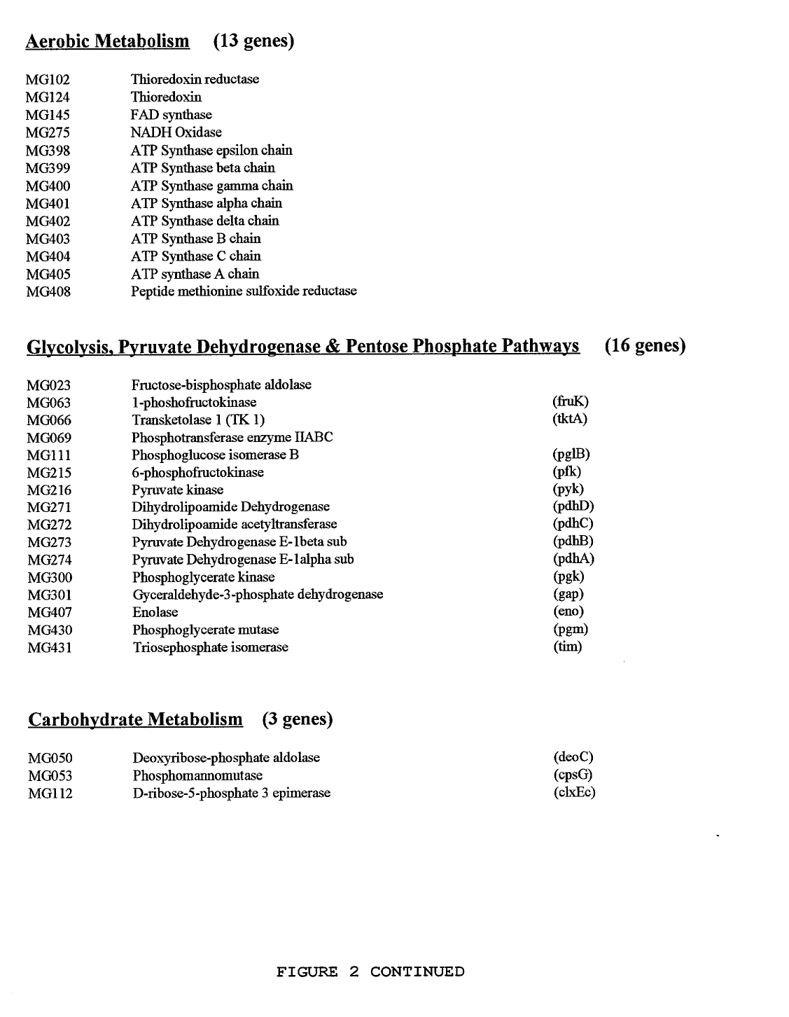

As used herein, the term “fundamental” when used in reference to a gene is intended to mean a gene that is important or essential to performance of a referenced activity. Therefore, a fundamental gene or set of genes are those genes that without which the congnate gene set or genetic operating system as a whole would inadequately perform a referenced nanomachine activity. A fundamental gene can include, for example, a gene that is indispensable to the performance of a nanomachine operation or activity encoded by the referenced gene set. A set of fundamental genes will include, for example, a substantially non-redundant threshold number of genes that are important or sufficient to perform a referenced nanomachine activity. Therefore, a set of fundamental genes will be composed of the least possible number of genes sufficient to perform a referenced operation or activity. Specific examples of fundamental gene sets for a viable nanomachine and for a replication competent nanomachine are show in FIGS. 1 and 2, respectively.

As with minimal gene sets, it is understood that fundamental genes of the nanomachine genomes and genetic operating systems of the invention are not restricted to genes derived from one species or even from a few different species. Instead, fundamental genes can be obtained from the same species, different related species, different divergent species or from various combinations thereof. Similarly, such species can include, for example, procaryotes such as Mycoplasma genitalium, Haemophilus influenzae and Escherichia coli, and eucaryotes such as yeast, nematodes, insects, other invertebrates, vertebrates, mammalian, including rodents, primates, and human.

It is also understood that fundamental genes within a minimal gene set derived from the same or different species can be modified to represent a different codon usage or preference. For example, the coding region for M. genitalium genes can be altered to encode E. coli type I, II or III codon preferences. Such modifications can be useful where the basic genetic operating system will function in, for example, an E. coli biosynthetic environment. Additionally, altering codon preferences also can be useful when, for example, fundamental genes originate from two or more different species. In such an example, orthologs or nonorthologous gene displacements from one species can be engineered to encode the same or substantially the same polypeptide from a heterologous codon preference. Therefore, all fundamental genes within a basic genetic operating system or genome can be normalized to a predetermined codon usage. Additionally, further modifications can be made in the codon usage to adjust for wobble and therefore frequency of amino acid incorporation. Other modifications to the encoding nucleic acid sequence well known to those skilled in the art which do not substantially affect the function of the gene or its gene product also can be introduced. It is also understood that various modifications described herein in reference to fundamental genes also are applicable to non-fundamental genes included in a nanomachine genome.

As used herein, the term “ortholog” is intended to mean a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

It is understood that the term is intended to include genes or their encoded gene products that through, for example, evolution have diverged in structure or overall activity. For example, where one species encodes a gene product exhibiting two functions and where such functions have been separated into distinct genes in a second species, the three genes and their corresponding products are considered to be orthologs. An example of orthologs exhibiting separable activities is where distinct activities have been separated into distinct gene products between 2 or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

It is also understood that orthologs can be created artificially by, for example, combining domains or portions of polypeptides from different species to create entirely new polypeptides with unique functions or combinations of functions. Such domains, either individually or when combined into unique polypeptides, can be considered orthologous to genes or gene domains related by vertical descent and responsible for substantially the same function in different organisms. Similarly, a unique combination of domains or portions also can be considered an ortholog to a second unique combination generated from different but orthologous domains. Functions of orthologs or orthologous domains include, for example, enzymatic, catalytic, signal transduction, structural and mechanical as well as other activities well known to those skilled in the art.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Other examples of paralogs include members of the hemoglobin (globin) family, members of the serine protease family, and immunoglobulin heavy chain gene products. Paralogs are proteins from the same species with significant sequence similarity to each other suggesting that they are homologous, or related through co-evolution from a common ancestor. Groups of paralogous protein families include HipA homologs, luciferase genes, peptidases, and others. Moreover, as with orthologs and orthologous domains, paralogs and paralogous domains similarly can be separated into distinct genes and gene products by, for example, evolutionary divergence or by genetic or recombinant manipulation.

As used herein, the term “nonorthologous gene displacement” is intended to mean a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene compared to a gene encoding the function sought to be substituted. Therefore, a nonorthologous gene includes, for example, a paralog or an unrelated gene.

The M. genitalium gene MG262 is one specific example of a nonorthologous gene displacement for the RNase H encoded function in H. influenzae and other species because it exhibits sequence identity to DNA polymerase 5′-3′ exonuclease and is distantly related to RNase H. Other specific examples of nonorthologous gene displacements include the M. genitalium genes MG264 and MG268 for the nucleoside diphosphate kinase (Ndk) encoded function in, for example, H. influenzae and E. coli. As with orthologs and paralogs, gene products of nonorthologous gene displacements are intended to be included within the meaning of the term as it is used herein.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal V and others compared and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarly to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

As used herein, the term “functional category” is intended to mean an operational classification of genes based on their purpose in cellular life. The term is therefore intended to group genes and their respective gene products according to functional contribution to a referenced biochemical process or activity. For example, genes that participate in replication processes will be classified as genes in the replication functional category. DNA polymerase is one specific example of a replication gene. Similarly, RNA polymerase is a specific example of a gene classified in the transcription functional category. An exemplary listing of functional categories and fundamental genes contained in each category is show in FIGS. 1 and 2 for basic genetic operating systems for a viable nanomachine and for a replication competent nanomachine, respectively. Although some genes can participate in more than one functional category, it is understood that a classification into a single category is a matter of convenience or simplicity for ease of description, and not a hierarchical distinction of importance in one category over another.

As used herein, the term “viable” or “viability” is intended to mean a that a host nanomachine is able to survive or exist in an environmental setting consistent with its engineered programming. Similarly, a basic genetic operating system containing a minimal gene set encoding gene products sufficient for viability also is intended to mean that the genetic programming encodes the requisite fundamental genes that enable a host nanomachine to survive or exist in an environmental setting compatible with the engineered genotype of the basic genetic operating system. Environmental settings can include, for example, natural, biochemical, physiological or industrial environments as well as in vivo, in situ or in vitro settings. Survival or existence can be, for example, passive, such as where biochemical process or selective reactions thereof are suspended until a favorable change in environmental conditions occurs. Survival or existence also can be, for example, active, such as where biochemical processes or selective reactions thereof continue to be at least partially active. Duration of survival can be from short, to long, to prolonged periods of time and include, for example, ranges of time from seconds and minutes to hours, days, weeks, months and years. The actual survival duration of a particular host nanomachine will depend, for example, on the engineered programming of the basic genetic operating system and the targeted host nanomachine application.

As used herein, the term “replication” or “replication competent” is intended to mean that a host nanomachine is able to create at least one duplicate copy of its genome in an environmental setting consistent with its engineered programming. Similarly, a basic genetic operating system containing a minimal gene set encoding gene products sufficient for replication also is intended to mean that the genetic programming encodes the requisite fundamental genes that enable a host nanomachine to duplicate at least one copy of its genome in an environmental setting compatible with the engineered genotype of the basic genetic operating system. Therefore, the term replication refers to biosynthesis of a host nanomachine's basic genetic operating system and, for example, other genes encoded in its genome. Genome replication can include, for example, regulated, conditional or constitutive modes of genome biosynthesis. In contrast, proliferation, reproduction or particle division can refer to duplication of a nanomachine particle envelope to produce two or more progeny nanomachines. In the absence of particle division, a replication competent nanomachine can accumulate, for example, 2, 3, 4, 5, 10, 20 or 50 or more nanomachine genome copies within a particle envelope. Inclusion of particle division fundamental genes within a replication competent basic genetic operating system can allow, for example, concomitant segregation of single or multiple copies of a nanomachine genome into progeny nanomachine particles.

As used herein, the term “devoid” when used in reference to a gene is intended to mean lacking or deficient for a functional gene. Functional gene as it is referred to herein means that it encodes for a active gene product, including for example, both nucleic acid and polypeptide gene products. A functional gene can be lacking or deficient by, for example, deletion or mutation of its coding region, one or more regulatory regions, or processing signals. Similarly, combinations of alterations in coding regions, regulatory regions or processing signals also can render a gene set, basic genetic operating system or nanomachine genome devoid of a gene. Therefore, alterations in a gene that render it deficient for a functional gene product can be small, such as by a single point mutation, or large, such as by large deletions, including all or substantially all of the encoding or regulatory region of the nucleic acid.

As used herein, the term “particle envelope” is intended to mean a partition that separates or compartmentalizes nanomachine components from non-nanomachine components. The term additionally includes other physical or chemical means which can control compartmentalization into a microenvironment. Such physical and chemical means include for example, electrostatic forces, hydrophobicity and micro encapsulation without complete partitioning. Nanomachine components include for example, a nanomachine genome, including a basic genetic operating system, encoded nucleic acid and polypeptide gene products and products produced therefrom. Products produced from encoded gene products include, for example, the multitude of metabolitic and catabolitic substrates, intermediates and products that can be synthesized by cellular biochemical pathways. Such molecules include, for example, amino acids, nucleotides, nucleosides, purine and pyrimidine bases, fatty acids, lipids, carbohydrates, cofactors and other organic molecules. An exemplary description of cellular biochemical pathways, including substrates, intermediates and products, that are synthesized by nucleic acid encoded gene products can be found, for example, in Lehninger Principles of Biochemistry, Nelson and Cox, Third Edition, 2000, Worth Publishers, New York and Biochemistry, Stryer, Fourth Edition, 1995, W.H. Freeman and Company, New York, both of which are incorporated herein by reference. In contrast, non-nanomachine components include, for example, environmental components. A particle envelope can be composed of various biochemical molecules and physiologically-compatible molecules known to those skilled in the art.

For example, a particle envelop can be composed of substantially the same molecules as naturally occurring lipid membranes. Alternatively, a particle envelope can be completely or partially synthetic so long as it maintains its ability to partition nanomachine from non-nanomachine components. Particle envelopes also can be formed by, for example, surface tension, where nanomachine components are held together in a droplet formed by surface tension or where aqueous media partitions separately in an organic solution. Separation to achieve a particle envelope also can be spatially, such as between organic and nonorganic solutions or between an aqueous solution and air. Similarly, micro-porous structures also can be used to form a particle envelope. Specific examples can include porous resin and a micromachined matrix. Additionally, all of the various types of particle envelopes described above, as well as other types well known to those skilled in the art, also can be modified with charged moieties to either enable or supplement separation of nanomachine components from non-nanomachine components by electrostatic forces. Similarly, pressure and vacuum forces also can be used to create or enhance the function of a particle envelope.

The invention is directed to biological nanomachines programmed by and synthesized from nucleic acid-based information. The use of nucleic acid-based information enables the accurate assembly of matter at the atomic and molecular level into precise functional structures and operational particle assemblages. Nucleic acid-based information allows bottom-up assembly of nanoscale machines and structures because the rules and processes for matter manipulation are inherently contained in the encoding nucleic acid and conferred on the gene products as well. Therefore, Nucleic acid-based nanomachines programmed with genetic operating systems circumvent top-down miniaturization approaches and requirements for multi-disciplinary nanotechnology. Instead, nanomachines programmed by Nucleic acid-based information harness biochemical rules and processes to generate constituent nanomachine components that self-assemble into functional biological and biologically compatible structures which can perform useful work and carry out a wide range of physiological and biochemical activities.

The invention provides a basic genetic operating system for an autonomous prototrophic nanomachine. The basic genetic operating system consists of a nanomachine genome encoding a minimal gene set sufficient for viability. Functional categories of genes within a minimal gene set can be transcription, translation, aerobic metabolism, glycolysis/pyruvate dehydrogenase/pentose phosphate pathways, carbohydrate metabolism, central intermediary metabolism, nucleotide metabolism, transport and binding proteins, and housekeeping functions.

A basic genetic operating system of the invention is a nucleic acid, or a functional equivalent of a nucleic acid, that can serve as a genome for a biosynthetic cell or nanomachine. Functional equivalents of a nucleic acid include, for example, a nucleic acid that contains one or more natural or non-naturally occurring nucleotides, which contain modified bases or bases other than adenosine (A), guanine (G), cytosine (C) or thymine (T) or uracil (U) and which is a substrate for template-directed nucleic acid polymerization. Modifications include, for example, derivatization and covalent attachment with chemical groups. Other bases can include, for example, pyrimidine or purine analogs, precursors such as inosine that are capable of base pair formation, and tautomers. Similarly, a nucleic acid functional equivalent also can contain modified or derivative forms of the ribose or deoxyribose sugar moieties, including, for example, functional analogs thereof. Those skilled in the art will know what natural or non-naturally occurring nucleotide, nucleoside or base forms can be used in a basic genetic operating system of the invention, including derivatives and analogs thereof, and also capable of supporting template-directed nucleic acid polymerization.

A basic genetic operating system encodes, for example, the required gene products that are obligatory to sustain rudimentary or foundational functions of cellular life. A basic genetic operating system differs from a complete genome, for example, because it duplicates or more closely approximates a genetic copy of genes, or functional fragments thereof, that are essential for basic cellular life functions. Therefore, a basic genetic operating system is a streamlined genome that contains all necessary genetic information required to sustain viability or other cellular life functions. As a streamlined version of a genome, a basic genetic operating system also is a simpler and more efficient genome because it lacks unwanted or unnecessary genetic information or nucleic acid structure.

As a streamlined copy of genes that are obligatory to sustain rudimentary or foundational functions of cellular life, a basic genetic operating system constitutes a minimal compilation of genes that are required for the biosynthesis and maintenance of cellular life functions. Cellular life functions include, for example, viability, replication, transcription, translation, cell division, energy generation, cellular homeostasis, adhesion, motility migration, environmental adaption, chemotaxis and immune and effector cell responses. Therefore, a basic genetic operating system can, by itself, substitute for, or function as, a cellular or nanomachine genome. However, and as described further below, a basic genetic operating system also can be combined with other genes and gene sets to augment the genetic instructions of the basic operating system. Inclusion of other genes and gene sets can, for example, additionally enable a host nanomachine to perform and maintain a wide variety of biochemical activities and operations in conjunction with those constituting fundamental cellular life functions.

One fundamental cellular life function is viability. A minimal gene set sufficient for viability includes, for example, genes that fall within a number of functional categories. Genes within each functional category can be grouped, for example, based on functional independence relative to another category as well as based on simplicity of description. However, those skilled in the art will understand that functional categories described herein also can be interrelated or interdependent for performance or maintenance of a nanomachine cellular life function. For example, genes within a minimal gene set corresponding to the functional category of transcription can be independent with respect to genes within the functional category of an aerobic metabolism because a nanomachine can produce a nucleic acid gene product using energy sources derived from aerobic pathways. For example, glycolysis, pyruvate dehydrogenase and the pentose phosphate pathways are pathways within an aerobic functional group that can generate, for example, ATP as an energy source in the absence of an aerobic respiration. Similarly, transcription can be independent with respect to aerobic metabolism when fundamental genes for anaerobic pathways are present to produce energy sources. Interrelated functional groups can include, for example, transcription and translation. Although both of these functional categories can operate independently, both also require the gene products of the other category to persistently maintain function and homeostasis. The constituent genes and gene products and their interrelationships or independence with respect to other functional categories and cellular life functions is described further below.

Functional categories of genes within a minimal gene set constituting the genetic programming sufficient to support viability as a cellular life function include, for example, about nine or less fundamental biochemical processes. Although interrelated, these process fall under the general groupings of biosynthetic, metabolic and homoeostatic processes. The biosynthetic groupings include, for example, the functional categories of transcription and translation.

The metabolic processes include, for example, energy metabolism, carbohydrate metabolism, central intermediary metabolism and nucleotide metabolism. Energy metabolism can further include the functional categories of aerobic metabolism and anaerobic metabolism. Glycolysis, pyruvate dehydrogenase and the pentose phosphate pathways are specific biochemical pathways supplying high free energy molecules such as ATP, NADH and NADPH under aerobic conditions. Some of these pathways, such as glycolysis, for example, also synthesize high free energy molecules under anaerobic conditions. The reductive citric acid cycle is a specific biochemical pathway supplying high free energy molecules under anaerobic conditions.

Function categories within the homoeostatic processes include, for example, transport and binding proteins, and housekeeping functions.

Those skilled in the art will know what fundamental genes are, or can be, contained within each category, including for example, those derived from procaryotic and eucaryotic sources. Exemplary listings of functional categories and constituent minimal gene set sufficient for a basic genetic operating system to direct autonomous nanomachine viability is shown in FIG. 1 and Table 4. Therefore, the functional categories constituting a minimal gene set sufficient for a cellular life function such as viability can be derived from a single species or multiple species. Similarly, fundamental genes determine to fall within a functional category also will include, for example, functional equivalents such as orthologs and nonorthologous displacements as well as functional fragments thereof.

Various combinations and permutations of functional categories, for example, such as those shown in FIG. 1 and Table 4 for a basic genetic operating system programmed to direct autonomous nanomachine viability as a cellular life function can be produced depending on the need and desired operation of the host nanomachine. For example, a nanomachine can be programmed to function under completely anaerobic conditions. In this specific example, the functional category specifying genes required for aerobic metabolism, which do not substantially overlap with fundamental genes for anaerobic metabolism, can be omitted from the basic genetic operating system. Alternatively, the functional category specifying non-overlapping genes required for anaerobic metabolism can be omitted for a nanomachine programmed to function under aerobic conditions. Similarly, a nanomachine can be programmed to generate macromolecules, such as nucleotides, by de novo biosynthesis. For the specific example of de novo nucleotide biosynthesis, the salvage pathway genes shown in FIG. 1, for example, can be substituted for a partial or complete set of genes specifying de novo nucleotide biosynthesis. Further, for example, if a nanomachine of the invention is desired to chemotax to perform a targeted application, then this functional category and its constituent fundamental genes can be included within a basic genetic operating system of the invention.

Numerous other combinations, substitutions and permutations of functional categories can be made in a basic genetic operating system of the invention to tailor the performance of an autonomous nanomachine to a particular application. Such other modifications of functional categories include, for example, anaerobic metabolic pathways, fermentation, stress related genes such as heat shock, DNA repair, RNA processing, secretion, glycosylation, glycoside synthesis and isoprenoid synthesis. Those skilled in the art will know which functional categories can be combined, modified or substituted to accomplish a predetermined activity, cellular life function or application. Additionally, as with the other functional categories, the genes within a particular biosynthetic pathway are well know to those skilled in the art. Similarly, using the teachings and guidance provided herein, those skilled in the art will know, or can determine, which genes within a biochemical pathway or physiological process are fundamental genes and included with a minimal gene set and which genes are dispensable to the efficient function and operation of a genetically programmed cellular life function.

A minimal gene set will include, for example, genes within a functional category that are fundamental to a biochemical process. Fundamental genes include those genes that are essential to the process, without which the activity cannot occur. Fundamental genes also include, for example, those elementary genes that augment the performance of a biochemical process to levels comparable to a cellular life form or comparable to a reference standard that is required for a targeted application. For example, fundamental genes required for protein synthesis can include all essential and elementary genes that are necessary for nanomachine protein synthesis to occur at a rate comparable to a procaryotic or eucaryotic cell system. Alternatively, if a targeted application can be accomplished by nanomachine protein synthesis rates less than comparable cellular levels, then the required fundamental genes can exclude some or all of the elementary genes and still be considered a minimal gene set, and therefore, a basic genetic operating system of the invention.

Those skilled in the art will know, or can determine, the performance of a biochemical process which constitutes activity levels comparable to similar processes of a cellular life form or comparable to a reference standard that is required for a targeted application. A specific example of a comparable cellular activity level includes protein synthesis rate under specified environmental, physiological or culture conditions. A specific example of a comparable reference standard includes accumulated protein synthesis of a specified gene product under specified environmental, physiological or culture conditions sufficient to achieve a predetermined target end point. Such end point standards can include, for example, accumulation of a predetermined amount of gene product or achievement of a specified activity, such as binding inhibition or regulation of a target molecule. Essentially any nanomachine activity, process, cellular life function, operation or attribute encoded by a minimal gene set will have a corresponding cellular life or reference comparison. Using the teaching and guidance provided herein, those skilled in the art will know, or can routinely determine, such cognate comparisons between nanomachines programmed by a basic genetic operating system of the invention and either procaryotic or eucaryotic cellular life forms.

Similarly, those skilled in the art will know, or can determine, fundamental genes that encode either an essential function or an elementary function within a minimal gene set. For example, an essential gene is indispensable to a cellular life function of a nanomachine and is therefore required to be encoded by a basic genetic operating system programmed for the reference life function. Specific examples of essential genes include those coding for RNA polymerase subunits. Related to essential genes are those that perform elementary or basal functions which can augment an activity of an essential gene or its gene product. As such, an elementary gene is dispensable but only at a substantial cost to basic nanomachine operation. A specific example of a fundamental gene encoding an elementary function includes genes coding for transcription factors such as transcription terminators. Removal of a transcription terminator from a basic genetic operating system does not substantially affect viability of a host nanomachine, although inclusion would augment at least resource utilization.

Those skilled in the art will understand that augmentation of a elementary process differs from optimization. The former referring to supplementation of a fundamental process encoded by a basic genetic operating system, whereas the latter refers to a substantial enhancement of fundamental processes or of overlying activities and functions additional to minimal gene set activities. Substantial enhancements can include, for example, the inclusion of multiple polypeptide species or isotypes, such as those related within a family, that each perform specialized, but related, subfunctions within a broader activity spectrum. Generally, substantial enhancements of a fundamental process can be categorized as gene or functional redundancy of a component molecule or functional category encoded by a basic genetic operating system.

A nanomachine of the invention is autonomous when, for example, it is capable of independently carrying out its cellular life function established by the nucleic acid programming contained within its basic genetic operating system. Similarly, a nanomachine activity or operation also can be considered as autonomous when, for example, the activity or operation can be performed independently due to instructions established by the nanomachine's basic genetic operating system. For example, a nanomachine of the invention is autonomous when it can execute its programmed function as engineered. Therefore, autonomy refers to the ability of a nanomachine to synthesize, perform, and maintain, for example, all molecules, activities, and processes that are engineered through nucleic acid coding and regulatory sequences into a basic genetic operating system of the host nanomachine.

For example, if a basic genetic operating system is designed to be a complete set of genetic instructions for glycolysis, then an autonomous nanomachine can metabolize glucose to its end products. In contrast, for example, a nanomachine can still be considered to be autonomous where its basic genetic operating system has a designed defect in the glycolysis gene set and where a glycolytic intermediate downstream from the designed defect can be exogenously supplied. Addition of the downstream intermediate allows the nanomachine to continue self-production of its encoded activities and operations despite having an incomplete gene set. Therefore, dependence on external or exogenous sources of required molecules that could be encoded into a basic genetic operating system of the invention does not preclude autonomy of a nanomachine so long as the basic genetic operating system has been engineered for such a predetermined dependence.

Similarly, a nanomachine of the invention is considered to be prototrophic when, for example, its basic genetic operating system contains a complete minimal gene set for an engineered cellular life function, activity or operation. A complete minimal gene set or functional category of fundamental genes includes, for example, those genes which are adequate for a host nanomachine to execute and maintain the engineered cellular life function, activity or operation in a self-sufficient manner. Therefore, a basic genetic operating system engineered for prototrophic functions and activities will be autonomous for the referenced function without requirements for exogenous supplementation of a deficient gene product in the minimal gene set or referenced functional category.

In comparison, a nanomachine of the invention is considered to be auxotrophic when, for example, its basic genetic operating system contains a designed gene deficiency in an otherwise complete minimal gene set. For example, an auxotrophic basic genetic operating system contains an incomplete minimal gene set for an engineered cellular life function, activity or operation. To be auxotrophic, however, an incomplete minimal gene set or functional category of fundamental genes will, for example, be able to be execute and maintain its engineered function with exogenous supplementation of a gene product of the designed gene deficiency. Similarly, an auxotrophic basic genetic operating system also can execute and maintain its engineered function with exogenous supplementation of a component downstream or functionally equivalent to the designed defect. Therefore, autonomy of auxotrophic systems of the invention are rescuable by design through the addition of an auxotrophic biomolecule. As such, a basic genetic operating system engineered for auxotrophic functions and activities will be autonomous for the referenced function with the exogenous supplementation of an engineered deficient gene product or a component that can rescue the designed deficiency.

The functional categories constituting a basic genetic operating system of the invention can be arranged in essentially any desired physical or functional order so long as all genes of the minimal gene set are present and operative. However, arranging the functional categories in relative order of importance can augment the efficiency of the host nanomachine operation. Similarly, arranging the functional categories in relative order of importance also can increase the quality of a particular nanomachine product or activity. Depending on the desired use of a nanomachine of the invention, the functional gene categories can be selectively arranged to optimize, for example, the genetic programming of the basic genetic operating system, nanomachine operation efficiency or genome size.

One arrangement of functional categories within a basic genetic operating system conferring viability on a host nanomachine can be, for example, in the relative order of gene product use to achieve a programmed cellular life function. To sustain cellular life, a nanomachine should be able to biosynthesize component macromolecules. As such, one relative order of use can follow, for example, the normal information to product flow of a cell, which would be from transcription of the genome to translation of the mRNA into polypeptide products. This order has the advantage in that genes encoding precursors and intermediates to the working nanomachine products are produced first, thereby preventing rate limiting steps in the production and activity of central nanomachine components. Therefore, a relative order of functional categories for efficient nanomachine operation can be genes constituting transcription and translation categories, respectively, followed by functional categories specifying nanomachine energy sources. Such energy sources can be fundamental gene sets sufficient for either or both aerobic metabolism and anaerobic metabolism. Additionally, pathways specifying energy sources also can be ordered relative to their use in cellular metabolism. For example, fundamental genes encoding the glycolysis pathway can be placed in a relative order within a basic genetic operating system earlier than genes specifying the pyruvate or pentose phosphate pathways, or earlier than non-fundamental genes such as those specifying the citric acid (TCA) cycle or the reductive citric acid cycle.

The remainder of the functional categories of genes sufficient to support viability, for example, of a host nanomachine can be in essentially any desired order depending on the targeted application of nanomachine and desired efficiency. One exemplary order of the remaining categories can be, for example, carbohydrate metabolism, central intermediary metabolism, nucleotide metabolism, transport and binding proteins, and housekeeping functions, respectively. The number of permutations and combinations of functional category order are many. Those skilled in the art will know what order and combination of functional categories can be made within a basic genetic operating system to achieve a desired result.

Ordering of functional categories can be based on several different criteria. For example, ordering can be accomplished with reference to physical order or temporal order. Any particular physical order can be accomplished by the architectural design and placement of a minimal gene set within a basic genetic operating system. Additionally, physical order can be with reference to any of a number of genomic markers. Such markers include, for example, an origin of replication, a particular gene or a particular gene set. Specific examples of ordering functional categories within a basic genetic operating system relative to a gene or gene set includes placing the first ordered functional category next to an expression cassette for the production of a biomolecule, or next to an indispensable gene set such as that for aerobic metabolism. Similarly, functional category ordering can be, for example, unidirectional, bidirectional, with respect to a single strand of the genome, with respect to both stands of the genome and all combinations thereof. Utilizing both strands of the genome has the advantage of efficient use of genome space.

Any particular temporal order can be accomplished, for example, by activation and repression of targeted genes and gene sets in a selected order. Selective activation and repression can be achieved, for example, by cis and trans acting factors or by conditional regulation of transcription or translation. Therefore, any desired temporal order of expression of functional categories or of their constituent fundamental genes can be achieved by selective activation of their respective promoters. Selective activation can be achieved by, for example, positive regulation or derepression of an inhibitor. The cis and transacting factors used for such selective activation can be, for example, either homologous or heterlogous elements or factors compared to the gene it regulates. Additionally, temporal order of expression also can be accomplished by a combination of selected activation and repression of genes and gene sets and physical order of particular target genes or their trans acting regulators. Other methods, well known to those skilled in the art for controlling the relative order of expression of functional categories or constituent fundamental genes include, for example, RNA processing, post-translational modifications such as phosphorylation, glycosylation, proteolytic cleavage, signal transduction cascades and clotting cascades.

Therefore, the invention also provides a basic genetic operating system for an autonomous prototrophic nanomachine that encodes a minimal gene set sufficient for viability which directs synthesis of functional categories in a relative order consisting of transcription, translation, aerobic metabolism and glycolysis/pyruvate dehydrogenase/pentose phosphate pathways. The relative order can be, for example, with reference to physical or temporal arrangement of functional categories.

Also provided is a basic genetic operating having a minimal gene set that is devoid of at least one gene selected from the group consisting of MG008, MG009, MG056, MG221, MG332, MG448 or MG449, an ortholog or a nonorthologous gene displacement thereof.

Although conserved genes between, for example, M. genitalium and H. influenza, the above genes are redundant in structure or function compared to other genes found within these and other species genome. For example, MG008 encodes furan and thioprene oxidase. MG262 encodes an exonuclease. MG009, MG056, MG221, and MG332 encode polypeptides with nucleotide binding domains such as ATP-, GTP-, NAD, FAD and SAM-binding domains, a permease or other conserved domains. MG448 and MG449 encode polypeptides with chaperone binding domains. Additionally, some of these genes are unnecessary for rudimentary functions and therefore more appropriate to be placed in an overlying genetic program operated from a basic genetic operating system of the invention. For example, those genes encoding chaperone and permease functions are not necessarily required for autonomous nanomachine operation.

The invention further provides a basic genetic operating system for a nanomachine genome that is sufficient for viability having less than about 140 kilobases (kb) in size. The basic genetic operating system can be about 152 or less fundamental genes, functional fragments, orthologs or nonorthologous displacements thereof.

A basic genetic operating system containing a minimal gene set sufficient for viability can be constructed to be any size so long as it can be packaged into a particle envelope or other partitioning structure. One advantage of engineering a basic genetic operating system is that it is a bottom-up approach to construction of the nanomachine genome. Similar to bottom-up nanomachine construction through biological self-assembly of matter at the atomic and molecular level, designing a minimal gene set specifying predetermined functions allows, for example, precise structures to be designed and synthesized. For example, genes can be arranged to conserve space by juxtaposition of fundamental genes with minimal inclusion of intervening genomic sequence. Regulatory regions such as enhancers can be moved from intergenic regions to introns, for example. Similarly, non-useful nucleic acid segments can be, for example, truncated or otherwise omitted, structural gene sequences such as introns, 5′ and 3′ gene flanking regions and untranslated sequences can be reduced or eliminated, genes can be overlapped or incorporated into genes transcribed and translated as polycistronic mRNA, and the primary sequence can be modified to incorporate optimal nucleotide usage to increase efficiency in translation of transcribed mRNA. Additionally, fundamental genes constituting a minimal gene set can be, for example, tailored to include only relevant functional domains. Therefore, a minimal gene set can consist of functional fragments of some or all of the fundamental genes that constitute one or more functional categories.

Those skilled in the art will know, or can readily design, given the teachings and guidance provided herein, a wide range of sizes for a basic genetic operating system sufficient to support a cellular life function such as viability. For example, a minimal gene set such as that shown in FIG. 1 or corresponding orthologous genes set forth in Table 4 which are sufficient to specify nanomachine viability, can be organized into a basic genetic operating system of about 140 kilobase (kb) pairs or less. For example, juxtaposition of intronless versions of these genes can result in a nucleic acid of about 137,589 base pairs (bp). Such a minimal gene set encodes about 152 fundamental genes for a total of about 45,863 amino acids. Inclusion of naturally occurring expression and regulatory elements, heterologous elements or combinations thereof, in a juxtapositional arrangement can be accomplished with minimal increase in nucleic acid size as these elements contribute minimally to overall size of the basic genetic operating system compared to the fundamental genes of the minimal gene set.

The size of a basic genetic operating system additionally can be reduced by, for example, employing any or various combinations of the architectural designs described above. For example, coding regions, noncoding regions, expression and regulatory sequences can be partially or substantially overlapped between some or all of the genes constituting a minimal gene set specifying a cellular life function or genes within one or more functional categories. Additionally, the constituent fundamental genes can be arranged on both strands of a double stranded nucleic acid to further condense a basic genetic operating system of the invention. Therefore, a basic genetic operating system of the invention programming non-replicative cellular life functions of a nanomachine can be substantially smaller than about 140 kb. For example, a basic genetic operating system sufficient for viability can be about 130 kb or less, 120 kb or less 110 kb or less and even 100 kb or less. It is also possible to reduce in half the size of such basic genetic operating systems to about 70 kb by, for example, substantial overlap and truncation of fundamental genes that constituting a minimal gene set. Other architectural designs well known to those skilled in the art similarly can be used to condense or optimize the structure of a basic genetic operating system of the invention.

A basic genetic operating systems of the invention also can include, for example, various structural features that facilitate the transfer of information into encoded polypeptides and the operation of cellular life functions of a nanomachine. Such structural features can include, for example, nuclear or cell membrane binding sites, binding regions for chromosome scaffolding, histone binding regions for chromosome condensation and, for example, non-coding intergenic nucleic acid. The presence of such intergenic spacer segments can allow, for example, efficient entry and exit of nucleic acid binding factors by reducing steric hindrance, binding site competition and topological constraints, for example. Additionally, the basic genetic operating systems of the invention can be designed as double stranded or single stranded genomic structures. Those skilled in the art will know which of various structural regions can be incorporated into a basic genetic operating system to achieve a targeted application as well as to increase or optimize its performance as a nanomachine genome. For example, if the nanomachine is to parallel procaryotic cellular life forms, then chromosome condensation is not necessarily important. However, chromosome condensation, anchorage and scaffolding can be advantageously utilized in basic genetic operating system that specifies fundamental genetic programming for higher eucaryotic cellular life forms.

As described above, a basic genetic operating system specifying basal cellular life functions such as viability can be accomplished, for example, with about 152 fundamental genes or less. They can be grouped, for example, in about 9 functional categories. The number of constituent genes within each functional category can vary, for example, depending on the targeted application of the host nanomachine. For example, the number of constituent genes can vary depending on whether the programming is for de novo or salvage pathway biosynthesis of a molecule or class of molecules. The number of constituent fundamental genes also can vary, for example, depending on whether the programming specifies viability within an intracellular or extracellular physiological environment or an extracellular non-physiological environment. Constituent fundamental genes also can vary depending on whether the programming specifies aerobic or anaerobic gene products for production of energy sources. Inclusion of membrane sorting, polypeptide secretion and intracellular trafficking and vesicle gene functions also can vary the number of constituent fundamental genes within a functional category. Similarly, and as described further below, the number of constituent genes within each functional category can vary, for example, depending on whether the basic genetic operating system specifies prototrophic or auxotrophic nanomachine autonomy. As set forth in Table 4 the number of constituent gene products also can vary depending on whether the basic genetic operating system is engineered from procaryotic or eucaryotic genes, orthologs or nonorthologous displacements thereof.

Generally, however, constituent genes sufficient to support viability can be grouped, for example, into about 14 genes in a transcription gene category, about 90 genes in a translation gene category, about 13 genes in an aerobic metabolism gene category, about 16 genes in a gene category constituting glycolysis, pyruvate dehydrogenase, and pentose phosphate pathways, about 3 genes in a carbohydrate metabolism gene category, about 3 genes in a central intermediary metabolism gene category, about 2 genes in a nucleotide metabolism gene category, about 10 genes in a transport/binding protein gene category and about 1 genes in a housekeeping function gene category. The category containing genes functioning in translation processes also can be further divided, for example, into two further subgroups. These translation subgroups can consist of about 13 genes whose gene products function in polypeptide modification and translation factors and about 52 genes whose gene products function in ribosome biosynthesis, assembly and modification. Similarly, there are about 10 fundamental genes encoding glycolytic functions, about 2 fundamental genes encoding pyruvate dehydrogenase pathway gene products and about 4 fundamental genes encoding gene products that function in the pentose phosphate pathways.

Exemplary fundamental genes and their gene product functions within each of the above functional categories and subgroups are shown in FIG. 1. Orthologous genes which can similarly substitute for those shown in FIG. 1 are set forth in Table 4 below. Given the teachings and guidance provided herein those skilled in the art will know or can determine, by for example, comparative genomics and gene product function, other orthologs or nonorthologous displacements that similarly can substitute for one or more of the fundamental genes shown in FIG. 1 or Table 4. Therefore, the invention provides a basic genetic operating system sufficient to direct autonomous prototrophic viability of a host nanomachine having about 152 or less fundamental genes that consists of substantially the same fundamental genes show in FIG. 1, Table 4, including orthologs or nonothorologous displacements thereof.

Although the invention has been described with reference to basic genetic operating system encoding a minimal gene set sufficient for viability, those skilled in the art will know that various other basic genetic operating system programming other cellular life functions can be engineered and synthesized given the teachings and guidance provided herein. For example, described further below are basic genetic operating systems encoding replication functional categories so as to confer replication competence as a cellular life function of a host nanomachine. Additionally, a basic genetic operating system can be engineered for autonomous nanomachine operation in an intracellular environment, such as is the case for M. genitalium, or an extracellular environment such as is the case from H. influenza, E. coli, other procaryotic cells and eucaryotic cells. Further non-replicative basic genetic operating systems can additionally include, or programming changed to encode, other cellular life functions such as polypeptide synthesis, membrane integrity, polypeptide folding, polypeptide trafficking, extracellular synthesis and transport, motility, fermentation and spore formation.

For example, protein synthesis machinery can be encoded in the absence of transcription functions for specific mRNA species. A host nanomachine can be supplied with exogenous mRNA for synthesis of one or more encoded polypeptides. Also a basic genetic operating system can include membrane structural genes, integral membrane or transmembrane polypeptides that augment the structural integrity of a lipid membrane particle envelope. In like fashion, polypeptide folding functions and trafficking functions can be encoded. For example, sec-dependent polypeptide secretion in procaryotes and signal recognition particle (SRP)-dependent tranaslocation in eucaryotes are two specific examples of folding and trafficking functions. Specific examples of extracellular synthesis and transport can be useful for nanomachine survival in certain environments and include, for example, translocation of molecules using ABC transporters, synthesis of glycogen, synthesis and secretion of glycopolymers such as dextrans and xanthan gum.

Additionally, selected pathways for aerobic energy production or anaerobic energy functions such as genes encoding the reductive citric acid cycle can be programmed. Briefly, the carbohydrate pathways for aerobic energy production can include, for example, glycolysis, the pentose phosphate pathway and the Entner-Doudoroff pathway. Glycolysis, or the EMP pathway is present in both procaryotic and eucaryotic organisms and functions to oxidize carbohydrate to pyruvate and to phosporylate ADP. This pathway also provides precursor metabolites for other pathways, including feeding into the pentose phosphate pathway via glucose-6-phosphate. The pentose phosphate pathway is similarly present in both procaryotic and eucaryotic organisms and produces NADPH, pentose phosphates, which are precursors to ribose and deoxyribose, and erythrose phosphate, which is a precursor to aromatic amino acids, phenylalanine, tyrosine and tryptophan, and phoshoglyceraldehyde. The Enter-Doudoroff pathway is found generally in procaryotic organisms and produces various energy molecules in the presence of specific carbon sources, such as gluconic acid.

Other aerobic energy functions include, for example, the pyruvate dehydrogenase complex and the Citric Acid Cycle. Pyruvate dehydrogenase complex is an enzyme located in the cytosol of procaryotes and in the mitochondria of eucaryotes. This complex functions to decarboxylate pyruvate to acetyl-CoA, CO₂ and NADH. Acetyl-CoA can enter the citric acid cycle, where it is oxidized to CO₂. The Citric Acid Cycle operates in conjunction with repiration to oxidize NADPH and FADH₂ and generally functions during aerobic growth. Under anaerobic conditions, procaryotes have a modified pathway called the reductive citric acid pathway where NADH is oxidized by an organic acceptor that is generated during catabolism.

Anaerobic energy production includes, for example, including or substituting for pyruvate dehydrogenase, fundamental genes encoding pyruvate-ferredoxin oxidoreductase or pyruvate-formate lyase, which function to breakdown pyruvate into acetyl-CoA under anaerobic conditions. Utilization of the reductive citric acid pathway will allow fermentation for example. Although not present in M. genitalium, these functions can be obtained from genes in other organisms such as E. coli. Briefly, to obtain anaerobic respiration, α-ketoglutarate dehydrogenase activity can be down regulated or the gene rendered non-functional, and fumarate reductase can replace, or be additionally included with, succinate dehydrogenase.

Further, fermentation cycles such as butyrate or butanol-acetone fermentation from C. acetobutyliciuum also can be programmed. Basic motility functions can be changed by encoding different flagella motors to be compatible, for example, with the host nanomachine environment. Such different flagella also can include a lipopollysaccharide sheath or be a spirochete flagella, for example. Spore forming functions can be included from organisms such as B. subtilis and can include genes such as SpoOA, SpoOF, KinABC and others. Other basic cellular life functions also are well known to those skilled in the art and can be included in a basic genetic operating system of the invention.

Any basic genetic operating system of the invention can be supplemented with additional genetic programming to, for example, supplement fundamental nanomachine activities or operation, or, for example, to customize a host nanomachine to perform essentially any desired function. Supplementation with additional genetic programming can include, for example, basic genetic operating systems containing fundamental programs specifying, for example, prototrophic autonomous functioning, auxotrophic autonomous functioning, non-replicative cellular life functions and replication competent cellular life functions. Such additional genetic programming can be conceptually analogized to computer application programs overlaid on, or run off of a computer operating system, where the latter can be conceptually analogized to a basic genetic operating system of the invention. By analogy, a basic genetic operating system of the invention can be engineered to contain controlling functions, nucleic acid sequences and nucleic acid structures for entry and execution of genetic subroutines containing instructions for any desired cellular life function, biochemical activity or operation. Such additional genetic programming can be simple, such as inclusion of an expression cassette for one or more gene products to be produced by the host nanomachine, or complex, such as inclusion of an entire biochemical pathway or network to confer sophisticated physiological responses. Therefore, the host biological nanomachines of the invention can be designed and tailored to perform one, two, several and even many additional activities and operations up to and including substantial functional mimicry of naturally occurring cellular life forms.

Additional genes that can be included can be obtained from any functional category, including those that constitute a minimal gene set as well as those which substantially enhance the functioning and operation of a host nanomachine. Such additional categories include, for example, those set forth in FIG. 1 for non-replicative basic genetic operating systems, FIG. 2 for replication competent basic genetic operating systems, orthologs for genes within these functional categories as exemplified in Table 4, or as known to those skilled in the art and nonorthologous displacements. Therefore, a basic genetic operating system sufficient for viability, other non-replicative cellular life functions, replication competence or other replication competent cellular life functions, for example, can be further supplemented with overlying genetic applications encoding non-fundamental genes for these referenced cellular life functions within any of the functional categories show, for example, in FIG. 1 or 2. Specifically, overlying genetic applications can contain, for example, non-fundamental genes within the functional categories for replication, transcription, translation, the various metabolic functional categories, a phosphotransferase system (PTS) category, a signal transduction and regulation category, a transport and binding protein category, a particle division category, a chaperone system category, a particle envelope category and a housekeeping function category. Other non-fundamental genes and functional categories well known to those skilled in the art also can be included in such supplemental programming to confer one or more predetermined activities onto a host nanomachine of the invention.

Specific examples of non-fundamental genes within the above functional categories include, for example, genes selected such as the M. genitalium genes termed MG020, MG022, MG034, MG039, MG041, MG046, MG051, MG061, MG062, MG108, MG121, MG129, MG183, MG188, MG368, MG429, an ortholog or a nonorthologous gene displacement thereof. MG020 and MG183 encode, for example, genes involved in amino acid metabolism. MG022 encodes a gene involved in transcription. MG034 and MG051 encodes a gene involved in nucleotide metabolism. Nine of the above genes encode activities required for the PTS system. These genes include, for example, MG039, MG041, MG061, MG062, MG108, MG121, MG129, MG188 and MG429. MG046 is involved, for example, in secretion and therefore, can be considered to fall within the translation functional category. Finally, MG368 encodes a gene involved in lipid metabolism. Numerous other genes also exist from both procaryotic and eucaryotic cells and organisms. Any other genes within functional categories of a basic genetic operating system of the invention also can be integrated into a basic genetic operating system to generate a nanomachine genome encoding a specified activity or operation additional to that encoded by its basic genetic operating system.

Similarly, a basic genetic operating system sufficient for viability or replication competence, for example, also can be integrated by genetic applications programing independent or substantially independent functions to those specified in the underlying operating system. For example, complete pathways and networks for various physiological functions can be incorporated, including for example, motility, chemotaxis, homing, apoptosis, cellular immunity, humoral immunity, innate immunity, cytokine production, growth factor production, cellular adhesion and cellular migration. Other activities that can be integrated with a basic genetic operating system can include, for example, drug resistance, drug sensitivity, temperature, pH and salimity resistance or sensitivity as well as modulation of a redox state. Additional genes within any of the fundamental categories such as transcription or translation can be added as well as genes encoding post-translational modifications, functions, or polypeptide foldings. Additionally, a basic genetic operating system also can be integrated with genes encoding structural polypeptides such as cytoskeletal and membrane skeleton polypeptides to increase structural integrity of a nanomachine particle. Numerous other additional programming can be incorporated into a basic genetic operating system of the invention to impart an attribute or confer an activity onto the host nanomachine. Those skilled in the art will know what additional functions are germane to a targeted nanomachine application as well as which genes are necessary or sufficient to accomplish a particular outcome.

Therefore, the invention provides a prototrophic or auxotrophic basic genetic operating system having one or more non-fundamental genes operationally linked to the basic genetic operating system. The basic genetic operating system can encode non-replicative cellular life functions, including activities sufficient for viability, as well as replication competent cellular life functions. Such non-fundamental genes can be, for example, within a functional category of a basic genetic operating system or any other gene or genes that are engineered to impart a predetermined activity, operation or function onto a host nanomachine of the invention.

As described above, one particular application that can be advantageously suited to the bottom-up design and self-synthesis of a basic genetic operating system and host nanomachine, respectively, is the designed incorporation of biomolecule expression and production. One or more expression cassettes, for example, can be engineered into a basic genetic operating system of the invention for modular insertion of a gene encoding any desired biomolecule. Similarly, insertion of two or more genes and complete pathways encoding multiple subunits of biomolecules, multiple biomolecules or, for example, complete biosynthetic pathways or networks for nanomachine synthesis of one or more biomolecules of interest can be routinely engineered into a basic genetic operating system of the invention by those skilled in the art. Expression of such biomolecules can be constitutive or regulated, for example. Regulated expression can be accomplished by, for example, any genetic, recombinant, enzymatic or signal transduction mechanism known in the art, including for example, inducible or conditional expression by exogenous or physiological stimuli. Therefore, biosynthetic regulation also can be tailored to a particular nanomachine application or operation.

For example, insulin can be a biomolecule produced by a nanomachine of the invention. The insulin can be constitutively produced if it is desirable to make pharmaceutical quantities ex vivo. Alternatively, a nanomachine can be engineered with an inducible expression elements that is activated by elevated glucose levels or can be activated with an exogenously administered modulator. As described further below, such nanomachines can be advantageously administered to diabetic individuals for the treatment of diabetes.

Biomolecules can include, for example, a therapeutic macromolecules such as a polypeptide, a polypeptide complex, a ribo-(RNA) or deoxyribonucleic acid (DNA), lipid, sugar, glycopolypeptide, glycoside polypeptide, polyketides as well as biosynthesizable organic compounds. Such organic compounds can include, for example, macromolecule building block monomers such as amino acids, purine and pyrimidine bases, nucleosides, nucleoside monophosphates, and nucleotides, aldehydes, ketones, fatty acids, sugars, steroids, hydrocarbons, polymers, alkaloids, hormones, cytokines, chemokines, cofactors, neurotransmitters and the like. Biomolecules also can be, for example, macromolecules or biosynthesizable organic compounds suitable for diagnostic or industrial applications.

The basic genetic operating systems of the invention, including, for example, non-replicative and replication competent forms, can be produced by any method of nucleic acid synthesis known to those skilled in the art. Such methods include, for example, chemical synthesis, recombinant synthesis, enzymatic polymerization and combinations thereof. These and other synthesis methods are well known to those skilled in the art.

For example, methods for synthesizing oligonucleotides can be found described in, for example, Oligonucleotide Synthesis: A Practical Approach, Gate, ed., IRL Press, Oxford (1984); Weiler et al., Anal. Biochem. 243:218 (1996); Maskos et al., Nucleic Acids Res. 20(7):1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999).

Recombinant and enzymatic synthesis, including polymerase chain reaction and other amplification methodologies can be found described in, for example, Sambrook et al., Molecular Cloning: A Laboratory Manual, Third Ed., Cold Spring Harbor Laboratory, New York (2001) and in Ansubel et al., (1999), supra.

Solid-phase synthesis methods for generating arrays of oligonucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science (1991) 251:767-777, and Winkler et al., U.S. Pat No. 6,136,269; Southern et al. PCT Application No. WO 89/10977, and Blanchard PCT Application No. WO 98/41531. Such methods include synthesis and printing of arrays using micropins, photolithography and ink jet synthesis of oligonucleotide arrays.

Methods for synthesizing large nucleic acid polymers by sequential annealing of oligonucleotides can be found described in, for example, in PCT application No. WO 99/14318 to Evans and also described further below in the Examples. All of the above references are incorporated herein by reference in their entirety.

The invention additionally provides an autonomous prototrophic nanomachine having a basic genetic operating system for autonomous prototrophic viability and a particle envelope.

Any of the basic genetic operating systems described above, such as those directing the synthesis and maintenance of basic cellular viability functions can be packaged into a particle envelope to produce an autonomously viable prototrophic nanomachine of the invention. Particle envelopes can include, for example, any semi-permeable partitioning biocompatible material that maintains separation of the basic genetic operating system or nanomachine genome, nanomachine macromolecular structures such a ribosomes and transcriptional apparatus, macromolecules and organic molecules from the external environment. A particle envelope can allow, for example, by diffusion, passive or active transport, pinocytosis, phagocytosis, vesicle fusion or other processes well known to those skilled in the art, the influx of nutrients, minerals and other molecules needed for the proper functioning and operation of the nanomachine. Similarly, a particle envelope can allow by, for example, the above processes well known in the art, the efflux of metabolic by-products and waste products.

Various biocompatible materials well known to those skilled in the art can be used as a particle envelope. For example, a particle envelope can be a lipid vesicle or a lipid bilayer similar to naturally occurring cellular membranes. Other biocompatible materials useful as a particle envelope include, for example, phospholipids, liposomes, lipoprotein micelles, and viral or phage envelopes. Alternatively, particle envelopes can be constructed from synthetic or naturally occurring materials such as filter membranes, Gortex™, polyamides, polyfluorenes and fluorocarbons. Combinations of the above biocompatible materials also can be used for nanomachine particle envelopes of the invention. Also, a basic genetic operating system of the invention can further be programmed, by inclusion of genes encoding for fatty acid and lipid biosynthesis, for example, to autonomously produce bilayer lipid membranes similar to naturally occurring cells.

Initial functional operation of a nanomachine can require, for example, the inclusion of starter molecules and macromolecules that are sufficient to achieve at least one round of transcription or translation. For example, nanomachine particle containing only a basic genetic operating system without essential cellular machinery, precursors and energy sources to initially transcribe or translate de novo the nanomachine genome can be inoperative. Therefore, starter components consisting of, for example, the above machinery, precursors or energy sources can be packaged within the nanomachine particle envelope in sufficient amounts to allow genome-directed synthesis and production of threshold amounts of nanomachine components. A threshold amount is an amount that is produced from a basic genetic operating system which is sufficient for autonomous nanomachine activity and operation. Because macromolecules and organic molecules can have finite half-lives, the initially packaged starter components will be exhausted or cured following initial operation of the nanomachine particle. Therefore, autonomous programmed functions will take over to replenish fundamental components and maintain prototrophic homeostasis of a nanomachine of the invention.

Starter components can be, or obtained from, for example, cell lysates, cellular fractions, recombinant production, biochemically purification, cellular-nanomachine fusions and other sources and methods well known to those skilled in the art. Generally, starter components can contain threshold amounts of each gene or end product component synthesized by a gene, pathway or network within the corresponding basic genetic operating system. However, nanomachine particles of the invention can be brought up to operation with only a few rudimentary activities and structures such as RNA polymerase, ribosomes and translation factors and an energy source. Exemplary amounts of starter components include, for example, femtomolar, nanomolar or micromolar quantities of essential fundamental gene products. Those skilled in the art will know that the actual amount and composition of the starter components can be adjusted depending on the need. For example, increasing the initial concentration of energy components such as ATP can allow corresponding decreases in number of different types of molecules within the starter composition because the nanomachine will have a larger initial reservoir before it has to start producing its own energy supply.

The invention further provides a basic genetic operating system for an autonomous auxotrophic nanomachine having a nanomachine genome encoding a minimal gene set sufficient for viability in the presence of an auxotrophic biomolecule.

As described previously, basic genetic operating systems that can direct autonomous nanomachine cellular life functions in the presence of an exogenous supply of a biomolecule are auxotrophic basic operating systems and host nanomachines, respectively. The teachings and guidance set forth above with respect to autonomous prototrophic basic operating systems and host nanomachines are similarly applicable to auxotrophic systems and nanomachines. One difference, however, being that an engineered deficiency is functionally complimented by exogenous supplies of a biomolecule that can rescue the design defect.

Therefore, auxotrophic basic genetic operating systems similarly can include, for example, minimal gene sets encoding the functional categories of transcription, translation, aerobic metabolism, anaerobic metabolism, carbohydrate metabolism, central intermediary metabolism, nucleotide metabolism, transport and binding proteins, and housekeeping functions. Such categories can additionally be synthesized in any desired physical or temporal order including, for example, a relative physical or temporal order of transcription, translation, aerobic metabolism and glycolysis, pyruvate dehyrogenase, pentose phosphate pathways, respectively. Similarly, as described in reference to a prototrophic basic genetic operating system sufficient for viability, an auxotrophic basic genetic operating system sufficient for viability also can be devoid of at least one gene selected from MG008, MG009, MG056, MG221, MG332, MG448 or MG449, an ortholog or a nonorthologous gene displacement thereof. Likewise, an auxotrophic basic genetic operating system can similarly be designed as a spatially condensed nucleic acid of about 140 kb or less in size. The design alternatives and considerations described previously are also directly applicable to auxotrophic basic genetic operating systems. Similarly, the design and incorporation of additional genetic programming overlaid onto, and run off of, a prototrophic basic genetic operating system are additionally directly applicable to an auxotrophic basic genetic operating system. Therefore, an auxotrophic basic genetic operating system can be engineered to include expression cassettes for the production of one or more biomolecules, biochemical pathways and networks.

The invention further provides a basic genetic operating system for an autonomous auxotrophic nanomachine having about 151 or less fundamental genes.

As described previously, a basic genetic operating system specifying basal cellular life functions such as viability can be accomplished, for example, with about 152 fundamental genes or less. However, for an auxotrophic basic genetic operating system, any one or more of these genes can be rendered deficient so long as the deficiency can be complemented or rescued by supplementation with a compound, molecule or macromolecule. Those skilled in the art will know which gene functions can be supplied by supplementation of the nanomachine external environment. For example, glycolysis metabolizes glucose to glucose phosphate via glucokinase. Elimination of the glucokinase gene can be rescued by supplying glucose phosphate rather than glucose in the external environment to maintain autonomy of such a system auxotrophic for glucokinase. Similarly, entire functional systems can be deleted if the components are added to the external medium or, alternatively, introduced into the nanomachine itself. For example, elimination of ribosome synthesis and protein synthesis machinery also can be designed into an auxotrophic basic genetic operating system and these functions can be rescued by supplying a cell-free or artificial extract to provide protein synthesis function. Such auxotrophic nanomachines can autonomously function for polypeptide synthesis directed by the auxotrophic basic genetic operating system using the externally supplied functions rather than internally synthesized translation machinary.

Therefore, the about 9 functional categories described previously similarly can constitute an auxotrophic basic genetic operating system of the invention. However, depending on the fundamental genes and categories selected, the number of genes can be, for example, 151 or less. As such, an auxotrophic minimal gene set will contain at least one non-functional gene within, for example, the constituent genes described previously which are sufficient to support viability.

Exemplary fundamental genes and their gene product functions within each of the functional categories and subgroups are shown in FIG. 1. Orthologous genes which can similarly substitute for those shown in FIG. 1 are set forth in Table 4 below. Given the teachings and guidance provided herein those skilled in the art will know or can determine, other orthologs or nonorthologous displacements that similarly can substitute for one or more of the fundamental genes shown in FIG. 1 or Table 4. Therefore, the invention provides a basic genetic operating system sufficient to direct autonomous auxotrophic viability of a host nanomachine having about 151 or less fundamental genes that consists of substantially the same fundamental genes show in FIG. 1, Table 4, orthologs or nonothorologous displacements thereof.

Any of the auxotrophic basic genetic operating systems described above, such as those directing the synthesis and maintenance of basic cellular viability functions, can be packaged into a particle envelope to produce an autonomously viable auxotrophic nanomachine of the invention in the presence of the corresponding auxotrophic biomolecule. Particle envelopes can include, for example, any semi-permeable partitioning biocompatible material that maintains separation of the basic genetic operating system or nanomachine genome, nanomachine macromolecular structures, macromolecules and organic molecules from the external environment. Particle envelopes also can include other physical, chemical or electric forces that can generate a microenvironment for separation of nanomachine from non-nanomachine components. As with basic genetic operating systems programmed for prototrophic cellular life functions, the auxotrophic basic genetic operating systems can be programmed similarly to direct the biosynthesis and maintenance of cellular life functions. Such cellular life functions include, for example, viability, replication, transcription, translation, cell division, energy generation, cellular homeostasis, adhesion, motility, migration, environmental adaption, chemotaxis and immune and effector cell responses. Other cellular life functions, biochemical or physiological activities or operations well known to those skilled in the art also can be programmed separably or together with the above cellular life functions.

The invention provides a basic genetic operating system for an autonomous prototrophic nanomachine having a nanomachine genome encoding a minimal gene set sufficient for autonomous prototrophic replication. The nanomachine genome can direct synthesis of the minimal gene set in a relative order of functional categories having the functions of replication, transcription, translation, aerobic metabolism and glycolysis, pyruvate dehyrogenase and pentose phosphate pathways, respectively. Also provided is a basic genetic operating system for a prototrophic nanomachine, further having functional categories of the minimal gene set for carbohydrate metabolism, central intermediary metabolism, nucleotide metabolism, signal transduction regulation, transport and binding proteins, particle division, chaperone system, fatty acid/lipid metabolism, particle envelope and housekeeping functions.

The invention also provides a basic genetic operating system for an autonomous auxotrophic nanomachine having a nanomachine genome encoding a minimal gene set sufficient for autonomous replication in the presence of an auxotrophic biological molecule. The nanomachine genome can direct synthesis of the minimal gene set in a relative order of functional categories having the functions of replication, transcription, translation, aerobic metabolism and glycolysis, pyruvate dehydrogenase, and pentose phosphate pathways, respectively. Further provided is a basic genetic operating system for an auxotrophic nanomachine further having functional categories of the minimal gene set for carbohydrate metabolism, central intermediary metabolism, nucleotide metabolism, signal transduction regulation, transport and binding proteins, particle division, chaperone system, fatty acid/lipid metabolism, particle envelope and housekeeping functions.

A basic genetic operating system of the invention specifying the genetic programming for replication competent nanomachines is a nucleic acid, or a functional equivalent of a nucleic acid, that can serve as a genome for a biosynthetic cell or nanomachine. Encoded within a basic genetic operating system sufficient for replication competence are, for example, the required gene products that are obligatory to synthesize and sustain foundational functions of the constituent components and processes of this cellular life function. Whether a basic genetic operating system provides the genetic information for any of various non-replicative nanomachines or for any of various replication competent nanomachines, a basic genetic operating system differs from a complete genome, for example, because it duplicates or more closely approximates a genetic copy of genes, or functional fragments thereof, that are essential for the engineered replicative or non-replicative cellular life function. Therefore, a basic genetic operating system is a simpler and more efficient genome compared to naturally occurring genomes because it lacks unnecessary or redundant genetic information or structure.

As a streamlined copy of genes that are obligatory to sustain, for example, replication competence, a basic genetic operating system constitutes a minimal compilation of genes that are required for the biosynthesis and maintenance of this cellular life function. A prototrophic basic genetic operating system will encode a complete minimal gene set whereas an auxotrophic basic genetic operating system will encode, for example, at least one non-functional gene within a minimal gene set whose function can be supplied by exogenous supplementation. Therefore, a basic genetic operating system specifying autonomous replication can, by itself, substitute for, or function as, a cellular or nanomachine genome sufficient to support autonomous replication for at least one cycle of replication. Additionally, and as described further below, a basic genetic operating system also can be combined with other genes and gene sets to augment the genetic instructions of the basic operating system. Inclusive of other genes, can, for example, enable a host nanomachine to perform and maintain a wide variety of biochemical activities and operations in conjunction with those constituting fundamental cellular life functions such as replication.

A minimal gene set sufficient to support either prototrophic or auxotrophic replication competence includes, for example, genes that fall within a number of functional categories. In a simple form, a replication competent minimal gene set will include, for example, a minimal gene set sufficient for viability and fundamental genes sufficient for replication of the genome. Where a genome is DNA such genes can include, for example, DNA polymerase and related elementary replication factors. In comparison, where a genome is RNA, such genes can include, the requisite reverse transcriptase or RNA polymerase required for the engineered replication mechanism.

More complex replication competent minimal gene sets, can additionally include, for example, fundamental genes required for nanomachine particle division and membrane biogenesis. In the absence of fundamental functions for particle division, a replication competent host nanomachine can replicate its genome but not substantially divide into daughter particles. A basic genetic operating system specifying fundamental functions for replication in the absence of particle division functions can result in production of a particle having, for example, two or more genomes in its intraparticle space. Inclusion of membrane biogenesis functions, such as fatty acid and phospholipid metabolism, in such a replication competent basic genetic operating system can allow a host nanomachine to expand in size and volume to accommodate the additional nucleic acid mass. Inclusion of fundamental genes sufficient for particle division or membrane biogenesis will result in protrotrophic basic genetic operating systems for these referenced activities.

Alternatively, such host nanomachines can be engineered and maintained as auxotrophs for the above fundamental functions of membrane biogenesis, particle division or both. Gene products or even nucleic acids encoding these functions which are, for example, separable from the basic genetic operating system can be introduced into the nanomachine to allow particle enlargement or induce particle division.

Although described with reference to membrane biogenesis and particle division in connection with replication competent nanomachines, such strategies and modes of operation are equally applicable for both non-replicative and replication competent nanomachine species as well as for a single auxotrophic fundamental gene, two or more auxotrophic fundamental genes, basic genetic operating systems engineered to be auxotrophic for pathways and networks. Given the teachings and guidance provided herein, those skilled in the art will know, or can routinely determine, various different combinations and permutations for prototrophic and auxotrophic basic genetic operating systems, their respective requirements for operation and modes of rescuing an auxotrophic phenotype.

Additionally, fundamental genes encoding augmentory rudimentary functions also can be included in a basic genetic operating system containing a minimal gene set sufficient for replication competence. Such augmentory rudimentary functions can include, for example, fundamental genes encoding polypeptide turnover and folding; purine, pyrimidine, nucleoside and nucleotide biosynthesis; chaperones, and regulatory functions. For example, the additional M. gennitalium genes set forth in FIG. 2 compared to FIG. 1, and the exemplary orthologs shown in Table 4 are examples of a fundamental genes that can be contained in a minimal gene set sufficient for replication compared to one encoding gene products sufficient for viability. Other examples of minimal gene sets that support autonomous host replication are described in, for example, in Mushegian and Koonin, supra; Koonin et al., supra; Hutchison et al., supra, and at NCBI URL ncbi.nlm.nih.gov/cgi-bin/Complete_Genomes/mglist, supra. The constituent genes and gene products and their interrelationships or independence with respect to other functional categories and cellular life functions is described further below.

Functional categories of genes within a minimal gene set constituting the genetic programming sufficient to support replication as a cellular life function include, for example, about fifteen or less fundamental biochemical processes. Nine of these functional categories include those described above for a minimal gene set sufficient for viability. Similarly, the fifteen or less functional categories also fall under the general groupings of biosynthetic, metabolic and homoeostatic processes. The biosynthetic groupings include, for example, the functional categories of replication, transcription, translation and particle envelope production.

Metabolic processes include, for example, energy metabolism, carbohydrate metabolism, central intermediary metabolism, nucleotide metabolism and fatty acid and phospholipid metabolism. Energy metabolism can further include the functional categories of aerobic metabolism and anaerobic metabolism. Glycolysis, pyruvate dehydrogenase and the pentose phosphate pathways are specific biochemical pathways supplying high free energy molecules such as ATP, NADH and NADPH under aerobic conditions. Any of these energy metabolism subgroups of fundamental genes are sufficient to supply adequate energy supplies for autonomous nanomachines programmed by replication competent or non-replicative basic genetic operating systems. Carbohydrate metabolism includes, for example, fundamental genes active in sugar conversion. Nucleotide metabolism includes, for example, de novo or salvage pathway synthesis of purine and pyrimidine bases, nucleosides and nucleotides.

Function categories within the homoeostatic processes include, for example, regulatory functions, transport and binding functions, particle division, chaperone functions and housekeeping functions.

Those skilled in the art will know what fundamental genes are, or can be, contained within each category, including for example, those derived from procaryotic and eucaryotic sources. Exemplary listings of functional categories and constituent minimal gene set sufficient for a basic genetic operating system to direct a replication competent autonomous nanomachine is shown in FIG. 2 and Table 4. Therefore, the functional categories constituting a minimal gene set sufficient for a cellular life function such as replication competence can be derived from a single species or multiple species. Similarly, fundamental genes determine to fall within a functional category also will include, for example, functional equivalents such as orthologs and nonorthologous displacements as well as functional fragments thereof.

As with non-replicative systems, various combinations and permutations of functional categories for a basic genetic operating system programmed to direct replication competent autonomous nanomachines, such as those shown in FIG. 2 and Table 4, for example, can be produced depending on the need and desired operation of the host nanomachine. The design considerations and engineering of non-replication competent basic genetic operating systems tailored for a particular nanomachine application are also directly applicable to replication competent basic genetic operating systems. For example, a replication competent nanomachine can be programmed to function under completely aerobic conditions, or alternatively, under anaerobic conditions as described previously. Similarly, a replication competent nanomachine also can be programmed to generate macromolecules by de novo or salvage biosynthesis. Further, for example, if a nanomachine of the invention is desired to exhibit particle-particle or particle-matrix adhesion, migration, motility, cytokine regulation, growth factor regulation, immune and effector mechanism or chemotaxis to perform a targeted application, then these functional categories and their constituent fundamental genes can be included within a replication competent basic genetic operating system of the invention.

Numerous other combinations, substitutions and permutations of functional categories can be made in a basic genetic operating system of the invention to tailor the performance of either an autonomous prototrophic or auxotrophic nanomachine to a particular application. Such other modifications of functional categories include, for example, those described previously with prototrophic and auxotrophic non-replicative systems. Those skilled in the art will know which functional categories can be combined, modified or substituted to accomplish a predetermined activity, cellular life function or application. Additionally, as with the other functional categories, the genes within a particular biosynthetic pathway are well know to those skilled in the art. Similarly, using the teachings and guidance provided herein, those skilled in the art will know, or can determine, which genes within a biochemical pathway or physiological process are fundamental genes and can be included with a minimal gene set and which genes are dispensable to the efficient function and operation of a nanomachine programmed with a basic genetic operating system conferring replication competence.

A minimal gene set will include, for example, genes within a functional category that are fundamental to a biochemical process. Fundamental genes for replication competence include, for example, those genes that are essential to the process as well as those elementary genes that augment the performance of a biochemical process to comparable cellular or reference standard levels. For example, a basic genetic operating system specifying replication competent programming can additionally include, for example, fundamental genes encoding de novo nucleotide biosynthesis compared to non-replicative basic systems. The inclusion of additional nucleotide metabolism functions can compensate for the added requirement necessary to replicate the nanomachine genome. Those skilled in the art will know, or can determine, fundamental genes that encode either an essential function or an elementary function within a minimal gene set. Similarly, whether in context of replication competent or non-replicative basic genetic operating systems, those skilled in the art also will understand that augmentation of a elementary process, and therefore includable as a fundamental gene, differs from optimization.

The functional categories constituting a replication competent basic genetic operating system of the invention can be arranged in essentially any desired physical or functional order so long as all genes of the minimal gene set are present and operative. However, arranging the functional categories in relative order of importance can augment the efficiency of the host replication competent nanomachine operation. Similarly, arranging the functional categories in relative order of importance also can increase the quality of a particular nanomachine product or activity. Depending on the desired use of an autonomous prototrophic or auxotrophic nanomachine of the invention, the functional gene categories can be selectively arranged to optimize or regulate, for example, the genetic programming of the basic genetic operating system, nanomachine operation efficiency or genome size.

One arrangement of functional categories within a replication competent basic genetic operating system can be, for example, in the relative order of gene product use to achieve the encoded replication and supporting functions. To sustain cellular life functions and enable genome replication, a host nanomachine should be able to biosynthesize, for example, component macromolecules sufficient for replication, transcription, translation and at least one pathway of energy production. One relative order of nanomachine use can be, for example, a relative order of fundamental genes constituting the functional categories of replication, transcription and translation categories, respectively, followed by functional categories specifying nanomachine energy sources. Alternatively, fundamental genes constituting one or more energy sources can be, for example, placed prior to or between the biosynthetic functional categories. Such energy sources can be, for example, fundamental gene sets sufficient for either or both aerobic metabolism and anaerobic metabolism, or a pathway thereof.

The remainder of the functional categories of genes sufficient for replication competence of a host nanomachine can be essentially any desired order depending on the targeted application of nanomachine and desired efficiency. One exemplary order of the remaining categories can be, for example, carbohydrate metabolism, central intermediary metabolism, nucleotide metabolism, regulatory functions such as signal transduction, transport and binding proteins, particle division, chaperone functions, fatty acid and lipid metabolism, particle envelope generation and housekeeping functions, respectively. The number of permutations and combinations of functional category order are many. Those skilled in the art will know what order and combination of functional categories can be made within a basic genetic operating system to achieve a desired result. Therefore, the invention provides a basic genetic operating system having functional categories described above and set forth in FIG. 2 and Table 4 arranged in all possible orders. Additionally, any of the fundamental genes within one or more of the functional categories can be separated and the resulting portions ordered within a basic genetic operating system separately from, or independent to, each other.

As with the prototrophic and auxotrophic basic genetic operating systems described previously, ordering of functional categories specifying replication competent basic genetic operating systems also can be based on several different criteria. For example, ordering can be accomplished with reference to physical order or temporal order. Any particular physical order can be accomplished, for example, by placement of fundamental genes or whole functional categories with reference to one or more genomic markers and in one or more directions as described previously. Also as described previously, various temporal ordering of fundamental genes or functional categories can be accomplished, for example, by activation and repression of targeted genes and gene sets in a selected order or by a combination of selected activation and repression and physical arrangements.

The invention also provides a basic genetic operating system for an autonomous protrophic nanomachine having a nanomachine genome encoding a minimal gene set sufficient for directing autonomous prototrophic replication, he minimal gene set being devoid of at least one gene selected from the group consisting of MG008, MG009, MG056, MG221, MG262, MG332, MG448 or MG449, an ortholog or a nonorthologous gene displacement thereof.

Further provided is a basic genetic operating system for an autonomous auxotrophic nanomachine having a nanomachine genome encoding a minimal gene set sufficient for directing autonomous replication in the presence of an auxotrophic biological molecule, the minimal gene set being devoid of at least one gene selected from the group consisting of MG008, MG009, MG056, MG221, MG262, MG332, MG448 or MG449, an ortholog or a nonorthologous gene displacement thereof.

As described previously with reference to basic genetic operating systems sufficient for viability or other non-replicative cellular life functions, although the above genes include conserved regions between, for example, M. genitalium and H. influenza, they also can be considered to encompass redundant structures or functions compared to other genes found within their respective genomes. Similarly, MG008, MG009, MG056, MG221, MG262, MG332, MG448 or MG449, orthologs or nonorthologous displacements thereof also can be considered, for example, to encompass redundant structures or functions compared to the compliment of genes found in genomes of other species as well. Additionally, some of these genes are unnecessary for rudimentary functions and, if desired to be included within a replication competent basic genetic operating system of the invention, more appropriate to be placed in an overlying genetic program operated from the underlying basic system.

A replication competent basic genetic operating systems devoid of MG008, MG009, MG056, MG221, MG262, MG332, MG448 or MG449, orthologs or nonorthologous displacements thereof, should include, for example, sufficient functional categories and constituent fundamental genes to direct the synthesis and maintenance of its host nanomachine components. Therefore, replication competent basic genetic operating systems devoid of one or more of the above genes can be constructed as, for example, simple, intermediate or complex versions of the replication competent basic genetic operating systems described previously. Similarly, any architectural design or arrangement of functional categories or constituent fundamental genes also can be engineered and constructed for a prototrophic or auxotrophic basic genetic operating system devoid of the above eight genes. Those skilled in the art will know, or can determine a suitable genetic structure for a particular targeted application of such replication competent host nanomachines.

Also provided by the invention is a basic genetic operating system for an autonomous prototropic nanomachine having a nanomachine genome encoding a minimal gene set sufficient for directing autonomous prototrophic replication, the nanomachine genome having less than about 250 kilobases (kb) in size. Further provided is a basic genetic operating system for an autonomous auxotrophic nanomachine having a nanomachine genome encoding a minimal gene set sufficient for directing autonomous auxotrophic replication in the presence of an auxotrophic biological molecule, the nanomachine genome having less than about 250 kilobases (kb) in size.

A basic genetic operating system containing a minimal gene set sufficient for viability can be constructed to be any size so long as it can be packaged into a particle envelope or other partitioning structure. Precise structures can be designed and synthesized, for example, to conserve or reduce space, partially or maximally miniaturize the genome linear or condensed size, increase structural or functional efficiency, optimize expression or regulatory element usage or tailored to include only relevant functional domains.

Those skilled in the art will know, or can readily design, a wide range of sizes for a basic genetic operating system sufficient to confer replication competence, given the teachings and guidance provided herein. For example, a minimal gene set such as that shown in FIG. 2 or corresponding orthologous genes shown in Table 4 which are sufficient to specify replication competence can be organized into a basic genetic operating system of about 250 kilobase (kb) pairs or less. For example, juxtaposition of intronless versions of all shown fundamental genes can result in a nucleic acid of about 248,124 bp. Such a minimal gene set encodes about 247 fundamental genes for a total of about 82,708 amino acids.

Inclusion of naturally occurring expression and regulatory elements, heterologous elements or combinations thereof, operationally linked to the intronless genes can be accomplished with minimal increase in nucleic acid size. All of the considerations and possible alternative engineering designs described previously in reference to non-replicative versions also are directly applicable for basic genetic operating systems programming replication competence. One additional consideration being, however, that the replication competent basic genetic operating system contain at least indispensable fundamental genes within the replication functional category.

Therefore, a basic genetic operating system of the invention programming nanomachine cellular life functions that are replication competent can be substantially smaller than about 250 kb. For example, a basic genetic operating system sufficient for replication competence can be about 240 kb or less, 230 kb or less, 220 kb or less, 210 kb or less, and even about 200 kb or less. It is also possible to reduce in half the size of such basic genetic operating systems to about 125 kb by, for example, substantial overlap and truncation of fundamental genes that constituting a minimal gene set. Other architectural designs well known to those skilled in the art similarly can be used to condense or optimize the structure of a basic genetic operating system of the invention.

As with the non-replicative basic genetic operating systems described previously, a replication competent basic genetic operating systems of the invention also can include, for example, various structural features that facilitate the transfer of information into encoded polypeptides and the operation of cellular life functions of a nanomachine. Additionally, the basic genetic operating systems of the invention can be designed as double stranded or single stranded genomic structures. The number of constituent genes within a functional category can vary, for example, depending on the targeted application of the host nanomachine. Considerations for which constituent fundamental genes to include have been described previously and include, for example, whether the programming is engineered for de novo or salvage biosynthetic activities, replication within an intracellular or extracellular physiological environment or an extracellular non-physiological environment or whether the basic genetic operating system specifies prototrophic or auxotrophic nanomachine autonomy.

Generally, fundamental genes sufficient to support autonomous prototrophic replication can be grouped, for example, into about 24 genes in a replication gene category, about 14 genes in a transcription gene category, about 94 genes in a translation gene category, about 13 genes in an aerobic metabolism gene category, about 16 genes in an a gene category, constituting glycolysis, pyruvate dehydrogenase and pentose phosphate pathways, about 3 genes in a carbohydrate metabolism gene category, about 13 genes in a central intermediary metabolism gene category, about 18 genes in a nucleotide metabolism gene category, about 4 genes in a signal transduction regulation gene category, about 23 genes in a transport/binding protein gene category, about 4 genes in a particle division gene category, about 11 genes in a chaperone system gene category, about 3 genes in a fatty acid/lipid metabolism gene category, about 3 genes in a particle envelope gene category, and about 4 genes in a housekeeping function gene category. Fundamental genes sufficient to support autonomous auxotrophic replication can contain, for example, at least one non-functional fundamental gene within one or more of these categories. Therefore, a basic genetic operating system for an autonomous auxotrophic nanomachine encodes a minimal gene set sufficient for autonomous replication in the presence of an auxotrophic biological molecule which contains, for example, about 246 or less fundamental genes.

The functional category containing fundamental genes functioning in replication processes include, for example, a DNA polymerase encoding gene, helicase, topoisomerase, and recombination and repair enzymes. Exemplary fundamental genes for replication are shown in FIG. 2. The transcription functional category contains RNA polymerase, basic transcription factors, nucleases and modifying enzymes, for example. The category containing fundamental genes functioning in the translation processes can be further divided, for example, into four further subgroups. These translation subgroups can consist, for example, of about 25 genes that encode tRNA synthesis and modification activities and amino acid metabolism; about 4 genes that encode degradation and polypeptide folding activities; about 13 genes whose gene products function in polypeptide modification and translation factors, and about 52 genes whose gene products function in ribosome biosynthesis, assembly and modification. There are about 10 fundamental genes encoding glycolytic functions, about 2 fundamental genes encoding pyruvate dehydrogenase pathway gene products and about 4 fundamental genes encoding gene products that function in the pentose phosphate pathway. Specific examples of constituent fundamental genes within the various functional categories sufficient for replication competence are shown in FIG. 2 and in Table 4.

Exemplary fundamental genes and their gene product functions within each of the above functional categories and subgroups within a minimal gene set sufficient for autonomous prototrophic and auxotrophic replication are shown in FIG. 2. Orthologous genes which can similarly substitute for those shown in FIG. 2 are set forth in Table 4 below. Given the teachings and guidance provided herein those skilled in the art will know or can determine, by for example, comparative genomics and gene product function, other orthologs or nonorthologous displacements that similarly can substitute for one or more of the fundamental genes shown in FIG. 2 or Table 4.

Therefore, the invention provides a basic genetic operating system sufficient to direct autonomous prototrophic replication of a host nanomachine having about 247 or less fundamental genes that consists of substantially the same fundamental genes show in FIG. 2 or Table 4, including orthologs or nonothorologous displacements thereof. A basic genetic operating system sufficient to direct autonomous auxotrophic replication in the presence of an auxotrophic biomolecule also is provided which has about 246 or less fundamental genes that consists of substantially the same fundamental genes show in FIG. 2 or Table 4, including orthologs or nonorthologous displacements thereof.

As described previously, any basic genetic operating system of the invention can additionally operationally incorporate overlying genetic programming to a impart predetermined activity or activities onto a host nanomachine of the invention. Nanomachines of the invention can be genetically programmed to perform and carry out a wide range of biochemically activities or operations by constructing a nanomachine genome that contains in addition to a basic genetic operating system predetermined genes encoding gene products having one or more activities which can execute the biochemical activity or operation.

As described previously in reference to non-replicative basic genetic operating systems, one particular application of a prototrophic or auxotrophic replication competent basic genetic operating system is the designed incorporation of biomolecule expression and production. One or more expression cassettes can be, for example, engineered into a basic genetic operating system of the invention for modular insertion of one or more genes encoding any desired biomolecule or biomolecules, biochemical pathway or network. Expression of such biomolecules can be accomplished by any method well known to those skilled in the art including, for example, constitutive or regulated. Therefore, biosynthetic regulation also can be tailored to a particular replication competent nanomachine application or operation.

Biomolecules include, for example, a therapeutic macromolecule such as a polypeptide, a polypeptide complex, a ribo-(RNA) or deoxyribonucleic acid (DNA), lipid or sugar, as well as biosynthesizable organic compounds. Biomolecules also can be produced for diagnostic or industrial purposes. Other exemplary biomolecules have been described previously.

The invention additionally provides an autonomous prototrophic nanomachine having a basic genetic operating system for autonomous prototrophic replication and a particle envelope. An autonomous auxotrophic nanomachine having a basic genetic operating system for autonomous replication in the presence of an auxotrophic biological molecule and a particle envelope is also provided.

As with the non-replicative forms, any of the replication competent basic genetic operating systems described above can be packaged into a particle envelope to produce an autonomous replication competent prototrophic or auxotrophic nanomachine of the invention. Auxotrophic nanomachines will function autonomously in the presence of an auxotrophic biomolecule that compliments the non-functional gene. As described previously, particle envelopes can include, for example, any semi-permeable partitioning biocompatible material that maintains separation, for example, of the basic genetic operating system, nanomachine macromolecular structures, macromolecules and organic molecules from the external environment. A particle envelope also can allow, for example, by processes well known to those skilled in the art, the influx of nutrients, minerals and other molecules needed for the proper functioning and operation of the nanomachine as well as for the efflux of metabolic by-products and waste products.

Various biocompatible materials well known to those skilled in the art can be used as a particle envelope. For example, a particle envelope can be a lipid vesicle, a lipid bilayer or constructed from synthetic or naturally occurring materials well known to those skilled in the art and as described previously. Further, combinations of natural and synthetic biocompatible materials also can be used for nanomachine particle envelopes of the invention. The particle envelope also can be synthesized from genes encoded by a basic genetic operating system and therefore self-produced. The use of lipid based membranes can perform both the functions of partitioning nanomachine components and serving as a particle envelope that can be homoeostatic regulated by inclusion of fundamental genes for fatty acid and lipid metabolism, for example. Additional fundamental genes encoding membrane components functions also can be included in a basic genetic operating system to augment envelope production or homoeostatic regulation.

Accordingly, a replication competent basic genetic operating system of the invention can be programmed by inclusion, for example, of genes encoding for fatty acid and lipid biosynthesis to autonomously produce bilayer lipid membranes similar to naturally occurring cells. Alternatively, a particle envelope can be partially or completely composed of non-biosynthesizable components. Particle envelope components that can be biosynthetically produced can be programmed into the nanomachine's basic genetic operating system. Non-biosynthetically produced particle components can be added, for example, at formation of the particle envelope as well as added later to supplement the envelope composition or produce desirable changed in the envelope composition.

Those skilled in the art will known that replication competence and particle division are separable for both prototrophic and auxotrophic nanomachines. For example, a nanomachine of the invention that is capable of autonomously duplicating its genome is a replication competent nanomachine. In the absence of particle division, a replication competent nanomachine can accumulate multiple copies of its genome. Therefore, replication competence does not require particle division. One advantage of replication competent, non-dividing nanomachines is that they increase expression levels of encoded genes by increasing genomic copy number. A useful application of a replication competent, non-dividing nanomachine can be, for example, for the expression of a biomolecule because each round of autonomous replication can increase the copy number of the biomolecule encoded gene and its corresponding rate of synthesis or accumulation. Inclusion of fundamental genes in a basic genetic operating system sufficient to program particle division can additionally confer onto a host nanomachine the ability to multiple in particle number. One advantage of replication competent nanomachines that also can undergo particle division is that they are self-reproducing and therefore capable of sustaining programmed functions over long periods of time. This reproduction phenotype can allow, for example, for the steady and long-lived synthesis of a biomolecule or execution of a programmed activity.

As described previously, initial functional operation of a nanomachine can be accomplished, for example, by the inclusion of starter molecules and macromolecules that are sufficient to achieve at least one round of replication, transcription or translation. Starter components consisting of, for example, replication, transcription or translation machinery, precursors or energy sources can be packaged within the nanomachine particle envelope in sufficient amounts to allow genome-directed synthesis and production of threshold amounts of nanomachine components. Autonomous programmed functions will take over to replenish fundamental components and maintain prototrophic or auxotrophic homeostasis of a nanomachine of the invention. Starter components can be, or obtained from, for example, cell lysates, cellular fractions, recombinant production, biochemically purification, cellular-nanomachine fusions and other sources and methods well known to those skilled in the art and as described previously.

The nanomachines of the invention can be used in a wide variety of therapeutic, diagnostic and industrial applications. An exemplary and non-exhaustive list of such applications includes, for example, the use of nanomachines as a bioreactor,; for bioremediation; for the production of a therapeutic biomolecule or as a therapeutic reagent; for the production of a diagnostic indicator or as a diagnostic reagent; as a delivery system; as an artificial tissues or organ system; as an energy conversion system; as a processing system; as an anabolic or catabolic system; for the production of biological films or coatings that may respond to the environment, and for cosmetic applications, including cosmeceuticals. Nanomachines of the invention can be employed in such applications in a variety settings including, for example, in vivo, in situ or in vitro settings. Depending on the targeted application, such nanomachine applications can be performed with any of the nanomachines described previously. Therefore, autonomous prototrophic or auxotrophic non-replicative nanomachines or autonomous prototrophic or auxotrophic replication competent nanomachines can be employed in, for example, the above applications to produce the programmed result. Similarly, any of such autonomous viable or replication competent nanomachines also can be employed in a wide variety of other applications well known to those skilled in the art given the teachings and guidance provided herein.

Briefly, nanomachines can be employed as bioreactors to perform a wide variety of biochemical reactions that are useful for production of compounds and for the treatment of solutions or materials. For example, nanomachines of the invention can be programmed and used in fermentation, for the production of ethanol, for example. Methods and substrates for fermentation are well known in the art. Esterification, methylation and numerous other chemical modifications and processes also can be performed using a nanomachine of the invention as a bioreactor. Given the teachings and guidance provided herein, these and other bioreactor methods well known in the art can be employed using as a substitute for procaryotic or eucaryotic organisms utilized in such methods a nanomachine of the invention.

Additionally, any of the nanomachines of the invention also can be employed in a bioreactor process for the production of a biomolecule of interest. For example, and as described previously, a nanomachine can be programmed to express from one to many different polypeptides, pathways or networks. Overexpression and regulated expression also can be accomplished as described previously to achieve, for example, a desired production of a target polypeptide or polypeptides. Therefore, the level of encoded biomolecule, expression or programmed synthesis from a nanomachine can be modulated depending on the need and targeted application. The biomoleucle of interest can be, for example, a therapeutic polypeptide or polypeptides, a diagnostic polypeptide or other biosynthesizable indicator; or an organic compound. For example, whole or partial biochemical pathways can be expressed by a nanomachine of the invention. The gene products synthesized therefrom can carry out the biosynthesis of various different molecules such as those described previously. Other examples include incorporation of pathways for the synthesis of polyketides, isoprenoids, glycosides, nitrogen fixation, sulfide oxidation, carbon fixation, pesticides, such as pyrrolnitrin, as well as for various physiological responses such as antigen presentation system that can be used in high throughput screens (HTS) screens.

Bioremediation is another useful application of the nanomachines of the invention. For example, the nanomachines can be programmed to perform a wide variety of environmental and industrial remediation activities. Environmental bioremediation activities can include, for example, the treatment of pollutants or waste, such as in an oil spill or contaminated groundwater by the use of a nanomachine programmed to break down the undesirable substances within the contaminant. Similarly, undesirable substances produced, or contained in, an industrial process, including food processing, is an exemplary industrial bioremediation activity for the nanomachines of the invention. A wide variety of other bioremediation activities well known to those skilled in the art are similarly applicable for use with the nanomachines of the inventions. Briefly, to substitute a nanomachine for a microorganism in a bioremediation process, one skilled in the art can incorporate the active genetic components that carry out the remediation process into a basic genetic operating system of a nanomachine. Once the genome has been tailored to a particular bioremediation activity, the nanomachine can be employed in the activity in substantially the same proportions as the original microorganism.

Any of the nanomachines described previously also can be directly or indirectly used for therapeutic applications. Such therapeutic applications can include, for example, expression of a therapeutic molecule at a defined location within an individual and delivery of macromolecules or organic compounds to a defined location within an indiviudal. Nanomachines of the invention also can be used in cell therapy-like applications, for example, where a nanomachine functionally substitutes for a normal cell type or generates a transient or prolonged supply a deficient product. Nanomachines further can be employed to supply a new cellular or molecular activity or operation to an individual that reduces the severity of a pathological condition. All of such therapeutic methods as well as others well known to those skilled in the art are applicable uses for the nanomachines of the invention.

When employed as a delivery system of therapeutic molecules, diagnostic indicators, organic compounds, and various physiological or industrial functions, nanomachines can be programmed, for example, to constitutively produce or regulate the production of the target biomolecule, activity or operation. Such methods of expression have been described previously and are well known to those skilled in the art, including therapeutic, diagnostic or industrial fields.

Artificial tissues or organs can be synthesized by nanomachines of the invention and employed in numerous therapeutic applications. The nanomachine biosynthesis of such structures can be performed, for example, in vivo, in situ or in vitro. For example, nanomachines can be programmed to synthesize, secrete and self-assemble extracelluar matrix polypeptides and other components which can be deposited within a tissue or on a biocompatable substrate. Such structures can be used directly or combined with other components such as growth factors to augment the function of the artificial tissue. The nanomachine produced tissues can be used directly by, for example, production at a targeted site or indirectly by production and transplantation into a targeted site. Similarly, organs such as blood vessels, bone marrow, and liver cell functions can be replicated using nanomachines as a basic cellular building block of these and other tissues. Such tissues can be, for example, produced at the desired site of tissue replacement, repair or supplementation or ex vivo and then transplanted into a recipient individual.

Nanomachines also can be used, for example, as a device to generate, store or convert energy or matter. For example, different forms of energy can be captured or harnessed through known biochemical or physiochemical or pathways and mechanisms. A basic genetic operating system can be programmed to include one or more pathways which can capture, for example, chemical energy or mechanical energy. Nanomachine pathways and components can convert these sources of energy into, for example, high energy molecules for storage, use or subsequent conversion into another energy type. High energy molecules can include, for example, ATP, NAD, NADPH, FAD, and other high energy bond containing molecules. Such molecules can be, for example, converted into other types of matter, used to produce work, or converted into chemical energy, radiant energy such as light or heat, or converted into mechanical energy. Therefore, a nanomachine can be programmed to function equivocally as a cell.

Useful biosynthesizable films and coatings can additionally be produced by any of the nanomachines of the invention described herein. Such films or coatings can be, for example, responsive to environmental changes.

Nanomachines can be further utilized in a wide variety of cosmetic and reconstructive applications. Such cosmetic applications can range from cosmetic or reconstructive surgical uses to exterior beautifying uses. For example, nanomachines of the invention can be employed in reconstructive surgery as supporting biocompatible structures. They can be seeded or grown into a variety of different structures either de novo, for example, or in conjunction of a natural or biocompatible supporting architecture. Such reconstructive prostheses can then be implanted in an individual using various methods well known to those skilled in the art. Cosmetic surgical applications include, for example, any of a variety of implants for augmentation of lips, cheeks, breasts and other anatomical body areas. As beautifying cosmetics or cosmeceuticals, nanomachines of the invention can be engineered to change physical attributes in response to various environmental stimuli. Such stimuli can include, for example, pH, osmolality, temperature and humidity. Attributes that can be modulated in response to such stimuli can include, for example, color, size and odor. Cosmeceuticals can therefore be constructed and used as temporary or permanent cosmetic accessories.

For any of the applications described herein, the use of a nanomachine of the invention will be substantially similar to methods well known to those skilled in the art which employ cells or cellular systems for the same or similar application. Such cells and cellular systems can include, for example, procaryotic cells, simple eucaryotic cells and complex eucaryotic cells. To substitute for a cell or cellular system, a nanomachine of the invention will contain a basic genetic operating system sufficient to support comparable non-replicative or replicative cellular life functions and, if necessary, additional genetic instructions to carry out the comparable activity or operation exhibited by the cognate procaryotic or eucaryotic cell employed in the method. Such a programmed nanomachine is substituted in a cellular or cellular system and treated in substantially the same manner, in comparable amounts and for comparable times as would be the treatment for the replaced cell, for example. Therefore, a nanomachine can be added to a method or used in a method in an effective amount which is sufficient to support a comparable programmed activity from the nanomachine as would occur in a cell or cellular system under substantially the same conditions.

It is understood that modifications which do not substantially affect the activity of the various embodiments of this invention are also included within the definition of the invention provided herein. Accordingly, the following examples are intended to illustrate but not limit the present invention.

### EXAMPLE I

### Design and Synthesis of a Basic Genetic Operation System for a Replication Competent Nanomachine

This Example shows the design and synthesis of a basic genetic operating system for a replication competent autonomous prototrophic nanomachine.

A replication competent nanomachine was engineered using the M. genitalium genome as the genetic source of fundamental genes. Briefly, an autonomous prototrophic basic genetic operating system encoding a minimal gene set that confers replication competence was electronically created from sequence data information available in public databases. The minimal gene set was engineered to contain the 15 functional categories shown in FIG. 2 and in Table 4. Specifically, the functional categories were replication, transcription, translation, aerobic metabolism, glycolysis/pyruvate dehydrogenase/pentose phosphate pathways, carbohydrate metabolism, central intermediary metabolism, nucleotide metabolism, signal transduction regulation, transport and binding proteins, particle division, chaperone system, fatty acid/lipid metabolism, particle envelope and housekeeping functions. Additionally, functional and structural genomic sequences such as an origin of replication were also included in the electronic design, engineering and synthesis. These genomic sequences were similarly derived from the M. genitalium genome.

The design and computer synthesis of the replication competent basic genetic operating system was performed by combining for each fundamental gene a nucleotide sequence corresponding to its mRNA region and required homologous expression elements. Fundamental genes within a functional category, or subgroups within a functional category, were then electronically arranged to produce a gene cassette corresponding to each respective functional category or subgroup within the replication competent basic genetic operating system. Finally, the gene cassettes were then electronically combined, along with other required genomic sequences, to produce the final computerized version of the replication competent autonomous prototrophic basic genetic operating system.

Following computer synthesis, the basic genetic operating system is chemically synthesized. Synthesis is accomplished by first electronically parsing the genome sequence into smaller oligonucleotide sequences that can be more efficiently synthesized. The electronic parsing is performed for both the sense and complementary antisense strands of the basic genetic operating system. Parsing also is performed by maintaining partial complementarity between the 5′ terminus of either the sense or antisense strand and the 3′ terminus of its corresponding complementary sequence so that adjacent oligonucleotides can be annealed with a complementary oligonucleotide to form an overlapping oligonucleotide assembly for both strands that span the genome. The size of each parsed oligonucleotide can vary, but generally, will be between about 50-100 nucleotides (nt) in length with an about 50% overlap between complementary sense and antisense strands.

Following electronic parsing, automated synthesis of the individual oligonucleotides using phosphoramidite oligonucleotide synthesis chemistry is then performed. Automated assembly of the oligonucleotides into the basic genetic operating system is accomplished by sequentially annealing and ligating partially complementary oligonucleotides to result in the complete physical synthesis of the replication competent basic genetic operating system of about 266,433 base pairs (bp) in length. All of the above steps are described in further detail below.

Briefly, the selected fundamental gene sequences were electronically reduced from genomic sequences to their respective mRNA sequences. Alternatively, fundamental gene sequences were electronically reduced to a minimum coding sequence by elimination in some cases, of some or substantially all of a fundamental gene's 5′ or 3′ untranslated region sequence, retaining for example, ribosome binding sites for individual fundamental genes or cistrons when necessary. Because M. genitalium is a procaryotic organism there was no need to include in the electronic reduction removal of intron sequences. The resultant electronic cDNA sequences were then further engineered to include functional expression elements such as promoters, enhancers, suppressors, and other cis acting transcriptional or translational sequences. Such sequences included, for example, at least an upsteam promoter and a ribosome binding site for each gene or cistron and any necessary transcription or translation termination signals.

All 5′ and 3′ expression elements and cis acting sequences were obtain from M. genitalium genomic sequence. The M. genitalium expression elements and cis acting sequences were then operationally linked by computer synthesis to their corresponding fundamental gene within the minimal gene set of the basic genetic operating system. Effectively, inclusion of homologous expression and regulatory sequences was electronically performed by maintaining about 100 nts or the segment defined as the intragenic region between the initiation of the gene and the end of the upstream gene in the 5′ direction. Similarly, about 100 nts or the segment defined as the intragenic region between the termination of the gene and the beginning of the downstream gene in the 3′ direction was maintained in each electronic version of the gene. nt region sequence 3′ to the translation stop codon also was maintained in each electronic version of the gene.

Following computer synthesis of each fundamental gene as described above, the constituent fundamental genes for each functional category or subgroup were electronically organized into a single contiguous sequence or gene cassette. The contiguous sequences for each functional category or subgroup correspond to SEQ ID NOS:1-18. For example, SEQ ID NO:1 shows the about 38,596 nt sequence encoding the 24 fundamental genes within the replication functional category. The genes are ordered in a 5′ to 3′ direction as they are listed in FIG. 2. A complete listing of each functional category or a subgroup thereof, the size of the gene cassette encoding the category or subgroup, the number of included fundamental genes and the corresponding SEQ ID NO is set forth below in Table 1. Except where otherwise indicated, the arrangement of each gene within a functional category or subgroup corresponds to a 5′ to 3′ direction in the gene order listed in FIG. 2.

TABLE 1
Summary of Gene Cassettes for Functional
Categories.

| Functional Category or | Length | Number of | SEQ ID |
|---|---|---|---|
| Subgroup | (nt) | Genes | NUMBER |
| Replication | 38,596 | 24 | 1 |
| Transcription | 22,684 | 14 | 2 |
| Translation-Part I | 38,459 | 25 | 3 |
| Translation-Part II | 7,400 | 4 | 4 |
| Translation-Part III | 11,138 | 13 | 5 |
| Translation-Part IV | 23,272 | 52 | 6 |
| Aerobic Metabolism | 10,809 | 13 | 7 |
| Glycolysis, Pyruvate | 21,247 | 16 | 8 |
| Dehydrogenase & | | | |
| Pentose | | | |
| Phosphate Pathways | | | |
| Carbohydrate Metabolism | 3,075 | 3 | 9 |
| Central Intermediary | 11,899 | 13 | 10 |
| Metabolism | | | |
| Nucleotide Metabolism | 15,051 | 18 | 11 |
| Regulatory Functions | 4,055 | 4 | 12 |
| Transport and Binding | 31,241 | 23 | 13 |
| Particle Division | 4,750 | 4 | 14 |
| Polypeptide Chaperones | 13,894 | 11 | 15 |
| Fatty Acid & | 2,556 | 3 | 16 |
| Phospholipid Metabolism | | | |
| Particle Envelope | 2,601 | 3 | 17 |
| Housekeeping Functions | 3,706 | 4 | 18 |
| Total | 266,433 | 247 | |

To produce the final genome, the above gene cassettes encoding each functional category or subgroup was consecutively arranged in a 5′ to 3′ unidirectional order starting from the origin of replication to yield a single, complete electronic representation of the basic genetic operating system for a replication competent nanomachine. The origin of replication was obtained from pBR322 or from E. coli as a 232 nt region located at positions 4,788,167 to 4,788,398 from Genbank Accession number AE005174. This origin of replication is set forth as SEQ ID NO:19. The above described nanomachine genome can be electronically parsed synthesized and assembled as described further below.

The above-described nanomachine genome represented by SEQ ID NOS:1-18 can be parsed electronically using a computer algorithm and corresponding executable program which generates two sets of overlapping oligonucleotides. For example, the oligonucleotides can be parsed using ParseOligo™, a proprietary computer program that optimizes nucleic acid sequence assembly. Optional steps in sequence assembly can include identifying and eliminating sequences that can give rise to hairpins, repeats or other difficult sequences. Additionally, the algorithm can first direct the synthesis of coding regions for each fundamental gene to correspond to a desired codon preference. For example, coding regions for fundamental genes specify E. coli codon usages instead of M. genitalium codons can be generated. For conversion of a fundamental gene sequence to another codon preference, the algorithm utilizes a polypeptide sequence to generate a DNA sequence using a specified codon table. The algorithm for this step is can be described as follows:

For the DNA sequence GENE[ ], an array of bases, is generated from the protein sequence AA[ ], an array of amino acids, using a specified codon table.

a. parameters

i. N Length of protein in amino acid residues

ii. L=3N Length of gene in DNA bases

iii. Q Length of each component oligonucleotide

iv. X=Q/2 Length of overlap between oligonucleotides

v. W=3N/Q Number of oligonucleotides in the F set

vi. Z=3N/Q+1 Number of oligonucleotides in the R set

vii. F[1:W] set of (+) strand oligonucleotides

viii. R[L:Z] set of (−) strand oligonucleotides

ix. AA[1:N] array of amino acid residues

x. GENE[1:L] array of bases comprising the gene

b. Obtain or design a protein sequence AA[ ] consisting of a list of amino acid residues.

c. Generate the DNA sequence, GENE[ ], from the protein sequence, AA[ ]

i. For I=1 to N

ii. Translate AA[J] from codon table generating GENE[I:I+2]

iii. I=I+3

iv. J=J+1

v. Go to ii

With or without specifying a codon preference for coding regions of fundamental genes, the parsing algorithm can generate a set of parsed oligonucleotides corresponding to the entire length of the sense and antisense stand of the nanomacine genome. The parsing can be performed on the entire genome, on the gene cassettes that constitute functional categories or on shorter fragments thereof, and will depend on the preference of the user. When polymerase chain reaction (PCR) is employed in the assembly process, for example, the parsing is performed on about 10-15 kb fragments of the genome because this size is within the extension range of polymerases used in the procedure. Therefore, parsing the nanomachine genome described above in 10 kb segments would result in 27 different sets of sense and antisense oligonucleotides. These sets can be assembled using the PCR method described below and then ligated together to yield the completed basic genetic operating system. The parsing algorithm can be described as follows:

Two sets of overlapping oligonucleotides are generated from GENE[ ]; F[ ] covers the sense strand and R[ ] is a complementary, partially overlapping set covering the antisense strand.

a. Generate the F[ ] set of oligos

i. For I=1 to W

ii. F[I]−GENE [I:I+Q−1]

iii. I=I+Q

iv. Go to ii

b. Generate the R set of oligos

i. J=W

ii. For I=1 to W

iii. R[I] GENE [W:W−Q]

iv. J=J−Q

v. Go to iii

c. Result is two set of oligos F[ ] and R[ ] of Q length

d. Generate the final two finishing oligos

i. S[1]=GENE [Q/2:1]

ii. S[2]=GENE [L−Q/2:L]

Following parsing into two sets of overlapping, partially complementary oligonucleotides, which represent the complete basic genetic operating system of the nanomachine, the oligonucleotides are then synthesized. In this regard, the computer output of the parsed set of oligonucleotides for both the sense and antisense strand of the nanomachine genome can be transferred to oligonucleotide synthesizer driver software. The synthesis of sequences of about 25 to 150 nt in length can be manufactured and assembled using the array synthesizer system and can be used without further purification. For example, two 96-well plates containing 100 nt oligonucleotides can yield a 9600 bp fragment of a gene cassette. Therefore, synthesis of an entire basic genetic operating system for the above replication competent nanomachine can be performed using about 28 pairs of 96 well plates. Once synthesized, the individual oligonucleotides can be maintained in the original plates or transferred to new multi-well format plates for oligonucleotide assembly.

Assembly can be accomplished using, for example, robotics or microfluidics well known in the art for manipulating large numbers of oligonucleotide samples. Robotics and microfluidics allow synthesis and assembly to be performed rapidly and in a highly controlled manner. Such methods are described, for example, in WO 99/14318 and in U.S. Application Serial Nos. 60/262,693 and 09/922,221.

For example, oligonucleotide parsing from the genome sequence designed in the computer can be programmed for synthesis where sense and antistrands are placed in alternating wells of an array. Following synthesis in this format, the 12 row sequences of the gene are directed into a pooling manifold that systematically pools three wells into reaction vessels forming the triplex structure. Following temperature cycling for annealing and ligation, four sets of annealed triplex oligonucleotides are pooled into 2 sets of 6 oligonucleotide products, then 1 set of 12 oligonucleotide products. Each row of the synthetic array is associated with a similar manifold resulting in the first stage of assembly of 8 sets of assembled oligonucleotides representing 12 oligonucleotides each. The second manifold pooling stage is controlled by a single manifold that pools the 8 row assemblies into a single complete assembly. Passage of the oligonucleotide components through the two manifold assemblies (the first 8 and the second single) results in the complete assembly of all 96 oligonucleotides from the array. The assembly module of Genewriter™ can include a complete set of 7 pooling manifolds produced using microfabrication in a single plastic block that sits below the synthesis vessels. Various configurations of the pooling manifold will allow assembly of 96,384 or 1536 well arrays of parsed component oligonucleotides. A similar strategy can be performed where pairs of oligonucleotides are pooled instead of triplets.

An algorithm which can be implemented in a computer program for assembly of oligonucleotides as described above can be described as follows:

Two sets of oligonucleotides F[1:W] R[l:Z] S[1:2]

Step 1

a. For I=1 to W

b. Anneal F[I], F[I+1], R[I]; place in T[I]

c. Anneal F[I+2], R[I+1], R[I+2] T[I+1]

d. I=I+3

e. Go to b

Step 2

a. Do the following until only a single reaction remains

i. For I=1 to W/3

ii. Ligate T[I], T[I+1]

iii. I=I+2

iv. Go to ii

Described further below is the assembly of parsed oligonucleotides corresponding to the basic genetic operating system described above following array synthesis of the oligonucleotide sets using a multi-well format. The method additionally employs polymerase chain reaction (PCR) in a two-step procedure to facilitate assembly.

Arrayed sets of parsed overlapping oligonucleotides are obtained by robotic instruments. Each oligonucleotide consists of 50 nts with an overlap of about 25 base pairs (bp). The oligonucleotide concentration is from 250 nM (250 μM/ml). 50 base oligos give Tₘs from 75 to 85 degrees C., 6 to 10 od₂₆₀, 11 to 15 nanomoles, 150 to 300 μg. Resuspend in 50 to 100 μl of H₂O to make 250 nM/ml. Equal amounts of each oligonucleotide are combined to a final concentration of 250 μM (250 nM/ml) by adding 1 μl of each to give 192 μl. Addition of 8 μl dH₂O follows to bring the volume up to 200 μl and a final concentration of 250 μM mixed oligos. The mixture is diluted 250-fold by taking 10 μl of mixed oligos and add to 1 ml of water (1/100; 2.5 mM ) followed by transferring 1 μl of this mixture into 24 μl 1×PCR mix. The PCR reaction includes: 10 mM TRIS-HCl, pH 9.0; 2.2 MM MgCl₂; 50 mM KCl; 0.2 mM each dNTP, and 0.1% Triton X-100. One U TaqI polymerase is added to the reaction. The reaction is thermoycled under the following conditions for assembly: 55 cycles of (1) 94 degrees 30 s; (2) 52 degrees 30s, and (3) 72 degrees 30s.

Following assembly amplification, 2.5 μl of the assembly mix is added to 100 μl of PCR mix (40×dilution). Outside primers are prepared by taking 1 μl of F1 (forward primer) and 1 μl of R96 (reverse primer) at 250 μM (250 nm/ml-0.250 nmole/μl) and adding to the 100 μl PCR reaction. This mixture provides a final concentration of 2.5 μM each oligo. Taq1 polymerase is added (1U) and the reaction is thermocycle under the following conditions: 35 cycles (or original protocol 23 cycles) for (1) 94 degrees for 30s; (2) 50 degrees for 30s, and (3) 72 degrees for 60s. The product is extract with phenol/chloroform, precipitate with ethanol and the pellet is resuspended in 10 μl of dH₂O and analyze on an agarose gel.

An alternative method for assembly of parsed oligonucleotides corresponding to the basic genetic operating system described above following array synthesis of oligonucleotide sets is provided below. The method assembles parsed oligonucleotides using a Taq1 ligation procedure.

Briefly, arrayed sets of parsed overlapping oligonucleotides of about 25 to 150 bases in length each, with an overlap of about 12 to 75 base pairs (bp), are obtained. The oligonucleotide concentration is from 250 nM (250 μM/ml). For example, 50 base oligos give Tₘs from 75 to 85 degrees C., 6 to 10 od₂₆₀, 11 to 15 nanomoles, 150 to 300 μg. The oligonucleotides are resuspended in 50 to 100 ml of H₂O to make 250 nM/ml.

Using a robotic workstation, for example, a Beckman Biomek automated pipetting robot, or another automated lab workstation, equal amounts of forward and reverse oligonucleotides are combined pairwise. Equal volumes (10 μl) of forward and reverse oligonucleotides are mixed in a new 96-well v-bottom plate to provide one array with sets of duplex oligonucleotides at 250 μM, according to pooling scheme Step 1 in Table 2. An assembly plate is prepared by taking 2 μl of each oligomer pair and adding to a fresh plate containing 100 μl of ligation mix in each well. This procedure gives an effective concentration of 2.5 μM or 2.5 nM/ml. From each well of these wells, 20 μl is transferred to a fresh microwell plate and 1 μl of T4 polynucleotide kinase and 1 μl of 1 mM ATP subsequently added to each well. Each reaction will have 50 pmoles of oligonucleotide and 1 nmole ATP. The reactions are incubated at 37 degrees C. for 30 minutes.

Initiation of assembly is performed according to Steps 2-7 of Table 2. For example, pooling Step 2 is performed by mixing each successive well with the next. Taq1 ligase (1 μl) is then added to each mixed well and the mixture is cycled once at 94 degrees for 30 sec; 52 degrees for 30s; then 72 degrees for 10 minutes.

Further assembly is performed according to step 3 of Table 2 of the pooling scheme and cycle according to the temperature scheme described above. Similarly, steps 4 and 5 of the pooling scheme are subsequently performed for further assembly and also cycled according to the temperature scheme above. Subsequent performance of step 6 of the pooling scheme is accomplished by transferring 10 μl of each mix into a fresh microwell and step 7 of the pooling scheme is accomplished by pooling the remaining three wells. The reaction volumes for each of these step within the pooling scheme will be:

Initial plate has 20 ul per well.

Step 2 20 ul+20 ul=40 ul

Step 3 80 ul

Step 4 160 ul

Step 5 230 ul

Step 6 10 ul+10 ul=20 ul

Step 7 20+20+20=60 ul final reaction volume

A final PCR amplification is then performed by taking 2 ul of final ligation mix and add to 20 ul of PCR mix containing 10 mM TRIS-HCl, pH 9.0, 2.2 mM MgCl₂, 50 mM KCl, 0.2 mM each dNTP and 0.1% Triton X-100.

The outside primers are prepared by taking 1 μl of F1 (forward primer) and 1 μl of R96 (reverse primer) at 250 μM (250 nm/ml-0.250 nmole/μl) and add to the 100 μl PCR reaction giving a final concentration of 2.5 uM each oligo. Add 1 U Taq1 polymerase and cycle for 35 cycles under the following conditions: 94 degrees for 30s; 50 degrees for 30s; and 72 degrees for 60s. The mixture is extracted with phenol/chloroform and precipitated with ethanol. The pellet is resuspend in 10 μl of dH₂O and analyze on an agarose gel.

TABLE 2
Pooling scheme for ligation assembly.
Ligation method - Well pooling scheme

| STEP | FROM | TO |
|---|---|---|
| 1 | All F | All R |
| 2 | A1 | A2 |
| | A3 | A4 |
| | A5 | A6 |
| | A7 | A8 |
| | A9 | A10 |
| | A11 | A12 |
| | B1 | B2 |
| | B3 | B4 |
| | B5 | B6 |
| | B7 | B8 |
| | B9 | B10 |
| | B11 | B12 |
| | C1 | C2 |
| | C3 | C4 |
| | C5 | C6 |
| | C7 | C8 |
| | C9 | C10 |
| | C11 | C12 |
| | D1 | D2 |
| | D3 | D4 |
| | D5 | D6 |
| | D7 | D8 |
| | D9 | D10 |
| | D11 | D12 |
| | E1 | E2 |
| | E3 | E4 |
| | E5 | E6 |
| | E7 | E8 |
| | E9 | E10 |
| | E11 | E12 |
| | F1 | F2 |
| | F3 | F4 |
| | F5 | F6 |
| | F7 | F8 |
| | F9 | F10 |
| | F11 | F12 |
| | G1 | G2 |
| | G3 | G4 |
| | G5 | G6 |
| | G7 | G8 |
| | G9 | G10 |
| | G11 | G12 |
| | H1 | H2 |
| | H3 | H4 |
| | H5 | H6 |
| | H7 | H8 |
| | H9 | H10 |
| | H11 | H12 |
| 3 | A2 | A4 |
| | A6 | A8 |
| | A10 | A12 |
| | B2 | B4 |
| | B6 | B8 |
| | B10 | B12 |
| | C2 | C4 |
| | C6 | C8 |
| | C10 | C12 |
| | D2 | D4 |
| | D6 | D8 |
| | D10 | D12 |
| | E2 | E4 |
| | E6 | E8 |
| | E10 | E12 |
| | F2 | F4 |
| | F6 | F8 |
| | F10 | F12 |
| | G2 | G4 |
| | G6 | G8 |
| | G10 | G12 |
| | H2 | H4 |
| | H6 | H8 |
| | H10 | H12 |
| 4 | A4 | A8 |
| | A12 | B4 |
| | B8 | B12 |
| | C4 | C8 |
| | C12 | D4 |
| | D8 | D12 |
| | E4 | E8 |
| | E12 | F4 |
| | F8 | F12 |
| | G4 | G8 |
| | G12 | H4 |
| | H8 | H12 |
| 5 | A8 | B4 |
| | B12 | C8 |
| | D4 | D12 |
| | E8 | F4 |
| | F12 | G8 |
| | H4 | H12 |
| 6 | B4 | C8 |
| | D12 | F4 |
| | G8 | H12 |
| 7 | C8 | F4 |

Another alternative method for assembly of parsed oligonucleotides corresponding to the basic genetic operating system described above following array synthesis of oligonucleotide sets is additionally described below. This method assembles parsed oligonucleotides using a TaqI synthesis and stepwise assembly.

Briefly, arrayed sets of parsed overlapping oligonucleotides of about 25 to 150 bases in length each, with an overlap of about 12 to 75 base pairs (bp), are obtained as described above and resuspended in 50 to 100 ml of H₂O to make 250 nM/ml. Similarly, manipulations of samples is performed using robotics as described previously.

Two working multi-well plates containing forward and reverse oligonucleotides in a PCR mix at 2.5 mM are prepared and 1 μl of each oligo are added to 100 μl of PCR mix in a fresh microwell providing one plate of forward and one of reverse oligos in an array. Cycling assembly is then initiated as follows according to the pooling scheme outlined in Table 3. In the present example, 96 cycles of assembly can be accomplished according to this scheme.

To begin assembly, 2 μl of oligonucleotides in well F-E1 is transferred to a fresh well. Similarly, 2 μl of oligonucleotides in well R-E1 is transferred to a fresh well and 18 μl of 1×PCR mix and 1 U of Taq1 polymerase are added. The mixture is cycled once under the following conditions: (1) 94 degrees for 30 s; (2) 52 degrees for 30 s, and (3) 72 degrees for 30 s. Subsequently, 2 μl of oligonucleotides from well F-E2 and from well R-D12 is transferred to the reaction vessel. The mixture is cycled once according to the temperatures conditions described above. The pooling and cycling is repeated according to the scheme outlined in Table 3 for about 96 cycles.

A PCR amplification is then performed by taking 2 μl of final reaction mix and adding it to 20 μl of a PCR mix comprising: 10 mM TRIS-HCl, pH 9.0; 2.2 mM MgCl₂; 50 mM KCl; 0.2 mM each dNTP, and 0.1% Triton X-100.

Outside primers are prepared by taking 1 μl of F1 and 1 ml of R96 at 250 mM (250 nm/ml-0.250 nmole/ml) and adding to the above 100 μl PCR reaction. This procedure yields a final concentration of 2.5 μM each oligonucleotide. 1 U Taq1 polymerase is subsequently added and the reaction is cycled for about 23 to 35 cycles under the following conditions: (1) 94 degrees for 30s; (2) 50 degrees for 30s, and (3) 72 degrees for 60s. The reaction is subsequently extracted with phenol/chloroform, precipitated with ethanol and resuspend in 10 ml of dH2O for analysis on an agarose gel.

For initial pooling of the oligonucleotides, equal amounts of forward and reverse oligonucleotide pairs are added by taking 10 μl of forward and 10 μl of reverse oligonucleotide and mixing in a new 96-well v-bottom plate. This procedure provides one array with sets of duplex oligonucleotides at 250 mM, according to pooling scheme Step 1 in Table 3. An assembly plate is prepared by taking 2 μl of each oligomer pair and adding them to the plate containing 100 μl of ligation mix in each well. This gives an effective concentration of 2.5 μM or 2.5 nM/ml. About 20 μl of each well is transferred to a fresh microwell plate in addition to 1 μl of T4 polynucleotide kinase and 1 μl of 1 mM ATP. Each reaction will have 50 pmoles of oligonucleotide and 1 nmole ATP. The reaction is incubated at 37 degrees for 30 minutes.

Nucleic acid assembly was initiated according to Steps 2-7 of Table 3. For step 2, pooling is carried out by mixing each well with the next well in succession. Specifically, 1 μl of Taq1 ligase to is added to each mixed well and cycled once as follows: (1) 94 degrees for 30 sec; (2) 52 degrees for 30s, and (3) 72 degrees 10 minutes.

Subsequently, step 3 of pooling scheme is carried out and cycled according to the temperature scheme described above. In like manner, steps 4 and 5 of the pooling scheme are then carried out and cycled according to the temperature scheme above. Step 6 of the pooling scheme is performed by taking 10 μl of each mix into a fresh microwell. Pooling the remaining three wells completes performance of step 7 of the pooling scheme. The reaction volumes will be (initial plate has 20 μl per well):

Step 2 20 μl+20 μl=40 μl

Step 3 80 μl

Step 4 160 μl

Step 5 230 μl

Step 6 10 μl+10 μl=20 μml

Step 7 20+20+20=60 μl final reaction volume

Following completion of the steps described above, a final PCR amplification is performed by taking 2 μl of the final ligation mix and adding it to 20 μl of PCR mix comprising: 10 mM TRIS-HCl, pH 9.0; 2.2 mM MgCl2; 50 mM KCl; 0.2 mM each dNTP, and 0.1% Triton X-100.

Outside primers are prepared by taking 1 μl of F1 and 1 μl of R96 at 250 mM (250 nm/ml-0.250 nmole/ml) and adding them to the above PCR reaction above giving a final concentration of 2.5 uM for each oligonucleotide. Subsequentlly, 1 U of Taq1 polymerase is added and cycled for about 23 to 35 cycles under the following conditions: (1) 94 degrees for 30s; (2) 50 degrees for 30s, and (3) 72 degrees for 60s. The product is extracted with phenol/chloroform, precipitate with ethanol, resuspend in 10 μl of dH2O and analyzed on an agarose gel.

TABLE 3
Pooling scheme for assembly using
Taq1 polymerase (also topoisomerase II).

| Step | Forward oligo | | | | Reverse oligo | | | |
|---|---|---|---|---|---|---|---|---|
| 1 | F | E | 1 | + | R | E | 1 | Pause |
| 2 | F | E | 2 | + | R | D | 12 | Pause |
| 3 | F | E | 3 | + | R | D | 11 | Pause |
| 4 | F | E | 4 | + | R | D | 10 | Pause |
| 5 | F | E | 5 | + | R | D | 9 | Pause |
| 6 | F | E | 6 | + | R | D | 8 | Pause |
| 7 | F | E | 7 | + | R | D | 7 | Pause |
| 8 | F | E | 8 | + | R | D | 6 | Pause |
| 9 | F | E | 9 | + | R | D | 5 | Pause |
| 10 | F | E | 10 | + | R | D | 4 | Pause |
| 11 | F | E | 11 | + | R | D | 3 | Pause |
| 12 | F | E | 12 | + | R | D | 2 | Pause |
| 13 | F | F | 1 | + | R | D | 1 | Pause |
| 14 | F | F | 2 | + | R | C | 12 | Pause |
| 15 | F | F | 3 | + | R | C | 11 | Pause |
| 16 | F | F | 4 | + | R | C | 10 | Pause |
| 17 | F | F | 5 | + | R | C | 9 | Pause |
| 18 | F | F | 6 | + | R | C | 8 | Pause |
| 19 | F | F | 7 | + | R | C | 7 | Pause |
| 20 | F | F | 8 | + | R | C | 6 | Pause |
| 21 | F | F | 9 | + | R | C | 5 | Pause |
| | F | F | 10 | + | R | C | 4 | |
| 22 | | | | | | | | Pause |
| 23 | F | F | 11 | + | R | C | 3 | Pause |
| 24 | F | F | 12 | + | R | C | 2 | Pause |
| 25 | F | G | 1 | + | R | C | 1 | Pause |
| 26 | F | G | 2 | + | R | B | 12 | Pause |
| 27 | F | G | 3 | + | R | B | 11 | Pause |
| 28 | F | G | 4 | + | R | B | 10 | Pause |
| 29 | F | G | 5 | + | R | B | 9 | Pause |
| 30 | F | G | 6 | + | R | B | 8 | Pause |
| 31 | F | G | 7 | + | R | B | 7 | Pause |
| 32 | F | G | 8 | + | R | B | 6 | Pause |
| 33 | F | G | 9 | + | R | B | 5 | Pause |
| 34 | F | G | 10 | + | R | B | 4 | Pause |
| 35 | F | G | 11 | + | R | B | 3 | Pause |
| 36 | F | G | 12 | + | R | B | 2 | Pause |
| 37 | F | H | 1 | + | R | B | 1 | Pause |
| 38 | F | H | 2 | + | R | A | 12 | Pause |
| 39 | F | H | 3 | + | R | A | 11 | Pause |
| 40 | F | H | 4 | + | R | A | 10 | Pause |
| 41 | F | H | 5 | + | R | A | 9 | Pause |
| 42 | F | H | 6 | + | R | A | 8 | Pause |
| 43 | F | H | 7 | + | R | A | 7 | Pause |
| 44 | F | H | 8 | + | R | A | 6 | Pause |
| 45 | F | H | 9 | + | R | A | 5 | Pause |
| 46 | F | H | 10 | + | R | A | 4 | Pause |
| | F | H | 11 | + | R | A | 3 | Pause |
| 47 | | | | | | | | |
| 48 | F | H | 12 | + | R | A | 2 | Pause |

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

TABLE 4
ORTHOLOGOUS FUNDAMENTAL GENES

| | | | |
|---|---|---|---|
| | | | EUCARYOTIC |
| M. genitalium | H. influenza | E. coli | (NCBI Accession Identification |

Replication

| | | | | | |
|---|---|---|---|---|---|
| MG001 | DNA Polymerase III | 0410 | DNA Pol III, beta chain | dnaN | |
| MG003 | DNA gyrase | 1688 | DNA gyrase, subunit B | gyrB | BAA33955 (Candida) |
| MG004 | DNA gyrase | 0672 | DNA gyrase, subunit A | gyrA | P30182 (Arabidopsis) |
| MG073 | Excinuclease ABC | 0656 | Excinuclease helicase | uvrB | T86424 (Human) |
| MG091 | ss DNA Binding Protein | 1384 | ssDNA binding protein | ssb | P32445 (Saccharomyces) |
| MG094 | Replicative DNA helicase | 0971 | Replicative helicase | dnaB | |
| MG097 | DNA uracil glycosylase | 1155 | Uracil-DNA glycosylase | ung | DDU32866 (Dictyostelium) |
| MG122 | DNA topoisomerase I | 0768 | DNA topoisomerase I | topA | P13099 (Saccharomyces) |
| MG203 | DNA topoisomerase IVsub | 0929 | DNA topoisomerase IV sub | parE | P41001 (Plasmodium) |
| MG204 | DNA topoisomerase IVsub | 0930 | DNA topoisomerase IV sub | parC | X74738 (Saccharomyces) |
| MG206 | Excinuclease ABC | 1194 | Excinuclease nuclease sub | uvrC | |
| MG244 | DNA helicase II | 0069 | DNA helicase | rep | HJBYDH (Saccharomyces) |
| MG250 | DNA primase | 1654 | DNA primase | dnaG | |
| MG254 | DNA ligase | 0512 | DNA ligase | lig | |
| MG259 | FKBP-like peptidylprolyl isomerase | 0961 | Adenyne-specific DNA methylase | hemK | U12141 (Saccharomyces) |
| MG261 | DNA Pol III | 0155 | DNA Pol III alpha subunit | dnaE | |
| MG262a | Formamidopyrimidine-DNA | 0362 | Formamidopyrimidine-DNA glycosylase | mutM | |
| | glycosylase | | | | |
| MG339 | Recombination protein | 0017 | Rec A | recA | L15229 (Arabidopsis) |
| MG358 | Holliday junction DNA helicase | 1445 | Holliday junction DNA helicase subunit | ruvA | |
| MG359 | Holliday junction DNA helicase | 1444 | Holliday junction DNA helicase subunit | ruvB | M96757 (Plasmodium) |
| MG379 | FAD binding protein | 1703 | FAD-utilizing enzyme | gidA | JU0182 (Cucumis) |
| MG420 | DNA Pol III sub | | | dnaXp | CAA91237 (Schizosaccharomyces) |
| MG421 | Excinuclease ABC | 1383 | Excinuclease ATPase sub | uvrA | CAC02927 (Leishmania) |
| MG469 | Chromosomal replication inhibitor | 0411 | Chromosomal replication initiator ATPase | dnaA | |

Transcription

| | | | | | |
|---|---|---|---|---|---|
| MG054 | Transcription elongation and | 0132 | Transcription antiterminator | nusG | |
| | termination factor | | | | |
| MG104 | RNase | 0278 | Exoribonuclease | vacB | P37202 (Schizosaccharomyces) |
| MG141 | N-utilzation substance protein | 0689 | Transcription factor | nusA | |
| MG177 | RNA pol | 0219 | DNA-directed RNA Pol alpha subunit | rpoA | P07703 (Saccharomyces) |
| MG209 | Pseudouridylate synthase | 1539 | PseudoU synthetase | yceC | Q09709 (Schizosaccharomyces) |
| MG249 | RNA pol sigma A factor | 1655 | RNA pol sigma-70 factor | rpoD | |
| MG278 | guanosine-3′,5′-bis(diphosphate) | 1135 | ppGpp 3′pyrophosphohydrolase | spoT | |
| | 3′-pyrophophohydrolase (transcriptional | | | | |
| | regulator) | | | | |
| MG340 | RNA polymerase | 1636 | DNA-directed RNA pol beta-prime | rpoC | P36594 (Schizosaccharomyces) |
| MG341 | RNA polymerase | 1637 | DNA-directed RNA pol beta-subunit | rpoB | P38420 (Arabidopsis) |
| MG346 | rRNA methyltransferase (SpoU family) | 0182 | rRNA methylase (SpoU family) | yibK | |
| MG367 | Ribonuclease III | 1151 | Ribonuclease III | rnc | XP_015448 (Human) |
| MG425 | ATP-dependent RNA helicase | 1369 | RNA helicase | deaD | P19109 (Drosophila) |
| MG463 | rRNA (adenosine-N6,N6-)- | 1671 | Dimethyladenosine transferase | ksgA | P41819 (Saccharomyces) |
| | dimethyltransferase | | | | |
| MG465 | Rnase P C5 sub | 0416 | RNase P protein component | rnpA | |

Translation - Part I Amino acyl tRNA synthetases, tRNA modification and amino acid metabolism.

| | | | | | |
|---|---|---|---|---|---|
| MG005 | Ser-tRNA Synthase | 1248 | seryl-tRNA synthetase | serS | CAB61772 (Schizosaccharomyces) |
| MG021 | Met-tRNA Synthase | 0683 | methionine—tRNA synthetase | metG | P22438 (Saccharomyces) |
| MG035 | His-tRNA Synthase | 1495 | histidine—tRNA synthetase | hisS | CAA94983 (Saccharomyces) |
| MG036 | Asp-tRNA Synthase | 1449 | aspartyl-tRNA synthetase | aspS | P14868 (Human) |
| MG083 | Peptidyl-tRNA Hydrolase | 1521 | peptidyl-tRNA hydrolase | pth | Q59989 (Synechocystis) |
| MG113 | Asn-tRNA Synthase | 0707 | asparagine—tRNA synthetase | asnS | P38707 (Saccharomyces) |
| MG126 | Trp-tRNA Synthase | 0057 | tryptophanyl-tRNA synthetase | trpS | YWBYM (Saccharomyces) |
| MG136 | Lys-tRNA Synthase | 0620 | lysyl-tRNA synthetase | lysU | P37879 (Cricetulus) |
| MG182 | Pseudouridylate Synthase | 1038 | pseudoU synthetase I | truA | P31115 (Saccharomyces) |
| MG194 | Phe-tRNA Synthase | 0716 | phenylalanyl-tRNA synthetase alpha chain | pheS | AAB51175 (Human) |
| MG195 | Phe-tRNA Synthase | 0717 | phenylalanyl-tRNA synthetase beta chain | pheT | |
| MG251 | Gly-tRNA Synthase | | | thrSp | P52709 (Caenorhabditis) |
| MG253 | Cys-tRNA Synthase | 1215 | cysteinyl-tRNA synthetase | cysS | AAG00579 (Human) |
| MG266 | Leu-tRNA Synthase | 0337 | leucyl-tRNA synthetase | leuS | P41252 (Human) |
| MG283 | Pro-tRNA Synthase | | | proSp | P26639 (Human) |
| MG292 | Ala-tRNA Synthase | 0231 | alanyl-tRNA synthetase | alaS | P21894 (Bombyx) |
| MG334 | Val-tRNA Synthase | 0797 | valyl-tRNA synthetase | valS | BG099272 (Human) |
| MG336 | Pyridoxal-dependent | 0700 | aminotransferase | | |
| | aminnotransferase | | | | |
| MG345 | Ile-tRNA Synthase | 0378 | isoleucyl-tRNA synthetase | ileS | P09436 (Saccharomyces) |
| MG365 | Met-tRNA Synthase | 0043 | methionyl-tRNA formyltransferase | fmt | P28037 (Rattus) |
| MG375 | Thr-tRNA Synthase | 0770 | threonyl-tRNA synthetase | thrS | P04801 (Saccharomyces) |
| MG378 | Arg-tRNA Synthase | 0977 | arginyl-tRNA synthetase | argS | AAK68226 (Caenorhabditis) |
| MG445 | tRNA (guanine-N1)-Mtase | 1336 | tRNA (guanine-N1)-methyltransferase | trmD | NP_014647 (Saccharomyces) |
| MG455 | Tyr-tRNA Synthase | 1003 | tyrosyl-tRNA synthetase | tyrS | Q09692 (Schizosaccharomyces) |
| MG462 | Glu-tRNA Synthase | 1408 | glutamyl-tRNA synthetase | gltX | P13188 (Saccharomyces) |

Translation - Part II Degradation and folding of polypeptides

| | | | | | |
|---|---|---|---|---|---|
| MG238 | Trigger factor | 0128 | peptidyl-prolyl cis-trans isomerase | tig | P20081 (Saccharomyces) |
| MG239 | ATP-dependent protease | 1588 | ATP-dependent protease | lon | |
| MG355 | ATP-dependent protease binding sub | 0276 | ATP-dependent ClpB protease ATPase | clpB | CAB38512 (Schizosaccharomyces) |
| MG391 | Aminopeptidase | 1098 | leucyl aminopeptidase | pepA | Q09735 (Schizosaccharomyces) |

Translation - Part III Polypeptide modification and translation factors

| | | | | | |
|---|---|---|---|---|---|
| MG026 | Elongation factor P | 1457 | Elongation factor P | efp | |
| MG089 | Elongation factor G | 1700 | Translation elongation factor G | fusA | P32324 (Saccharomyces) |
| MG106 | Formylmethionine deformylase | 0042 | N-formylmethionylaminoacyl-tRNA | def | |
| | | | deformylase | | |
| MG142 | Protein synthesis initiation factor 2 | 0690 | Translation initiation factor IF-2, GTPase | infB | NP_009531 (Saccharomyces) |
| MG143 | Ribosome-binding factor | 0694 | Ribosome-binding protein | rbfA | |
| MG172 | Methionine amino peptidase | 1114 | Methionine aminopeptidase | map | |
| MG173 | Initiation factor 1 | 1670 | Translation initiation factor IF-1 | infA | |
| MG196 | Translation initiation factor IF3 | 0723 | Initiation factor 3 | infC | |
| MG258 | Peptide chain release factor 1 | 0963 | Peptide chain release factor 1 | prfA | |
| MG282 | Transcription elongation factor | 0734 | Transcription elongation factor | greA | |
| MG433 | Elongation factor | 0330 | Translation elongation factor Ts | tsf | |
| MG435 | Ribosome releasing factor | 0225 | Ribosome releasing factor | frr | NP_011903 (Saccharomyces) |
| MG451 | Elongation factor TU | 0052 | UDP-n-acetylglucosamine | tufA | Q00080 (Plasmodium) |
| | | | pyrophosphorylase | | |

Translation - Part IV Ribosome synthesis & modification

| | | | | | |
|---|---|---|---|---|---|
| MG012 | Ribosomal prt S6 modification | 0932 | Ribosomal prt S6 modification | rimK | |
| MG070 | Ribosomal prt S2 | 0329 | Ribosomal prt S2 | rpsB | |
| MG081 | Ribosomal prt L11 | 1639 | 50S Ribosomal prt L11 | rplK | P17079 (Saccharomyces) |
| MG082 | Ribosomal prt L1 | 1638 | Ribosomal prt L1 | rplA | P96038 (Sulfolobus) |
| MG087 | Ribosomal prt S12 | 1702 | 30S Ribosomal prt S12 | rpsL | CAB97965 (Leishmania) |
| MG088 | Ribosomal prt S7 | 1701 | 30S Ribosomal prt S7 | rpsG | |
| MG090 | Ribosomal prt S6 | 1669 | 30S Ribosomal prt S6 | rpsF | P15938 (Saccharomyces) |
| MG092 | Ribosomal prt S18 | 1667 | 30S Ribosomal prt S18 | rpsR | |
| MG093 | Ribosomal prt L9 | 1666 | 50S Ribosomal prt L9 | rplI | |
| MG150 | Ribosomal prt S10 | 0192 | 30S Ribosomal prt S10 | rpsJ | P35686 (Oryza) |
| MG151 | Ribosomal prt L3 | 0193 | 50S Ribosomal prt L3 | rplC | P34113 (Dictyostelium) |
| MG152 | Ribosomal prt L4 | 0194 | 50S Ribosomal prt L4 | rplD | P12735 (Haloarcula) |
| MG153 | Ribosomal prt L23 | 0195 | 50S Ribosomal prt L23 | rplW | S78414 (Rattus) |
| MG154 | Ribosomal prt L2 | 0196 | Ribosomal prt L22 | rplB | P41569 (Aedes) |
| MG155 | Ribosomal prt S19 | 0197 | Ribosomal prt S19 | rpsS | P39697 (Arabidopsis) |
| MG156 | Ribosomal prt L22 | 0198 | 50S Ribosomal prt L22 | rplV | |
| MG157 | Ribosomal prt S3 | 0199 | Ribosomal prt S3 | rpsC | P05750 (Saccharomyces) |
| MG158 | Ribosomal prt L16 | 0200 | 50S Ribosomal prt L16 | rplP | T38231 (Schizosaccharomyces) |
| MG159 | Ribosomal prt L29 | 0201 | 50S Ribosomal prt L29 | rpmC | P42766 (Human) |
| MG160 | Ribosomal prt S17 | 0202 | Ribosomal prt S17 | rpsQ | Z46260 (Saccharomyces) |
| MG161 | Ribosomal prt L14 | 0204 | 50S Ribosomal prt L14 | rplN | AAK18863 (Caenorhabditis) |
| MG162 | Ribosomal prt L24 | 0205 | 50S Ribosomal prt L24 | rplX | |
| MG163 | Ribosomal prt L5 | 0206 | 50S Ribosomal prt L5 | rplE | NP_015194 (Saccharomyces) |
| MG164 | Ribosomal prt S14 | 0207 | 30S Ribosomal prt S14 | rpsN | P10633 (Saccharomyces) |
| MG165 | Ribosomal prt S8 | 0208 | 30S Ribosomal prt S8 | rpsH | P39027 (Human) |
| MG166 | Ribosomal prt L6 | 0209 | 50S Ribosomal prt L6 | rplF | CAA91503 (Schizosaccharomyces) |
| MG167 | Ribosomal prt L18 | 0210 | 50S Ribosomal prt L18 | rplR | |
| MG168 | Ribosomal prt S5 | 0211 | 30S Ribosomal prt S5 | rpsE | P05753 (Saccharomyces) |
| MG169 | Ribosomal prt L15 | 0213 | 50S Ribosomal prt L15 | rplO | |
| MG174 | Ribosomal prt L36 | 0215 | 50S Ribosomal prt L36 | rpmJ | |
| MG175 | Ribosomal prt S13 | 0216 | Ribosomal prt S13 | rpsM | |
| MG176 | Ribosomal prt S11 | 0217 | Ribosomal prt S11 | rpsK | Q08699 (Podocoryne) |
| MG178 | Ribosomal prt L17 | 0220 | 50S Ribosomal prt L17 | rplQ | P22353 (Saccharomyces) |
| MG197 | Ribosomal prt L35 | 0724 | 50S Ribosomal prt L35 | rpml | |
| MG198 | Ribosomal prt L20 | 0725 | 50S Ribosomal prt L20 | rplT | |
| MG232 | Ribosomal prt L21 | 0297 | 50S Ribosomal prt L21 | rplU | |
| MG234 | Ribosomal prt L27 | 0296 | 50S Ribosomal prt L27 | rpmA | |
| MG252 | rRNA methylase | 0277 | rRNA methylase (SpoU family) | yjfH | S48881 (Saccharomyces) |
| MG257 | Ribosomal prt L31 | 0174 | 50S ribosomal protein L31 | rpmE | |
| MG311 | Ribosomal prt S4 | 0218 | ribosomal protein S4 | rpsD | CAA18654 (Schizosaccharomyces) |
| MG325 | Ribosomal prt L33 | 0367 | ribosomal protein L33 | rpmG | |
| MG361 | Ribosomal prt L10 | 0060 | Ribosomal protein L10 | rplJ | |
| MG362 | Ribosomal prt L7/L12 | 0061 | Ribosomal protein L7/L12 | rplL | P05387 (Human) |
| MG363 | Ribosomal prt L32 | 1292 | Ribosomal protein L32 | rpmF | |
| MG363a | Ribosomal prt S20 | 0381 | 30S ribosomal protein S20 | rpsT | |
| MG417 | Ribosomal prt S9 | 0847 | 30S ribosomal protein S9 | rpsI | CAA21965 (Candida) |
| MG418 | Ribosomal prt L13 | 0848 | Ribosomal protein L13 | rplM | P39473 (Sulfolobus) |
| MG424 | Ribosomal prt S15 | 0732 | Ribosomal protein S15 | rpsO | CAC37508 (Schizosaccharomyces) |
| MG426 | Ribosomal prt L28 | 0368 | Ribosomal protein L28 | rpmB | |
| MG444 | Ribosomal prt L19 | 1335 | Ribosomal protein L19 | rplS | |
| MG446 | Ribosomal prt S16 | 1338 | 30S ribosomal protein S16 | rpsP | U33335 (Saccharomyces) |
| MG466 | Ribosomal prt L34 | 0415 | 50S ribosomal protein L34 | rpmH | |

Aerobic Metabolism

| | | | | | |
|---|---|---|---|---|---|
| MG102 | Thioredoxin reductase | 0570 | Thioredoxin | trxB | NP_010640 (Saccharomyces) |
| MG124 | Thioredoxin | 1221 | Thioredoxin | trxA | P38141 (Saccharomyces) |
| MG145 | FAD synthase | 0379 | Nucleotidyltransferase | yaaC | NP_010522 (Saccharomyces) |
| MG275 | NADH Oxidase | | | lpdp | P09623 (Sus) |
| MG398 | ATP Synthase epsilon chain | 1603 | ATP synthase F1 epsilon subunit | atpC | |
| MG399 | ATP Synthase beta chain | 1604 | H+-transporting ATPase beta-subunit | atpD | P48413 (Cyanidium) |
| MG400 | ATP Synthase gamma chain | 1605 | ATP synthase F1 gamma subunit | atpG | |
| MG401 | ATP Synthase alpha chain | 1606 | ATP synthase F1 alpha subunit | atpA | P48413 (Cyanidium) |
| MG402 | ATP Synthase delta chain | 1607 | ATP synthase F1 delta subunit | atpH | |
| MG403 | ATP Synthase B chain | 1608 | ATP synthase F0 subunit b | atpF | |
| MG404 | ATP Synthase C chain | 1609 | H+-transporting ATP synthase C chain | atpE | |
| MG405 | Adenosinetriphosphatase | 1610 | ATP synthase F0 subunit a | atpB | |
| MG408 | peptide methionine sulfoxide | | | msrA | NP_010960 (Saccharomyces) |
| | reductase | | | | |

Glycolysis, Pyruvate Dehydrogenase & Pentose Phosphate Pathways

| | | | | | |
|---|---|---|---|---|---|
| MG023 | Fructose-bisphosphate aldolase | | | gatY | P14540 (Saccharomyces) |
| MG063 | 1-phoshofructokinase | 1573 | 1-phosphofructokinase | fruK | P25332 (Saccharomyces) |
| MG066 | Transketolase 1 (TK 1) | 0439 | Transketolase 2 | tkt | P23254 (Saccharomyces) |
| MG069 | Phosphotransferase enzyme IIABC | | | crr | S74697 (Synechocystis) |
| MG111 | Phosphoglucose isomerase B | 0973 | Glucose-6-phosphate isomerase | pgi | NP_009755 (Saccharomyces) |
| MG215 | 6-phosphofructokinase | 0400 | 6-phosphofructokinase | pfkA | P16861 (Saccharomyces) |
| MG216 | Pyruvate kinase | 0970 | Pyruvate kinase | pykA | NP_014992 (Saccharomyces) |
| MG271 | Dihydrolipoamide Dehydrogenase | 0640 | Dihydrolipamide dehydrogenase | lpd | P09624 (Saccharomyces) |
| MG272 | Dihydrolipoamide acetyltransferase | 0641 | Dihydrolipoamide acetyltransferase E2 | aceF | P10515 (Human) |
| | | | component | | |
| MG273 | Pyruvate Dehydrogenase E-1beta sub | | | | U09137 (Arabidopsis) |
| MG274 | Pyruvate Dehydrogenase E-1alpha sub | | | | NP_000047 (Human) |
| MG300 | Phosphoglycerate kinase | 1647 | Phosphoglycerate kinase | pgk | Q27685 (Leishmania) |
| MG301 | Glyceraldehyde 3-phosphate | 1138 | Glyceraldehyde 3-phosphate | gapA | P00359 (Saccharomyces) |
| | dehydrogenase | | dehydrogenase | | |
| MG407 | Enolase | 0348 | Enolase | eno | U09194 (Mesembryanthemum) |
| MG430 | Phosphoglycerate mutase | | | yibO | NP_013374 (Saccharomyces) |
| MG431 | Triosephosphate isomerase | 0096 | Triosephosphate isomerase | tpiA | Q07412 (Plasmodium) |

Carbohydrate Metabolism

| | | | | | |
|---|---|---|---|---|---|
| MG050 | deoxyribose-phosphate aldolase | 0528 | Deoxyribose-phosphate aldolase | deoC | AAK68302 (Caenorhabditis) |
| MG053 | phosphomannomutase | 0740 | Phosphomannomutase | yhbF | NP_014005 (Saccharomyces) |
| MG112 | D-ribose-5-phosphate 3 epimerase | 1370 | Lytic transglycosylase | yfhD | NP_012414 (Saccharomyces) |

Central Intermediary Metabolism

| | | | | | |
|---|---|---|---|---|---|
| MG013 | 5,10-methylene-tetrahydrofolate | 0027 | 5,10-methylene-tetrahydrofolate | folD | Q04448 (Drosophila) |
| | dehydrogenase | | dehydrogenase | | |
| MG038 | Glycerol kinase | 0108 | Glycerol kinase | glpK | S36175 (Human) |
| MG047 | S-adenosylmethionine synthetase | 0584 | S-adenosylmethionine synthetase II | metX | NP_013281 (Saccharomyces) |
| MG222 | SAM-dependent methyltransferase | 0542 | SAM-dependent methyltransferase | yabC | |
| MG228 | Dihydrofolate reductase | 0316 | Dihydrofolate reductase | folA | U03885 (Paramecium) |
| MG245 | 5,10-methenyltetrahydrofolate synthase | 0275 | 5-formyltetrahydrofolate cyclo-ligase | ygfA | P11586 (Human) |
| MG293 | Glcerophospphoryl diester | 0106 | Glcerophospphoryl diester | glpQ | |
| | phosphodiesterase | | phosphodiesterase | | |
| MG299 | Phosphotransacetylase | 0612 | Phosphotransacetylase | ptap | P38503 (Methanosarcina) |
| MG347 | SAM-dependent methyltransferase | 1469 | SAM-dependent methyltransferase | yggH | |
| MG351 | Inorganic pyrophosphatase | 1555 | Inorganic Pyrophosphatase | Ppap/ | P28239 (Saccharomyces) |
| | | | | ppa | |
| MG357 | Acetate kinase | 0613 | Acetate kinase | ackA | |
| MG380 | SAM-dependent methyltransferase | 1611 | Glucose-inhibited division protein, | gidB | P38892 (Saccharomyces) |
| | | | methyltransferase | | |
| MG394 | Serine hydroxymethyltransferase (folate | 0306 | Serine hydroxymethyltransferase | glyA | P37291 (Saccharomyces) |
| | cycle) | | | | |

(Nucleotide Metabolism:
Purines, Pyrimidines, Nucleosides, and Nucleotides

| | | | | | |
|---|---|---|---|---|---|
| MG006 | Thymidylate kinase | 1582 | Pyrimidine kinase | ycfG | AAC73211 (Human) |
| MG030 | Uracil Phophoribosyltransferase | 0637 | Uracil phosphoribosyl transferase | upp | U10246 (Toxoplasma) |
| MG049 | Purine-nucleoside phophorylase | 1640 | Purine-nucleoside phophorylase | deoD | BC003788 (Mus) |
| MG052 | Cytidine deaminase | 0753 | Cytidine deaminase | Cddp/ | P32320 (Human) |
| | | | | odd | |
| MG058 | Phophoribosylpyrophosphate Synthase | 1002 | Ribose-phosphate pyrophosphokinase | prsA | P38689 (Saccharomyces) |
| MG107 | 5′-guanylate kinase | 1137 | Guanylate kinase | gmk | KIBYGU (Saccharomyces) |
| MG118 | UDP-glucose 4-epimerase | 1480 | UDP-glucose 4-epimerase | galE | P04397 (Saccharomyces) |
| MG171 | Adenylate kinase | 1478 | Adenylate kinase | adk | P26364 (Saccharomyces) |
| MG227 | Thymidylate Synthase | 0321 | Thymidylate Synthase | thyA | U03885 (Paramecium) |
| MG229 | Ribonucleotide Reductase 2 | 1054 | Ribonucleoside-diphosphate reductase, | nrdB | P42170 (Caenorhabditis) |
| | | | beta chain | | |
| MG231 | Ribonucleoside-diphosphate Reductase | 1053 | Ribonucleoside-diphosphate reductase | nrdA | CAB72517 (Campylobacter) |
| MG268 | Deoxyguano-deoxyadeno kinase (I) | | | | |
| | sub 2 | | | | |
| MG276 | Adenine Phophoribosyltransferase | 0639 | Adenine phosphoribosyltransferase | apt | TAU22442 (Triticum) |
| MG330 | Cytidylate kinase | 0628 | Cytidylate kinase | cmk | U10120 (Mus) |
| MG382 | Uridine kinase | 1266 | Uridine kinase | udk | L31784 (Mus) |
| MG434 | uridylate kinase | 0479 | Uridine 5′-monophosphate kinase | pyrH | P37142 (Daucuc) |
| MG453 | UDP-glucose pyrophosphorylase | 0229 | Glucosephosphate uridylyltransferase | galU | P32501 (Saccharomyces) |
| MG458 | Hypoxanthine-guanine | 0565 | Hypoxanthine phosphoribosyltransferase | hpt | P00492 (Human) |
| | Phophoribosyltrnsfrse | | | | |

Regulatory Functions

| | | | | | |
|---|---|---|---|---|---|
| MG024 | GTPase | 1520 | GTPase | ychF | P38746 (Saccharomyces) |
| MG335 | GTPase | 0530 | GTPase | yihA | |
| MG384 | GTPase | 0294 | GTPase | yhbZ | P38860 (Saccharomyces) |
| MG387 | GTPase | 1150 | GTP-binding protein | era | P32559 (Saccharomyces) |

Transport and Binding Polypeptides

| | | | | | |
|---|---|---|---|---|---|
| MG015 | Transport ATPase | | | msbAp | P34712 (Caenor habditis) |
| MG033 | Glycerol uptake facilitator (permease) | 0107 | Glycerol uptake facilitator | glpF | CAB69639 (Schizosaccharomyces) |
| MG042 | Spermidine-putrescine transport | 0750 | Spermidine/putrescine transport ATPase | potA | CAA17820 (Schizosaccharomyces) |
| ATP-BP | | | | | |
| MG043 | Spermidine-putrescine transport | 0749 | Spermidine/putrescine permease | potB | |
| | permease | | | | |
| MG044 | Spermidine-putrescine transport | 0748 | Spermidine/putrescine permease | potC | |
| permease | | | | | |
| MG045 | Spermidine/putrescine periplasmic | 0747 | Spermidine/putrescine-binding periplasmic | potD | |
| | binding | | protein | | |
| MG065 | Transport ATPase | | | | |
| MG071 | Cation-transporting ATPase | | | | |
| MG077 | Oligopeptide transport permease | 0535 | Oligopeptide permease | oppB | |
| MG078 | Oligopeptide transport permease | 0534 | Oligopeptide permease | oppC | |
| MG079 | Oligopeptide transport ATP-BP | 0533 | Oligopeptide transport ATPase | oppD | P33311 (Saccharomyces) |
| MG080 | Oligopeptide transport ATP-BP | 0532 | Oligopeptide transport ATPase | oppF | P33311 (Saccharomyces) |
| MG119 | Carbohydrate Transport ATPase | 0240 | Galactoside transport ATPase | mglA | CAC00467 (Leishmania) |
| MG120 | Sugar permease/ribose transport | 1625 | D-ribose ABC transporter | rbsCp | CAC08238 (Schizosaccharomyces) |
| | permease | | | | |
| MG180 | Amino acid transport prt | 0593 | Dipeptide transport ATPase | dppF | S51433 (Saccharomyces) |
| MG187 | Glycerol-3-phosphate transport ATPase | | | ugpC | P21449 (Cricetulus) |
| MG247 | Permease | 1400 | Membrane protein | ygiH | |
| MG270 | Lipoate-protein ligase | | | lplA | NP_012489 (Saccharomyces) |
| MG287 | Acyl-carrier protein | 1288 | Acyl carrier protein | acpP | ASYP (Spinacia) |
| MG322 | Na+ ATPase subunit J | | | | |
| MG333 | Acyl carrier protein phosphodiesterase | 0769 | Acyl carrier protein phosphodiesterase | acpD | |
| MG410 | Phosphate transport ATPase | 0784 | Phosphate transport ATPase | pstB | P13568 (Plasmodium) |
| MG411 | Phosphate permease | 0785 | Phosphate permease | pstA | |

Particle Division

| | | | | | |
|---|---|---|---|---|---|
| MG224 | Cell division protein | 0555 | Cell division, GTPase | ftsZ | P29516 (Arabidopsis) |
| MG297 | Cell division protein | 0184 | Cell division, signal recognition particle | ftsY | P20424 (Saccharomyces) |
| | | | GTPase | | |
| MG353 | DNA-binding protein | | | | |
| MG457 | Cell division protein | 0737 | ATP-Zn dependent protease | ftsH | P39925 (Saccharomyces) |

Polypeptide Chaperones

| | | | | | |
|---|---|---|---|---|---|
| MG019 | Heat shock protein | 0647 | DnaJ chaperone | dnaJ | NP_014335 (Saccharomyces) |
| MG048 | Signal recognition particle GTPase | 1244 | Signal recognition particle GTPase | ffh | P37107 (Arabidopsis) |
| MG055 | Preprotein translocase subunit | 0131 | Preprotein translocase subunit | secE | |
| MG072 | Preprotein translocase | 0325 | Preprotein translocase, putative helicase | secA | Q06461 (Antithamnion) |
| MG138 | GTP-binding membrane protein | 1153 | Membrane GTPase | lepA | P34617 (Caenorhabditis) |
| MG170 | Preprotein translocase | 0214 | Preprotein translocase subunit | secY | |
| MG201 | Heat shock protein | 1209 | Heat shock protein | grpE | CAA17799 (Caenorhabditis) |
| MG210 | Prolipoprotein signal peptidase | 0422 | Lipoprotein signal peptidase | lspA | |
| MG305 | Heat shock protein | 0646 | DnaK Chaperone | dnaK | P41753 (Achlya) |
| MG392 | Heat shock protein | 1665 | GroEL Chaperone | groL | P40413 (Saccharomyces) |
| MG393 | Heat shock protein | 1664 | GroEL Co-Chaperone | groS | |

Fatty Acid and Phospholipid Metabolism

| | | | | | |
|---|---|---|---|---|---|
| MG114 | Phospatidylglycerophosphate Synthase | 1260 | Phospatidylglycerophosphate Synthase | pgsA | P06197 (Saccharomyces) |
| MG212 | 1-acyl-sn-glycerol-3-phos | 0149 | 1-acyl-sn-glycerol-3-phos acetyltransferase | plsC | P33333 (Saccharomyces) |
| | acetyltransferase | | | | |
| MG437 | CDP-diglyceride Synthase | 0335 | CDP-diglyceride Synthase | cdsA | NP_009585 (Saccharomyces) |

Particle Envelope

| | | | | |
|---|---|---|---|---|
| MG059 | LPS-heptosyl-2-transferase | 0399 | Complement SmpB | smpB |
| MG060 | Lipopolysachharide biosyn protein | | | yibDp |
| | motif | | | |
| MG086 | Prolipoprotein diacylglyceryl | | | lgtp |
| | transferase | | | |

Housekeeping Function

| | | | | | |
|---|---|---|---|---|---|
| MG125 | Hydrolase | 1140 | Hydrolase | yidA | |
| MG265 | Hydrolase | 0013 | Hydrolase | yigL | NP_011974 (Saccharomyces) |
| MG295 | ATP-utilizing enzyme (GuaA family) | 1308 | ATP-utilizing enzyme | ycfB | P00966 (Human) |
| MG383 | NH3, ATP-dependent NAD synthetase | | | proSp | CAA19255 (Schizosaccharomyces) |

What is claimed is:

1. A basic genetic operating system for an autonomous prototrophic nanomachine comprising a nanomachine genome encoding a minimal gene set sufficient for viability.

2. The basic genetic operating system of claim 1, wherein said minimal gene set further comprises the functional categories of transcription, translation, aerobic metabolism, glycolysis/pyruvate dehydrogenase/pentose phosphate pathways, carbohydrate metabolism, central intermediary metabolism, nucleotide metabolism, transport and binding proteins, and housekeeping functions.

3. The basic genetic operating system of claim 2, wherein said nanomachine genome directs synthesis of said functional categories in a relative order comprising transcription, translation, aerobic metabolism and glycolysis/pyruvate dehydrogenase/pentose phosphate pathways.

4. The basic genetic operating system of claim 3, wherein said relative order further comprises a relative temporal order.

5. The basic genetic operating system of claim 3, wherein said relative order further comprises a relative physical order.

6. The basic genetic operating system of claim 1, further comprising a minimal gene set being devoid of at least one gene selected from the group consisting of MG008, MG009, MG056, MG221, MG332, MG448 or MG449, an ortholog or a nonorthologous gene displacement thereof.

7. The basic genetic operating system of claim 1, wherein said nanomachine genome further comprises less than about 140 kilobases (kb) in size.

8. The basic genetic operating system of claim 1, wherein said minimal gene set sufficient for viability further comprises about 152 or less fundamental genes.

9. The basic genetic operating system of claim 8, wherein said fundamental genes further comprise about 14 genes in a transcription gene category, about 90 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 3 genes in a central intermediary metabolism gene category, about 2 genes in a nucleotide metabolism gene category, about 10 genes in a transport/binding protein gene category and about 1 genes in a housekeeping function gene category.

10. The basic genetic operating system of claim 8, wherein said about 152 or less fundamental genes further comprise substantially the same fundamental genes show in FIG. 1, orthologs or nonothorologous displacements thereof.

11. The basic genetic operation system of claim 1, further comprising one or more genes selected from a replication gene category.

12. The basic genetic operation system of claim 1, further comprising one or more genes selected from the group consisting of a translation gene category, a central intermediary metabolism category, a nucleotide metabolism gene category, a phosphotransferase system (PTS) gene category, a signal transduction regulation gene category, a transport/binding protein gene category, a particle division gene category, a chaperone system gene category, a fatty acid/lipid metabolism gene category, a particle envelope gene category and a housekeeping function gene category.

13. The basic genetic operating system of claim 1, further comprising an expression control region for the production of a biomolecule.

14. The basic genetic operating system of claim 13, wherein said biomolecule further comprises an RNA.

15. The basic genetic operating system of claim 13, wherein said biomolecule further comprises a polypeptide.

16. An autonomous prototrophic nanomachine comprising a basic genetic operating system for autonomous prototrophic viability and a particle envelope.

17. The autonomous prototrophic nanomachine of claim 16, wherein said particle envelope further comprises a membrane.

18. The autonomous prototrophich nanomachine of claim 16, wherein said particle envelope further comprises a biocompatible material.

19. The autonomous prototrophic nanomachine of claim 16, wherein said basic genetic operating system further comprises an expression control region for the production of a biomolecule.

20. The autonomous prototrophic nanomachine of claim 19, wherein said biomolecule further comprises an RNA.

21. The autonomous prototrophic nanomacnine of claim 19, wherein said biomolecule further comprises a polypeptide.

22. A basic genetic operating system for an autonomous auxotrophic nanomachine comprising a nanomachine genome encoding a minimal gene set sufficient for viability in the presence of an auxotrophic biomolecule.

23. The basic genetic operating system of claim 22, wherein said minimal gene set further comprises the functional categories of transcription, translation, aerobic metabolism, glycolysis/pyruvate dehydrogenase/pentose phosphate pathways, carbohydrate metabolism, central intermediary metabolism, nucleotide metabolism, transport and binding proteins, and housekeeping functions.

24. The basic genetic operating system of claim 23, wherein said nanomachine genome directs synthesis of said functional categories in a relative order comprising transcription, translation, aerobic metabolism and glycolysis/pyruvate dehydrogenase/pentose phosphate pathways.

25. The basic genetic operating system of claim 24, wherein said relative order further comprises a relative temporal order.

26. The basic genetic operating system of claim 24, wherein said relative order further comprises a relative physical order.

27. The basic genetic operating system of claim 22, further comprising a minimal gene set being devoid of at least one gene selected from the group consisting of MG008, MG009, MG056, MG221, MG332, MG448 or MG449, an ortholog or a nonorthologous gene displacement thereof.

28. The basic genetic operating system of claim 22, wherein said nanomachine genome further comprises less than about 140 kilobases (kb) in size.

29. The basic genetic operating system of claim 22, wherein said minimal gene set sufficient for viability further comprises about 151 or less fundamental genes.

30. The basic genetic operating system of claim 29, wherein said fundamental genes further comprise at least one nonfunctional gene selected from a minimal gene set of fundamental genes consisting of about 14 genes in a transcription gene category, about 90 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 3 genes in a central intermediary metabolism gene category, about 2 genes in a nucleotide metabolism gene category, about 10 genes in a transport/binding protein gene category and about 1 genes in a housekeeping function gene category.

31. The basic genetic operating system of claim 29, wherein said about 151 or less fundamental genes further comprise substantially the same fundamental genes show in FIG. 1, orthologs or nonothorologous displacements thereof.

32. The basic genetic operation system of claim 22, further comprising one or more genes selected from a replication gene category.

33. The basic genetic operation system of claim 22, further comprising one or more genes selected from the group consisting of a translation gene category, a central intermediary metabolism category, a nucleotide metabolism gene category, a phosphotransferase system (PTS) gene category, a signal transduction regulatio gene category, a transport/binding protein gene category, a particle division gene category, a chaperone system gene category, a fatty acid/lipid metabolism gene category, a particle envelope gene category and a housekeeping function gene category.

34. The basic genetic operating system of claim 22, further comprising an expression control region for the production of a biomolecule.

35. The basic genetic operating system of claim 34, wherein said biomolecule further comprises an RNA.

36. The basic genetic operating system of claim 34, wherein said biomolecule further comprises a polypeptide.

37. An autonomous auxotrophic nanomachine comprising a basic genetic operating system for autonomous auxotrophic viability in the presence of an auxotrophic biomolecule and a particle envelope.

38. The autonomous auxotrophic nanomachine of claim 37, wherein said particle envelope further comprises a membrane.

39. The autonomous auxotrophich nanomachine of claim 37, wherein said particle envelope further comprises a biocompatible material.

40. The autonomous auxotrophic nanomachine of claim 37, wherein said basic genetic operating system further comprises an expression control region for the production of a biomolecule.

41. The autonomous auxotrophic nanomachine of claim 40, wherein said biomolecule further comprises an RNA.

42. The autonomous auxotrophic nanomacnine of claim 40, wherein said biomolecule further comprises a polypeptide.

43. A basic genetic operating system for an autonomous prototrophic nanomachine comprising a nanomachine genome encoding a minimal gene set sufficient for autonomous prototrophic replication, said nanomachine genome directing synthesis of said minimal gene set in a relative order of functional categories comprising replication, transcription, translation, aerobic metabolism and glycolysis/pyruvate dehydrogenase/pentose phosphate pathways.

44. The basic genetic operating system of claim 43, wherein said functional categories of said minimal gene set further comprise carbohydrate metabolism, central intermediary metabolism, nucleotide metabolism, signal transduction regulation, transport and binding proteins, particle division, chaperone system, fatty acid/lipid metabolism, particle envelope and housekeeping functions.

45. The basic genetic operating system of claim 43, wherein said relative order further comprises a relative temporal order.

46. The basic genetic operating system of claim 43, wherein said relative order further comprises a relative physical order.

47. The basic genetic operating system of claim 46, wherein said relative physical order further comprises relative to an origin of replication.

48. The basic genetic operating system of claim 43, further comprising a bidirectional order.

49. The basic genetic operating system of claim 43, further comprising an expression control region for the production of a biomolecule.

50. A basic genetic operating system for an autonomous protrophic nanomachine comprising a nanomachine genome encoding a minimal gene set sufficient for directing autonomous prototrophic replication, said minimal gene set being devoid of at least one gene selected from the group consisting of MG008, MG009, MG056, MG221, MG262, MG332, MG448 or MG449, an ortholog or a nonorthologous gene displacement thereof.

51. The basic genetic operating system of claim 50, wherein said minimal gene set further comprises the functional categories of replication, transcription, translation, aerobic metabolism, glycolysis/pyruvate dehydrogenase/pentose phosphate pathways, carbohydrate metabolism, central intermediary metabolism, nucleotide metabolism, signal transduction regulation, transport and binding proteins, particle division, chaperone system, fatty acid/lipid metabolism, particle envelope and housekeeping functions.

52. The basic genetic operating system of claim 50, further comprising one or more genes selected from the group consisting of MG020, MG022, MG034, MG039, MG041, MG046, MG051, MG061, MG062, MG108, MG121, MG129, MG183, MG188, MG368, MG429 an ortholog or a nonorthologous gene displacement thereof.

53. The basic genetic operating system of claim 50, further comprising an expression control region for the production of a biomolecule.

54. A basic genetic operating system for an autonomous prototropic nanomachine comprising a nanomachine genome encoding a minimal gene set sufficient for directing autonomous prototrophic replication, said nanomachine genome being less than about 250 kilobases (kb) in size.

55. The basic genetic operating system of claim 54, wherein said minimal gene set further comprises functional categories selected from the group consisting of replication, transcription, translation, aerobic metabolism, glycolysis/pyruvate dehydrogenase/pentose phosphate pathways, carbohydrate metabolism, central intermediary metabolism, nucleotide metabolism, signal transduction regulation, transport and binding proteins, particle division, chaperone system, fatty acid/lipid metabolism, particle envelope and housekeeping functions.

56. The basic genetic operating system of claim 54 further comprising about 247 or less fundamental genes.

57. The basic genetic operating system of claim 56, wherein said fundamental genes further comprise about 24 genes in a replication gene category, about 14 genes in a transcription gene category, about 94 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 13 genes in a central intermediary metabolism gene category, about 18 genes in a nucleotide metabolism gene category, about 4 genes in a signal transduction regulation gene category, about 23 genes in a transport/binding protein gene category, about 4 genes in a particle division gene category, about 11 genes in a chaperone system gene category, about 3 genes in a fatty acid/lipid metabolism gene category, about 3 genes in a particle envelope gene category, and about 4 genes in a housekeeping function gene category.

58. The basic genetic operating system of claim 56, wherein said about 247 or less fundamental genes further comprise substantially the same fundamental genes show in FIG. 2, orthologs or nonothorologous displacements thereof.

59. The basic genetic operating system of claim 57, further comprising one or more genes selected from the group consisting of a translation gene category, a transcription gene category, a nucleotide metabolism gene category, a phosphotransferase system (PTS) gene category, and a fatty acid/lipid metabolism gene category.

60. The basic genetic operating system of claim 59, further comprising one or more genes selected from the group consisting of MG020, MG022, MG034, MG039, MG041, MG046, MG051, MG061, MG062, MG108, MG121, MG129, MG183, MG188, MG368, MG429, an ortholog or a nonorthologous gene displacement thereof.

61. The basic genetic operating system of claim 54, further comprising an expression control region for the production of a biomolecule.

62. A basic genetic operating system for an autonomous prototrophic nanomachine comprising a nanomachine genome encoding a minimal gene set sufficient for autonomous prototrophic replication of about 247 or less fundamental genes.

63. The basic genetic operating system of claim 62 wherein said fundamental genes further comprise about 24 genes in a replication gene category, about 14 genes in a transcription gene category, about 94 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 13 genes in a central intermediary metabolism gene category, about 18 genes in a nucleotide metabolism gene category, about 4 genes in a signal transduction regulation gene category, about 23 genes in a transport/binding protein gene category, about 4 genes in a particle division gene category, about 11 genes in a chaperone system gene category, about 3 genes in a fatty acid/lipid metabolism gene category, about 3 genes in a particle envelope gene category, and about 4 genes in a housekeeping function gene category.

64. The basic genetic operating system of claim 62, wherein said about 247 or less fundamental genes further comprise substantially the same fundamental genes show in FIG. 2, orthologs or nonothorologous displacements thereof.

65. The basic genetic operating system of claim 62, further comprising one or more genes selected from the group consisting of a translation gene category, a transcription gene category, a nucleotide metabolism gene category, a phosphotransferase system (PTS) gene category, and a fatty acid/lipid metabolism gene category.

66. The basic genetic operating system of claim 63, further comprising one or more genes selected from the group consisting of MG020, MG022, MG034, MG039, MG041, MG046, MG051, MG061, MG062, MG108, MG121, MG129, MG183, MG188, MG368, MG429, ortholog or nonorthologous gene displacement thereof.

67. The basic genetic operating system of claim 62, further comprising an expression control region for the production of a biomolecule.

68. An autonomous prototrophic nanomachine comprising a basic genetic operating system for autonomous prototrophic replication and a particle envelope.

69. The autonomous prototrophic nanomachine of claim 68, wherein said particle envelope further comprises a membrane.

70. The autonomous prototrophic nanomachine of claim 68, wherein said particle envelope further comprises a biocompatible material.

71. The autonomous prototrophic nanomachine of claim 68, wherein said basic genetic operating system further comprises an expression control region for the production of a biomolecule.

72. The autonomous prototrophic nanomachine of claim 71, wherein said biomolecule further comprises an RNA.

73. The autonomous prototrophic nanomacnine of claim 71, wherein said biomolecule further comprises a polypeptide.

74. A basic genetic operating system for an autonomous auxotrophic nanomachine comprising a nanomachine genome encoding a minimal gene set sufficient for autonomous replication in the presence of an auxotrophic biological molecule, said nanomachine genome directing synthesis of said minimal gene set in a relative order of functional categories comprising replication, transcription, translation, aerobic metabolism and glycolysis/pyruvate dehydrogenase/pentose phosphate pathways.

75. The basic genetic operating system of claim 74, wherein said other functional categories of said minimal gene set further comprise carbohydrate metabolism, central intermediary metabolism, nucleotide metabolism, signal transduction regulation, transport and binding proteins, particle division, chaperone system, fatty acid/lipid metabolism, particle envelope and housekeeping functions.

76. The basic genetic operating system of claim 74, wherein said relative order further comprises a relative temporal order.

77. The basic genetic operating system of claim 74, wherein said relative order further comprises a relative physical order.

78. The basic genetic operating system of claim 77, wherein said relative physical order further comprises relative to an origin of replication.

79. The basic genetic operating system of claim 74, further comprising a bidirectional order.

80. The basic genetic operating system of claim 74, further comprising an expression control region for the production of a biomolecule.

81. A basic genetic operating system for an autonomous auxotrophic nanomachine comprising a nanomachine genome encoding a minimal gene set sufficient for directing autonomous replication in the presence of an auxotrophic biological molecule, said minimal gene set being devoid of at least one gene selected from the group consisting of MG008, MG009, MG056, MG221, MG262, MG332, MG448 or MG449, an ortholog or a nonorthologous gene displacement thereof.

82. The basic genetic operating system of claim 81, wherein said minimal gene set further comprises the functional categories of replication, transcription, translation, aerobic metabolism, glycolysis/pyruvate dehydrogenase/pentose phosphate pathways, carbohydrate metabolism, central intermediary metabolism, nucleotide metabolism, signal transduction regulation, transport and binding proteins, particle division, chaperone system, fatty acid/lipid metabolism, particle envelope and housekeeping functions.

83. The basic genetic operating system of claim 81, further comprising one or more genes selected from the group consisting of MG020, MG022, MG034, MG039, MG041, MG046, MG051, MG061, MG062, MG108, MG121, MG129, MG183, MG188, MG368, MG429, an ortholog or a nonorthologous gene displacement thereof.

84. The basic genetic operating system of claim 81, further comprising an expression control region for the production of a biomolecule.

85. A basic genetic operating system for an autonomous auxotrophic nanomachine comprising a nanomachine genome encoding a minimal gene set sufficient for directing autonomous auxotrophic replication in the presence of an auxotrophic biological molecule, said nanomachine genome being less than about 250 kilobases (kb) in size.

86. The basic genetic operating system of claim 85, wherein said minimal gene set further comprises functional categories selected from the group consisting of replication, transcription, translation, aerobic metabolism, glycolysis/pyruvate dehydrogenase/pentose phosphate pathways, carbohydrate metabolism, central intermediary metabolism, nucleotide metabolism, signal transduction regulation, transport and binding proteins, particle division, chaperone system, fatty acid/lipid metabolism, particle envelope and housekeeping functions.

87. The basic genetic operating system of claim 85, further comprising about 246 or less fundamental genes.

88. The basic genetic operating system of claim 87, wherein said fundamental genes further comprise at least one nonfunctional gene selected from a minimal gene set of fundamental genes consisting of about 24 genes in a replication gene category, about 14 genes in a transcription gene category, about 94 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 13 genes in a central intermediary metabolism gene category, about 18 genes in a nucleotide metabolism gene category, about 4 genes in a signal transduction regulation gene category, about 23 genes in a transport/binding protein gene category, about 4 genes in a particle division gene category, about 11 genes in a chaperone system gene category, about 3 genes in a fatty acid/lipid metabolism gene category, about 3 genes in a particle envelope gene category, and about 4 genes in a housekeeping function gene category.

89. The basic genetic operating system of claim 87, wherein said about 246 or less fundamental genes further comprise substantially the same fundamental genes show in FIG. 2, orthologs or nonothorologous displacements thereof.

90. The basic genetic operating system of claim 88, further comprising one or more genes selected from the group consisting of a translation gene category, a transcription gene category, a nucleotide metabolism gene category, a phosphotransferase system (PTS) gene category, and a fatty acid/lipid metabolism gene category.

91. The basic genetic operating system of claim 90, further comprising one or more genes selected from the group consisting of MG020, MG022, MG034, MG039, MG041, MG046, MG051, MG061, MG062, MG108, MG121, MG129, MG183, MG188, MG368, MG429, an ortholog or a nonorthologous gene displacement thereof.

92. The basic genetic operating system of claim 85, further comprising an expression control region for the production of a biomolecule.

93. A basic genetic operating system for an autonomous auxotrophic nanomachine comprising a nanomachine genome encoding a minimal gene set sufficient for autonomous replication in the presence of an auxotrophic biological molecule of about 246 or less fundamental genes.

94. The basic genetic operating system of claim 93, wherein said fundamental genes further comprise about 24 genes in a replication gene category, about 14 genes in a transcription gene category, about 94 genes in a translation gene category, about 13 genes in an aerobic metabolism gene category, about 16 genes in a glycolysis/pyruvate dehydrogenase/pentose phosphate pathways gene category, about 3 genes in a carbohydrate metabolism gene category, about 13 genes in a central intermediary metabolism gene category, about 18 genes in a nucleotide metabolism gene category, about 4 genes in a signal transduction regulation gene category, about 23 genes in a transport/binding protein gene category, about 4 genes in a particle division gene category, about 11 genes in a chaperone system gene category, about 3 genes in a fatty acid/lipid metabolism gene category, about 3 genes in a particle envelope gene category, and about 4 genes in a housekeeping function gene category.

95. The basic genetic operating system of claim 93, wherein said about 246 or less fundamental genes further comprise substantially the same fundamental genes show in FIG. 2, orthologs or nonothorologous displacements thereof.

96. The basic genetic operating system of claim 93, further comprising one or more genes selected from the group consisting of a translation gene category, a transcription gene category, a nucleotide metabolism gene category, a phosphotransferase system (PTS) gene category, and a fatty acid/lipid metabolism gene category.

97. The basic genetic operating system of claim 94, further comprising one or more genes selected from the group consisting of MG020, MG022, MG034, MG039, MG041, MG046, MG051, MG061, MG062, MG108, MG121, MG129, MG183, MG188, MG368, MG429, ortholog or nonorthologous gene displacement thereof.

98. The basic genetic operating system of claim 93, further comprising an expression control region for the production of a biomolecule.

99. An autonomous auxotrophic nanomachine comprising a basic genetic operating system for autonomous replication in the presence of an auxotrophic biological molecule and a particle envelope.

100. The autonomous auxotrophic nanomachine of claim 99, wherein said particle envelope further comprises a membrane.

101. The autonomous auxotrophic nanomachine of claim 99, wherein said particle envelope further comprises a biocompatible material.

102. The autonomous auxotrophic nanomachine of claim 99, wherein said basic genetic operating system further comprises an expression control region for the production of a biomolecule.

103. The autonomous auxotrophic nanomachine of claim 102, wherein said biomolecule further comprises an RNA.

104. The autonomous auxotrophic nanomacnine of claim 102, wherein said biomolecule further comprises a polypeptide.


SEQUENCE LISTING (19 sequences)

SEQ ID NO: 1 - DNA, 38596 bp, M. genitalium
taaaacaaaaaaaacaagtattaatttaaacacaattaatgtgaatgaatttccaagaataaggtttaatgaaaaaaacgatttaagtgaatttaatcaattcaaaataaattattcacttttagtaaaaggcattaaaaaaatttttcactcagtttcaaataatcgtgaaatatcttctaaatttaatggagtaaatttcaatggatccaatggaaaagaaatatttttagaagcttctgacacttataaactatctgtttttgagataaagcaagaaacagaaccatttgatttcattttggagagtaatttacttagtttcattaattcttttaatcctgaagaagataaatctattgttttttattacagaaaagataataaagatagctttagtacagaaatgttgatttcaatggataactttatgattagttacacatcggttaatgaaaaatttccagaggtaaactacttttttgaatttgaacctgaaactaaaatagttgttcaaaaaaatgaattaaaagatgcacttcaaagaattcaaactttggctcaaaatgaaagaacttttttatgcgatatgcaaattaacagttctgaattaaaaataagagctattgttaataatatcggaaattctcttgaggaaatttcttgtcttaaatttgaaggttataaacttaatatttcttttaacccaagttctctattagatcacatagagtcttttgaatcaaatgaaataaattttgatttccaaggaaatagtaagtattttttgataacctctaaaagtgaacctgaacttaagcaaatattggttccttcaagataataaatttagtttgtggcaaaagcttctgtactgtttatttaatggaagaaaataacaaagcaaatatctatgactctagtagcattaaggtccttgaaggacttgaggctgttagaaaacgccctggaatgtacattggttctactggcgaagaaggtttgcatcacatgatctgagagatagtagacaactcaattgatgaagcaatgggaggttttgccagttttgttaagcttacccttgaagataattttgttacccgtgtagaggatgatggaagagggatacctgttgatatccatcctaagactaatcgttctacagttgaaacagtttttacagttctacacgctggcggtaaatttgataacgatagctataaagtgtcaggtggtttacacggtgttggtgcatcagttgttaatgcgcttagttcttcttttaaagtttgagtttttcgtcaaaataaaaagtattttctcagctttagcgatggaggaaaggtaattggagatttggtccaagaaggtaactctgaaaaagagcatggaacaattgttgagtttgttcctgatttctctgtaatggaaaagagtgattacaaacaaactgtaattgtaagcagactccagcaattagcttttttaaacaagggaataagaattgactttgttgataatcgtaaacaaaacccacagtctttttcttgaaaatatgatgggggattggttgaatatatccaccacctaaacaacgaaaaagaaccactttttaatgaagttattgctgatgaaaaaactgaaactgtaaaagctgttaatcgtgatgaaaactacacagtaaaggttgaagttgcttttcaatataacaaaacatacaaccaatcaattttcagtttttgtaacaacattaatactacagaaggtggaacccatgtggaaggttttcgtaatgcacttgttaagatcattaatcgctttgctgttgaaaataaattcctaaaagatagtgatgaaaagattaaccgtgatgatgtttgtgaaggattaactgctattatttccattaaacacccaaacccacaatatgaaggacaaactaaaaagaagttaggtaatactgaggtaagacctttagttaatagtgttgttagtgaaatctttgaacgcttcatgttagaaaacccacaagaagcaaacgctatcatcagaaaaacacttttagctcaagaagcgagaagaagaagtcaagaggctagggagttaactcgtcgtaaatcaccttttgatagtggttcattaccaggtaaattagctgattgtacaaccagagatccttcgattagtgaactttacattgttgagggtgatagtgctggtggcactgctaaaacaggaagagatcgttattttcaagctatcttacccttaagaggaaagattttaaacgttgaaaaatctaactttgaacaaatctttaataatgcagaaatttctgcattagtgatggcaataggctgtgggattaaacctgattttgaacttgaaaaacttagatatagcaagattgtgatcatgacagatgctgatgttgatggtgcacacataagaacacttctcttaactttcttttttcgctttatgtatcctttggttgaacaaggcaatatttttattgctcaacccccactttataaagtgtcatattcccataaggatttatacatgcacactgatgttcaacttgaacagtgaaaaagtcaaaaccctaacgtaaagtttgggttacaaagatataaaggacttggagaaatggatgcattgcagctgtgagaaacaacaatggatcctaaggttagaacattgttaaaagttactgttgaagatgcttctattgctgataaagctttttcactgttgatgggtgatgaagttcccccaagaagagaatttattgaaaaaaatgctcgtagtgttaaaaacattgatatttaagtagtgttaaaaacattgatatttaatttggttagtataaatggcaaagcaacaagatcaagtagataagattcgtgaaaacttagacaattcaactgtcaaaagtatttcattagcaaatgaacttgagcgttcattcatggaatatgctatgtcagttattgttgctcgtgctttacctgatgctagagatggacttaaaccagttcatcgtcgtgttctttatggtgcttatattggtggcatgcaccatgatcgtccttttaaaaagtctgcgaggattgttggtgatgtaatgagtaaattccaccctcatggtgatatggcaatatatgacaccatgtcaagaatggctcaagacttttcattaagataccttttaattgatggtcatggtaattttggttctatagatggtgatagacctgctgcacaacgttatacagaagcaagattatctaaacttgcagcagaacttttaaaagatattgataaagatacagttgactttattgctaattatgatggtgaggaaaaagaaccaactgttctaccagcagctttccctaacttacttgcaaatggttctagtgggattgcagttggaatgtcaacatctattccttcccataatctctctgaattaattgcgggtttaatcatgttaattgataatcctcaatgcacttttcaagaattattaactgtaattaaaggacctgattttccaacaggagctaacattatctacacaaaaggaattgaaagctactttgaaacaggtaaaggcaatgtagtaattcgttctaaagttgagatagaacaattgcaaacaagaagtgcattagttgtaactgaaattccttacatggttaacaaaactaccttaattgaaaagattgtagaacttgttaaagctgaagagatttcaggaattgctgatatccgtgatgaatcctctcgagaaggaataaggttagtgattgaagtaaaacgcgacactgtacctgaagttttattaaatcaactttttaaatcaacaagattacaagtacgcttccctgttaatatgcttgctttagttaaaggagctcctgtacttctcaacatgaaacaagctttggaagtatatcttgatcatcaaattgatgttcttgttagaaaaacaaagtttgtgcttaataaacaacaagaacgttatcacattttaagcggacttttaattgctgctttaaatattgatgaggttgttgcaattattaaaaaatcagcaaataaccaggaagcaattaatacattaaatacaaagtttaagcttgatgaaattcaagctaaagcagttcttgacatgcgtttaaggagcttaagcgtacttgaagttaacaaacttcaaactgaacaaaaagagttaaaagattcaattgaattttgtaagaaagtgttagctgatcaaaaattacagctaaaaataatcaaagaggaattgcaaaaaatcaatgatcagtttggtgatgaaagaagaagtgaaattctctatgatatctctgaggaaattgatgatgaatcattgataaaagttgagaatgtagtgataactatgtctacaaatggttatctaaaaaggattggagttgatgcttataatcttcaacatcgtggtggagttggggttaaagggctaactacttatgttgatgatagtattagtcaattattggtctgttcaactcactctgacttattattttttactgataagggtaaggtttatagaattagagctcatcaaattccctatggttttagaacaaataaaggtattcccgctgttaacttaatcaaaattgaaaaggatgaaagaatttgttcattgttatctgttaataactatgatgatggttatttctttttctgtactaaaaatggaattgttaaaagaacgagcttgaatgaattcatcaacatcttaagtaatggtaagcgggctatatcttttgatgataatgacactttgtattcagtaattaaaacccacggaaatgatgagatttttattggttctaccaatggatttgttgttcgcttccatgaaaatcaactcagagttctttcaagaacagcaagaggtgtatttggtatcagtttaaataaaggagaatttgttaatggactatcaacttcaagcaacggtagcttacttttatcagtcggtcaaaatggaataggtaaattaacgagcatagataaatatagactcacaaaacgtaatgctaagggagttaaaactctaagggttactgatagaacaggccctgttgttacaacaaccactgtttttggtaatgaggatcttttaatgatttcctctgctggtaaaattgtgcgtaccagtttacaagaactttcagaacaaggtaaaaacacttctggtgttaagttaattagattaaaagataatgaacgtttagaaagagtaactatctttaaagaagagttagaagacaaagaaatgcaactagaagatgttggatccaaacaaattacgcaataattaaacattaaccgtcaacaacaagaaactttatagagcaatgaaaaatcaaaccaagagtaatagtttatttcaactttccactaactatatacctactggtgatcaacctgaagcaattaagaaattatcagaatttaaaactaagcagcaggttttattgggggccacaggcacaggtaaaacctttacaattgctaatgtaattcaaaacagccaactcccaacagttgttattgctcataacaaaaccctagcaggtcaactcttcaatgaattaaagcaactgtttcctaaaaatgcagttgaatattttatctcttactttgatttttatcaacctgaagcttacttacccagtaaagggatctacattgaaaaaagtgctacagtcaatgaagcgattaaacgcttaagagtctcaacactgcattcactttcaacaagaaaagatgttattgtagtaggttctgttgctagtatttatcccacctcatctcccagtgattttgttaagtattgcttgtggtttgtggttggcaaagattatgatttgaaaaccattaaagataggttagttagtcttaactatgttgttaataaacaacaattaaccccaggaaaatttcgctttcagggtgatgttttggaggtatttcctggttacagtgatgcttttgtgatcagaatctccttttttgatactaaagtagaacaaatttgtcaaattgacccactaacaaataagattttaaaccaactctttgagattaagataggtcctgctgatgaatatgttgtaaaccaatctgatcttgatatagcaattaaaaatattaaacaagaacttcaggaacgagttaattatttcaataagcaaaatcttgttgaaagagcacaacgtttagccaccattactaaccatgatctcaatgatctgaaggcttggggattttgtagtggagttgaaaactatgctagacacttagagttgaggatggctaactcaaccccttacagtatctttgattattttaagggggattggttactggttattgatgaatcacaccaaactttaccgcaacttaatgggatgtataacactgatctttcaagaaagcaaagcttaattgattatggttttcgactcccctctgcacttgataacagaccgctctcatttgctgaattacaacaaaaaatgcaaaaagttatttatgtttcagcaactccaagagataaagagattagtttaagtcagaataatgtcattgaacagttagttagaccaacttacttggttgatcctattatcgttgttaaaccaaaagataaccaggtggaggatctcattgaagagattatcaaccaacgccaaaacaacacaagaacatttgttactgttttaactattaagatggctgaaaacctcactgaatacttaaaggaacgcaaaattaaagttgcctatatccataaggacattaaagcattggaacgtttattgttaattaatgacctgagaagaggtgaatatgagtgtttagttgggattaaccttttaagagaagggttagatgtccctgaagttgctttagtttgtatctttgatgcagatatcccaggactacctagggatgagagaagtttaatccagattattggacgtgctgctagaaatgaacatggtcgagttgttatgtatgctaaccatgttactgaacagatgcaaaaagccattgatgaaaccaaaagaagaagaactgttcaaatggaatataacaagctacataataagacaccaaaaacagttgttaaaccccttacctttgttcaaccaatcaaattaaaagctaagagtaatgcagaaaaaaatgctgcattaatcaaacaattaaccaaagaaatgaagaaagcagcagctaatcaaaattatgaacttgccattgagattagagattccatatttgaattggaaaaagaaattggtagtaaaattaaagtatagtcgttttcaaaatcaatttaaaaaaggagcaaaaccttagatgaaccgggtcttcttgtttggtaaactcagttttactcccaaccgtttacagacaaaaaatggtacgttaggagctactttttccatggaatgtcttgattccagtggttttaataatgccaaatcattcattagagtaactgcttgaggtaaagttgctagttttattgttgctcaaaatcctggggtgatgctttttgtagaaggaagattaactacatataaaattactaacagtgaaaataaaaacacctatgctttacaagtaactgctgataagatctttcatcctgatgaaaaaactaccaatgaagaacctattaaatcaactgtagttgattcaccctttatgaatcccaaagcaagtgttacagaagctgagtttgaacaagcattcccccatcaagatgaaactgattttaacaacattacccctatatttgaaaatgatgtccaactagaggaggaaagtgatgattaatgcaaatgatagcaacattgaacgtgctgaaagacgtttgatgcaagcagttgctcaaaacagtgagggcattgatctaattttcaataaacttgaaccaattgatttttttgcaacccctttcaaactcatttttcaaactgcaaaagaaaactaccaattaaataaccctattattggttctggtttactagaagcggttaagtttaaacttgatgctaatgatcaatccactaaaagtgaacttgaaattttattcacaaagatcttattaatccgtttaccacctaaccaaacagagattaaaacactggttgatgttgttaaaaaagcttctatttttcgcaggttacaacagtttgctaagcgtgtttacaacgaggaatttaagttaaaagaagatcgttttgaaggctatttacaagctattcaagatgattttgtcaagattatccacagtgcttttagtaacatctttgcttttagctatgatgagattgccaatcaagaggaagcattaattaaaaaggttcaccgtggggaattgatcatcagtggactttcaagtggatttttaaaattagatcaacttacatcaggttgaaaaccaggagagttaatagtaatagcagctcgcccaggtagaggtaaaactgcccttttgattaattttatggctagtgcagctaaacaaattgatcctaaaactgatgtggtcctcttctttagtttagagatgcgtaaccgggaaatttaccaaaggcacttaatgcatgaaagtcaaactagttacacactaaccaaccggcaaaggattaataatgtctttgaagagttaatggaagcatcttcaaggatcaaaaacttacctattaaactctttgattacagtagtttaacactccaagagatcagaaaccaaattactgaagtgagtaaaaccagtaatgttaggttagtaattattgactatttacaacttgttaatgctttaaaaaataactatggtttgacaagacaacaagaagtgacaatgatctctcaatcacttaaagcattcgctaaggagtttaatacccctattattgctgcagctcaactttctagaaggattgaagaaaggaaagattccagaccaattctttctgatttaagagaatcaggttcaattgaacaggatgcggatatggttttatttatccatagaactaatgatgataaaaaagaacaggaagaggagaacacaaacttgtttgaagtggagcttatcttagaaaagaacagaaatggtcccaatggcaaagttaaactaaactttcgcagtgacacttcttcttttattagtcaatattcccctagttttgatgaccaatacagttaatgatcagttacttttacctagataaatttaaagtttatttgtggtaatggatgatctattccaaagaatggttagctgtgttctaccgtcatgaagagcttttattgatgaggaagttaaaaaaccttattttcaagctttattagaaaaattaaaggctttaaaagcaacaataattccaaaaccagaacttattttccgtgtttttagcttctttaagccaattgatacaaaggtaattatctttggtcaagatccctatcctagtcctaatgatgcttgtggacttgcttttgcatccaataattccaaaacccctgccagcttaaaaagaataattttacgtttagaaaaagaatatccttcgcttaaacaagaaagtagttgacaacaaaacttcctattgaattgagcagaacagggcgttttattactaaatggaattttaacaactactgtatttatacgcaacgcccataaaaattggggttgggaggagtttaactgtaatttgctaacttttctaaaaaatcaaaacattaaaccgctgttggtatttctgggtgttcaaactaaaaactttgttgttaagagtattggtaatgttgatggatttgagcatttatcatatccccatccctcaccactaagtggtaatttgtttctaacaaaccctaacgatctgtttaaaacaattaacaattggttgaaacaacataaccaaaaaataattaactgagcagttgttaaaaatgctagttttgaccaattaagttaaaacaaaaaccttatttatagttaagtaagtagttttattaatgattaaaaacctggtggtgattgaatcacccaataaagttaaaacattaaaacaatatcttcctagtgatgaatttgagatagtctcaaccgttggtcacatcagagaaatggtgtataaaaactttggttttgatgaaaatacctatacccctatctgagaagattgaactaaaaataaacagaaaaatcccaaacagaaacacctgctcagtaagtttgagatcatcaaatcaatcaaagctaaagctagtgatgcacaaaacatttttttagcttctgaccctgatagagaaggggaagccatctcttggcatgtctatgatttattggatcaaaaagataaagctaagtgcaaacgaatcactttcaatgagatcactaaaaaagcagtagtagatgcattaaaacaaccgcgtaacatcgatcttaactgggttgaaagtcagtttgcccgccaaatccttgacaggatgataggttttagattatcaagattattaaatagttatctgcaagcaaagtctgcaggtagagttcaatcagtggctttgcgctttcttgaggaaagagaaaaggagatagctaagtttgttccgcgtttttggtggacagttgatgttttattaaacaaagaaaataaccaaaaagtagtttgtgcaaacaagtctattcctttggttttaagagaaattaaccctgaattaagtgctagtttaaaactggattttgaagctgctgaaaacgtatcaggaattgactttttaaatgaagcttcagcaaccagatttgccaaccaactgactggcgaatatgaagtttattttattgatgaacctaagatttactattcatctccaaacccagtttataccaccgcttcacttcaaaaggatgcaattaataagttaggatggtcttccaaaaaagtaacaatggtggcccaaagactgtatgaagggattagtgttaatgggaaacaaactgcattaattagttatccaagaactgattcaattaggatttcaaaccaatttcaatcagagtgtgaaaagtacattgaaaaggagtttggaagtcattatttagctgataaaaataagttaaaaagacataaaaaggatgagaaaatcatccaagatgcccatgaagggatccatcctacttacattactattacccccaatgatctgaaaaacggggtgaaacgcgatgagtttctcctttatcgtttaatatggattagaacagttgctagtttaatggcagatgctaaaacatcaagaactattgttcgttttataaaccaaaaaaacaagttttatacctcttcaaaatcacttttatttgatggttatcaaaggttatatgaagagattaaacctaatactaaagatgaactttacattgatcttagtaagcttaaaattggtgataaatttagttttgaaaagatcagcgttaatgagcataaaaccaacccaccaccacgttacacccaagctagtttaattgaagagcttgaaaaatctaacatcggtcgtccctctacttataacactatggccagtgttaatctagaaaggggctatgctaacttagtgaaccgatttttttatatcactgagcttggtgaaaaagttaataatgaactttccaagcattttgggaatgtaattaataaagaatttaccaagaagatggaaaaatctttggatgaaattgctgaaaacaaagtaaactatcaagaatttcttaagcagttttgaacaaattttaaatctgatgttaaactagctgaaaattcaattcaaaaagtgaaaaaggaaaaagaattggttgaaagagattgtcctaaatgtaatcaaccgttggtatatcgttacaccaaaagaggtaatgagaagtttgttggttgtagtgattttcctaagtgtaaatacagtgagtttagtaatcctaaaccaaaactaaccttggaaacacttgatgaattgtgtcctgagtgtaacaataaactggttaagaggagaactaaatttaacgctaaaaagacctttataggttgcagtaatttccctaactgccgttttatcaaaaaggataatgctgctgaatttaaacaataacggcagcaaagctaatttcaattttaaattcaaactttagatgaaaagtaactacagtgcaactaacatcaagatcttaaagggtttggatgcagttaaaaagcgtccggggatgtacattggttctactgatagtaagggtctgcaccacatgctatgggaaattcttgctaacagtgttgatgaagttttagctggttatgcaaccaatattactgttactttagatctcaacaacaccattactgttagtgatgatggcaggggtattccctatgagatccaccaagacagtaacatctctacgatcgatacagttttcacctttctccatgcaggggggaagtttgatgatcagtcatacaaactagcagggggattacatggggttggtgcatcagtggtcaatgccttaagtgatcatttagaagtaacagtgaaaagaaatggtcagatctaccaatcagtttatcaagctgggggtaagatcatccaaaaagccaaaaagattggtgatacaactagccatggtaccactgttagtttccatgctgaccctaaggtctttaaaaaggctcaatttgatagcaacattattaaaagcaggttaaaagagctaagctttctgtttgctaaactaaagctcacttttactgatcaaaaaactaataaaaccactgttttttttagtacctcaggactagttcagttccttgatgaaattaataatactgtagaaacacttggccaaaaaacactgattaaaggtgagaaggatgggattgaagtggaagtggttttccagtttaaccaatcagatcaagagacaatcttatcatttgctaactcgattaaaacctttgaaggagggagtcatgaaaatgggttttgtcttgccattagtgatgtgatcaacagctattgcagaaagtacaacttactaaaagaaaaagataaaaactttcaacttagtgagatcagacaagggttgaatgctattatcaaagttaacttacctgaaaaaaacatcgcttttgaaggacaaactaagagtaagttgttttcaaaggaagtgaaaaacgttgtttatgaattggtccaacaacactatttccagtttctggaaagaaacaacaatgatgctaaattgatcattgataaactactcaatgctagaaagattaaagagcaaatcaaacaacaacgtgagttgaaaaaaagtttatcaagtccccaaaaagagaagatcttatttgggaagttagcaccttgtcaaaccaaaaaaaccagtgaaaaagagttgtttattgttgaaggtgatagtgctggtggcactgctaaaatgggccgtgatagaatttttcaagctatcttacctttgcgcggcaaggtgttaaatgttgaaaaaattaacaataagaaggaagcgatcactaacgaagagatcctcactttaatcttttgtattggtacagggattttaactaacttcaacatcaaggacttaaagtacggaaagatcatcattatgactgatgcagataatgatggcgcacacatccaaatcctcttacttaccttcttttataggtacatgcaacccttaattgaactgggccatgtctatctagctcttcctcctttatataaactggaaaccaaagatagaaaaacagttaaatacctctggagtgatttggagttggaatcagtcaaactaaagcttaataacttcactttacaacgatacaaaggacttggagagatgaatgctgatcagttgtgagatactactatgaatccaactaccagaaagctagtgcaagtaaagcttgatgatctaattaacgctgaaaagcaaatcaacatctttatgggtgaaaagagtgatttgcgcaaacactggattgaagccaacattaactttagtgtggaaaactaaatggatcaaaaaaacaacaacctctttcaaaaggcaattgaagaagtctttgcagttagctttagtaagtatgctaaatacatcatccaagatagagctttacctgatctaagagatgggttaaaaccagtacaaagacggatcttatatgggatgtttcaaatgggcttaaaacccaccactccctataaaaaatcagcccgtgctgttggggagatcatggggaaataccacccccatggtgatagttccatttatgatgcaattatcagaatgtcccaaagctgaaagaacaactgaacaactgtttctatccatggtaacaatggttcagtggatggggataatgctgcagcaatgcgttacacagaaacccgcttaagcttgtatggatttgaactattaaaagacattgataaaaagttagttagttttatcaataactttgatgatagtgaaaaagaaccaacggttttaccaaccttactgcctaacctctttatcaatggtgcgagtgggatagctgctggatatgcaactaatattgctccccataacactaatgaactattagatagtctttgcttgcgaatagaccaacctaattgtgaacttaaacaaattttaaaaattgttaaaggtcctgattttccaacagggggtaatgtttattttgaaaagagtttaagtgatatttatcaagcaggcaaaggtaaatttattatccaagctaagtatgaagttaacaagaacttaaaccagattgaaattacccaaatcccttatgaaacactgaaagctaacattgtcaaacaaattgaagagattatctttgacaataaactatctgctattgaaagtgtcattgatagttcagatcgcaacggcattaggatcattattaaacacaaggactttttgcctgctgagaagatcatggcctttttgtttaaacacacccaactccaagtgaactttaaccttaataacaccgtgattgctaaccgctttcccatccaaattggtttactaagttacctcgatcattttttaaagttttgtcatgaactaattattaataaagctaagtatgaacttgagcttgcaagcaagcgcttggaaattattttaggactaattaaagcgattagtatcattgataaaatcatcaaattaattagatcagcagttgacaaaagtgatgcaagagaaaagttaattgataactttaaatttacttttaaccaagcagaggcaattgttagtttgcgactttaccaactaactaacactgatatttttgaacttaaccaagaacaaaatgaacttgaaaaaactgtgattagttcagagcaactaattgctagtgaaaaagcaagaaacaaactcctaaaaaaacagtttgaaggttataaaaagcagtttcaccagcaacgaaggtcacaaatatgtggctttattaaccaaaaaaaggtggaggaaagtgagctaattgaaaacaaaacttatggggttttaatcactaaagctggtaactaccataagtttgaatctaaccaactattaaaaagcaccactgattttaaaagtgagagtgacacaattatctttgcacaaactattgctaataccgaccaaatttttattgtcacttcactaggtaacattattaatatccctgtttataaattagctttcaattccaaaaataaactagcaagtttagttagtaaaaaaccaatccttttggagtatgaaacgattgtttttgttggaacaatgaacagtgtaaaccaaccaatccttgttttaacttccaaactaggaatggttaaacggattgatttaaccaaacttaacattaagccacttaaagctactttgtgtatctcactccgtgataaagaccatttagtaagtgcatttttacaacaagatgataaactgatctgtttagtgtctgatcacaactattacactgtttttcacaccaatgagatcccattaattagtagtaaggggatgggagtgaaggggatgaagttaaaactagaggatcaaattaagtttgttgttgcttttgaagctaatgaaccgttagtgatgatatgtagtgatggtagtgtcattaacttaaaacaaactgaactagttgtagttagcaggatggcaactgcaaaaaaactgcctgttaagaaagcaattaactattgttttagtgatgcaactaacacccagttaattaattttcagggtaagaacggtagtaaattaattacaactagtgaactgaaccagatgagtaaaactgcaattagtcaaaccaggtttaacaaacttaattagtgcacctcaccaaaaaaaccacccgtgcttttattttcagatgggtttatgtatggggcaatgtatgcaaactgatactaaggaaaaataccaacaggtaattagtaacattgaacagttttttaatgaccctagtgtggtaattaactatttaaaagctgcagaaaaaaaggcaagtgataatcaggaatttgaaaaggcccagcagtttctaacactgcaaaaagcagttttagagttaacaaaaacccaccataccactatcattaaacaaaaatcaagccatgattttattgggtatgtctttcaaaataacgttttggccattaccattttttgttatgaaaaaggggagttaactgataaagaacaagcagtgtttaccctagagcaaactgacattgtggaagttgaaagtgctattatcacctttatctaccaccactataaaactaccccacttccaagtaagattactgtttcacttgatgaaactaacctaaaacttattagtgatagcttaaaaattggtgtttttaagcccaagaatggtaatgaaaaactgatcttacaaactgttattgataatgccaaacatgcacttgcaaccaagtggttgaagtttactagtaactatgataaaacccagctccacaaggatttagcacaacttctaaatactgattatatccatagtcttgagattattgatgtgtcattctatgatcaaaaccatgttgttggttgcatgttaaggtttgaagatggtaaaaagatcaaacacttatcaagaagatacaacattaacagtttaaaaaaaggtgatactaaccacattgctttacttgtttacagaaggatcttaagtgcgatgcaaaccaaagctaacctcccttttagtgatcttttaattattgatggtggtaaagcacaaattaaaagtgttaagcaagtttttagtctcttcagtaatgttaaaccacccattatcattggactagttaaaaacaaaaaccaccaaactgatcacattatgttatctgatttccaagttaaaaagatagcaattaactccccactctttcactatttagcaacaatccaaactgaagttgatggttttgctaaaagaagtgcttttaataagttaagtaaccaccaactgcaaaacccgttgctacaaatcccaggagttggcaagataactgcccaaattctctttgataactttcaaacgctcaataacataaaattagcttcagttaatgagttaagccagtttattaaaaaaccattagcacaaaagattaaaacttactttgcaaaacaaactgattaatagctcttgatgaagtggaaaaactcattaattaataacaatgaatgaacaacaaaaacaagcaattagttgtggaaaaggggttaatgttgtttattctggagcaggtactggtaaaacaacaattattactaatcgctttgcatacttggttaataaagaaaaagttgatcctagcagaattttagcaatcacctttactaagaaagctgctaaggagatgcagtttagaatcttgaaactaatagatagttctttagctgagaaaacaaatatctatacatttcacagcttttgcaataagtttttaattcaaacattaaaaaagcgctttatcatcgatgatgatattagctatttcctaaaggaatttttagctgattcaaaactcgatatcaacctagcgaaacaaattattgataactttaaaaatacttttgctgattttgaaataaataagttggatcaagatgaaaggttaattagtttatgtgagcattcacttctaaataaagatgaagaatattccactttaaaaacccaactgattaatgcattcattagctatgaaaagaataagatattaaacaataaacttgattttcatgatcttttaattaaaacttgtaatttattgagtaatgataatgatttacttaatcagtggagtgaacagtttcagcatattttagttgatgaatttcaagataccaaccaaatccaatatgaactgatcaagatgttagtaactaaaaataaaaacttgtttttggtaggtgataataaccagatgatttaccgctgaagaggggcggtaaacgggatcataactgctttaaagcatgactttaatgttccgaaaagcaatgaattctttattaatcaaaattaccgttgcgatcagaatattttagcagttgctaaccaaattcttttaaaaattatggcctatgaaaaacaagttaaaactgaaaaaaatctcttgttttcaactttaaattctgataaaaaacctgtttattttcaagctgaatcagttgaaaatcaagccaattggatcttcaataaaatcaaagcactaaaccaaacagaaaagattaattttaaggatatggccatcttgtttagaaagaacagagatattactactatggttgaattgattgaagcggatggaacaattcccttacctaaacaaaagagttattttaaccaactagtaaaactccagcgggttttaattgcgatttcaaccagaacaaatcttgatattaaaagagctttgcaagccctaaaaatttgatcaaatgatttaaaggaattgtgaaaacagagtgataaaacaaacctatttgattttcttaaatgatcagaattaaatcaaaaaaaccatagttcaaaacttaaagctactggttattttaatctgctgattaagttagcagaggatcagcaaattaaccttttgtttactgaactgtttaaaaaactcaaagtggatcaaactattgaaaatctgctttgaaaaaaactaactgaatttcaaaaagataaaactgaatttagcttatcagagtttattactagcttagcattggaatttgactcaattattgaaaacagcagtgatacaatcaatttgctaaccgttcatgcagcaaaaggacttgagtttgaagctgtatttatttatggcatgaatcaaggggattttcccttatttttaagtcaaaatcaaaatgacgaacaacatttaattgatgaattaaaactgttttatgttgctatcacaagagcaaaacgttttttgtttatcactgcggttttacaaataaataacaattctataaaaccatctagttttttaaattacatcaataaaagtgagtatttagacattgctactattaactatgtattagagcaggatgatgatttttttgattcaactaaaaaaacagactatacaaagaaactaagaaaagaaagtttagacattatagtgggtgatttagttactagtagatactttggaaaaggagttgtagttgaagtgagagacaaagaggttttagtagcttttaaagacacacgctatgggatgaaatggatcttaaaaaaccataaatcactaacaaaagctttatattaacgaattgctatcaaagaattaccacaatgattcatgaaaaatggtcaataaaagcaacagtttagatgaacttttaaagcagattaaaattactgaaattattcaacactacggggttaaaatccaaactaagggtaatagtctacttgctttatgtccttttcatgatgataaaaatccttctatgtccatttccagttctaaaaacatctttaagtgttgggcttgtaatgcagctggcaacggaatagcgtttatccaaaagcatgaccagttagattgaaaaactgcacttaaaaaagcaattgaaatttgtggaattaagttagaaaattggaacagtaatttactaacaaaagttgatccaaaacaaaaacgatattgggagataaacaatgctttaattacttattatcaaaccagattaaaaagagaaacaaacccaaatgggatgaattatttagttgaaaaaagaaagcttaataaaacattaattgaacagtttcagctaggacttgcttttcacaatgaagataagtatctatgtgaaagtatggaaagataccccttcattaatccaaagataaaaccgagtgaattgtatcttttttcaaaaactaaccagcaaggtcttggcttttttgactttaataccaaaaaagctacctttcaaaaccagattatgatccctatccatgactttaatggtaacccggttggtttttcagcaagaagtgttgataacatcaacaaactgaaatataaaaatagtgctgatcacgaattttttaaaaaaggggagctgttatttaactttcacaggttaaataaaaacctcaatcaactctttattgtggaaggttattttgatgtttttacactaacaaactccaagtttgaagctgttgcattaatgggattagcattaaatgatgtgcaaattaaagccattaaagctcactttaaggagttacaaaccttagttttagcacttgataatgatgctagtggtcaaaatgctgtgtttagcttaattgaaaaacttaataacaacaattttattgtggaaattgttcagtgagaacacaactataaagattgggatgaactgtatttaaacaagggtagtgagcaagttatattacaagcaaacaaaagacaaaatctaattgaatatcttgttagtttttttaaaaagcaacaacttgatcaaagggttattactaataaaatcattgcttttttaacaaaaaaccaaacaattttaaacgaccatagttttttaatttttctcattaaaaatttggttaaactacttgaatatagtgatgaaaaaactctgtatgaaacagttttaaaacacaaagaaaaacttgtatctaagtttgataacaaccgtttttacataaatacttcaggccatgctcaaccaccacaagaattgcaaaaaaccactgcagcactagtgcaaacagcttttgaagaagcagttaatgagttgtgaaaacctgaaatctttgcgtttgctttaattgataaacgctttttggttgaattaaaacaatcccatctagatgaagtttttaaggaatgtaactttaatttgtttgatgttgaactttttattgaaaaagcaaggatctattggagtgaaaatcaaactgctaactgagttggttttgaaagtgttttagatcaaaattaccttttaaacaataaagcaaggttattggaaattaaagatatttttttagatgaattaacttgttatcaagctaatgattttcaaaactatctaaagacctttcaaacgttattaaaacaacaaaagcagcgcttaaaaaatttaaagttaacgctataaattggtatcaactaagtcaggatttttcttatttatagtagtgatggatgtgaaattgaagattcaacagctggttaacttaataaaaaactatgactatcactactatgttttaagcgaacctttaattgatgattttgagtatgatatgttgtataagtcactccaacaattagaaaaagatcatcctgatttaatccaaattgattcccctacccaaagggtgggaggagaagctgtgaagggttttaaaaagttaaaccataacagtccaatgctctctttggaaaatgctttttcaactaaagaaattgctaattttattgataatattaactttcaaacaaactcaaaaaatgaatttgtagttgaacctaaaattgatggagttagtatctctctaacttataaaaatggtgttttagttcatgctttaaccagaggagatggaagtgttggggaagatgttttaaataatgttaaaaccattaaatctatccctttaacaatccctttcacaaaaacaattgagattaggggtgagatttttgttgataaaaaaacttttttagcaattaacaatcaacttgaaaaaccatttgctaatgcaaggaatctagcagcaggtacaatacgtaatttaaacagtgaaatcactgcacagcgcaaattaagggcattattttattacatccctaatggtttggaagagtcaatcactactcaaactatggttttagaacagcttaagcagtgaaaattcccagttagtgataccatcagggtttttcaaaacaaatttcaattaattaattacttggaagcgtttgacaaaaaacgagaacagttaacttttaatcttgatggtttagttattaaactaaacagcttgcttttttatcaacaattaggtgctacaagtaaatcaccacgttgggcaatagcatttaaatttagtcctaaatttgttcaaactaaattaacagcagttcttataacgattggtagaactggtagagtgaactatactgctaaattagaaagtgttaatttagatggaacaaaagtaacagctgctactttacataactttgattacattaaaactaaagacattaggatcaatgacactgttgttatctataaagctggggaaattatccctaaagtactaaaggtaaatcttgaaaaaagaaaaaatgacactatcataattcaagagcaaaaatattgtccttcatgtaattcaaaactagtcaaaatagttgatgaagttgatcagtattgtaccaatgaaacttgtaaggagcgaaacatccagttaattaactattttgtttctaaaactgctatggacattaacgggttgaatattaatactattaccaaactttatgaacacaatttggttagatctatagttgatctttatgatttaaaagacaagaaaaaccaagttttaaaattagatctgaagattggtgataaacttttcaacaagttagttgataacattgaaaattcaaaacaaaaaggaatggctagattactaacaggacttggtattaagcatgttggtaatgtattagctaagaatttagctaatcattttaaaaatatcaaagcattacagcatgctagcttagagaacttaattagtttaaatgatgtaggaataacagtagctgaatcattgtataactggtttcatgaccctaaccatttgcagttaattgaacaacttgaattaagacaagtaaaaacagatcaattaccactgaaaattaactttgaaactaacagtatttattttcaaaaacgctttcttattaccggtagctttaacattagtcgtgaccaaattaaggatttattatcagctaagtttgattgccagtttgcaagtgaagtcaaaccaacagttgactttgttattgcaggaaacaaaccaactttaagaaaaatcaatcacgccaaagaactgaacattcctatcattaatgaagcaatttgaacatagtgatgaaaagaaacaacagttggaaaagtttattagttagatgactctgtatgagttttttttaaatcaaaagttagtttaccaatccagtccccattttaacggggtatttttaacaatattggaacactatggttttcaatttaaaacaattgataaactctgaaaaagtaagcttctaattactagtgagttaactgataaaatcaaacaacaattaaagtgttattttattgaaaagatccctttgccctatttgttgggaacaattcaactaaggaagcttacttttaaaactaagaaaggagtttttattcctcgaattgatagcttagcactaattgcaagtgttaacttaaaaaaaataaaaactgcacttgacctttgttgtggttcaggtactttagccattgctttaaaaaagaagtgtgatacacttgatgtttatggtagtgatattgatatccaagcattaaaactagcgcaacaaaatgcattaattaataacgttagtattaattgaattgaagcagattgatttgattgttttaacaagataaaaactccgattgatttaattgttacaaacccaccttatctgaaaaaaacacaactaaataaaacattaaattatgagcctaagcacagcttggtttttcaaaataaaaatagttattttgcatacaagcagttgtttaatctattactaacaaaacgatcaattaaacagttaatttttgaatgttctttatttcaaaaagaaaggctattaaatttgttttcaatctttaaatcaaggccgatttttaactttcaaaaacagtttattggtatgaaagttgataatcaaaaactcccagtagttgatattaaaaataccaaaactattaagcaacttttaaaaatggggctagcaggaattgtaaatactgatacacaaatgggattaattagttattcagagtctactcttgacaaaattaaacaacgtgcacttaacaaacattatgtatcaatgtttgggttagaagaattaaagaagttaccaaaaaaactacaacaaattgctagttacttttgaccaggtagttatacctttattaaaaataacaagagctacagggttcctaaaaacttgggcttattaaacctttttaatgcaattggtagggttttttgtactagtgctaatatcagtaatcaaaaaccatacaccaaattaagtgattatcaaaacgatagttactgaataaagcaaccttgttttattattagaagcacttctaaagtgcaatcaaataacacaccttcacttgtctataatttagatacaaaacagttggttcgcaccacagctaaacaaacaaaacagtttcataaattaataactaaacaccagttagctatctaaagaaatagaacgcaaaatgaaacgttctagtcgctaattgatgtttgttaatttacatacaaattcatactataactttctcaattctgccctttctcctaaaaagctagttaatctagcaattaatgatcagcaaaaagctgttgctattacagatcctaatctttttggcgctgttgaattttttataacttgtaagcaaaataatattaaaccaattattggtttaaacttaactgttgaataccaaaaaaatgatgttaagttattactaattgctaaatcaaataaaggctttcaaacgttgaacaaaatagcattaattcaacaaaaacttgaaattaattctttagttgatcaactaacagatattgcagtaattatctgttctttaacaacatgaaaatctacttataaggatgtttatcaagcaaaaggaattgaaataaatcaaaccccgattgccattcttgcaaatgctgttaactgtgaaaaaactaatagcgatcaagtagttttaacagttttgaaacaaatgaaacaaaaccaaacgggaaaaataactacatttgattgggatcttaaacaaaaattaaatcaaatttcaattaatgaaaatttaaaagtaaagagtgaaattcaaccttttttagatcaaaaaactgcacaacaattattcagtgaaacagaacttaataatctgaatgatctagttaatagatgtgaattagatttggagcacctaaaagctgcttcactttctttaactgataatgatgcagcagttttagaaagtttgtgccaaaccaatttaaaacagtttttagataaaaatcaagatctaaataaaaaagcctatcagctacgtttagagaaggaattaaatgttatcaataaacttaattttgctagctattttttagttgtcaatgatcttgttaattatgcttttaaaaaggacatcttaattggttctggtagaggttctgcagtaggatcattagtggcttttttattaaacattaccaagatagacccagtccaacaccagcttattttcgaacgttttatctcaacccaccgtcaagatctacctgatattgatattgatatcatggagaataaaagagcagaaatgataaattatctgtttgaaaaatatggcaaagaaaactgtgcacaaattgttacttttcaacgttttaaaacccgttctgctgttaaagaagttgctaaattatttaatgattatggcattagtgacatgatcctaggagtgttacctaaagatcaaactataacattcactgatcttaaagctactgaagatagtgctttacaactttgtttacaacagtttggtttaattgttgaattagcactagcaatagttgattttccaagacaatcaagtatccatgcttcaggcatagttatcgcttcaaattctttgattaaaaccattcccttgttacagcttgacaataatcactttttaactcaagtttcaatggaatggttaagtttttttaatctcaataagtttgatctgcttggtttaattaaccttactatgattagcgatgtaattacccaaattaaaccatctaaccagaccgttaaccagtttttaaataccatttcttgaactgatcaaaacacctttataaacttagtaaatgaagatacactaggaatctttcaacttgaatcgtttggcatgaaaaaattactggttcagattaaacctaaaaccattaatcaactagcaattgttctagcgctttacagaccaggtgcacaggataacattaacctttttattaaccgcttgcacaatggttatgatcaatctgacattgatcctaggattttacccattgtgaaaaatacctatggagttttaatttttcaagagcagatcattaacatcgttaaagttgtggctaactactctttagaagaagcagatagcttccgtagagccatttctaaaaaggatgttaaattgatccaaaaaaataagcgtaacttctttgaaagagcagttcaaaataactttgatttaaagactactaccaaaatttttagctacatagaacgctttgctaactatgggtttaacctttctcatgcgttgggttatgcactgctttcatactgaacagcttgacttaaaactaactatcctgtttatttttatttatggttattaaaccattttcaatctagtaaagacaaacaaaaactaattattagaactttagaaaaaagtggtattgaaatttatccacctcttttaaataaagctcaaccaaatagtgttatagaaaataaaaaaatttatttaggtctaaacctaattaagggaattaatgacaggtacatccaaaacttacaaaaagtgcaacatttaattcaaactcaaaataacttacaactaactgatgtagtaagttggtgtttggataaaaccattggtgatatccctttaaaagatttacttttattaaaaactatgggctgttttgatttttttgaatacacttatgactttaatgatgcaaaggatttttgaattaaaagcgatcacctattgtttaccagaatgcctttagaaaaaaaggatagtaatttttgaattaaacaattttttaccaattagacaaaattaatactttagttgaaaaattttcaaaaatataatgcctgaacttcctgaagtaactactgttattaatgaacttaaagaaactgttttaaataaacctttagatcaagttcaagttaacctaagaaaggttttgaaaaatattgatcctcaattgctgaataaacaattaaaaaatcagttttttactgatattaagcgtaagggtaaatatatcatttttcttttaagtaatggtttgtatttagtttcgcatttacgtatggaaggtaaatacttttttgaagaaagaggtagtaaatttaatcaaaagcatgttttagtagaatttcattttgatgatggtagtcaactcaattatcatgacaccagacaatttggaacgttccatttgtatgaaaagttagaacaagcagcacaattaaataaacttgcatttgatcctctagaagctggttttgactataggaaaatcttccaaaaagcacaaaattcaaaacgtaaagttaaaacttttattttagaccaaacagtgattagtggaattggcaatatttatgcagatgaaatcttatttgcaagcaaaattaatcctgaaacaatggttgatcaactaacaattaaagagatagagattttatgtaaaaatgctaccaaaattttagctaaagcaatagttatgaaaggtactaccatcagcagctttagttttaaaaaagatcatactggaggctatcaaaactttttaaaagttcacactaaaaaagatcaaccttgctcagtttgtaaccaattaattgttaaaaagaagattaatggaagggggagctatttttgtttaaactgtcaaaaaatcacaaccaaagtttctacaaaactcaatccataatttttatttttttgcacttatttataaggtgtaatgaaagatggctcaaaaagaaataattaataagaaaaatactcaaaaaaatagtagttttattgaaagtaataatttgacaagttttgatttttttgatgcaaagaaaaacagtgaaattgaaacaatttcaactggaagtttaaatttagatgaagcattagggtctggtggtctacctttaggtaggatagtagaactatatggaaatgaatcatctggaaaaacaactattgcactaaatgcagtcgctagttttcagaaagcaggtaaaacagcatgttatattgatgctgaaggtgcacttgatttagcatatgctaaatcaattggtattgatctaaataaacttttgattgctcatcctaggcatggtgaaaacgcttttgctcttatcgaatcattaattaaaacaaacaagatatctttaattgttattgactctgtagcagcgttaattcctaaacaagagttagaaggcacaattgaagaacaaactattggcttgcatgcaagaatgatgtcaaaaggtttgcgaagaatacaatcgatattaccagattctaaaacttgtgttttattcattaatcagttacgcgaaaaaccaggagtgatgtttggaaataacgaagttacaacaggaggaaaagctctaagattttatagttcattaagaatggaagctaagcgtgttgaattacttaaggataaattcaacaattatgttggcataaaaacaaaagtaatggtatctaaaaataagattgctaaaccttttggtgttgctatattagaaatcatgtttaaccgtggttttgtacatgaacatgaagttattgacttagcacttaaatttaatgttgttgtaagagctggaaattcttattcttttaacaatgaaagcattgctgttggtaaagaaaaattattaaatgttttatcagaaaagccagcattatttgaacaaataaaagaactaactgttcaacaattggctaataaaaattcatttcaacaaacagctagctaacatgagagtttcaatttaaccaaaaagacaagcatatagaatgcactgtcaaagacaattttggtcgtgagaaactaataaatttgatttttcaaattggtgatgctattgaaacttatcatactactttaataagattcaaaattcccaagcattgtttgaatgcaagggatcaaatcaaaaaaataatggagggcaaataaaaatgattacatctatctttggaaaagttacttttgtaggcaaaagaaaaataattgttgagcacaactggatttcatattgatttaatacaaaagaaaaccataaatttgaaaaaaatttggaaaaaaataagcaaattttttgtcatattattaaaaaaattgtcgctaaccaaattatagaagaggcttttgcctttaatactctagaagaaaaagagtggttctgtagattaatagaactcaatggtattggtagtaaaactgcacttaatttgctcaataatgaccttgaggaaattaaacaatacattctggaaaataactacagtgcattatgtggtattaacggtgtaaataacaaaatagctcgtgcacttttatcacttgaaatatttgaaaaatctgaaaataataaaaatattaaaggagttcaagttgctgatggttatgatgaattgtttgaaacactaaagtcacttggttacaaacaacaagaaattcaggatgcactaaaaatgatagaagtaaaacctgattttgatataagtcagttagttgcagaagtaattaaattaatgtcttttaagaataatgaaattacaaataaaaccgcctaatacctttgatgaatttgtaggaaaacaagaaataattagtcaaattcaattaagtattaaagcatctaaattaaataaaacacaactagatcatatcttgttatatggcccacctggtgtgggtaaaactactttagccagattaatagcaaatgaattgaaaacaaagttgcaaattattcaaggtggacatttacaaaaaccaagcgatttcttaaacgcaatttcactcattaaaaaaggtgatgttctttttatagatgagatccatgccgtagcacctaatgtcatggaactaatgtatccagttatggatgtgttcaaaatacaagtattaattggcaaggattttaattccaagatagttgaaatgaaggtaaatccttttactctaattggtgcaactacacaacttggtaaaatcatcaatcctttagaagatagatttggcgttatcttaaacattaactattattcaaatgctgaaattgaaaagatggtaagtatctatggaaagcaaatgaagttagagctaaattcaaatgaaatttcagctatcactgaacatagtaaacaaacaccaagaattgcaattagaatagttagaagaatatttgaacaaaaaattgttaataaaaaaatagaccttgagggtttgtttaagaatttaatgatttataaaaatggtctgcaaagtattgatgtccaatatcttgaggttttaaatcgccaaaatgaaccacaaggaattaagtcaattagttccatgttaggtatagacagacacactatagaaaataaaattgaaccttttttgttgcgtgaaaatatgattcaaaaaaccaaaaaaggcaggattattacaaatagcggaagagaatatttagttaacttttaagataaaaaacattactttaaattatatttaatgatgcaaaatgtcttttatcataacagtaataggtgctgggcatgctggattggaagccgctttcattgtaagcaaattcaacatcaaagtaaaccttttagttcttgatataaatcatttaggttcttgtccatgtaatccttcaattggtggacctgctaagggaattgttactagggaaattgatgttttaggaggtatgcaagcaattgctgctgataacaatgccttacaatataaattactaaatagttcaaaaggacctgctgtgcaagctatcagagcacaaattgacaaaataggttataaaaactggtttcaaagtcaagttaaattaaataaaaacattaatctaattcaatctgaagcaatcaatttaattgttagaaatgaaaaaataaaaggcgttattttaaaagacggaagtgaacttttaagtgatgcggttattatcactaccggaacgtacctaagatcaaaaacatactgtggtaatacagttaaaaatcaaggacctgatcaatctaaaaatagtgaaaaattaagcacaaacttaattaacagaggttttaaaacaattcgtttaaaaacaggaactccgccaagaattttaaaaacttcacttgactataatcaaatggaattagaaattaataataatcaaaaccttgcttttagtactacaaataaaaatttcttaccacttgaaaaacaaataccttgttacttagttcataccaatcaaaaaattcacgatctaatccttaaaaacttaaaaaaatctgcaatgtttaatggtagtatttcagcacaaggaccactttattgtccaagcattgaagacaaagtttttaagttctctcaaaaacctcgtcaccaaatttttgtagaacctgaatcattgagtctagatactatttatttagcaggattatcaacttcttttacaccagaaattcaaaaagaaatcatccagcttttacctggttttcaaaatgcagaaattaaaaagtttggttacgctattgaatatgatgcttttctatctaatcaactaaaaccaacacttgaaacgaagttaatagaaaacttgtattttgctggacaaattaatggcactagcggttatgaagaagctgctggtcaaggtttgatggcaggaattaatgctgctttaaaattattaaaaaaaccaccatttattttgcaacgtaatgaggcttatattggggttatgattaatgatttagttactaaaacaatcagtgatccataccgtttgttaacatccagagcagaatatagactatgattgagaaatgacaatgttcaagaacggctcattaaaaaaagctttgaacttggtttaacagataaaaaaacatatgaattgttccttaaaaaggaaaagaaaaaacaggaattaatttcatttttaaaaaacactcaagtaggcaaggttaaagcattgaaattcactaataaaaataccgctcaatcactttatgacttcaacaaacgaagtgaaataaatttagataaattgatcaaagatcttcctgaaaaataccaattagattcagaaacacttaaacaaattgaaattgaaattaaatatgagggttacataaagaaaaatgaaaagtattttaagggtttagataaattaagcaaaattaaaattcctcatacttttgattaccataaggttaagaatttagctagtgaagctatttttaaactatctaactttaagcctagtaatttagcaattgcaagtcaaatagctggagtgaactttaatgacattatagccataaaacattttttaaaaacttatgaataacctaataataagttagttaaaaatttttcaattaaaaacattgactttgaaaccggaatgaaaaaataatgattttataagggttaaaggtgctagagaaaataaccttaaaaacattaacattgatatccctaaaaatcaatttgttgttattactggtctatcaggatcaggtaaatcttccttagcatttaacacaatttatgctgaggggagaagaagatatttagagtctctatcttcttatgcacgccaatttttaggtaacagtgataaacctgatgttgatcttatagaaggattatcaccagcaatttccattgatcaaaaaaccacttcacataacccacgttcaactgtgggtacagtaactgagatctatgattatctaagacttttatgagctagaattgggaccccttattgtcctaatggtcatggttctattcaaacgcaaacaattaaccaaattgctaatcagatttttgatttacctaataaatcaaaggtgcaattattagcacctactgttaaaaatcagcgcggcatttttacaaatgaatttattaaatacaagcaattaggttttcttagagtcttagttgatggccagatttacaccttagatgatgaaattaaacttgataaaaatactaaacacaacattagtgtagtgatcgatagaattatcatcaataaagataatcaaacttattcaaggatagttgatagcattgaaaccattgataggttaactaatggcaagatagaagttcttaaggaagatggaacaatattaaatttcagcaaaaatcatggttgtgataaatgtggtttttctattagtgaattggaaccaagattattttcctttaactcccctttaggttcatgttcatattgcaaaggacttggttttagttatgaacctgatgtagacaagataattgctgattctaaactttctattaaccaaggagccattgatatttttaaaaatattgtgcatggaacttctttggattgacagcgctttttatctttagttaatcactataaaattccattagataaaccaattgaacagttagataagtcacaacttaatttaattttagaaggaagtgatgaacctattgaaataaaaacaatttccaattcaggtgctaagaatatccgctttgagcattatgaagggatagctaatttaattaaaagaagacacctagaaacaaacagccaagtaagtagagaatgatattctgcatacatgtctgaaataacatgtaaaaagtgtcatggaaaaaaattaataaaagacgctttaagtgttaagttaggaggaattgacattattagctttactgaactttccattgataaaagtattgattttctattaaaactagagttaaatgatgagcaaaagaagatcggtgaattagctttaaaagagattattaatcgtctttcttttcttaaaaatgttggtttagattatcttaatcttgcaagaagagcttctacgctgtcaggtggagaagcacaaagaattagattagctacccaaattggttctcaacttactggtgttttatatgtaatggatgaaccttctattggattgcatcaaaaagacaatatgcgtttaattaaaacaatgatggtaatgcgtgatttaggtaacaccttattagtagttgagcatgacagtgaaacaatgttagcggcagattatttaattgatattggtcctaaagcaggtaatgaaggtggtgaattagttgcttgcggtacacctttacaagtaatggaaaactcaaactcaattactggacaatatcttagtggtaaaaaacaaatctccattccaaaaaatagacatagtggtaatggtaaaacaattattatcaagggtgctaaagttaataatttgaaaaatattaatgtcaccattcctttaaataaattggttttgataacaggggtttcaggttctggaaaatcctctttaattaatcaaacattagttccagctttagaaagaattctttatcgtaaaggtgttaaaaaagatacatataaggaaataattggtgctaacaacattgataagataattgttgtctctcaagacccaattggtagaacaccacgttctaatcctgcaacctatattagtgtttttgatgatattcgtgatttatttgccaacacaaaagaagctaaagcaagaggatatacaaattcacgattttcttttaatgttccaggtggtaggtgtgataagtgttttggtgatggtgtgattcgcattgaaatgcattttttacctgatgtttatgtcaaatgtgaagtatgtaatggcaagaagtacaattcacaaacactggaaattaaatatttgggaaaatcaatttttgatgttttacaaatgtcttgtaaagaagcttatgaattttttaaagctatcccaaatatatcacgtaaactaaggttgttatgtgatgttggtttagaatatttgcaattaggtattaatgtcacttttctttcaggtggggaagcacagagaattaagttatctaagtttttacaaaaaaaatctactggtaaaactttgtttgttttagatgaaccctctactggcttacatttagaagatataaacaaactattaacaataattcaaagaatcattaagaatggtgatacagtagttgttatagaacataacttagatattattaaggttgctgactatatcattgatttaggtcctgaaggtggtgacaatggtggtcaaattgttgctcaaggaacacctgaacaacttataaaccaagttaataaatcttatactgcccaatatttgtccaaaattttaaaaccagattcaatttaaaaattatgcaccaagttttttatcaaaaatatcggccaatcaatttcaaacaaaccctaggacaagaatcgataagaaaaatcttggtgaatgctattaacagggataaactacctaatggttatatcttttcaggtgaaagaggaacaggtaaaactacttttgcaaagataatagcaaaagcgataaactgcttaaattgagatcaaattgatgtttgtaatagttgtgatgtgtgtaaaagtattaacactaatagtgccattgatatagttgagatagatgcagcttctaaaaatggtattaatgatattagagagttggtagaaaatgttttcaatcatcccttcacatttaaaaaaaaggtttatattttagatgaagcacacatgttaaccacccaatcatggggtggcttgttaaaaactttagaagaatcaccaccttatgttctttttatttttacaactactgaatttaacaagattccattaacaattttgtccagatgtcaaagcttcttttttaaaaaaataactagtgatttaatccttgaaagattaaatgatatagcaaaaaaagaaaagattaagatagagaaagatgcattaataaaaattgctgatttatcccaaggttcattgcgtgatgggcttagcttactagatcaattagcaatttctctgatagtgaaaaaattagtattactgatgttgaaaaaacatttaatatcgttgatagaaatgcaaaatttacttttattaaagcagttttatcaggagatataagaattttaactatcattcttgacaatgttatagaagaataatggaacaatttaatgcctttaaatctctattaaaaaagcattatgaaaaaacaataggttttcatgataaatacattaaagacattaatcgtttcgtatttaaaaataatgttcttttaattcttttagaaaatgaatttgctcgtaattccttaaatgataattctgaaattattcatttagctgaaagtttgtatgaaggaattaaaagtgttaattttgttaatgagcaagatttcttttttaacttagcaaaattagaagaaaatagtcgtgatactctttatcaaaattctggattgagtaaaaactatacttttcaaaactttgtaattagtgaaggaaataaaagagcttatgaagcaggcgttagattagctgaaactcaagataacgaattttcaccgctttttatttacggagaaaccggtcttggtaaaactcacctactacaagcaataggaaatgaaaaatttcgtaattttccaaatgccagagtaaagtatgttgtttcaagtgattttgcccaagaagttgttgatgctttttatcaaagggataaaggtatagaaaaactaaaaaaaaattatgaaaatttagatttagttttaatagatgacactcaaatatttggcagaaaagaaaaaaccttagaaattcttttcaatatttttaataacctagttctaaataaaaaacaaattgttttagtttctgataaggctcctgatgaactaattgatattgatgcaagaatgatttctcgctttaaatcaggattattactaaagatagaaaagcataatttgtcttcactttgtgaaatacttactgttaaattaaaagaaaaagatcctaacatccaaataactaatgaggcaagacatgatgcagcacaaatttcaggtaacgatgtgcgtgctttaaatggaattgcaacaaagttattattttttgctaaaacttcaaaacaaaatttaataaatactgaaaatttaaaagaaattctttttgaagaatttgagaagtttcataaaaagagctttgatccttatttattaatagagaacgtttgccgtagatttaatgttcctatggacagtgtactttcagaaaatcgtaaagcagaacttgtccgtgttcgtgatgtgtgtaattaccttttgcgtcaaaagtacaacatgcaatttcaacaaattggcaaaatatttaagagaagtcattcaagtgtattaatggcagttaaaagagttgctaaaatgattgaaaatgacagttcattacgggatgtaattacatcattagtaatttag

SEQ ID NO: 2 - DNA, 22684 bp, M. genitalium
ttaattaataaaaaacaacgcggaaaagctttaaatttatgcaagctagtgaattaacaccaaagtggtatgtagctcctgttagtattaaagatgaagctgttgtaaaaaatctaaaagctaaaattcaagctttaggatttaatcatgagattgttgatgttaaagttctaaaagaaagggaagttcatgaagaagtttattcattaaaatcaggaaaacttcctcgttccttaaagaacactacttttaacaaatggtttgttcttgatgattaccgttatcttagggtaaaaattagtgaaaaaaatctccttggtagatacatctacataaagatgatttatagtgaagatgcttgaagaattgttcgtaacttccctgggatcactggcatagtaggttcttctggcaggggtgctttacctatccctttagatgaaaaagatgctaataatttagaacaaatgcttaaagggatatcaatcaatcctagcaaacgaattatgctaacaaatactgccattattgaaatggacagtgataaatttgatgaaaaatttcaatatatcttaaaacaaaaacaagccattcaaaaaccaaaagaggatgaagattcagaaattgttgatgctgaaaaactaaaagaagcttttaaaaaactacaaaatagtcaagaacaagatgaatggaaagaaaaagcaacgattattcaaagtgagcaaaccaaacttgatccatcagtattagttccttttttgggcaaatatgaaattcttgatactgacaataaagttgaacaactctttgaatttagtgttggtaatttagtagaggtacatttaactgatactattcatgttcaaggacagataaaagcactttatcaaggtacagttaacaaagcagttgtagagatagaattaacatctaaaacccaattaattaacttacctttagaaaatttaagctttgttgagtttgagtaataattcttgggttgatatttagttttgcaccaagataacaatgaaggttttaactgaactccaaaagcagatatttaccattgtcaaaaaggaaaatggtaaacctattccccctggaatagtggtaagaatgatggaaaatagtcctaattttccaggtaaacatctcatctatcgggccattgatgatctgcttgattgagccatcttaaggaaagctggtggggttacaaaccagctattagttaactatgaacctgctgagcctttacttgataaaaaactacaagggattttaaccttaggaaataagaatagtggttttatccgctctttggatgatgataaaactgtgtattatgtccattactctaatttaactggagctttagatggggatcttgtggagttttgtaaattagataaaccccaatttggtgataagtttgatgctgcagttattactattctaaaaagagcaagaatcttgtatgcaggtaattttttagtagatcaaaatgagtttgccttggaatacaaaattgttgctgataaccctagattttatttaactatgattgtaaatcctgattctatcccaaataacttagcatctaacaccaagatagcttttcaaattgatgagtatgatcctgataacaacttatgtaaggtttctgtacaacaagttttgggtaacaatgatgatccgctaattaatataaaagcaatcatgttggacaattccattgtctttgaaactaacgatgtagttgaacagcatgctaacaagttaagttttgatactgaagaacaacataaagcttaccgtcaggatttaactgatttagcttttgtgactgttgatcctacaacatcaaaagaccttgatgatgctatttatgtcaaaacaataccaacaggttttgtgctttatgtagctattgctgatgttgcacactatgttaatagaaatagtgaaatagacattgaagcaaaacacaaaacaagctcaatctatctacctggtcattatgttgtgcccatgctacctgagcaattgtcaaatcagctctgttctttaaatccagcacaaaaacgttatgttgttgtttgtgagattagttttgataatcagggaaggattaaaacaaacaagctttacccagcaacaattatttccaaaaatcgttttagctatgatcaggttaacaagtggttaaataataaatcagaattaaactgtgatgaaacagttatcaacagcttaaaagcagcttttacactaagtgatctaattcaagcgcaacgtcaaaaacgcggtacaattgatctttcacacaaagaaactgagatagttgttgatgaacattattttcccattaagataaattttttggttcacgataaagctgaaaccatgattgaaaatctcatggtagtggccaatgagacagttgcttgggtgttaactaacaacaaaattgctttaccatacagagttcacccaagaccaagcaaaaagaagttacaaagtttgattgaaacagttggtgagttgaacataactaaaccccaatttaacttagatactgtcacttcaagccaaatagctagctgattaaatgaaaacaaagataatcctagttatgagatctttgtaatcctcttattaagaacactaggcaaagctttttatagtgttaatcccctgatgcacttcagcattggttctaaccactatacccactttacttcaccgattagaaggtatatagatctaaccattcacaggttgttgtgaatgcatctttttactcccgatcaattcactgataatgaaagagatcaactcaaacaagagttggaaaaaattgctgatacagttaatgatacagagattaaaattatcaattgtgaaagaaatgccaatgattatctaacaacgctgttattatcaaaacaaattggcaaaaccttcagcggatttatttcagcaattactagctttggaatttttatgagaatggatgaaaataactttgatgggttaatcaaaattacaactatccctgatgatttctttatttttgaaaaggaaaaaatggtattgaaaggaagaaaaactaataaggtttataaaattggcgatcgtttggaagctaaactaagtgagattgatttcatccaaaaacgtgctattttaacactcatataattagcagtgcatcaaaaccagcatgaagaaatataattagatgaagataactttcatttctggacaagaagtgtcgttaggcacttcttttttattgttttcaaaaaaaatagttatgaatgaattaaaccaacccttacttgctattattaaaaatgttgctaaaaccaaaaacctttctatagaagaggtggttttttgtttgaaaacagctttagaacaagcctataaaaaacaccttaactttgttaatgttgaagttaacattaactttgataaggggattattaatgttgaacaactctttaatgttgttagtgatgaaaatgaagattatgatgactttcttgaaatccctttacaagcagctaacaaaataaacagttcattgcaattaggtgatgtgttgcgaaaaccaatccccttaaaaaacattagtagtgatcttatcaataagatgattgctatctttaaccaaaagattagtgaaacaaactttaaagcagtaatgagtgagtttagtagtgaggttggggaagtgattgaagcgaaagttgaagatattgatactaacaaagaaggtggtttaaagggttatattattaaccttgaaactacaaagggttatatctccaagcgggaattgtcaaaaggggagcgcttagagataggtaaaaaatacctctttgttatcaaagaaatccaacggcaagcatcgttatgaccaattactttatcaagaagtgatacccgcttactacagtttttgttaacttcaaatactccagaaattgaaaatggtacgattgtaatcaaaaagattgaacgttccccaggagtgaaatcaaagatagcagttatctccaatgatcctgcagttgacccagttgctgctatcttaggacctaagggtgagaagattagggggattagtgaggaatttaatggtgagattattgacattgtcttttggaatgaagacaagttaaagttcttaattaatgccattttacctgcagaagtcattggttataacatcttgcaggatgatgagcgtgatactagtattgaagttgttgtacctgcaaaccaaattgctaatgtttttggttttaaaggtgtaaacattaggttaattagtaatttaacaggttgaaatagtgttgatgtttacagtgaaaaagatgcaagtgaagccaacattaaattcacgaggttaagctttgaacctgaagggttgtttggcatcaaaaaaagaagggaaaagatcattagtaatgatgctactgataaagtcttttacacctctaaagacaatgtgatagatgatgagattattgttgatttagctaaagatctaatggttgataataaacaaaaacaacctgagcaagttgcaaagcaagttgttgaaaaatcacaattagaaaaacaagttactccaaaagaaaaagagaaagttcaaccaaaagctaaggttcattctaatagccattccaaaaaaccagctaaacctaatcagattttttctatcactgttgatgctagtgataagaatcttaaaaaagatcaagttgataataaccaaacaaacccccaaacaaaacaaacatttgatagctttgatgatctttaacattattaagaaataaaccaattagtgatctataaaaacaatggaaaaatttttaaagtacgaaattaaggttaacaacaaccaaccaaccaacactaaccctaactatgggatctttgaagtagcaccgttagaatcaggatttgggattaccattggtaatgcgatgcgccgagtgttacttagttgtatcccaggcgctagtgtgtttgccattgccattagtggggtaaaacaagagtttagtaatgtggagggtgtgttggaagatgtgactgaaatggtgttaaacttcaagcaactagtggtgagaatctctgatcttttgtttgaagatggggagatgatcgaaccacccttagaaaggtgaccagttttaaaagttactgctgaaaaaaagggtgcagtatatgcaaaggatcttgagtgtccagctggttttgaagtgattaataaggacctttatctcttctctttacaaaaggacatgaaactaacagtcagtgtttatgttaaacagggtaggggctttactagctttcttgaaaacagagaattgatcaattcgcttggcattattgctacagatgctaacttttccccggttttacactgtggttatgaagttcaagaggtgaaaacttccaaacaaaagttaactgaccatctcacctttaagattgctactaacggtgcaattaaagcagtggatgcgtttgctatggcagcaaagatcctaattgaacacttaaacccaattgtaagtgtcaatgagtcaattaagaatttaacaattatccaagagaaagcagaggaaagaaaggtgaaatcatttgccaagcaaattgaagaacttgactttactgttagaacctttaactgtttgaaaagaagtgggatccacacactccaagagttactatcaaagtcattaactgacattagagagattagaaacctaggtaagaaatcagaacgggagattatcaaaaaggtgcaagagttaggtttaaaattccgttcttaatttattaaaaaaccattagcacaaaagattaaaacttactatgaaacagtgttttgttgttacaactaccaaacgcttagatagtcttttagctagcttactgaacctttcaagagtaaaggtagtgaagctgatcatgaatggacagattaaagttaatgaaaaactaacttttaaaaacagtttaatagttgcaaaagatgatgtaattaaagttgagattcatgatgagacaactagtgatttcattactagtgttgaaccttataacttaaagcttgaggttctttttgaagacaaggatttgatggttattaacaaaccatcaggtttgttaacccatcccaccactttcaatgaaaaagccagcttgttagctgcttgtatctttcacaacaacaaaaaccctgtttacttagtgcacagattggaccgtgatactagtggggcaattgttgtctgtaaaaaccaagcaagcttattaaatttgcaaaatcaactgcaaaatcgcaccttaaaacgttattatgtagcactagtccacttcccttttaatgccttaactggttcaattaatgcacctttagcaagggttaataacaacaaggtaatgtttaaaatagcccaaactgctaaagcaaagcaagcaataactaagtttaaagtgattaatcagaatgaaaaagcagcactaattagcttggaattgttaacaggtagaacccaccaaattagagtgcatctgaaatttatccaacatccagtttataatgatccactgtatggaattaaaagtgaaaagaaagatagctatggtcagtttctccatgcaaacaggatctgttttatccatcccactttaaacaaacctatggactttcacgccccacttgaacctaagttttcaacgaaacttaagagtttaaacttatctttaaccgatccactccatgttctttttaagtaacttaagataattgaaacattaaataagataattacaagctatatgtccactgacaaaaaaacgctaggcgaaaaacccaattcaaccaaaccagaactatctgaagaattaattgctgaacttaaaaaacagcgtattcttgaaaagaatcgtccttacaaaaagatgatttatgttgacaataaagtgcaacgcaaacaccgtcatgaaaacatcgcttttctcaaaacccttcatgaaaataaggagagtgatgttcctaaaaaaagaaggggtagaaaacctaaacacgctcctttaaaagaaaaaaataatctgaagttatttgatatcttagaaggatcgttaaaaagccacattgaaaatgatgacaccaacacagtcatcaaccttctaacagaagcttgagaaaagaaaagcaaaaagaaacaaaaaaacatcacgctttcaaataaggaaattattagtgttctcgctaagtttgaactacctgaagatgaaattatctatgttttggatgaactacgtgataaggggattcaactccaacacgatgttgaagagcacatccatgaatttcgtgctaaccaagacctttcaattattgatgaagatattgaagagttaacaagtaagaacatctctaaccgtgataaggttgatgataatgttaggttctttttaggatcacttgacttttctaaaatgttagattttgaatctgaacagcggattgccaaggttttaaatagtactgatgaagagtcacgtaagtatgcaattaatcagttggttacttcaaacttaagactagttgtttctattgccaaaaaacacctagaaagagggttggattttaatgatttaattcaagagggtaatttggggcttttaaaagctatttccaaatttaactgatctttagggaataagttttcaacttatgctacttgatggattaaacaagcaattacaagagcaatagctgatcaagcaagaacagtaaggatccctgttcatatggtagaaaccattaaccgcttagctaaagcagaacgggctttgtatcaagagttagggcgagaacctactgatgaggagttagctgaaaagatgggaggacaagctgaaggatttaatgttaaaaagattgctgaaattaaacggttaagtttagatccagtttcgcttgataaaacagttggacatgatgaagagtcccagtttggtgattttgttaaagacacagacgctcaaactcctgacgagtttaccgaaagccgttcaaattcagaaaaaattgatgaattgttgaacaataatctttctgaacaagaagagttaattgttagaatgcggattggcatgcccccttacaatgaacctaaaacacttgatgaagtaggtcaaaagattttgatccctagagagaagatcagacaaattgaaaacaaagcaattagaaaattgagacatgcagttagaaacaatcctattagtatgtcatttctaagaattaatgaaaaaaaggattagactatttttaataacttaaattttatataaatattaagtaatggcaaccattcaggaaatcgagtgtgattttttagctaaaatagcacaaaaatttactaatgcagagattgaattaattaacaaagcattctatcacgctaaaacttggcatgaaaaccagaaacggcttagcggtgaacctttttttatccatcctttaagaacggcattatcactagttgaatggaacatggatcctatcactatttgtgctggtttgttacatgacatcattgaagatacagaccaaaccgaagctaatatagcaatgatttttagcaaagaaattgctgagcttgtcactaaggttacaaagattaccaatgaatctaaaaagcaacgtcatctcaaaaataaaaaggagaatcttaacttaaaaagctttgttaacattgcaatcaattctcaacaagagataaatgtaatggtactaaaactagcagatcgacttgataacatcgcttccattgagtttctccccattgaaaagcaaaaggtaattgcaaaagaaactttagaactttatgcaaagattgctgggaggattgggatgtatcctgttaaaacaaaattagcagatctttcatttaaggtgttggatttaaaaaactatgataacaccctgtcaaagattaacaagcaaaaggtcttttatgacaatgagtgggataacttcaaacaacaattaaaaaaaatcttagcgcaaaatcagatagaataccaacttgaaagtcggattaaaggcatttactctacatataaaaaactaactgttcatgaacagaacatcagtaagatccatgatctttttgctatccgcttaattactaaatcagaacttgattgttatcacatccttggtttaattcaccttaattttttaattgacagtaaatacttcaaagactatattgcctcacctaaacaaaacctttaccaatcaattcataccactgttcgtttaaaagggttaaatgttgagatccaaattagaacccaacagatggacaatgttagtaagtttggcttagctagtcactggatctacaaagaacagaaagagggattgttagcacctgctttgcaacttaattacctagtgacaaaacaaaaacactcacatgattttctaaaaaggatttttgggactgatattatcaagattaatgttagtgctagtcatgaacctaatgtaattaagcaaattaatgttgatagcaacaataaactccttgatattgcttttgaaaactatcccaagcaatttgctaaattaaccaaaattgaaattgatggggttgagatcaattcttttgatactagtgttgaaaatgagatgctgattgaattttactttggcaagaataacaatttgaaatcaaagtgaattaggtatatgaataaccctatataccgtgaaaaggtaaaaaagagcttggctaaactagctaaatctggtagatacagtgagttagctttttatgaaaaagaactgggtgaaaaacagttaaaacttgctagtgaaactgaaatccaaaaacgcttaaacaccctaagaattaaaaaaatgagtgattacttagcgttaattgagtgtactaactttactaatgatgaacatttgttgtttctagctaaaaacaacgacaagtgaaataaactaacaaaaccacttaagtttgctttttcaaaagtagtttttcacaactcttactttgaacaaattgaaggtatttttatcaccaaaatagtgattgaaccatgttgtagtaagatccctgatatgcctgaacaagtaactggtatcttaactaaaaacattttaagtgttcaccgttatggttgtaagaatttacaaaataaaaagcagttaaaaattatcccgttatattgaaatatccagcagttaaaactaaaaccacgtaagtttcgcagttacattaacattaacggagtgtggagtgaaaaaaccattaataaaatctgtcaaacaattattaatggtgatggttatattgaaaaaataattcccaagatcaacaaacaaaaagatgaatttgatttaaacatcaccctttttgttaataactaccaacaacttctcaccttaatggaccaaattaccactaagaatatcagctttagttgaaaatacctttagagtggctttttaatgttttgatccgcttttcaccccacaaatgtcacaaaaatctaatttctttcaaaaacgttattcccctacagctaccagaaggtattatggcaaaattgagaccaattttatccaaccaaatttagctgatattcagattaaaagctaccaaaaattcttagatcatgatcttgaaaaattaattgcctcatattttccaatcaaatcccctaatgatcgctacactatcaattttaggggattacacagaactgaaccagaacgtgatgaagcacaatcacgtgctcaatctaaaacttatgaagttggtatttatgctgatcttgaattagttgataatgataaaggaacagttaaaaaagcacggaaatcaaagaaaaatattgctagcaatacaaatggtgtatttttagctagcatgcctttaataacccatgatggggtttttattatcaatgggatagaaaagtttgttatttcccaaataacccgttctccagggatatacatgctaacaaaatcccaactaaaactatccaactcccgtaaaagagtacaggaaggttatgtttgtgaggttttacctgctaatggttcagtgatgcttatatacatctccaataaaaaaaagattgaagatgcttttgtacaaattcttttaagagatgcggtaagagaaggtgctaagattttccctattacaacacttttaaaagcgtttggtttaaataatcgtgagatccttaaaatctttaaaaacaatgaatttatcaaacgttcattggaagcggaaatttacaatgctaaggattttttaagcaatgttgatcctgaaatcaaaaaccttttaaaagaatttagagatggtaaaactgatttaagaagaaaagggatcgcttctgatcagaaattaagatcacttgttaatgaatatgtaacgcttgagaaacaatataatgcgctaaaacaaacaagtccaaatgattctagtttaactgcacttgaactggaaatggaaaacaaaatggatagtgttattactgaaagagctgctaaacacattgtcaatgaactttctatttcactgcgtgatattgaaaacactgaagagtgtcatgaagtgagtttccatgcacttttatgtgcgcgtttttttagaaacaagaggtacaacctctctaatgcagggagatacaaagtatctagaaagttacgtttaacagaacgtatttatcaaaaaactctggcatgtgatttgttcttaaaagatggcaagctacttttaaaaaaaggtactttacttttaaaagaagagattgacaaaatcaaacaagctgctaagaacaatgaaattagttttgttaataaaatgcaacttacaactgatggtaaggctgttgatttagcaaaagaatcactcttttatgaaacgatagatgtatatatcactaatgataatcttagtgtttcagtaccagttatagggatccataacgaaaatgatctgaacaaagcaatgactctcagtgatttcatcgcttcaattagttatgtgattaatttaccttatggaattggtaaatatgatgatattgatcaccttggtaataagcgggttaagttaattaatgaattaattactgctaaattagaaagcggcttcactagaatggagcgctttttaaaagaaaagttaactattgctgatggagttaaccgtggccagcaaattaatgaagagggtcaggttattgaacaaggtgaaaaaaaggaattaactattaaatctttaatcaactcaaaaccaattcaaattgtgattaaagacttcttcaatacccaccaattaacccaatttttagaccaccaaaaccctttatcagaattgagtaataaaagaaggatttcagcaatgggacctgggggaatatcaagagaggaccctaatttagatatccgtgatgtgcattattctcagtacggtagaatttgccctattgaaacacctgaagggatgaacatagggttgatcatgtctttagctagctttgctaagattgatgaaaacggatttttaatggcaccttatcgcaaaatcaaagctggggtaattactgatgaggtggaatatttaactgcgcttagagaagatgaacatattattgctgagatctcttcacttgtcaatattagtaacgataacaagatcttagataaggaaattattggtaggtatcgatctatgcaaggactttatgatcctttaaagattgattacattgatgtagcaccacaccaagttgtttccattggttcttctttaatcccctttttggaaaatgatgattcagctagagcattaatgggaaccaacatgcaacgtcaggcctatcctttaataaagccatatgctcctgcagtaggtactggtcaagaacacaaaattgctagtgattcaggtttaacaatgtcctctccttgctcaggtgttgttagttatgttgataacagtaagattattattacaagtgatagttctaaaaaagagacagttaacctagttaaatttgaacgttccaaccaaaatacttgttataaccacaaaccaattgttgaaataggccaaagggttaataaggatgaaatcattgttgatggccctgctgttaataagagtgagttggcattaggacagaatgttttagttgcttttacaacttgaaatggttataactatgaagatgcaattgtcatttcagaacgattagttaaggaagatattctcacttcattaaccattaatgagtatgttgctcaatgtttgtctactaaaaatggtgatgaacaaattacccgtgatatccctaatgttagtgatgcaaacaaacgctatcttgatgagaatggcatcattatggtgggtgctgaagttaaagaaggggatgttttggttggtaaagtttcccctaaaggtcaagtcgaagtctctcctgaagaaaagctatttaaagccatcttccctgaaagtgttcaaaacgtgagagactcttcacttaaagtttcccatggtggggatggtattgtttcagctgtaaaacgtttttcaattgctaatggtgatgaacttaatgatggtgtgattgaaatgatcaaggtttatgtggttcaaaaacgtaagattcaaattggtgataaattagctggtagacacggaaataaaggagttatttctaaagtggtgcctattgaagatatgccccatttagaagatggaaccccagttgatattctgctcaacccccttggtgttcctagtcggatgaacataggacaaatttttgaaacccacttgggttatgcagcacacaagctagcagttcgttctttaattagtagttgttttgatcaaaataaagctaaggagtttgccattgaaatcaatcaacctcaagcaagggttgaaagattaattaaaggtttaaaaaaccaaatcaatgatcgcaatattaaaagtgaaaaagaagcacttgaaaaactcgataacagtgacattagtttagttttgaaagagatagggatgtcttttgatgatcttatttacaaaattgcaacccctattttccaaggagtgaacttcttagatctccaagatgttatgcaagaagcaggattagatccccaaaaaaatcagggtaagtttaaactcattgatggtaggagtggaatgccatttgaaagacctatttcacttggaattatgtacatgatgaagctgaatcacatggttgatgataagatccatgctcgtgctgttggcccttattctaagatcactcaacaaccattaggtggtaaatcgcaaaatggtggacagcggtttggtgagatggaagtgtgagcattagaagcttatggagctgcttataacttgcaagaacttttaaccattaaatctgatgatgtacaaggaagaaatagggcttatgctgctattgttaaaggtgcagctttcccagagcctggtatccctgaatcatttaaattattgacaaaagaattacagggcttggctttatctgtttcatttatctatgatgacaacacccaacaagactccaataatgtttccatcttgcaaagtgatggggaacaagatgaatttttcaatgattttgaatttgacactgagggttattagaaattaacaatgacaacaacaagacgtaataaaagaaataacaagctttataaaaacattaaagcaattaaactttccatcgcttccaatgacaccattttgaactgatctgaaggggaagttacaaaagctgaaaccattaactataaatcattaaaaccagaacctggaggcttgtttgatgaagcaatctttggacctgttaaggactatgaatgtgcttgtggcaagttcaaaaagattaaataccgtggtgtgaggtgtgatcgctgtggggtgtgagttactgaatctattgtacgtagagaaaggatgggacatattgcacttgtgagtcctgtagctcacatttggatgtcaaaagaattaccatctccttccaaaatatcattagttttaaacatctcttacaaagaggttgaacaggttttgtactttgttaactacatagtacttgatacaggtaagatcaaagatgataaaatcatgccttttaagttcaaagaagttttggacttaactggtaagggttcactttcaacacgacaaaaaatgcgtcgtgtgataggttatatcttcagaaatctcattaaaagtaagagtagtgaagattaccgtaagggaaaaatcttttatgaaagtttaaaaaacagctctctccccttttctctaaatgatgcttttaattacattaagaagtacactggttttagggttggaataggggctgaagcaattttggaattgcttaataaaatcgatcttaacttggaatttagcaggttaaatgatgctttaagaaaagccaagaaagatagtgttgaagatgctaaagttaagaagatcttaagacaactggaaactattagttggtttagaaattctaagcttcatcctaaaaacatgatcttacatactgttccagttatcccccctgatatcagacctattatccaacttgatggtgctaagtttaccaccagtgacatcaacaatttttaccgcagggtaatcattagaaatgaccgattaagaaggattttggaagatggtactgtaccttctattgttgttaacaatgaaaaaagacttttacaagagtctgttgatgctttatttgataactcttcacgtcataaaccatcactttccaaagacaaacggtcattgaaatctttaacagatcgtttaaaaggaaaacaaggtttatttagacacaacttacttggtaaaagagttgattattcaggtagaagtgtaattgtggttggccctgaattgaagatgtatgaagttgggatcccagcactaatgatcttaaagctgtttaaaccctttattatccatggattgatcaataagtttgatgaaaatggtaatgagattagaccaattgccgcttccatcagacaagctgaagatatgattaaaaaccaggatgatcttatctggggaatagtttatgatgttatcaaagatcgtcccgttttactaaatcgtgctccaaccctacataggttagggatccaagcatttgaaccaagaattgttgatggtaaagcaattagattacacccattagtaactactgcatttaatgctgattttgatggtgatcagatggcagttcatgttcctttaagtgagaatgcagttaatgaagcaagagctgttctgcttgcatcaaaacatatcttaggtttgaaagatggaagacctattgtaactcctactcaggacatggttttaggtaactattatctaaccacagagagaaaaggacagttgggagaggggattatcttcagcacagtttatgaagcacgtgctgcttatgaaagtcaaaaggttcatttacatgctattgtagggataagtactaaagcatttcccaacaagaagtttgcatgccaaggaactttaataacaacagttggtaagattatctttaatgatgttttaggcaataatgttccttatattaatgacggggaatttgatgaaaatgcatgtcccgaaaagttcattgtgaaacagggagaagatgtaagacaatcaattttaaagcatcaaattatccctgcattttccaaaaaggttatttccaagttaatcgatctactctatcttttattggaatttaaagaccttcctaaaacacttgataatatcaaagcacttggctttaagtactctactttttcttcaactactgtttcagtatttgatatccctaagtacaccaataaacaaaattactttgatagtgctgatcaacaggtgctgaaatacaaacagttttataacaagggtttgttaaccgatgatgaacgttataaacgcgttgtgaagttatgaaacaatgtgaaagaaaaagtatctgatgagatccaaaacttaattaaacaagaacagtaccgtgataattccattgtggtaatggctgattcaggtgctagaggtaacatttctaactttacccagttatttgggatgcgaggcttaatgtctaaaagctttaactatgaaagaaataaccaatctaagatcattaaagatacgatagaagttcctattaaacactccttttttgaaggtttgaccattaatgaatacttcaactcttcttatggagcgagaaaagggatgacagatactgcaatgaaaacagcaaagtctggttatatgacaagaaagctagtagatgctactcatgaattaattattaaccatgatgattgtggaacaagaaaaggaattgttgttgaagcaattgttgaaaccaaaaccaaatccttgattgaatcattatttgacaggattgttaatcgctactcaattactcctatagttgatcctgaaacacaaaaaactattgtagaagctaacagtcttattacaacgcaattagctaaacagatttgtgcaacatctattaaagaagttttagttagatctgttatctattgtgaaagggaaaatggtatctgtcaatactgctttggcattgacttgtcaactggtaagttggtggaattgggaactgctgttggggtgattgctgctcaatcaattggtgaacctgggacccaattgacaatgcgtactttccatactgggggagtttcaactgaaaacaacttagcacaaggctttgaacgtttaaaacagatctttgaagtagttactcctaaagattttgaaaaagcagttatctctgaagtgaaaggaacagttaaatcaattactactgttcaaaacgctcaggaagtagtgattaaatcaaacgttgatgagaggatttatactatccctttcagtgctcaaatacgtgttcatgttggtgatcaagtttcaccaggttctaagattacagaaggttctgttgatattaaacaacttttgcgaattgcaggtatccaaagggtaagacaatacatgattgtagagatccaaaaagtgtataggattcaagggatagatattgctgataagtatgtagaaattattatcagacaactaactaatttgttgcaagtaacagatgcaggtaacagcaatttatttgttggtcagttagtgcatagccattatctcaatgaactaaataagagcttacttttagctggaaagatgcctgttattgcaattaatcaggtgtttggaattgatgaagcggcaagtaaatctaactcctttttaagtgctgcatccttccaagataccaaaaaaatcctaactgatgctgctgttaagaaccaagtagactatcttttaggtttaaaagagaatgttattattggtggaaaaattccagcaggaacaggatttttaactgatgaagagttaactttcttaggtagcaaaacagttgctgaagagtattaaatcagagtaattttattaatatttatctaacttacatctgatgtataaatcagtaataaacatagttttattttgtccagaaattcctaataacactggcaacatcgtacgtagttgcactgcttttaaagctaatctacacttaattaaaccttatggctttttcttaaatgataaaaggatggttagagctggtttaaattgttgagataaaattcaattatttgaacacaaatcatgagaacatttcttacaagcaaccactgaaaataaaactatttggcttttaactaaaagtggtgataaaactcctgatcaaatttgcatgacaaataaattaccaaacgaactttactttgtttttggtcaggaaacaaagggattacctaaaacaatcatggataactttaaacaaaaccaaattagaattcccatttgaaatagtgttagaagtattaatcttgctaatgcagttgtctgtattttgtatgaatattcaaagcaaaatcaatactctaatttagataaacagtgcgcttaatctgatcttattaatgcaattaaaacttctttgaataactatgaagaataaagttttgaaactaaaaaataataagatttttgataaaaaactagcaacttttttaaagaatttagatatttttcctaataattgagaattttttgaaaaagcttttattcacgcttcttacatcaatgaacatgaagatgttagtgaaagttacgatcgcttagagtttttaggtgatgctttaattgactttgttgttgctaaaaaactatttgaactttatcctaaatataacgaaggtcttttaacaagaactaagattgaaattgttaagggtgaaaatcttaatcgtattggtatggagctaaaattaggtgattttgttaagttaagcaatggtgctgaactaactgaaaacactgttggtgatgtacttgaagctttggttggcgccatttatgaagatatggggatgaaaaaagcaactgaatttgttgaaaaatatatttttgaaagaactttttctgagattttaaaatacgatttcttttcactttttcaagagcaaaaattacctgaaccaagagttagggtaagcttaacttcaaataatttggtacttagtataattgaacttgatggtgatattatttgatcacaagctatcccaaataacaaaaattacgatgataaaagtgttttagagcacaacgcaatggcttcttttacaagttttttaaaaagtagtaaaggaagccatttttttagtgatttaaaagagaagatagaaaatcaaaagatgtgtaagaaactagctattaaacctaaaaaaaattagaatctatacatctaaacaaattaacaaagccatttaacttatggactcaacctttcatgagcttgggatctctcaaactttaattgaaacgcttaatgcgcttcatattaataagccaacaaaaattcaacaaatctctatccctcagtttttatcagaaaaaaacttaatagttcactcgccaacaggaactggtaaaactgctgcttttgcaattcccataattgagaagctattaaaagaagatcaaacagcaaaaccaactttagtaattgctccaacaagagaattagtagaacagattaaaaccacattttcaaatattgctaaaaataaaaaactaagaattattagtttaattggtggtgtacctgcttgaaaacaaatcaaaaaaatcaaaacaaatccccaaataatagttggtactatgggtagaattatggatcttttagagcgtaaagcaattcattttagcgatttagaacacctaattattgatgaagttgatttaatgttagaccgtggttttaaaaaacaaatttttaatttactagaacaaatcaattcctttaaacaaattgctgtttattcagctagttacaaccaagaagctattaacattgccaagcaaattactaataatgggatctttattggatcacctgaatttaataaagacgcaaataccaataatgataaactaatcaaacaatttgtttgttatctattttcagatcaaaaaaagcaagctttatacagccttataaaaacagcacaagttaagtcaatcattgttttttgtgacactaaaaaactagttgatgatcttcatgtatttttaagaaaaaatgaattaagaacttttgcacttcatggtgataaaaaacaatttattagagagagaaatcttaaaatctttgccaatacaaaacaacccacgattctagtaactactgatcttattggtcgtggtatccatgttgaagcaatcgatatggttatcaattattcagcttgtttaaatctagaagcttatataaatagaatgggaaggactggcagaaacaatcataaagggacatgtgtaactttctgcacctcacaagaaaagaaagtctttctgaaaatggttgagaaaatcactgataatcgaatagctgaatgtaaacaaatggaaataaagttaattcctttaaaaaataaagctaaaactaaaaaaggtggtatttcacttgattgtgttcagaaaatatatgccaatgcaaaaccatatgaccgtaataaacgtgtccctttagcaagtgatcttttcaaaagtcgtatgcgccagcctgaaaaagctatgcaaaagcaaaaaattcatgacaatgactgacaaagtaatatgtaactaattttttccattacaaatgttttggtaaagattaaagagttgacaaggactcaactcttgcgctctaatgctagtctttaaattttgcttttgaataatgttaagtaaataatcaactgcaaaaaaatgctttaagttattaattaacatcttccttctttgattgaaacattgctttaaaaacagaccaaacttaaagtcataactaaccgatttatttttttctaataatattaaggttgaatccaccttaggtttaggtttaaaagcatgcctatcaattttaaaaactgttgtaatagtcaaatagtattgacaaaaagcaccaaaggcactataatcactagaattaacctttgccagaagccgattagcaaactctttttgtgtcattaaaacaaagcttcgaagctttgattctaaaaacttattgattattggagatgtaatgctatatgggatattaccacacaataatggacttaaattttcaaaaaaactattaaagtcctttttgagaatatcgcctttaactagttggtcttcagttaatatcttttcaactagaagatattcaattaagcgtttatcaatttctatccccttgtaaggtattttgagttttaacaaataatttgttaacgctcctttaccaacacctatttcaacaattgcttgtggatttaaatttttaacaaaagcaaaaattcttttaatgacgcttaaattaaccgtaaaattttgacctaatttacgtgaaggaaaaaaactattcacgctctaaaatcaaatctaatttggaataattttgctttctcaaggacgtaaagttttaaggcaaagacgttttaaaaatcgtgctcaactcacggtttccagtgagcgttaaaaagagtcacagcttaagagaacgcaaggtttttacaaccattcttcaaagtaaaaccaggttctttggtacctttattaacgcttattttattaagaataatcattctacttgaagggttgcaatatcaattgcaaaaactaaatataagctagcagtacaacgtaacctaattaagcgtcagatccgtagtatctttcaacaaattagtaataatttagaaccttgagatattttagttattgtcaacaaaggctttattgaattaacatttaaagaaaaacaaaaactttttttgcaactattaaagcggataaaagaagtagatgcctatcaaactagcgcaaacaaataa

SEQ ID NO: 3 - DNA, 38459 bp, M.geniitalium
gtagtgttaaaaacattgatatttaatttggttagtataaatgttggatccaaacaaattacgcaataactatgatttctttaaaaagaaactgttagaaagaaatgtaaatgagcaattattaaatcagtttattcaaactgataaactaatgcgcaaaaacttgcaacaacttgaacttgctaaccaaaaacaaagcttgttggcaaaacaagttgctaagcaaaaagataataaaaagctattagctgaatcaaaagaacttaagcagaagattgaaaacttaaataatgcttataaagattcacaaaacattagtcaagatttacttctaaattttcctaatattgctcatgaatcagttcctgttggtaaaaatgaatcagcaaacttagaacttcttaaagaagggagaaaaccagtttttgatttcaaacctttaccacatcgagagttatgtgaaaagttaaatttagttgcttttgataaagctactaagattagtggaactaggtttgttgcatatacagataaagcagctaaactacttagagcgataactaatctaatgattgaccttaataaaagcaagtatcaagaatgaaacctgccagttgttattaatgaattaagtttaagatcaaccggacaactacctaagtttaaagatgatgtttttaaactagaaaacacccgttattatctttctccaactttagaggtacaacttatcaatttacatgctaatgaaatttttaatgaagaagatttacctaaatactacactgcaacaggtattaactttcgtcaagaagcgggtagtgctggtaaacaaaccaaaggaactattagattgcatcagtttcaaaaaactgagttagttaagttttgtaaacctgaaaatgctatcaatgaattggaagcaatggttagagatgctgaacaaatcttaaaggcacttaagttaccttttagaaggttattgttatgtactggtgatatgggctttagtgctgaaaaaacatatgatcttgaagtttgaatggcagctagcaatgaatatcgtgaagtttcttcttgttcatcttgtggtgattttcaagcaagaagagctatgattcgttacaaagatattaacaacggtaaaaacagttatgttgctactttaaatggaacagcattatctattgatagaatttttgctgcaattctagaaaattttcaaacaaaagatggcaaaattcttatcccacaagcattaaaaaaataccttgattttgacacaatcaagtaagcaagaattataattaacactctaaggatgcaagtgataaatgaagcgttgttatattacaacccctatctactacgcatcaggtaagccacacataggtcatgcttttaccactattttggcggatgtaattaagcgttttaaaatccaaaacggatatgaggcttttttgcttgttggcagtgatgaacatggcaataaaatagaaagtaaagctaaaagtttaaatttagatcctaaaacatttgttgatattaacgctcaagcttttaagttaatgtgaaagacccttaatattagttttgatcactttattagaacaactgatgaaatccataaacaacaagttcaaaaaacatttcaagatttatatgacaaaaaactaatttatcaaagtgaatgaaaaggggcatattgtgttgagtgtgaacaaaattactttacttttaataaacaaacaatgttatgtgaaataggtcataatctcagtcttgtccaagaaccttgctgatttatttctttttcttctactaaaaattgaattgaaacaacgataggaaaaaatcaacttaacattattcctaaatcacgtgcttctgaattaaaaaataactttataaacaatggtttaaacgatttagcattaacaagaaaaaatgttacttgaggaataaaagttccttttgatccaaatcaaacaatctatgtttggtttgatgcattgttttcttatatcaccaatttaggatttagaaatggtgatcctaattttataaagtgatgaaataatgacaataaagaaagagaagttatccatcttatatcacgtgaaatcaccagatttcactgcatctattgaccgatttttctacacttacttgatattaagttaccaacccaatttttatcacatggctggatagttgatggtgaagggagaaagatgtcaaaatctttaaacaacgttatctctccagaacaattaattgatcaatttggtgttgatggtacaagatattgtttattaaaagagatgcgtttagataaagataatcgttgtagtgttagcatcttaaaagagatttataatgctgatcttgccaatagttttggaaaccatgtttcacgtacttttggcatgattaaaaagtatctaaacggcaaattagaataccaaattattactgataatgcacttcaaaaaataatgattttaatagatgaatcaatcgttcaatttgatcattactttaacagttatgaattttatagagcgattaatctacttttaaaaattgtttttgaattaagtaaattaattgatgatttcaaaccatgagaattgtttaaaaatcaggaattctcacttttaaaacaactactttttacttgtgttaggtgtgtgcaggtatgctatgtgttgttaacacctatcttagtaaatactgcttcaaaagtttttcatttatttaatttcgctgatgatgcctgtagaaaagatcaattaagagatgcaactttattaaaaaaaattattatctctaattcaatggaagttttatttaaaagagtagattaaatatttacccatataatttcgaaattataattaatgacacatgaacttcttgcaaaaaccaaggggagttaaagattggtttggtgatgaattagtttattttaattggattgttaaaaaaataagatctttagcatttaattggggttttagtgaagttaaaactccgttgtttgaaaatgcacaactttttcaaagatctaatgctaatgctgatattgttcaaaaagaactataccagttttttgataaatctcaaagagaattagctttaagacctgaagctactacaccaatagtaagacttgcttgtgaaaacaaattaatgcaagaagcaaattttcccttaaagttattttgcattggttcaatgtatcgttatgaacgtccacaaaacaataggtttcgtgaacattggcaatttagttgcgaagtatttggtttttccaacctgtttatctttttagatacacttttgtttgctaactctttgcttgaagcacttggaattactggatatgtgcttaaaattaataatcttgctaactttgaaacacttagtaagtgaaataaagccctaaaagattatttaactccatataaattagaactaactgagctttctcaaaaaagattagaaaaaaatcctttgagaattttagatgacaagatagatcaaaaaaaatcatttgttaaaaatgctcctaaaattactgattttttagatgcaagtgcaaaacaagattcagaattgttaaaaacacaactaaaaaaacacaatattagttttgaatgaacagacaatctagttagaggattggattactatactggatttgtgtttgaatatgtaaaaaatcaagacacaattttagcaggtggagtttatgataacttagttgaagaattaagtagtaatccaactcccgcattaggttttgcttgtggaattgaacggttaattaactgtttagaaattgataaaaaagcatttattttgaatactaaaccaaagcagatgttagtaatttgcttatttgaagaagcgcttgaagaattggtttgactagctaaattatgaagggaatataaccaagtaactatttatcctaaggttattaaagttgataatgggattagattagcaaatcgcttgggttatactttcattggcattgttggaaaaactgattttgacaaaaaagctattacaatcaaaaacttagtatctaaacaacagaccatttacacttgaaatgaacttggagaacgaaatgtgttttaaatgtgttttaaccaacgaattttaattggctcaatttcaactgaacaactcaataaaacaatagttattattgggtgaattaaacggattaaaaagttaggtgaaattaactttattatcgttggtgataaatcaggaactatccaagtaacttgcaaagataaagaacagattcaacaacttacaagagaagacatagttattgttaaagccaaattacaacgcttagatagtgttagatttgaactgataaatccaactattaaacttttttcaaagtcaaaaactcctcctttaattattgaagatgaaactgatgctttagaagaagttaggttaaaataccgttaccttgatctgagaagacgtttgatgcaaaaacgattgttattgcgtcatcaatttatattagcaattcgtaactgatttaaccagcagggttttattgaaatagaaacacctaccttatccaaatcaactcctgagggagcacaagactttttagttcctgcaagaattagaaaagattgtttttatgctttagttcaaagtccacaaatctataagcagctcttaatgattgcaggagttgaaaaatattttcaaattgcaagggtctatcgtgatgaagatagcagaaaagatcgtcaaccagaacacacacaaattgatttcgagatctctttttgtaaccaaaaaatgattatgaatctagttgaaaaactcttttttagtgttttcttagatgtttttcaaatcaaaataaaaaagacttttcctgtttttaaattttcagaactttttgaaagatttggtagcgataaaccagatttacgttatggttttgaaataaaagatttcacctcgctttttcaagatcatcagaatcagttcactaaattaattgaagcaaaaggcattattggtggtattgaacttactaatattgagttaagtacagacaaaattaaagcattaagaaaaattgctaaggaccatgatgtgagtttagaagttcataataaaaataattcaacattaaaaacttcaattaaatgtgatgaaaaaaacactcttctgttagtagcaaataaatctaaaaagaaggcatgaactgctttaggagcaattagaaatgagttgaaataccacttggatattgtcaaacctaaccaatacagcttttgttgagttgttgatttccctctctatgattttgatgagaaaacaaatcagtgaatatcaaatcacaacatcttttcaaaacctaaacaagaatgaattgataattttgaatcaaataaaaacgaagcattaagcgaacagtttgatcttgttttaaatggttttgaaattggtagtggttcaataagaattaatgatccaattgttcaaaaaagactaatgaattctttgaacattgacccaaataagtttgcttttcttctagaagcttatcaatatggtgctcctgttcatggtggaatgggactaggtattgatcgtttaatgatgattcttaatcaaactgataacatcagagaagtaatcgcttttcctaagaataatcatggtattgaagtccatacaaacgctcctgataaaattgacaaagaggaggttaaatgatggataaaagaactagtgaaatagcctgaaaaaaacaggttctttttttatttctatttagtaaatgcccacctataaactaattgttggtttaggtaacttaggtaaaaagtatgagaaaactcgccataatgctggttttatggtgttagatagactagctagtttattccacttaaactttgataaaaccaacaagttaggtgattatctttttattaaagaaaaagcagcaatcttagcaaaacctgctacctttatgaataatagcggtctttttgtgaaatggttacaagatcactttcaaattccgcttgcaaacataatgatagtccatgatgaaatagcgtttgatttgggagtaattaggcttaaaatgcaagggagtgctaacaatcataatggcataaaatcagtaattagacatttagatactgaacagttcaatcgtttacgctttgggattaaatcacaaaatacgagtaacatattgcatgaacaggtaatgagtgaattccagaatagtgaactgactaaactggaagttgcgattacaaagtctgttgaactgttgaagcgttatattgaaggagaagagttacaaaggttaatggaatattatcatcatggctagatgaaatcagttacagtcaagcagttactacaaaccccacgaaaatttaataacaagcagattaaactatcaggttgggttaaaaataaacgtgctagtgctaacatcatctttctagcaattagtgatggctctagtattaataccctacaagcagtagtaaaacaagaagataacccccaggttttctcactgttacaaactgttaatttagcaagtgctgttatggtttgaggggaaattatcttaaccccaaaagctaaacaaccactggagttgaaattaaagcaggtgagtttattagcacaagcagagtctgattatccactgcaaaaaaaagaacatagtcaagagttttttagaagtaatgcgcatctaagagtaagagcaaaaacttactttgcagtgatgaaaataaggagtgttttgtcacacgcaatctttgaatacttctttaaaaatgattttatcttagtgcaaagccctattttaactagtaatgattgtgagggagcgggggaaacatttgtaattaaagatagtgaaactttttttaataaaacgacttttttaacagtaagtggccagtttggagcagaagcttttgcgcaagcatttaaaaaggttttcacctttggtcctactttcagagctgaaaaatcccatactaatcgtcatcttagtgagttttggatgatcgaacctgaaattgcatttgctaacttaaaagatttaatgcagttaatacaaaacctaattaaattcttaattaaaaaagtgatggaaaatgctagtgatgaactaaatgttttagcaaagcaatttagcaatgacattattagcaacttaaagacaatcattagtactaaaaaatttccaatcattgaatacagcaaagcattagcgattctaaaggaatctagtgatacaaaaaaaactaattttgaactaaacgactttagttttggtattgacttaaaaacagaacatgaacgctttttgtgcgaacaatattttcaaaatcaaccgctttttgttattaactatccaaaggagttaaaggcattttacatgaaaacaaatactgacaataaaactgttgctgcagttgatcttttattaccaaagattggtgagatttgtgggggaagtgaaagggaaagtgatttaaaccaacttaagaataggtgtcaatctttaaacattgacacaaaaagtttgaactgatatcttgatatgaggaaatggggttattttgctagtgcaggttttggtttgggctttgatagattattagcttatatatgtggattggaaaacatcagagatgctattccctttccccgtgtacatggcaccattaacttctaattcattaaaataacctttaattaccctatcaattctaataatgataaagcgcgcaattacagggattcaagcttctggaagacaacacctaggtaactttcttggcgtaatgcaaggtttaaaacaactccaaagtcaataccaactgtttttatttgttgctgatcttcatgctattactgttgattttgaaccaacaatgctcaaagataacaacttgcaacttgttaaaactttattagcactaggacttgattatggaaaagtgaacttatttttacaaagtgatctgatggaacataccatgttaggttatctaatgctgacacaaagtaatctaggtgaattacaaagaatgacccaatttaaaacaaagaaattagcgcaaaaaagaaatagtaataacaccattactatcccaactggtttgttaacttacccagtgttaatggctgctgatatcttgctttatcaacctgatattgttccagttggtaatgatcagaagcagcacttggaattaaccaatgatttagctaaacgtgtagcaaaaaaatttaagttaaaactgaaattacctgtatttatagaaaacaaagataccaacaggatcatggatctatcaaatcctttaaaaaagatgtccaaatcaaatcctgatcaaaatggtgttatctatctggatgatagtaaagaaacaatcatcaaaaaagtgcgcaaagccacaactgatagttttaataagattcgttttgctaaaaaaacccaacctggtgttactaatttacttgttattttaactgcacttttaaaagaagaagttaaccataatttaagtaaaaaaatcggctctgatcttgttaaatattatcagaataaaagttatttagatttaaagaatgacctcagtagtgctgttattaatgtcatagaatcacttaaatttaaaaaagcacaaattactgatgaaatggtattaaaagtcctaaatgatggtaaaaaccaagctaaaaaagttgctgatgaaacattaaaaatgttttataaagcatttggtttaacatctaatcagctttttgattaagcttaaaattaagataaataattttccaattttgttttcaatgagtgatcgtttaaatgatcaagcccaacatcgcttgcagaaacttttaaggttaaaacaaactaataatgacccttatttagtaacaaaaactagtctaacccattcttcaaaaagctttcaagttgaatttgaaaaatgttcagaagaagagttgaagaaaaaagcaactgtctcactagctggaaggatcattgctattagacaaacctttttaattattcaagattttgatggtcaagtccaactttacatcaataaaaaaatccatcctaagttatttgattactttaatgaatttgttgatattggtgatcaaattgttgttagtggtaagccaatgttaactaaaacaaaggtattaactttagctgttgaagagatgaaaatcattgctaagtgtttattggttccacctgaaaagtgacatggacttactgatattgaaacccgcgctcgcaagcgctttcttgatcttacctataacttagcaatgcgtgatgtttttctgaaacgcactaagattattaaatcaatccgtagctttcttgatcaaaatggttttattgaagttgaaacccccactttacaagctgttttaggaggagctaatgctaaaccctttaaaacccattacaatgctttaaaagcggatttttatctcagaattgctaatgaaatagcattaaaaaaactcattattggtggatttaacaaggtttatgaaatgggtaaaatgttccgtaatgaaggggttgatactacccacaatcctgagtttaccagtattgaaatatatcaagcttatgcagattttgaagtcatgcttgtgcttgttgaaaagctgattcaatcactttgtgaaagcttaaaccaatttagctttaactgaaataacaaaacgattaatctaaaaacaccatttcataagataacaatggttgaacttattaagaaagttacagggatcgattttaattcagtaaaagatgatcaatctgccattttattagcagaaaaacatcatgttaaactagcaaaacaccaacaaaataagcaacacatcattaatttgttttttgaacagttttgtgaacaaacattaattgaacctacctttgtaacccattatccaaaagcagtttctcctttagcaaaacaagatccttcaaatcctgaattcacccaacgatttgaactttttattaatggtaaagagattgctaatgcttacagtgagctaaacgatcctttagaacaaagaaaaaggtttgaacaacaacttgaagaaaaacagcttggtaatgatgagacaagtgaacttgatgaatcgtttttagaagcattaagttttgggatggtaaacactgctgggcttgggataggtattgatcgtttggtaatgttgttatgtgaatgtaattctatccgtgatgttgttttcttcccccagttgcgtgaacataaatagagagttaggtttaaaattccgttcttaataaaatagagctgtggctaggtacttggggattgttagttatgatggcagttactttaaagggtgagcgattcaaccaaacctagctactatccaaggtttattggagcaaagtttttcattaatcattggcagaaagataaaggtaattggttcaggtagaactgataaaggggtacatgccatcaaccaaacctttcatgttgatattaatggtgaaattaatctcaatttgttaattagaaaaattaaccagttgattaagccccactgtatagttaaaaccttggtattggttaacgatagctttcatgcgcggtttcaagttaaaaccaaggtgtatgaatatctgattaactgtgggaatttaaatccgttgcaatttaactatgtttggcagttaaaccagcaattggatcttgaaaaactcaaagctgatgccactttatttttaggtaagaaaaactttcttagcttcagtagttcgattcacactgattcaattcgcacaattagtaaaattaccatacaaaaagaaactaaccaactagttagactaactttttttggcagtggttttctcaggagtcaagtgaggatgatagttgcttgtttagtgaatttaaacactaataaaatggcacttgaaacagttgcaaaattgtttgaacaccccaagaaagggagttgtgttgttaaagcccctagttgtggtttgtatctgaaaacagtggtatatgaaaaatagaatgattttttgatctatatagcacaattaatataacttcatgattgatcaaaacaagttaattactaagtgaaaaaaagcatttgcaaaagctaagaatttaactactttagttaatcttaagaacactttacacaacagtgatttaaagccattactccaaaagattaaaaccgctacaaaactaagtgaaaaaagtagtttaggtaagctttatcaatcacttgatattcaactaactgatctgttaactagttacaaaaaaacctttgaaataaataaccaagttagtcaaaaaccttcacttgatgtgatgctaccagcaacagagtttaccaatggttctaataacgcactatatcaggttattgataatttagttgaatactttaaaagctttttattcacaattaattttgatagtgaactgaccagtattagtgactgttttgatcttttaaatatccctaaagatcattccagtaggaatgaatctgattctttttatatcgataaaaccagtttattgagaacccattgtactgctaccacgctaaaagcagtcagaacttctaaaaaaactaataatcctgatatcagggttgtctctttaggagcggtttttcgtaatgatagtgatgatgccacccactcccatcagtttacccaacttgattttatgtggattaaaaaagggctttcattagctaatttaaagtggtttattaacaatatgatcacccatttctttggggaaaatacttttactaggtttagactatcccacttcccattcactgaaccctcgtttgaaattgacattaggtgttggttatgtcaaaatggttgttctatttgtaagcaaaccaagtgaattgagatcttaggggcggggatcatccatccccaggtgatgaataacatgggaattggggatactgaaaatattactgggatagcagcaggaattgggattgaacgcttagcaatgttaaagtatgggattgatgatatccgtgatttttatgataacaactttaagtttttaacccagtttactgactaataacaactttaagtttttaacccagtttactgactaaaatatgttgatatcaaaaaaaacacttggcgttttaatccctgacatctttagtttttctaatgatcaaattgcccaaaagttagaacaaatggggattgaagtggaatcaattaagcagtttaacagccctgattacctccaacttgcaaaggttgtatcaatccaaccccatccccatgacaacaagctttttatctgtgaattacaaattgataaaaacaagtttattaatgttgtttccaatgctaataacattaacaatcctgataatatcaacaagtttgtcattgttgcaaaaaaaggaactgagttactcaacgggttaattgttaaaacccaaaatattaaagggatcatttcagaagggattttatgtagctatattgacattaaccccttcagtagacagatcattgaaaaaacagaagttgctgatgcgattatcattgatcatgttagcaatgatcatgactgaaaccaatacctctcgtttttaagtttggatgatgtgatctttgatgttaaaaccccaactaacagagcagatcttcatagcttaatctttttagcaaaagaacttggggtacttttgaaaaccaaaacctttttaaaacaaaaaagtagtgttgttaaccatgacttttttaagtttcccctaaatttaaaaaacaagttaaaagcgaattattttggcggtttgttcttaagacaaattaaccaacatagttcaccttgaacagttaaaggactgttaattaaccaaatgatcaaaccagttaactattatgttgataaagctaacttagtaacagtgttcaccgctcagccaatccattgtcatgatgcagatagaattgttggtaacattgaacttaaacaagcaacccataatgaaacttttgttggacttgatgacaagcaatatgagattgaaccaggggatattgttgtttgtgatgagaagggcattattgcactggtagggatcattggttcaaagcgcacaatggtccaacctacaacaactaacatcttttttgaagttgttaactgtaacagtgaaaccattaaacaaactgccaagcgctttttgatcaataactttgccagtaagtttatggttaaaccgattagcttattagctactgataactgtttaaactacttacaaaacagtttactaaccactgataacattggcaaaattagccacttttcaagttcgcttaaagttgaaccatttagtaaaaagctcacagtgaatttccataagatacgccaactaattggcattgaaaaaaaggaactaactgatcaaaccattaaaaaaagcctcagtcaactagggtttaaagttgacaaccaacttctcaaaatccccagttacagacaagacattaatacctgacaagacattagtgaagagattgtgaagttaattgatatcaataagttaaaaccaattgggatcactagtagttttaactttgaaaagtccagttactttaacacttttaatgctttaacaaaactaagaaaaaagctacaaacacttggttttcacaacgttattacctaccagttaactgatcaaaaaagtgcaaaaacttttaatttgtttaacttagaaaatttcatcaccattaaaaacccagtgtcccaaaaccattctgtaatgcgtgttagcttaattgattcactgttaaaagtgctaaaaaccaataacaactataagaatgaactggtgaacatctttgagttttcctttattaaaacccaaaacaatagtgaactgcacctggcagtattatgagttgaaaaactgtttacttctagtttcaatcctatgcaagggataagcaatgatgtttttactatgaagggattagcaaaactcattgttgctaacttagggtttagttgtgactttgaaccacttgatgatagtgactattttgttaataatcaaagtttaaaaatagtagtttttaacgaacagatcggttttattgggctaattaaagaatcattgttaaataactatgatctgaacaataaacccatttattgtcttgaaatcaacttagataggatgctctcttctctaaacaggattgaaaaaaactaccttggttacagtaaactacaacctgtttgcaaggatcttacctttagttttaccaaccctgctagtcactttgatcagtttgctaacatgatcaaaaggataactggcattgaaagttgaaagttaattagtgtctttgaaactatgcaaaacaaccaactgatcactaagtacaccgttcgttattttctgaaaaatgatgctaacaaaccactaactaaccaaacaattgaacttatcactaataacttaaaactccagtgtgaaaaactaaaaattaaattagatatttagttgtttgcttgaaaaaactaatttttaagattaattaacaatggctaaagtttacaaccaagaagtttatgttcagtttctcaaacaacatggttttgtatttcagagtagtgaaatttacaacggtttaaacaatagttgggattttggtccattaggtgcagttttaaaacaacaaatcaaacaagctttatataacttttttattaaaaataaagctgatgttcttttagttgaaacccctattattctcagcgaattggtttgaaaagcatcaggacatttagctaactttgttgatactttagttgattgtaagagttgtaaataccgctttcgtgttgatcaaattaatgctgaaataaaagctaaaaaggattggaatagttttaaagttaactgtcctaattgtcataaccaaaattgatcagaagtgagggattttaacttactttttcaaactgaaatcggggttgtaaacaacgataaacgccttgtttttctccgtcctgagactgctcaaggtagctttattaactttaaaaatatcttgcaagctaagaagcgtaatttaccttttgctattgcccagtttggtaaaagctttcgtaatgaaatcaccccaggtaacttcttgtttagaactagagagtttgaacagtttgaaattgagtggttttgtaaacctgatgatgcaaattcgctgtttgaaaaacaattaataatggtagaacagtttctacaaacagtgttaaaaattaacccagaattgttaaaaaaacatgaatatgatcaatcagaattggctcattatgccaaaaaaactactgactttttgtttaattttccccacggattaaaggagttatgaggcttggctaacaggggtgattttgatctaaaacaacaccaagagttttcaaaaaagagcatgagtttttttgatagcgaattaaaccaacatttcttacctttcataatcgaacctgcggttggcattgaacggttattttatgcactaattgtcagtagttataggagagaaattattaatgaggaagaacgggaagtattgagtttaccatttgacttatgtcctgaacaaattattgttttaccacttgtaaataaacttaaaaaagaagcattttctgtatttgaaacgctagcaaaaacaaggtgaagagtgtgctttgagacaactggtagtattggtaaaaggtatcgaaaagcagatgcaattggaataaagtatgcagtcacttttgactttgaaagtttagaagataatgcagttaccatcagagatagagatactttagttcaacagcgaattgctatcaaagaattaccacaatgattcatgaaaaatggtcaataaaacattcctatcattaatgaagcaatttgaacataggtttatgattgttgacagtgttagtcaaaaaccaacaacactagttcaaaaaaccattaacatttatctctgtggacccacagtttataacgatttgcacttaggcaacaccagaccattaattgtttttgatgttttaaatagagttttaaaaaaggctaaatataccgttaattttgttcaaaacatcactgatattgacgataagatcatcaagattgctcaacaacaagaagtaagcgaatcagttgttacaaaacaacaaatcactgcttacaaatcacttttaaaaaaactaaatattctgcctattaaacatattcaaatcactgaaaaaatcgataaaatccctgactatattgatcaattagtaaatcaaaaccatgcttatgtttcaactcaaaacaacgtttattttgcagttaattcactaaagcaatatggttatctagctaaccgaatggtgcatttagaagaaactgatactgataaaaagaacaaattggattttgtactttgaaagattactactgcagggattaaatgaaatagtaagtggggacttggcagaccaggttgacatgttgaatgtgccttcttaattgattattgtttcaaaaatgaactcacgatccacggaggaggagttgatttaaagttcccccaccatgaaaatgaaaatgccttacacatggctttatataaccagcccattaccaaacattggatgcatattggtcatttgatgattgaaaaccaaaagatgtcaaagtcattgcagaacttcttgttagcagttgattttcttaactttcatgattttcgtgttttgcgttggatcttttaccaaaaacactatttgcatcctattgatctaaaccaatcattgattgaaaaagctaataatgatattcaaaggattgcaaaaacacttaatgttgctagaacctgattagtttattcagaacaatctgagttgattagtcccaagcaatatgatccagttttttcagctttacttgataatctcaactttgccaatgcagttgctgctatctgaaaactaataaaaaaaattaatacaagtattaaaactaaggactttagtgtgctgagagaacaacttagtttcttggaatgatcaattgatttattaggaattagctttaaatctatccatactaaacttaatgtgcgtttaattaaagagtgatcaatattacacaaacaaaaagcaatggataaagctgatcaaattagaaaaaaactaattaaaaaaatgttgctgtaaaactaaatatgcaatcaagcgttcttatcaaagcaattagatgtacaatcacaatttaattgaagaaaagtggttaaaaaaatgaaaaaacaaagatgttaaccgctttgaaagcgatagtaacaaaaagaaatattatgtccttgacatgttcccttatccctcagcagcaggattacatttaggacatgttagagcttatactatcactgatgtaataagtaggtattacaaagctaaaggatttaatgtgatccatccgattggttttgatgcttttggtttacctgctgaacagtatgctattaactctaatcaaaaccctggcagttgaacagatcaaaacattaataactttattaatcaattaactagttttggttttgattatgactatcatttaagtctcaaaacaactgatccacgttattacaaatacacacaatggatcttcagtgagctgtttaaagcaaacctagcggaattagttgatattgatgttaattggtgtgaacagctaggtactgtattggctaatgaagaagttttaattgatagtaatggcaacgcagttagtgaaaggggttcattttcagttgaaaaacgcaagatgaaacagtgagttttgaaaatcactacttttgctgatgcacttcttgaaggcttagatacacttgattgacctgaaccaattaaagagatgcaacggaactgaattggtaaaagtaaaggtgttactattaactttcaactaaaagatcataaggaagctattgcaatttttacaactaaaccacaaacaatttttggggttagttttcttgcagtttcaaccaaccattggttagcaaaaaagatagcagaaacaaataaaaaagtagctagttttttaaaaaaacaactccagaaaaccacaactttaaagcaaaaagcaactttatatgatgggatagatttattaacaaatgctattcaccctcttacaaatgaattgatccctgtctatgttgctaactatgtaattgaaggatatggaacagatgctattatgggtgttggagcacacaatgaaaatgataacttcttcgcacgtaaacaaaagttgaaaattatcaacgtcattgataaaaaagaacggctgcaaaattcatttgcatataacggattaacaactaaagaagcacaagtagctattactaatgagttaatttcacaaaataaagcgaaattaacaactgtatataaactgcgtgattggatcttcagtagacagcgttattggggcgaaccttttccaattatttttgatgaaaataacactcctcatttggtagaacaactccctgttgaattacccttacttgagaattacaaaccagatggaagtggtaattctccactaatgagaaatcaagcttgggtaaacatagtcaaagataacatccattaccaaagggaaactaataccatgccccaatgagctggttcttgttggtattatctgggttatttaatgttgattaaaaaccctaatttttgaccaattgattcaaaagaagcgaagaaattatttgatcaataccttccagttgatctttatgttgggggtgcggaacatgcagttttacaccttttgtatgcccgtttttgacacaaatttttgtttgacaagaagctagtatcaacaaaagaaccatttcaaaaattaattaatcagggtatggtgttaggtcctgatggtaaaaagatgtccaaatccaaaggtaataccattaaccccacaccacttgttgattcacatggagcagatgctttaaggttgtacttaatgtttatgggcccaattagtgctagtttaacttgaaatgatgaagggttaaacgggatgagaaggtgattggatcgagtttataacttcttttttaatcatgctgttgttactgatcaagttagtcaagagacaatctttgcttacaatttgtttttaaaaaacagttattgtcatcttgacaaacatgaactaaatctggtgattagtgaaatgatgatctttttaaactttctctataaaaccaaaaaaattagcttaaattatgcaaagggatttttaacagtactgtcgttttttgcgccctttcttgctgaagaattgaatgaaaaatgtggacttgaaccatttgttgttaaacaagcgatttctttagttgattatcaactttttgagactgctaaaactaaggttattctttcaattaatggcaaatttaaagcagctaaagaatttactaaaggtagtttagagatagatgttttagaatcatttaaacaggataaagagataaatgacattctcaaccaaccgattgagagggtagtttatgttcaggatcgaattattaatgttcttttaaaaaaatagggagtaattagtcgcaaccgttaagattactttttgctagatgacaaaaaaaacacaagatctcactagttggtatgaccaactgctagttaaagcaaagttaatttgtcatggtgaagttaaaggtacagtttgttttttaaataacagttgaggcttatggatggaaatccaacagctttacaatgatgcaattgcaaataaaaatcaattgtctgcaattgctctaactaaattccaaccaactactagtttttgttatcaagtattccaagtacaactccctaccctttctttttacagtgaatatcaaaaggaaaaaacccatatcaaaggttttaatcctgagctttttttaattaatcaagttggtcaaaaacaactcaatgatcctttggttttacgacctactagtgagattgctttttgcaacttatggaaaaaacaagagttatcttaccatgatctacctttaatttataaccagtgaactcaggtttttcgtgcagaaaaaaacaccagaccttttttgagaaacagtgagttttactgacaagaaactcatgggctttttgtggatcagagccaatctgaacaagctgctattagcttttgaaatttatatcaggatttaattattaacaaactttgtatccctgcttttgttggtttgaaaagtgaaagtgaaaaatttgcaggtgctaaaaacacatggacaatagaagcaattatgcctgatggacaaagtttacaatgtgccactagccatgatttaggtgacacttttacaaagagttttactatcagctatcagagtaaaactaaccaaaaaatgactccaagtagttttagttgtgggatgtcaactaggatcttaggagcaatttttttaacccacagcgatgattatggtttggttttaccttggtatctagcaagtaaacaagtcaagttatacctgtttgataaaaacaataaccctaaaacaagagctttagcttttttagtgaaggattttttagaaaaactcaaaattcgctttagttttatagaaattaacaatcaactaggtaaacaacttttaaaaggagaaatagaaggtattccattacagatgattgttgataatgaaaaaactattaacatcttcaaccgcttaacacgtttaaaaaccagcttaacatttgcaaatctccaaactgaatttgttaatttagttaacaactaccatacagagatgtatagaaaagcaaatgatttagttgaacaaaaactagcaagagtacaaactttaaaggaaattgaacaagcattcaaaaataaaaaggctgttttatgtaccgtgaagttaactggtgaacttgaacaacacttaaagacaaaataccaagttagtgttaggtgtgtttttaaaaagtcagatgtaacacaaaactgtccttttacaaatcaaccttgttttgattcagttttaattgcacgtgcttactaaaatcgtactaattgctttaattactatgctttttatcactatgaattgaacaactgataaagtaaggcaaacctgattagattattttgcaaagaaagaccatctggttttagcttcaaaatcactaattccgatcaacgacccatcattattatgaatcaattcaggagttgctactttaaaagattatttcagtgctagaaaaacaccaccatctaaacgccttgttaatgcacagatatgtttaagggtaaatgatattgaaaatgtgggttttacttcaagacatcaaactttgtttgagatgcttggaaatttttcaatcggtgattattttaaaacagaagcaattgattttgcttttgatcttttagttaattattatcagctagatcctaagcgtttttatatcactgtttatgaagatgatgaaactacttataaaagatgaattaagcataaaattgataaaaatcacattattaagtgtgacaaaagtcgtaacttttgagacttaggtttaggaccttgtggaccttgcactgaaatctattatgatcgtggtgagaaatttgatcctaaaaaaattggtgaaaaacttttctttgaggacattgaaaatgatcgttatgttgagatatgaaacattgtttttagtcaatttaataatgatggtaatggcaactatacagaacttgctcaaaaaaatattgatacaggtgctggaatagaaagacttgtttcagtattacaaaatagtccaaccaattttgatactgacatctttttaaagctaatcaaaataattgaagctttttgtccatttaaatatgatcccaactcttactttacattcgatcctcaaaaagtgaaagaacagagttattttcggattattgctgatcactttaaagcaatcacttttaccatttcagaaggagttttacctggtcctaatgagagaaattatgtagtaagaagacttttaagacgtgctttaatagcttgtaagaaattgcaattaaacttagcatttattgaaaagataatagatgaaatcatcgcttcatatgagaattattatcaacatttaaaagctaaaaatgaaactgttaaacaggtagttttaaaagagattaatgcatttaataaaacgattgatttaggtttagtgctgtttgaaaaaagtgttaaaaacaatactctaactccccaattaacatttcaattgaacgaaacatacggttttcctgttgaaataataagagaactagttaatcaaaaaggtttaactattgattgaacagtatttgatcagttaatggccaaacatcgttctatctctaagcaaaataaccaaactataaattttgaaaaacaaaatattaatttagttaatttcaaaactaaaagtacttttttttatcacaaaaataaaattaatgctaaggtaattggtctttttgatgaaaattatttaccagttaaagaacttaataatcaaagtggttatgtagtttttgaccaaacagttttatatgctacttctggaggacagagatatgatgaaggaagttgcattaatcattctaataataatgatcaaaaaatcagttttcaaggtgtatttaaaggacctaataaacaacacttccactactttttagtaggtagttttaaactcaatgatcaagtaactttatcacatgatgaaacttgaagaaaacttgctgctaacaaccatagtttagaacaccttttacatgcagctttacaaaaagaaattgatccacttattaaacaaagtggtgcttttaaatctgcgcaaaaagcaactattgactttaatttgaatcgtcatttaacaagaaatgaacttgagaaagtagaaaataaaattcgctctttgattaaacaaaaaataagctcaaaagagatttttactgattttgaagggagtcaaaaactaaatgcaattgcttattttgaagaggaatattctcaacatgaaatattaagagtgatccgctttggtgattatagtgttgagttgtgtggtggcactcatgtagctaacactgcttcaattgaagattgttttattactgatttctattctttaggagctggaagatgaaggattgaaatcattagcagtaatgaaactattaacaactatttaaaagcagaaaatcaaaaattaatccaattaaaatcagaacttgaaaaagttctatctttgattgatagttcaatttttaaagttgagttaaaagaattgcaacaaaggctagataaatttatcttacctgaaaaaattacccaattaagagatgcatctgatactttattagctttaaaaaatgatattaaccagttaaaaacaaaaaactataaagtatcacagcaagctttagctttatcaattaaaaagcaattattatccttagtagatgaaaataaaagttatgtaattgccacttttaatgacgtagaacctaaactattgctacaaacactacatgatgttttcaatcaaaatcaaactaaaaatttcttgataattaatcaattcaatgaaagtaattcatttattgttataggaaataaaactaccaccattattgaaaaattaagaaatagttttaatttaaagggcggaggcaatgataagttatttagaggttcttttcaggataatgttacccctcaaaagcttaatgaattgtttcaaaataaagctttagttcacaaaaaaattttcgaattattcgttgaagatgaaagataaatttagttttcaaaaaaactatgatttcaacttagttagtgatgggctttatgaaatttgaaataatgctggtttttttaaacctaaagataaaaacaattcttttacagcaattcttccccctccaaatctaacaggtactcttcatattggtcatgcttttgaggttagtattactgatcaaatcatgcgttttaaaaagatgcagggatttagtattaactgaattcctggctttgatcatgctggcattgctactcaaacgaaatatgaaaaaatagcattaaaagaaaatcaaaaatattttgatgcagatgatgataaaaaatctgaaatgatcatgaattgggcattaaatcaaagcgaaataattaaaaatcaactaaagagtttaggagtttgcttaaattgatctgaaactaaattcacgctttcagaacaagctaataaaattgttaacaattgttttaaaaacctttatgaaaacggttttatttatcaagcatacacgcttgttaattgagatacaaaattgaatactgctatatcaaatattgaagttatcaataaacctgttaatcaacatcttcattatgttgtttataaactagcgaatgatagtaaacaagaactaatagttgcaacaacaagaccagaaactatctttgctgatgtttgtctattggtaaacccaaaagataagcgctatactaatttctgaaataaattagtagttaaccctttaacaggaaaacaaattcctgttgtaacagatagctatgttgacattaaatttggtacaggaatattgaaatgtactcctgcacacgactttaatgactatgaaatcaacactaaatataaatttgattttctaagctgcattgacagtaacggtattctcaatcaaaatgcaagtaaatttcaaggccttagtgttttacaagcaagaaataaaattgttaaatgattagaaaaaaataaattacttgttaaatcaataccattaactagtaatgttggtttttctgaacgcagtggcactgttgtagaacccatgctttcaaaacagtgatttgttgatttaccaaagttaaaagatcacttatatttaaaaaaatatcctgattttattcccaaacgctttaataagcaagtgtcaaattggttgaataaactcaaaccatggtgtatttcaagacagttaatttggggtcataaaattcctgtttgatttgaaaacaatacaggtgaaatagttgttggtgaaaaaccttcaaaaaatttacaaaactacactagatcaaaagatgtacttgatacttgattttcttcttcgctttgacctttaatttgtttgaattgagaacaggatgactcttttcatgaaactgagcttttagttacaggttatgatattctatttttctgagttttaagaatgttatttaactccttttttgaaactaaaaaactgccatttaaaactgttttaatccacggtttagtacgcgatgaacaaaatcgtaagatgtcaaaatcactgaataatggcattgatcctgttgatcttattagaaattatggagcggatgcagtgcgcttatttttgtgttcaaatcacactccaggagatgatctaattttcagcgaacaaaaaataaaaagtgcatgaaattttttaaataaattgtggaatgttactaagtttgttatccaactagaaaatgatcaagaaattagttatgacttggacaaactttcattaagtgaaacttgaatcttagctaaattagataaagtaattcaaaaaataactaagctactagataaattccagttagcattagcaaaccaaattcttgttaaatttgtttgggatgatttttgcaatactttcattgaagcaattaaaaaagaaccaaatcaactaaaaccacagcttttttatactgctaaatcagttttatctaatattgctattttgcttagtatcactgttccttttttatctgagcgtatttatcagcaatttaacaataaaagtgttatgcaagcaacatgacctcttgcaactaaaattaaaattcccaaactttttgatcttgttttagctgctattaatgacttacgcaattacagaaaacagtacatgcttaattcacaacaaaaactagttgttatcttatctggtaaaaatgctgttgatgttaaacaatactttaactttagttgaattgaactgaaaattgaaactaataaaaaagttagttttaaataccaaattgttgatgatacaacccaaagacttaaatctctacagaagcaacaagctttttttgaaagtgaagtaaaacgtagccaagctattgttaaaaataaaagctttctagaaaaagcacccaaagaaaaggtaaaaagtgaatttttaaaattggaagaatatcaaaaaaaacttactgaaaccaaccaattaattgctaaattaactaaagctcattagaagaatctttatcttgttaaattaatacttaactggtttaatgtctgcaattaaatttaatcctagttcattcagaaaaaactttaaatggtttgaaaataacaaaaattggattaattttgataatgctgctacttccattgcacttgatgttgtggctgaagcaagcaaagaatattaccagtatttttgtgtcaatcctcataacaaaaatcctgaaattaaccaaaaacttattgctattattgaagaaacaagagatttattagcaaaatttttcaatgctaaaaaaaatgaaataatttttacaagttctgcaactgaatcgcttaacttattcgcctttggattaagctctttagtaaaaagtaatgatgaaatcattctcaaagaagatgaacatgctgctaatgtttttccctgagtaaatctagcaaaagaaaataaagccaaactaaaaataattaaaaaaacaccaaataaatcttgaactgatgcttttttaaaagcttgtacaccatcaacaaaactattagttataactgcaacatctaatctttttggaaatagtattgactatgaaaaaatttctaaacacttaaaaaaaatatcaccaaatagctttattgttgtagatgcagtacaagctgtaccacaccataaaatcgatattacaagtgctaatattgattttttaactttttctacacataaattttatggacctactggtcttggcattgcctttatcaaaagcgaattacaatcacgactaaaaccctttaaattaggtggtgatatttttaaatcattggataataactttaagataatttttaaagaaggtccttccaaatttgaagctggaacgctaaatattatggctatttatgctttgaataaacagttaaaattcatgcaaaaagaatttaatttcagtgaaatggtgttttacagcaaacaattaaaaaatttagcttatcaactgctaagtcaaaatcctaatatcgttttagctaatcatgatcaagatgttcctatctttgcttttaagcataaatatattaattctgcagatctagcaacttttttaaacattaaaaaaataattgttagacaaggatccatctgtgttggtaaatttaaaaataaagagagttttttacgtgtttctctactccattacaacacaaaagaggaattactttatttagaaaaattattaaaaactagtaagaattccattattaatgaactaatatattagatgtaagttagataaatattaataaaattactctgatttaatggacttaaaaaagacattgttaatgcctaaaacatcctttgcgatgcaggcaaatttatctactagtgaaaagaattttcatgatttttgaaaagataaaaaagtctttcaaaaattaaaaaaacagaataaaggaaaacagataaaaatactgcatgatggaccaccttatgcaaatggtagtattcatgtgggacatgctcttaacaagattttaaaagacttcattttacgtagttggttatatgaaggatatgatgttgtttttattcctggttgggattgtcatggactaccaatagaacatgcagttagtaagaaaaaccctagtagttatagcaatctttcaactgttgaaaaaagaaaattatgtcatcagtttgcactttcacaaattgcagttcaaaaagaacaatttcaaagactgggacttttaaatgattttcaaaactgttattacacaatagatgagagttttcaatttaaggaacttgaactatttttacaagcaattaaaaaagggctcatttttcaagatttaaaaccaacttattgatcaccaatttcaagaacttcacttgctgaagcggaaattgaatataaagaagttaattcaattgcactttatttaacttttaaagtttctaaaagtgattttttagatgaaaatgctaatttattagtttgaacaacaactccttgaacactaccaactaatcaagcaattgccattcatcctgattttgattatcttctttttgaatataaccaacaaaaatttgttatcttggaaaaattatttgaagtttttacaaataagttaaattgaacaaatgcaattaaactaaaaaaattcaagggttcaaatttaaaaaattcaagctattctcattgtttttataacaaggttttaccagttctaatgggaatacatgttgttgataatgagggaacaggtattgttcacagctcccctgcatttggaattgatgatttttatctttgtcaaaaaaacaagattaaagaagttttgatttctattgatgagaaaggtgtatttaataacttacttaatgataaagaacttgagaattgtttttatcttaaagcaaatgatctaattattaatcgtttaaaacaaaacaatagctttattttttctgaagttatttcccaccgcgaaccacatgattgacgctcaaaaactccagttatataccgtgcttccaaacaattattcattaaaactaaatcaataaaaaagcagttaaaaaaacaaattaatcaagttaattttttaaattcaaaaaatcaattgagattaaaagagatgcttttacaacgtgatgaatgatgtatctcacgtcaaagagtatggggcttgcctataccaattgtttatgcaaataacaaaccattgttagatttttcaacaattcaatacacaattaaacaattgaaaaagcatggtattgatagttgatttgaaaaagatgtaacttgttttttaaaacctgataaaaccaaaaaatgagttaagtatcacaaggagattgatacattagatgtttgatttgactcaggttcttcctataatgttttggaaataaataaatatggttcaatagctgatctttatattgaaggttctgatcaatatcggggttggttcaactcttcttcaaattgcggaattattcaaaatgatttaatcccttttaaatcacttgtttcacatggttttacacttgatgaaaatggcaataaaatgtcaaagtcattaggaaacatagttgatcctttaaaaatttgtgatcaatatggagcggatatattaaggttgtgagttgctaatactgattgacaaattgataacaaaataggtgttaatattcttaaacaagttgctgaacaataccgcagaattagaaatagtttactacgttttattttgggtaatattaatggatttaactttacatcaatggatgattataagttttcactagaagacaaaatagttatccataaaactaattcactagtagaacaaattgagaaatttttagagaaatataattttttaggttgcctaaaagtgattaataagtttgttttatgactatcaagctgatactttgaaataattaaagacaccttatattgtgatgctaaaaataatcctaatcgtttagctaaacaagctgttttaaactatatttttacacaactaatcagttttttaaatatctttattccccacactgcagaagatgcttgaaaaaactattcattcaataaaaaaccaataagtgtgaacctctttacaaaaccgactgtttttaaagttgctaactctaagaatttaggaaatatctataaaacttttactagtattaaaaatgctgctttcaaagaaattgaaaagctaagaaaagaagggttgatttctaaaaataatcaaattgaattaaccgttggaattaataaaaaaatacccaaaaaattaaaggataatctcgcactttgacttaatgtaaacagtgttaatttaacaaataatgaaaatgaaattaaagttaaaaaaactaaaaaaacaatgtgtgaaagatgctgaaattttcaaacaatcattaagcaaaaattagatcataatttgtgctcacgttgttttaaagtgtgttaagtatattattatcttgattatattgtaactagaaccatatatgtttaaaattgttttctttggtacttcaacgctttcaaaaaaatgtttagaacaacttttttacgataatgattttgaaatttgtgctgttgtaactcagccagacaaaattaatcatcgtaacaataaaatagtaccttctgatgttaagtctttttgtttggaaaaaaacataactttttttcaaccaaaacaaagcataagcataaaagctgatctagaaaaattaaaagctgatattggtatttgcgtttcatttggtcagtatcttcatcaagatattattgatctttttccaaataaagtaattaacttacatccttctaagttaccactacttcgtggtggtgcaccattacattgaaccattattaatggttttaaaaaatctgcattgagtgtaattcaattggttaaaaaaatggatgcaggtccgatttgaaaacaacaagattttttagttaataatgactgaaatactggtgatttatccatatatgtagaagaacattcaccctcttttttaattgaatgtactaaagaaattctcaataaaaaagggaaatgatttgaacaaataggtgaacctacttttggattaaacataagaaaagaacaagaacatcttgatcttaatcagatttacaagagttttttaaactgagtaaaaggtttagctcccaaacctggtggttggttaagctttgaaggaaaaaacatcaaaattttcaaagctaaatatgttagtaaaagtaattacaaacatcaattaggagagatagttaatatatctcgaaaaggaattaatattgctttaaaaagcaatgaaattatttcaattgaaaaaattcaaatacctggaaaaagggtgatggaagtaagtgaaataataaacggaaaacatccttttgttgttggtaaatgtttcaaatagagaattagctttaataattttgttaaaagagttagaagagatgcttgcaggagttcttttattacaaatatggttaaaatgcaaatatgctaatgttcagtttggtgaacatggttttaatggagatgaattttatttagatttttatataaatgagaatttttctacaaaacagtttgcaaaaatagaatctgatttaaactctctttctagtaaactagaagggatttctcaaaaatttgtttctttagatgaagcattaagtttttttgaaaatgatcagtttacaaaaaacttattaaaaaaaagcaacttaaacaaatttaaaatcaccttttttgaaaataaacatttttgaatagaagatttaactttaacttttattaaaaaaagttttattaagctattaaatgtaagcgtaaattattttttgggagatccttcacaattacaacttcagaggattaatggcatttttgctcaatcaaaaaaagaattagaacaattaataaaagaaaatgaagaacgcttgaagaaggatcacagatctttaggtaaacaattagagttatttagctttgacccactgatcggtgcaggtcttcctatttgattagcaaagggtacaacactaaggaatataatcggtaattttgtgcatcaccagcaactattgtttggttttaatactgtttgttctcctgtattagctaacatagagctttttaaaattagcggccactatcagcactataaggaagatatgtttcctgctattaaacttgatagtcaagcaatgatgcttcgtcctatgacatgtcctcatcactgtctgattttcaaacaaaaacgatattcatataaaaaaatgccacagcgcttttcagaagattctattttgcatcgttttgaagcctctggaggattaataggattagaaagagtgaggtgcatgactttacttgataatcacattttttgtcgtgcagatcaaattaaaagtgagattaaaaacgcatttaatttaattcaaaaagttaataaaaaatttggatttatatttgataggatagatctttctctacatgatcctaaaaatcaatcaaaatttattgataatcctggtttatgaagagaatctgaaagccaaatggagaatgttttaaaagatttaaatatccaatatcaaaaagagataggagctgctgctttttatggaccaaaaattgattttcagttcaaaacaatctttaaaaaaatgattactattgccaccattcaactagattttttactaccagaaaaatttgatctaacttatatagataaaaaaaatacactaaaaaaaccagttattatccatgttggaattattggaacttatgaaaggtttattgctgctttacttgaaaaaacaagtggtaattttcctttatggttagcacctgttcaagccgtaattattcctgttaatatccaaaagcatttaaaggcagcaaaaaaactttataacaaattgctaaaagaaaacatccgtgtaaatttagatgataatcaagatcgcttagctaaaaaagttagacaagcaatcattgaaaaaattcctttacaacttattgttggagataaagaaatagagaatttagagaagttgacatgccgtggttttaaaggtgaaaaaatcaccagaattagctttaataattttgttaaaagagttagaagagatggataggatttaatattagccatttaggttattaattagttttagaatgttttttatcatcaatgatttaaaagaatgcattagcgctttaaagcttaaatttgatgaccaaaaggaacttgttaaactagttaaaaataatagttttaatggtttttcttcaactattattttccaactaaaaagtgaaaatcataaaaaaattgcagatagtattgttgagtgatttttaaaaaataaaaaggataactaccaaaatgtttttattgctaacaataattttataaactttcaaattagctatcaaaagtacttagaatacttgataaaaacaccttgctttactaagaaaaatataaagattttaattgaatctgtatcagcaaatcctaccggaaggatccatttaggtcatgtgagaatagctttttttggtgatgttttaaacaatttagccaagctgttgggttatacaacagtctgtgaatattgggtaaatgattatggacaacaagcacgagtttttagctttagtgtttatcaaagtttgcagttaaaaaaaaatattgctatccagcaacatcctgatggatatagtggaatagtaatagataaaattgctagtgaaattgaaaattttccagttgataatttaaattttgaagagttttgtaaaacatcattcttagatcattttttagttaattgcacccaaaaagttttgtctttaattaaaagtgatttgaataaaatccatgtttttattgatagttgaaaatttgaaagcgaaattgttaaaaaaacaaattttaatgatcttttagaacaacttaaaccaaatagttatttttatcaagataatgcactctgactaaaaactacgctttatggagatgataaggatagagttttaattagaagtgataaaagagcttcttattttggaactgatgttgcttatcacttagaaaaattacaacgtggctttgacattctatttaatgtttgaggcactgatcatgaaggacatattaaaaggatgtattgtgcatttgatgctttaaaaaataccactaaaacttctttaaaaatttttgcattacaactggttactctctataaaaataaagagctagtacgtttgtcaaaacgtgctggaaatgtaatcacaattgaaacaatgctttcaatgattagtgaagatgctgctagatgatttatgttatctcaaaataatggcacaattatcaaaattgatttagatatagctaatttgcaaaactctgctaatccagtttattatgttcaatatgcgtttgcaagaatgaatagtattcttagaattgcaaattctgatcaattaaaagaaattactgattgcagtcttttgattaatgaaaaagagatatcacttttaaatcaacttgtctattatccttttatgttgcaaaaagctatggaaacaggcgaattgcacttattaactaactttttatatgaaactgctagtttatttcattcctggtataaagtttgcaaaattaatgatgataaaaattcacttttatcagcacaaagacttgctttattgagatcattacaatttatagttaaacaaatccttgatgttttgaagatttcaacaccacaacaaatgtaatacagacctgatttatacaaaaaatacttagaaaataaaagtgaaaatcactgttttaacactttttgaaaacactatttggccttacttaaatagttctattatgttacaagctcaaaaagcaaatttagttcaatttgaagtagtaaattgaagaaatttttgcaatgataaacataaaactgtggatgatatggcttatggtggaggaagtggcatggttttaaaagctgaacctattattaattgtttaaatttttataaagccccaaattctcatgtagttttactctccccagaaggtgaacaattttctcagaattgtgctaaaaaacttacaaaatacgaacacttaattttgttatctggtcactatgaaggttttgatcaaagaatttataaatatattgatcaaattgtttctttaggtgattttgttttaagtggtggggaacttgtagcactaagtgttattgatgctactgttagattaattaaaggagttattaatgatcagagtcttatttgtgaatcattcaatgataatctattagattttcctgtttatacaaggccatacgatttaaaaggcgataaagttcctgaagttttactttcaggagatcaccaaaagattgaatcatttcgtaaagaacagcaaatcttaaaaactgcaaaatacagacctgatttatacaaaaaatacttagaaaataaaaatgaaaaaaataaataagccagtttcagtttgtgcaacagttttataaatcaatgccatgttaattaatatagataatattttagtaaaaatgttaaataacatattgcaatttctcaaagaaagagaactttattcacaagctaattttgaaacagaactagataaccatttaaaagagaaaaaaaataacttttatgttggttttgatccaactgctaattctttacatattggcaattatgttttaattcacattgcaaaattattaaaagacatggggcatactccgcacatagttctagggagtgcaactgctttaattggtgatcctactggcagaattgaattaaggaaaattttagaagaaaaagaaattgtaaaaaacaccaaaacaattaaaaaacaaatcaaacagtttttaggtgatgtaattattcatgaaaacaaagtttgattagaaaaacttaattacattgaagttatccgtgaattaggtgcttttttttcagttaacaagatgttaagcacagacgcatttagtgctaggtgagaaaaaggactaactctaatggaattaaactatatgatcttacaagcatatgacttttattatctacataaaaaccataatgtcactttacaaataggtggaagtgatcagtgggctaatattttggctggtgctaacttaattaaaagaaaaaataatgctagtgtttttggattaactgctaatttattagttaaagctaacggagaaaaaatgggtaaaactagtagcggagcattatgacttgatgaaaataaaactagtgtttttgatttttatcaatactggattaaccttgatgatcaaagcttaaaaaagacttttttaatgctaacaatgcttgataaaaaagtaatagatgaattgtgtaatttaaaaggcccaaaaattaaacaaaccaagcaaatgctagcctttttaattactgaattaatccatggcactaaaaaagcaaaagaagcacaacaacgatctgaactaatatttagtaatcaaccagatcttgatattaagttagtaaaaacaagcactaatctaattgattatttagttgaaactaaatttattaaaagtaaatcagaagcaagaagattaattagtcaaaaaggtttgacaattaacaataaacacgttttagacttaaaccaaataattgaatgaaaagaagagttacaaattattagaaaaggtaaaaaaagttttttaacaattaaaactgttaattcttaggataaagaaagtgcaataaacttaattaagcaatttattaatggaaaaaattagaacacgttatgcaccatccccaacaggatatctgcatgttggtggtacaagaacagcaatctttaactttttactagccaagcactttaatggtgagtttattatcaggatagaagatactgatactgaaagaaacataaaagaaggaattaattcacaatttgataacttgcgttggcttggagtcattgcagatgaatcggtttataaccctggcaattatggtccatatctgcaatcacaaaaactagcagtttataaaaaactagcatttgatttaattgaaaaaaatctggcatatcgttgcttctgtagcaaagaaaaattagagtcagatagaaaacaagccattaataaccacaaaacccctaaatacttaggtcattgtcgtaatttacattccaagaaaattactaatcacttagaaaaaaatgatccttttactatccgcttaaaaataaacaatgaagctgaatatagttgaaatgatctggttaggggacaaattactattcccggcagtgcgttaacagatatagttattcttaaagctaatggtgttgctacttataactttgcagttgttattgatgattatgatatggaaattactgatgttttaaggggagctgagcacatctctaacactgcataccaacttgctatatatcaagcattaggttttaaaagaattccccgctttggtcatctttcagttattgttgatgaaagtggcaaaaaactttctaaacgtgatgagaaaactactcagtttattgagcagtttaaacaacaaggctatctacctgaagcattattaaatttcttagcactcttaggttgacatccacagtacaaccaggagttttttaatttgaaacagttaattgaaaactttagtttaagtagagttgttagtgctcctgctttttttgatattaaaaagctgcaatgaatcaatgctaattacattaaacaattaactgataatgcttatttcaatttcattgataattacttggatgttaaggttgattatttaaaagataaaaacagggaaataagtttactttttaaaaatcaaataacccatggtgttcaaataaacgaattgataagagaatcttttgccactaaaataggtgttgaaaacttagctaagaaaagtcatattttgtttaaaaacatcaaactttttttagaacagcttgccaaatctttacaagggttggaagaatgaaaagctgagcaaattaaaacaactattaacaaagtaggagcagtgtttaacttaaaaggtaaacaactttttatgccaataaggttaatttttacaaataaggagcatggacctgatttagcacatattattgaaatttttgataaagaaagtgcaataaacttaattaagcaatttattaatgcaacaaaccttttttaa

SEQ ID NO: 4 - DNA, 7400 bp, M. genitalium
aggaaaattaaaattaagttagcactagtagatacaaaagatgaagttatacaaagttcttaacagtaaaacaactgataaaagtctttgtttggaagttgagattgatccaaattactgacaagctacccaaaaaaaactagtaggtgaaatggctaaatcgataaaaattaagggttttcgtcccggtaaaatcccccctaatttagccagtcagtcgattaataaagctgaattaatgcaaaaaagtgcccaaaacgtcatgaacagtatttatgaatcagttcaacaagaagagatcgttgctagtaatgataatgtcattgatgattatcctaccattgatttcaaaacgatcactgaacaaaactgtgtacttttgttttactttgatctgatccctaactttcaactccctgattacaaaaagataaaagatttaacaccacttaccaagttaactgaagctgaatttaacaacgaaattgaaaagctggcaaaaactaaaagcacaatggtagatgttagtgataaaaaactagctaatggtgatattgctatcattgatttcactgggatagttgataacaaaaaactagcatcagcttcagcacaaaactatgaattgacaattggttcaaatagctttattaagggttttgaaaccgggttaatagcaatgaaagttaaccagaaaaaaactttagcactaacttttcctagtgattatcatgttaaggagttgcaatcaaaaccagttacatttgaagtagttttaaaagcaattaaaaaactggaattcaccccaatggatgaaactaatttcaaatcctttctccctgaacaattccaaagctttacttctctaaaggcatttaagagttattttcataagctaatggaaaacaaaaaacaagagacaattctccaggagaataaccaaaaaattcgtcagttcttacttactaataccaaacttccttttcttccagaagcgttaattaaactagaagctaaccgcttgttaaagctccagcaaagccaagctgaacaatataaaatcccctttgaaaaactcttaagtgcttctaatatcaccctaacagagttacaagatcgcaacataaaagaagctaaggaaaatgttacctttgctttggtaatgaaaaagatagctgacattgaaaagattaaggttgataataacaagattaaagctgaaattgaaaatgttattgctgttgaatatccctttgctagtgatgaaatgaaaaaacaactcttttttaatatggaacaacaaaaggagtttgtggaatcaattatcatcaacagattaacaacaactaaaatcgttagctattcaactcattagcactcaaagcttgtgagtgctaagaaatgtgttaaaatttattgaaattccctaattaacttttaaatatgcccgttacgaagaaaagtcagatcttagtagttagaggtcaagtcatttttccttttgttccctttagtttggatgttggcaggccccgttcgcgtaagatcatcaaagcgcttaaaactctgaaaaccaaacgtttggttttagtaacccaaaagtttactggtgaacaaaaccctgagtttaatgacatctatcatgtcggtacactctgtgagattgatgagatagttgatgttccaggggttgatagtaaaacagtagactaccgtattaaaggcagaggtttacaacgggttttaattgaaaaattctcagatgcagatattaatgaagttagttaccaattacttaactccacagttaaagatgaagctaatgttgacaggttcttacagcgaatctttcctgaaaaagaagaaattgaacagttaatggaaggagctgagaagtttttagaacttgaaaacatcagcaaaacagttaatgttcctaagggtttaaagcaacttgatattatcacctttaaactggctaatcttgtccctaacactgaaagtattaaacaagctatcttagaggaaaatgagatagcaaaccgattggaaaagattatccaagcagggattgaagatttacagaagatccaagattatggtagatctaaaaacaaggaaactgagtttgataaacttgacagtaaaattacccgcaaaattaacgaacaactctcaagacaacaacgtgatttctatcttcgtgaaaagctaagaattatccgtgaagagatagggattagttccaaaaaagaggatgaagttgctagtattagaaagaaactggatgaaaacccttaccctgaagccattaaaaaacggattttaagtgaacttgaacactatgaaaactcttcctcctcttcccaagaatcaaccttaaccaaaacttacattgatacgcttttaaacctgccttgatgacaaaagagcaaagataacagtgatgttaaaaacttaattaagacgttagataaaaaccacactggtttagataaggttaaagaaaggattgttgagtatttagcagtacaactaagaacccaaaaaaacaaaggtcctattatgtgtttagtaggtcctcctggggttggtaaatcaagtctagctaagtctattgcagaagcattagataagaagtttgtcaagatctcattagggggagtacatgatgaatcggaaatcagaggtcaccgtaaaacttacttaggttctatgccaggaaggattttgaaagggatgacccgtgctaaggtaattaatcccctctttttacttgatgaaattgataagatgacctcctccaaccaaggttatccttcaggtgctttacttgaagtattagatccagagttaaataataagtttagtgataactatgttgaagaagattatgatctttctaaagtaatgtttatcgctactgcaaactacatagaagatatccctgaagctttacttgataggatggagataattgaactcacttcctatacagaacaagagaagattgagatagcaaaaaaccacttaattaagcgttgccttgaggatgctgatcttaacagtgaagaattgaagttcactgatgaagcaatcagctacatcattaagttttacacaagagaagcgggggttagacaattagaacgattaatccaacaagttgtaagaaagtacatagtagcaatgcaaaaagatggcatcaaacaagaaacgattgatgtaaacgctgttaaaaaataccttaagaaggagatctttgatcacactatgcgtgatgaagtgtctctacctggaattgtcaacgggatggcatacaccccaactggaggggacttacttcccatagaagttacccatgttgctggtaaaggagagttgatcttaactggtaatttaaagcaaacaatgcgagaaagcgctaatgttgctttaggctatgtaaaagctaatgcagagcgttttaacattaatcctagtttgtttaaaaagattgatattaacatccatgttccaggtgggggaattcctaaggatggacctagtgctggtgctgctttggtaactgcaatcatctcatcattaactggtaagaaagtagatcctacagtggctatgacaggagagatcactttaagaggcaaagtgttggttattggtggggtgaaagaaaaaactatctcagcttaccgcggtggggttacaactatctttatgcctgagaaaaacgagcgctatttagatgaagtacccaaagagatagtagataaacttaacattatctttgttaaggaatacagtgatatctacaacaagcttttcagttagttttatataatttttgcattaaaaggagtgagcaattaaaatgaatattaatttcacacctgctggtgaaaatcgtaattttttgcaagaaattggtcgtaatattaacgatgaagtattaaaaaataaggtcgatcctattattggaagagataacgaaattcgtcgtttaattgagatattaagtcgtaaaagcaaaaacaatcctgttttaattggtgaacctggagttggtaaaaccgcaatagtagaaggttttgttagaagagttgttagtaatgatgtacctttaaatttaagggatgtagaaatttatgaactatctctttctggattaattgctggcactaaattccaaggtgaatttgaaaaaagaattaataccattcttaagcaagtaaaagaatcaaatggcaggattattctttttattgatgaaattcaccaaatagttggattaggacgtaattctagcagtggtgcaatggatattgccaatatattaaagccgatgctagctcgaggagaaataaaagtaattggcgctactactctaaaagaataccgggaatacattgaaaaagatggcgctttagaacgtagatttcaaaaaattcttattaacgagcctagtagtcaagaggcactaacaattatgcgtggtttaaaaacacgttgagaactctttcataacatcactatttttgatagtgctttagtagctgctgttgaaatgtcaactcgttatattaatgaacgtaatttacctgataaagccattgatcttattgatgaggctgctgctaagatcaaaacagaaatgtcatctgaaccagttgcaatagatagtcttaaacgtgaaataatcaatcttgagacagagtatgcagctcttaaacaagataaggaaaatgataacaaacaatcaaagaaagaatatttagagaaactaaaaaaacaattagatgctcttaaacaaaagcgtgattcacttataaatgaatgaaaaaaggaaaaggctgattttgaaaacattaataagctcaaaaaagagattgaagaatttcaaaccaaactagagacataccaaagtgaaggaaattatgaaagtgcatctaaaattctgtactctgatatcccaagacttaaaaaagaactggaaagtgcacaacaaaaatatgcaacttctaagcacgatttatttaaaactgaagtttctgaaaatgaaattgctgaagttatttcacaaacaacaggaattccacttaaaaaactattagaaagtgaaaaggataaacttttgcacttaggtgatgaaatcaaaaaaagagttaaaggacaagatgaagccatcgatgctgttgttaacactgtaattagaggtagagtaaatataaatgacccaaacaaacctattggttctttcatctttttaggttctactggtgttggtaaaactgaacttgccaaatcattagcagaagttctttttgacaatgaaaaagctctgattcgttttgatatgagtgaatatatggaaaaacattcagtagctaaattaattggtgcacccccagggtacataggttatgaacaatcaggtttgctaactgaagcggttagaagaaaaccttatagcgtcttgttatttgatgaaattgaaaaggcacatcctgatgtaactaatgttttattacaagttttagatgatggtactttaaaagattcacaaggaagggttgttaatttcaaaaatactttgataattatgacttctaacctaggttcaaattttcttttagaaggaaaaaaagatttggccattcaaagtctaaagaaacatttccgtcctgaatttataaatcgtattgatgagatagtatttttcaatgttcttgagaaagatacagttttatcgataatcaacagcttgttggcacaactttcaaaacgcttgaataaacaaaatttattttttaattttgattcaaatctaacagagtttatctataaaagtagttttgatcaacagtttggtgcaagaccaattaagcgctttattgatcatagtgttgcaactttaatagctaaatatatccttcagggaaagataaaaaaaggtgttggatacaacattgcagttgttaaagacaatattaccattacacaaaataataagtcttaaaaagataactccttttcagaataataaatagagatatagaatgagaataaataaaccttttagtgatgatagcaacacagttgtttttgtgagttcaaaaacatatggtgtaaaagaagaagctgcacataatcctaatgttgaatttggtgttgttttaccaactgattttcctgctttcaaccgtgctttagttcaatttcttaaaagaaagaaaaccaaattaaacattaatcttgacagtcttatagaactttataagaaaaatgaaaatagtggttgttttcatactgcgataaaaactgttattacaagtgttactttttgtgaaactactcctttcacaatgaaaaccaaacctgaaaaaaatgttgaagttgctgttcaatgtgctgttgaatatcacaacttagttaaagaatatgaaacagtaggcgaatatgttaacctagcaagagaattacaagacactccttcagatctactttattcagaagtatttgttaaacattttgaaaaggctgcaagtaaattgcctgtaaaaataaaagttcttaaacaatcagatctaattaagaaaaaaatgggtttacttttaggggttaatcaaggctctgaaagagaagcacgtttacttgttattagttatcaagctaataaaaattccaaagaaaaacttgcttttgtagggaaaggaattacttatgattcaggcggaatgaacattaaaacaggtgattatatgcgtggcatgaaatatgacatgagtggtgcggctatagtatgttctactgttttggcattagctaaaaacaaggttaaaaccaatgttgttgcagtagcagctcttactgaaaatcttcctggtgctaaggcgcaacgtcctgatgacattaagatagcatacaatggtaaaagtgttgaaatagataacactgatgctgaaggaagattggttttagctgatgctattacatatgctgctaaggatttagctgctacacatattattgatgtagcaacccttactggtttaatgtcatacatattgagtactacctatacaggtattttcagtacttgtgatcaccagtgagaatcttttaaaaaagcagcatgtagtgcaggtgaacctgtatgaagattacctatgcaccctgattatttaaaacctttacagctaacaaaacttgctgatttgcaaaattctactagtgcaagaggtgctggatcttcaagagcagcttgtttccttgcagaatttagagaaggtgtatctttgatccattgtgatattgcatcaactgcttccattgagaaccttggacaaggtgttttagtgagaaccttgtacgaacgtgctagtcagcttgcaaataaataa

SEQ ID NO: 5 - DNA, 11138 bp, M. genitalium
atttaaaaccagaaaatacattaaatatcctaagtaatttatggcagaaatgatagaagcaaaaaatcttcgtaatgggcaaaccatcttcggtcctaacaaagagattttattagtactggaaaatacatttaacaaaaccgcaatgcgccagggaattgttaaaactaaagttaaaaacttaagaactggggctattgtttggcttgaatttactggtgacaaattagaacaagtaattattgataagaaaaaaatgaatttcttatacaaagatggtaataactttgtttttatggatcaaaaagactacagtcagattgagattaatgaaaaaaaattagagtgggaaaaaaatttcattactgaagaaattgaagttactgttattacttatcaagatgaaattctaggagttaatttacctgatttagttcctattgaagttgagtttgctgaagatgctattcagggcaatactgctaacatggcaagaaaaaaagcacgccttgtaactggttatgaacttgatgtaccccaatttattaatactggtgataagattgtaattgccactgttgatggcaattaccgtgaaaggtttaacaaataataacaaagcctttgcccacatgcgttggtagttatttattatgtcaagaacagttgatttaaaaaacttccgtaactttggcattatggcccatattgatgctgggaaaaccaccacatcagaacgtattttgttccattcaggtagaattcacaagattggtgaaacccatgatggtgaatcagtgatggactggatggaacaagaaaaagaaaggggtattactatcacctctgcagccacttcagtgagctgaaaaaactgcagcttaaacttgattgacactcctggccatgttgactttacagttgaagtggagcgtagcttaagggttttggatggagcaattgcggtattggatgctcaaatgggagtagaaccacaaactgaaacagtatgaagacaagcttcacgctatgaagtaccacgggtaatctttgttaataagatggataaaaccggtgctaactttgagcgctctgttttatcaattcaacaacgcttgggagtgaaagctgttcctattcaatttcccataggtgctgaaaatgatttcaatggcatcattgatatcatcactaaaaaagcttatttttttgatggtaataaagaggaaaatgctattgaaaaaccaattcctgaacagtatgttgatcaagttgaaaaactttacaacaacttagttgaagaagttgctagtttagatgatcaactcatggctgattatctagatggtaaaccaattgaaattgatgcaattaaaaatgcaattagaaacggggtaattcactgtaagtttttcccggtattgtgtggttcagcatttaaaaacaagggaattaaactcttacttgatgcagtggttgattttctcccttcacctgttgatgtcccacctgctaaagcaattgatgcaaacaacaaagagatatctattaaagctagtgatgatgctaactttattggcttagcatttaaagttgctactgatccttttgttggtagattaacttttattagggtttatgcaggagttttaaaatctggttcttatgttaagaatgttagaaaaaacaaaaaggaaagggtatcacgtttagtgaaaatgcacgcacaaaatcgcaatgaaattgatgaaattagagcaggggatatctgtgcagtaattggcttgaaagatactactactggagaaactttaactgatgataagcttgatgtgcaactagaagcaatgcaatttgctgaaccagtgatctctttagcagtagaacctaaaactaaagcagatcaggaaaagatgtcaattgctttatcaaaactagcagaagaagatcctacttttaaaacctttagtgatcctgaaacagggcaaactattattgctggaatgggtgagttacaccttgatatcttagttgataggatgaaacgtgaatttaaggtagaagttaacattggtgcacctcaagttagctttcgtgaaacctttaaatcaactagtgaagttgagggtaaatacatcaaacaatcaggtggtagaggtcaatatggacatgttaaaatccgttttgaacctaataaagataagggctttgaatttgttgataagattgtgggcggaaggattccaagggaatatattaaaccagttcaaactggtcttgaaaatgcaatgaattcaggtcctttagcaggttacccaatgattgatattaaagctaccttatttgatggttctttccatgaagttgactcaagtgaaatggcttttaaaattgctgcatccttagctttaaaagaagcaggtaaacaatgtaacccagttttacttgaacctattatggcaatagaagttactgtacctgaacagtactttggggatacaatgggtgatatcagttcaagaagagggatcattgaaggtactgaacaacgtgataatgttcaactaataaaagcaaaagtacctttaaaagagatgtttggttatgccactgatttacgctctttttcccaaggtaggggtaattatgtaatgcaatttagccattatgctgaaactcctaaaagcgttgttaatgagataattgctaataaaaaatagcagttgttctttgaaatgatctaataaggctttaacaagggtgcttttgccaacaccgctaggaccagtaatgacaaaaattctcccctgattgttcacttcaattgttagaataattttaactttactgtttttatcaatgacttttcaaccaactaaaacttggcttgtttttgatgataatgctttgattaacaagccaactgaagctgttaattttccaatagatgagcaaattgaaacctgtattaaaaagatgattgcatatgttgatgcttcatatgatggtaaagcacaagaatatgacattattccaggaattgggatagctgctaaccagattggctattgaaaacaactgttttacatccacctcaatgatttaaacaaagaaaaaaagtgcctactgatcaatcctaaaattattgatcaaagtgaaaataaagcatttttagaaagtggtgaggggtgtttaagtgttaaaaagcagcacaaaggttatgtaattcgtagtgaatggatcactattaaaggttatgattggtttgaaaaaaaagagattaccattaaagcaactggactatttggaatgtgtttacagcatgaatttgatcacttacagggacgctttttttaccaaagaattaaccctttgaatccatggtttaaaaaaccagaatgaaaagtgattaatcctactttgaagacaagtaatggataacttactgcaatgattaccgccttacaacaattagcacaagatgaaaaaaaatagagctttcaatcaggttaaaaaaacaaagtttgacggtaggattaaaaccagtgccaaacaccagttacgtaatgttaaaaccggggttaaagatggtgtttttatctataaaggtcctttaactgttagtgagtttgcaagtaaaactaatatcgctgttgctaacattatcaaacacttttttttaaatggtttggcactaacagttaattcagttttaacaaatgaacagttagcagatgcatgtgttaactttgggtttgactttaagatggaaactgaagttacccatgaaaatattgtagctaacatccagtttgaagatagtgatgatttattgcaaccaagaccacctattgttactatcatgggtcatgttgaccatggtaaaacttcgcttttagacacaattagaaaaactaatgtaactgctaaggagtttggcggaattacccaaaaaattggtgcttatcaggtgaaaaatcaccaaaataaaacgattacttttattgatactcctgggcatgaagcatttactttaatgcgtgcaaggggtgcaaaagtaactgatattgtggtgttggttgtggcagcggatgatgggattaaaaagcaaacagaggaagcaattagccatgctaagagtgctaacactcctatcattgtttttattaacaagatggataaaccaactgctaaccctgatctggtgatccaacaactcaataagtttgatttagttcctgaggcttggggtgggaaaactatctttgtaatgggtagtgctttaactggtcaagggattaatgagttgcttgataatatcttgttgctaggggaagtggagggttatcaagctaactataatgcccattcatctggttatgcaattgaagtacaaacttcaaagggacttggccctattgccaatgtcattgtaaaaaggggtactttaaagttaggtgacattgtggtgttagggcctgcatatggaagagttagaacgatgcatgatgaaaatggtaatagcttaaaacaagcaaccccttcaaaacctgtgcagatctcagggtttgacattatgcctgttgctggggaaaagttcattgtttttgatgatgagaaggatgcaaagttaattgctaacaagtttaaagaacaacaaaaacaaaaagctaacaacttaacagttaatcaaaccttaaaagaacagattaaaaacaaggaaattaagatattaaatttgatctttaaagcagatagtgatggttcattgcaagctattaaacaagcagttgaaaacattaatgttgctaagatctcacttagtatcatccatgctgcagtggggcagatatcagagagtgatattatgctagcaaaagcatcaggggctttattgtttagtttaaacttaggtttgagtcaaactgtaaaaaacattgctagtttacaaggggtaaaattagaagttcactaccatatccctaaactagcagaggagattgaaaacatcttaaaaggtcaattagatcctgtttatgaagagattgaaataggtaaagcggaagttttacaactctggttccactctaaaatcggtaatattgcaggaaccattgttaaatcaggaaagataaaaagagggaatttatgtaagttattcagagataaagagattatctttgaaggcagaattgactctttaaaaaatgaaaaaacgcctgttaatttaatagaaacagggaagaattgtgggatagttattaatggttgcaatgatattaagattggtgatatcattgttgcttatgaaaagcagatagttaaagatggcaagctatagaaaacaacggattgaaaatgatatcatccgcttaattaatcgcacgattattaatgagatctatgatcctgttgttaagttaggtcatgttagccatgtgaagttatcagctgatttttttcatgcagtagtttatcttgattgttatgatcgtagtcagattcaaactgtagttaatgcttttaaaaaggctcagggcgtttttagtcaaatgttagcacaaaatttgtacctagctaaaagtgtaaaactccactttgtgaaggatgatgcaattgacaatgctttgaaaatagaacagataattaactctttgaaaaactaaaagaacaactcaagacaatcaaacaatttattagataaagatgatctatctcaaatctgcaaatgaagttgcagggattaaaaaagcatgtgcaatcttcaaagcagttaaggcatattttacaattgaaaagttacttggcaaaaagttggttaccattgatcgtttaatcaaacaattcattgaacaaaaacaagctaaatgtgcgtttcatggttatctaggtttccctggttttaactgtctatcgttaaaccaaacggttatccatggagttgccgatcaaactgtttttaaagatagtgataaactaacgcttgacattgggatagactatcatggttatctttgtgatgcagctttcactttacttggtaataaagctgatccaaaggcagtaaaactgttaaatgatgttgaacaagcatttagtaaggtaattgaacctgagctatttgttaacaatccgattggtaatttatccaatgcgatccaaacttactttgaaaacaagggctattttcttgtcaaagagtttgggggtcatggttgtgggattaagatccatgaagatcctttaatcttaaactggggagagaaaaaccagggcgttaggttacaagaggggatggtaatctgtattgaaccgatggttatgactgatagtagtgagataacaatggcagctaacaactggaatgtactaactttaaagagtaagtttaactgtcatgtggaacagatgtatcacatcacaaacaacggctttgaatgtttaactaactaatgaaaaacgataaactctttctaacaggtaagatactggaaattatccatggtgataagtaccgggtgatgcttgaaaacaatgttgaggttgatgcacatctagcaggtaaaatgaagatgaaaagaaccaagattctccctggggatgttgttgaggtggaattttctccctatgatttgaaactaggtaggataacccaaagaaaataatttaattgatgaaaacagtactaatttagggatagttaagatggaaaacgctttaaagttagcacaagaaaaacagttagatctagttctaattgctccaaacccaaccaaaccgatcgttaagttgttggactttggcagatatacctatgatttaaagcgtaagaaaagacaagccaagaaaaaccaaacaatcatccaaaccaaagaagttgttgtcaaaccaacgattgctaaacatgatttagaatttagagcaaaacagagtaagaattggatagaaaaaggtcatcatgtcaagtttatagtccgtgcctttggcagggttagcaccaggatagagttaattgaaaaggtgtttgatgacttttaccagttagttaaagatgtagttgagatccaaaaacctttaaccgcttcttccaaaacgatgtacgctgctctattagtacctttaaaaagatagtgaatatattaatatttattggtctttgtcaaaaaataagatgcaactaaaaaagccccattttcaaccaaataaaattgctaattgtattgtgatcgggggaatgattgctttaggaaaaaccaccattgctaatacattagctaaccacattcaagctgcaaaagttgtttgtgaattggaaactaatgaccagttggttgaacttttactagcaaagatgtatgaacgtagtgatgaattgctctattcacctttgtttcagctttattttacgcttaatcgctttggtaaataccagaacaattgcaacactatcaatccaaccatttttgatcgttctatctttgaagactggttgtttgctaagcacaacatcattcgtcctgcagtcttttcatactataaccaactgtgaaatagattagcaaaagaactagttaataagcatggggttcctaatttatatgtcattttggatggggattgaaaattatttgaaaaaagactatttatgcgtaaccgcaaagtagagattgataactttactaaaaatcaactttactttcaaaatttacacagggtttacactggatttatggaagcggtttgtaatgattttgggattaattactgtattatagatgcaaaactaccaatagtaactattattaaaatgatccttgaaaaattaaagttacaaaagttagattgaaaatttatctaaattaaataataaaagtgtttttgctgcttttaattttgtcatggaactaaataaaaattacctaactcaagaaggatttaagcaactggaaaaagaacttgaaaacctaatccaagttaaacgtcctgagattatcagactcttacaagaagcacgtgatcagggtgatttaagtgaaaatgctgattatgatgcagctaaagcacagcaaggtgagattgaaactaggattgctgaaattcaagatatattagccaacgctaagttaattagtgaccaccaagcaaaaaccaaagtaacaaaagttagcttagggagcactgttgagatctatgattacagttctaaatcgaacgaaaaatacacaattgtaggtacacttgaagcaaatcctgaagaacacaaaatttccaatgaatcaccccttgcccttgcaatctatgggcgtttaattggtgatgaatgtgatgttgttggtattgaagttccctatcgtgttaaaatcctgaagatcagcaacagataatatttactatcttattagtaatattaagcttagtgcaataatggcaacgaaaatagagctaataaaagaattgcgtaaatcaacacaagcaagtgttatggattgtaaacaagctttggaaaaaaataatgatgattttgagaaagctgttaagtgattaagagaaaatggcattgttaaatcaaccaaaaaattaaataaggttgcaagtgaaggaattattgttttaaaaagcaatttacacaaggcaattatggttgagataaactcacaaactgattttgtagccaaaaatcaagagttaaaagaattttcagatttaatgcttgaaaaaatatttgaaaaagtaaatccaaaaacagaattagttgaaattgaaaaaattcaaattaataatgatgaaaaagttagtgaaaaactagcattaattgcttctaaaactgatgagaaaatagtacttagaagagtagttgtatttgaaactaaaactaatcaaattttcacctatttacatgccaataaaagaattggggtaattattgagattcaaggaaaactcaacgaagatgatggtaagcatttagcaatgcatattgctgctaattcaccacaatttattgatcaaagtgatgttaatcaaacatgacttcaaaatgaaagaaatattatccgttcccaagcagaattagaggttaaagaaaatcctaaaaaagcaatttttttagaaaaaactattgaaggtagagttaacaaattactaattgatacctgcttaattaaccaaaaatacttaattgatgaaactaaaacaattggtcaatttttaaaagaaaaacaagctaaggttcttaaatttattaggtatgaagtgggagaggggattataaaggaaactgttgattttgttagtgaagtaaatgcacaaatcaaacaataagggattataaaggaaactgttgattttgttagtgaagtaaatgacaaaagcacattacattgatttttttaaacaagcagctgataaaaaaattcaatgattaaaagaagagttaacaaagattagaacaggtaggccaaatcctaaaatctttgataatcttttgattgaaagttatggacaaaaaatgcctttaatatctttagctcaagtgactattaatccgccaagagaaataatcataaaaccatttgatcctaagagtaatactaatgctatttacagtgaaattcagcgggcaaacattggtgttcaaccagttattgatggtgaaaaaattcgtgttaattttccccaaattactcaagaaactcgcttagaaaatattaagcacgttaaaaaaataatagagcaaatttatcaagaactgagggttgtaagaagagatgcattacaaatgattaaaaaagataatcacaatgaggatttagaaaactctttaaaagctgaaatagaaaaaattaacaaaaattattctaatcaattagaagagattcaaaaagacaaagaaaaagaattgctaacaatttaaatcttaaacttatttaaaattaacaacataatttaaacaaatggcaagagagaaatttgaccgttccaaaccacatgtcaatgttggtaccattggtcacattgaccatggtaaaaccactttaacagctgctatctgtacagttttagcaaaggaaggaaaatcagctgcaacgcgttatgatgaaattgataaagcccctgaagaaaaagcaaggggaatcacaattaactctgcacacgtagaatattcttctgacaaacgtcactatgcccatgttgactgtcctggacatgctgactacattaaaaatatgatcacaggtgctgcacaaatggatggagctattctagttgtttcagcaactgatagtgtgatgccccaaacccgcgagcacatcttacttgcccgccaagtaggggttcctaaaatggtagtttttctaaacaagtgtgatattgctagtgatgaagaggtacaagaacttgttgctgaagaagtacgtgatctgttaacttcctatggttttgatggtaagaacactcctattatttatggctcagctttaaaagcattggaaggtgatccaaagtgggaggctaagatccatgatttgattaaagcagttgatgaatggattccaactcctacacgtgaagtagataaacctttcttattagcaattgaagatacgatgaccattactggtagaggtacagttgttacaggaagagttgaaagaggtgaactcaaagtaggtcaagaagttgaaattgttggtttaaaaccaattagaaaagcagttgttactggaattgaaatgttcaaaaaggaacttgattcagcaatggctggtgacaatgctggggtattattacgtggtgttgaacgtaaagaagttgaaagaggtcaagttttagcaaaaccaggctctattaaaccgcacaagaaatttaaagctgagatctatgctttaaagaaagaagaaggtggtagacacactggttttttaaacggttaccgtcctcaattctatttccgtaccactgatgtaactggttctattgctttagctgaaaatactgaaatggttctacctggtgataatgcttctattactgttgagttaattgctcctatcgcttgtgaaaaaggtagtaagttctcaattcgtgaaggtggtagaactgtaggggcaggcactgtaacagaagttctagaatag

SEQ ID NO: 6 - DNA, 23272 bp, M. genitalium
tttacagttttagcaataataaaaaatctttgaatattgcatgaaaaaaataaacgttgtttacaatccagcatttaatccaattagctctaaattaaatcaaactcaacttttaaaaaatgctagtgaagagttagatatagaactaaaattctttactagttttgatattaatacaactaaagcaaaagcaaatttacccttcatatccaacaaaattctttttatggataaaaatattgctttagctagatgactagaaagcaatggttttgaagtaattaacagttcaattggaattaacaatgcagataataaaggacttagtcacgctatcattgcacaatatccattcataaagcagattaaaacacttttaggacctcaaaattttgacagggagtgaaatccagtaatgctcgatgtttttattaatcaaataaaacaaagtatggagtttcctgttattgttaaaagtgtttttggttcttttggtgattatgtttttttgtgtttagatgaacaaaaattaagaaaaactttaatgtcttttaatcaacaagcaattgtgcaaaaatacattacttgctctaaaggtgaatcggtaagagttattgttgtgaacaataaagttataggtgctttacatacaactaataatagtgattttcgttctaatctcaataaaggggcaaaggcagaacgcttttttttgaataaggaacaagaaaatttagcagttaaaattagtaaagtaatgcaacttttttattgcggtattgattttttgtttgatcaagacagatcattgatcttttgtgaagtaaatcctaatgtgcaattaacaagaagctcaatgtatttaaatactaatcttgcaattgagcttttaaaagcaatttagtattattgcagttttactgcataatgtaaaattacacagcatgtcagatacaaatactgaaaaacctgagttagtttcccttaataagttaagtgagatgcgcactaacatcgggatggttaaacgttattgaaacccaaagatgggattctttatcgaacctgaacgtaagcataataacgatttattgaagcttgatctacagtaccaagcgttaaaaactgcttataacttcattaaggatgttgttaaaaatcacggacaaatcctttttgttggaacaaagaatgattatgttaaaaaactggtaattgatattgctaaaagagttaatgttgcatatattacccagcgctgattaggtggtactttaactaactttaaaaccctttctatctcaattaacaaactcaataaattagttgaacagcaaaagcaaaatgcaaatgatctaaccaagaaagaaaacctgttactttcaagagagattgaaagacttgaaaagttctttggtggggtcaaaaatttaaaaagacttcctaatctaatagttatagatgatcctgtttatgaaaaaaatgcagttttagaagcaaacagcttaaaaatccctgttgtggcactatgcaacaccaacaccaatccagagctagttgactttattattccagctaataaccaccaaccccaaagtacttgtttattgatgaatttactagcagatgcgatagcagaagcgaagggttttgaaaccttgtatgcttacaaaccagatgaacagatccaaattgaaattcctcccaaacaagaacgccaagttattaaccgttccaataccagaaacatcactaaccagcgcttaaacattaaccgtcaacaacaagaaactttatagatggctaaaaaaacagttaaatgtgggagctaattaagcgtttgtaccacagcaaaatatggctaaaaaaacagttacaagaatcgctaagattaacctaattggcggacaagcaaaacctggccctgcgcttgcttctgtagggattaatatgggtgagtttaccaaacaatttaatgaaaaaaccaaggatagacaaggtgaaacgatcccttgtataatcactgcttttaacgataaatcatttacttttgtcttaaaaactacccctgttagtaacttaattaaacaagctgctaaactagaaaaaggtgctaaaaatgcaaaaactattgttggaaaaatctccttacaacaagctaaggagattgcgcaatacaagttagttgatcttaatgctaacacagttgaagcagcattaaaaatggtgttaggtacagctaaacagatgggaatagaggtaactgattaatgaaaaaactatcaaaaaggatgcaagctgttaccaagctcattgataaaaacaaactttatcctatccaagaagcatttgaattaattaaaaaaacagcaattactaagtttgtcagttcagttgatattgctgttagtttaaaccttgatactactaaagctgaacaacagttaagaggtgcaattgcttttccttttagtattggtaaatctatcagaattttagctatcactgatgatgagaaaaaagctagtgaagcaggtgctgattttgttggtgggcttgataagatagaagcgataaaaaatggctgattagattttgatctaattatcacttctcccaagttcatgggagcattaggtaaactaggaaaactattaggaaccaggggattgatgccaaacccaaaaactgaaacagttactgatgatgtagttagtgctattaaagcttataaaaagggtaagaaagaatatcgaactgattcatttggcaacatccacctctctttaggtaaaacagataccaaaactgagcacttggttgctaatgccatggctttaatagatttaattaagtctaaacgtcctagcacagtcaaaggtacttacattaaaaatattgctttgacaacaacaatgggaccaagtttaaaagtaaagctacctgattaaacaaccagctagattttgttagaatacttcagttgtctatatggctacaatagcgcaattaattagaaaaccacgccaaaaaaagaaggttaaatcaaagtcacctgcactccattataacctcaaccttttaaacaaaaaaactaccaatgtttactcaccactaaagcgtggtgtttgcaccagggttggcaccatgacccccagaaaacctaattctgcactaagaaagtatgctaaggttagacttacaaatggctttgaagtacttgcttatatcccaggagaaggtcataacctacaagaacacagtgttactttattaagggggggtagagtaaaagatctccctggagttagataccatattgttcgtggtactttagatacagttggtgttgacaaaagaagacaacaacgttctgcatatggcgctaaaaaaccaaaaccaaaatcttaacttgatcagttaaataatgagaaaaaatcgtgctttaaaaagaactgttttacccgatcctgtttttaacaacacactggttacaaggattattaatgtcatcatgaaagatggcaagaagggtttagcacaacgcatcttgtatggtgcttttgagatcattgaaaaacgcaccaaccaacaacctttaactgtctttgaaaaagcagttgataatgttatgccccgcttagagttaaaagtgagaagaattgctggttctaactaccaagtaccaactgaagttccccctgacagaaggattgctttagcactaagatggattgtgatctttgctaacaaaagaaatgaaaaaacaatgcttgaacgtgttgctaatgaaattattgatgcttttaataacacgggtgctagtgttaaaaagaaggatgatactcacaagatggcagaagctaacaaagcctttgcccacatgcgttggtagaaatataactttactcaatgagtttttccaaaaagtttttatgcactacaatatcattcttttagttgatggtacgcttagtttagaacaagctaaccaagttgaacaaaaacaccaaaaattgcttgaaaaggcaactgaatttaaaagtgaatacttaggtttaaaagagttggcttaccccattaaaaagcaactttctgctcactattacagatggagttttcatggtgaaagcaattgtactaaggagtttaaaagagctgctaacatcaataagcagataataagagagttaattattaacagagaaaaagactatggttatttaggttcagttaaccctaaaaaacaacaactgtctttgcagaagctaaccaagtataatgagattattgctagtgaaaataatcctgataacccagatgcgcctgtcacttctggtctagcttctgttaaaccacggctatcaagagttgaaaaacaaaaggaacgtgaacttgaaaagtgaacggttgttcaccaatcaggtaactttgatactgtacagatcaatccttatcgtcctaggataaaacgctttttacaaaacaaccaacaaacctcccaagctaataataaccaacctcgttttcaaaatcaatttaaaaaaggagcaaaaccttagacccctatatttgaaaatgatgtccaactagaggaggaaagtgatgattaataaagaacaggatttaaaccaattagaaaccaaccaagaacagagtgttgaacaaaaccaaactgatgaaaagcgcaagccaaaaccaaactttaaaagagcaaaaaaatattgtcgattttgcgccataggtcaactaaggattgattttattgatgatttggaagcaatcaaacgctttctcagtccctatgcaaagattaatcctagaagaattacaggtaattgcaacatgcaccaacgtcatgtagctaatgctctaaaacgagcacgttacctagctttagtgccatttattaaagattaaatatgaagataattttgaagcaagatgttgctaaattaggcaagcggtttgatgttgttgaagttaaagatgggtttgctatccattttttatttcccaaaaaactagctgcacctttaacaaagaaagcaattgctaaccgtgatttgtttttaaaacaacaacaagaacaataccaaaaaaatcgtgccttagctgaaaaattgaaactagtaattgaacaaacaccattaacttttcaactcaaacaacacgatggcaagccatatggttcaatcatcaccaaacaaataattaatttagcaaaacaacaaagacttgatttacagcgctttatgtttaaagataatgtgcgcttacagtttggtgaacacaaactaattttgcacctttttgaggagataactgcaactttaactgttatagtgaaccctgaaaatgggacaacaaactagccgtagtcaatttattaactaactagtgaactagattttgatgaatagcgctgtaaaatatcctgagctgaagatcaaacttgagtcttatgatagcacccttttagatctcactattaaaaagatagttgaggttgtaaagggtgtgaacattaagattaaaggtcctttacctttgcctactaaaaaggaagtgatcaccattatccgctctccccatgttgataaagcatccagagagcagtttgaaaaaaatacccacaagcgcttaatgattcttgttgatgttaatcaaggagggattgatagtttaaaaaagattaagatcccagttggggttacactgcgtttttcaaaataggttatggatgtaaggggaatatttggtgttaaagtagggatgagtcagatctttactgagcaaaatgagtgcttacctatcaccattgtttattgtgaagctaatcaggtggctgggattaaaacgattgctaaagataattacaacgccactctattaagctttcaaactgttgatgaaaaacaacttaacaaacctaaacaagggttcttttccaaacttaaactagaacctcataaatatctgagggaaatcagaaagatgcaagggtttgagttaggtaagaagatcaccccccaggagttgtttaagataggtgaatatgttgatgtcacttcactcaccaaaggtaggggttttacaggagcgattaaaaggtgaaactttaagataggtcctttgggtcatggggcgggttatccccaccgctttcagggttctgtgcaagcaggtagaggtggtagtagtgcgcagcgtgtttttaagggtaagaagatgtctgggcattatggtcatgaacaagttacgatccaaaacctctttattgttggctttgatgaaatcaataagttagtgttagtttcaggcgcaattgctggtcctgagggtgggattgttttaattaaaactgcaaaaaagaaaactggcaagataaaagatataaagttagcagtacaaactgttaaagccccacaactaaaagcaccaaaaaagcagaaaactaaggttgaaaccaaccaggttaacccaaaaattgaagaagagaaaactaagtaatggctaaacttaaagtaatccagtttgatggtagttttaaaggtgagatccaacctgctaaccacctccttttaaaaaaagcagtgatccaaccagtgtttgatgctatcttattagaacaagcagcatgtagacaaggcactcactctactttaactaagggtgaagttagtggtgggggtaaaaaaccatataaacaaaagcacactggtaaagctagacagggttcaataagaaacccccattatgtggggggtggtgttgtttttggtcctaaacccaaccgtaactacaaactaaaactaaacaaaaaggcttatcaacttgctttaactagtgcctttgcacaaaagcttaacaacaaccaagtgatagttgctgaagccaagttgtttgaacaaaccaatgccaaaactaaaaagatgctgacgtttctcaagaatgccaaactaactgagcaaaaactcttgtttgtgattgatactatctcaaaaccactgttgttgagtactaacaacctaaagcagatagtagtcaaacagtttaataaagtatcagtaagagatctacttttagctaaaactatcatcattgaaaaagctgcttttacaaaactggaggaacgacttaaataggctatggatgtaaccaacatactcttaaaaccagtcttaactgaaaagagttatctcaaccagatgggggaattgaaaaaatatgtctttgcaattaaccctaaagctactaaaaccaaagtaaaactagcgtttgaaattatctatggggttaaacctttaaagattaacacgctaattagaaaaccagtgaccattagaaatggcactaaataccctgggtttagtaagctagcaaaactagcagtaatcaccttacctaagggaatggatattgccattactggtgagaaaacaaccaagaaagaaacaaaggatcaataatggcaattaaaaagattattagtcgttctaacagtgggattcacaacgccactgtcattgactttaaaaaactccttaccaattccaaacccgaaaagtcgcttttagttactttaaaaaaacatgcaggaagaaacaaccagggcaagatcactgttcgccaccacggtgggagacataaacgtaagtaccgtttaattgattttaagcgttaccactatgacaatttaaaagcaactgttaaatcgattgaatatgatcctaaccgcagttgttttatctcccttttacactatcagaatggggttaaaacttacatcattagtcctgatgggattaaggttggtgatcaagtttattcatctgatcatgccattgatatcaaactaggttattgtatgccccttgcttttatccctgaaggaacccaagttcataacattgaacttaaccctaagggtgggggtaagatagcaagaagtgctggaagttatgcgaggatcttgggtcaagatgagactggtaaatacatcattctccagttaatctcaggggaaactaggaagtttttaaaggagtgtagagctacagttggtgttgtctctaacttagatcataaccttgttgtaattggtaaagcagggagaagtcgtcataagggaatcagaccaacggttagaggttcagcaatgaaccctaatgaccacccgcatgggggtggggaagggagaagcccagttggcagagatgcaccaagaaccccttggggcaaacgccatatgggtgtgaaaacacgtaacatgaaaaaacattcaactaacctgattattagaaacagaaaaggagaacaatactaatgtcaagaagtagtaaaaagggcgcatttgttgatgctcacctcttaaaaaaagtgattgaaatgaacaaacaagccaagaaaaaaccaattaagacttggtcaagaagaagtactatcttccctgagtttgtgggtaacaccttcagtgtgcataacggtaaaacctttattaatgtttatgttactgatgatatggtaggtcataagttgggtgagttttccccaactagaaactttaaacaacacactgctaaccgttagttatgattgcttttgctaaacaatacagagttcacatctccccccaaaaagcacggttagtgtgccagttaattgtgggtaagaagattaatgatgcgcaaaacatccttttaaatacgccaaagaaagctgcttactttttaactaagttactaaatagtgcgattagtaatgccactaataaccacgggatgagcggggatcttttgtatgtatttgaatgtgttgctaaccaaggacctagcatgaaaagaacaatcgctagagccaaaggttcagggagtgttttaaccaagcgttcttcaaacctagttattaagttatctgataatcccaatgaaagaaaattactcttaacccaacaaaaggaactggtgaaaaaaagaacaatgggtcataaaaaagagaaagcaaagcaaaagcaaaaacaacaataactatgggacaaaaagtaaattcaaacggcttaaggtttggcattaataagaactggatctcacggtgaactgccagttccaaccaacaaacagcaacctgattagtacaagatgagaagatccgtaacctcttttttatcaactatcgcaacgctcaggtgtctaatgttgagatagaaagaacccaaacgactgttgatgtttatgtctatgcagctcaacctgctttattgataggcagtgaaaacaaaaacatccaaaagattaccaaaatgatccaaatcattgtgggcagaaagattaaacttgatcttactatcaatgagatcggctctccgatgttatcaagtaggatcattgcccgtgatattgctaatgcgattgaaaacagagtaccactccgttcagcaatgcgccaagctctaaccaaggttttaaaagcaggtgctaatgggattaaggtattggtatcaggcagattaaatggggcggaaattgcccgtgacaagatgtatattgagggcaatatgcctctttcaactttaagagcagatattgactatgcctttgaaaaagcaaaaaccacctatggcattattggggtgaaagtatggattaacagggggatgatctatgcaaagggtttaaacagaaccccagcacacatcctccatccccaaaagaaacagctaaaaaccccaactatcaaaaaaaccaattcagtaatagcaaaacaaaaactcactggtagtgatattgaaactgctagtttaaaagcacttactgataataatcaaaaccacgaatagttaagatgttacaaccaaaaagaaccaaatacagaaaaccacataacgtcagttatgaaggacacactaagggcaatggttatgttgcttttggtgagtatggaattgttgctactaagggtaattggatcgatgcgagagcaattgaatcagcgcgggttgctatctcaaagtgcttgggtaaaactggaaagatgtgaatcaggatcttcccccacatgtcaaaaaccaaaaaacccttagaagtgaggatgggttcagggaaaggtaaccctgaattttgggttgctgttgttaaaaaggggacagtgatgtttgaagttgctaacatccctgaacaacagatgatcaaagccttaacaagagcaggccataaactccctgttacctgaaaactaatgaaaagagaggagaacagttaatgacaatcgctaaggagctgaagcaaaagagcaacgaagagttagtgaaactagtaattaagcttaagggtgaactcttagaataccgctttaaacttgcccatggtgaacttgacaaaccccatctgattgccaaggtgagaaagttattagcagttgtacttactattctcactgaacgcaaactcaactgacaagttgaaaaagataagtacaagttactttcaagaaaaaccaatgaacttattgttaacagttgaaagcaaaaactatcaactaaacctgaatccaaacaagaaactaaaaaggctgaagttaaacctaaggttgaatcaaagcctgaatccaaacaagaaactaaaaaggctgaagttaaacctttaaaacaagaaactaaaaaagttgaagttaaacctaaagttgaaccaaaacctttaaaacaagaaactaaaaaggttgaagctaggattgaaactaagactaaagttgaatcaaaacctttaaaacaagaagttaaaaaggttgaagctaaaaaatctgtttcaaaaccccaaaaaccagttaaagccaaaatgattaaaacaaaggagaaaaaacaataatgaagcgcaaccaacgtaagcagttaattggcacagttgttagcaccaaaaatgctaaaacagcaactgtcaaagtaacatcacgctttaaacatcctttgtatcacaaatcagttattcgccataaaaagtaccatgtccataactttggtgaacttgttgctaatgatggtgatagggtacaaattattgaaacaagacccctttccgctttaaagcggtgaaggattgtcaaaatcattgaaagagcaaaatagtttatggttagttttatgacaagattaaatgtagctgataatacaggcgctaagcaagtaggtattatcaaagttttaggtgctacatacaaacgttatgcattccttggtgatgttgttgttgtatcagttaaagatgcaatccctaatggcatggttaaaaagggtcaagtgttaagagcagtcattgttagaaccaaaaagggacaacaacgccaagatggtacccacctaaagttccatgacaatgcttgtgtgcttatcaaagaagataaatccccaaggggaacaagaatctttggaccagttgctagagagttgagagaaaaaggttacaacaagattttaagcttggcggtggaggttgtttaatgcaaaggattagaaaaggtgataaggtagttgtgatcactggtaaaaacaagggtggtagtgggatagtgcttaaggtattaaccaagcaaaacaaagcgattgttgaggggatcaataaggttactgttcacaaaaaagaacaagtcaacaagcgcagcaaacaaacaaacccaactactaaagaagcccctttaccattaaataaacttgctttatttgatcagaaggccaaacagcaaacaattggcaagatcaaataccaaattgatcctaaaaccaaacaaaaaacaagagtctttaagaagactaataatgccatttaactgttatgaataaccttgaaaaaacctataaaactgagttagttaatcaactccaacaacagttgggcttttcttccattatgcaagtccctaagttaacaaaaatcgttgttaacatgggagttggggatgcaattagagacaacaagttccttgaatcagcactaaatgaactgcacctgattactggtcaaaaacccgttgctactaaagctaagaatgctatctcaacttacaagttacgtgctggccaattaattggttgtaaagttactctaagaaataaaaagatgtgatcctttctggaaaaattaatctatattgctctgcccagagtaagggactttcgcggtttatcactgcgctcttttgatgggaaaggtaactatacgattggcattaaagaacagattatcttccctgaaattgtctatgatgatatcaaaagaattaggggttttgacatcactattgtcacttccaccaacaaagatagtgaagcacttgctttactgagagcactaaagatgccgtttgtaaaagaatagatatggctaaaaaatcattaaaagtaaaacaatcccgtcccaataagtttagtgtacgcgactacaccaggtgtttaaggtgtgggcgtgctagagcagtgttaagccactttggtgtgtgtaggttgtgtttccgtgaacttgcttatgcaggagcaatcccaggagttaaaaaagcatcatgataatcaataaagttcccaaagcccattttgatccagtttctgatcttttcactaagatcaacaatgctagaaaagctaagcttttaactgttaccaccatcgcttctaagttaaagatagctatcttagagattttgattaaagagggctatttagctaactatcaggtgttggaaaataaaactaaaaccaaaaaactagttagtttcacattaaaatacacccaaagaaggatatgttctattaatggggtgaaacagatctcaaaaccaggattaagaatctatcgttcctttgaaaaacttccccttgttttaaatggtcttggtattgcaattatctccactagtgatggagtgatgactgataaagtagcaaggttaaagaagattggtggggagattttagcttacgtttggtaaaaaattatgtcaaaaataggaaatagatcaatcaaaattgatcctagtaaagtgagtttaatgcaaacaacaacactgcttactattaaaggaccattaggggaaaacaccattaaactacccaaaaacttacccttaaagtttgttgttgaaaatgacactattaaagtaactaataacaacaacttaaaacaaactaagatcttacacggtactttcaatgcgttagttaacaacgcagttattggggttaccaagggttttgaaaagaaactcatcctagttggggttggttatcgtgctaatgtggaagggcaatttctcaacttacaattgggctattcccatcctattaaggagttgatcccaaaccaacttactgttaaagtagagaagaacactgaaatcaccattagtggaataaaaaaagagttagtaggtcagtttgccactgaaatcagaaagtgaagaaaacctgagccttataagggtaaaggggtactttactttaacgaagtaattgttagaaaacaaggtaaaactgcagagggcaagaaataagatgacaagaaacgataaaagaaggattagacacaaacggattgtcaaaaagattaggttaactaaccttaacaacagggttgtactaattgttatcaagagtttaaaaaacatctcggttcaagcttgggactttagtaagaacgttgttttaacatcaagttcctcacttcaactaaaattaaaaaatggcaacaaggagaatgctaaactagtgggaatggatattgcaaccaaactcatcaaactaaaccaaaaggatgtggtttttgatactgggggtagtaagtaccatggtaggattgctgctttagcagaaggagcgcgagctaagggtttaaatttttaaagctatgaatgatcaaaaaactactaacactggcttgttaacttccactcttaaaaccaagcccaaacacaaccttaaaccttccagtgaagccattaaaaaagcagtgtccaaaaaggaaggtcattacaaaaacaagcgctttcaaaaacataactttaataacaaaagtgagtttgaagagaggattgtcaaactcaaacggatctccaaaaccacaaaaggtgggagaaacatgcgctttagtgtccttgttgttgttggtaacaaaaagggcaaggttggttatgggattgctaaggcattggaagtaccacttgccattaaaaaagcgattaaaaaagcccataactccattcatacagtagagatccataagggttcaatctaccacgaagtgattggtagaaaaggtgcatctaaggtgttgttaaaacctgcacctttaggaactgggatcattgctgggggagcgatccgtgcaattgtagagttagctggttttagtgatatctataccaagaacttgggaagaaacacccccattaacatgatccatgccactatggatgggatcttaaagcaactctcacccaaaaaagtggcattattaagaaataaaccaattagtgatctataaaaacaatggaactacaccaattaaaaagtgtctctaaaagccgtaaccacaagtccaaagtggtaggtaggggccatggctcgggattaggtaaaacatcatcacgtggtcaaaagggacaaaaagcaagaaaatcaggtttaactaggttaggttttgaagggggacaaacacccctttaccgccggttgcctaagtatggggttgctaacaaagggatcttaaaaaaaaggtgggttgttttaaatttgaacaaagttgctaaactcaatctcaaaacagttactagagcaactttgattgaaaaaaaggtaattagtaaaaaaaataacctccctttgaagttaattgggaacacaaaactcactactcccatccactttgaagtgcaaaaaatctccaaaaatgctttaaatgcagtgcaaactagcaaaggtagtgtgaaaattatcacctaaaaactaggtaggataacccaaagaaaataattaaaatattatgaaggttagagcaagcgtaaaaccaatttgtaaagattgtaagatcatcaaacgtcaccgcatcttaagggtgatctgcaaaaccaaaaaacacaagcaaaggcaaggataatggcacgaatcttagggattgatatccccaaccaaaaacggatcgagatagctttaacatacatctttgggattggtttgtcaagtgcaaaaacaatcttaaaaaaagcaaagattaaccctgataaacgcgttaaagatctgagtgaagaggaacttgttgcgattagaaacgcagcaagcggttacaagattgagggtgatttgagaagagagattgctttaaacatcaaacacctaacagagatcggttcttgaaaagggattagacacagaaaaaacctgccagtaagaggacaacgcactagaaccaacgcaagaaccagaaaaggccctagaaaaacagtggctaacaagaaaattgaaagtaagtaatggctaagaaaaaaaagattaatgttcccagtggtttgatccatgtctcctgttcacctaacaataccatagtatcagccactgatcccagtggtaatgtcttgtgctgagcgagcagtggtacagtaggattcaaaggttttagaaagaaaaccccttactcagcaggggtagcagctgataaggtggctaaaactgtgaaagagatgggaatggggagtgttaagatgtatctgaagggaacaggtagaggaaaagacaccacgattagaagctttgctaatgctgggattacgatcacagaaatcaatgaaaaaacccctattccccacaatggctgcaagctcctaagcgtccgcgctaatcaaaacaacaacttatggaaaaatttttaaagagttaggtttaaaattccgttcttaataaaatagagctatgtcatacattaataaagaggggaaaaccacagcttgaagagtgatgacagtgcgtcagcaagtgagtgcagtgttaagttatggaaagattcaaaccactttaaaaaaagctaagaacacccaaaaaaggttagagaagattattaccattgctaaagttgataactttaacaaccgcagggctgttaaaaagtggttattaaataccaattcattagatgtagatcaactcacaaaccacctttttaaaaaagtagcaccacgttttttaaagcgtaatggtggttatagtagagtgttaaagttgggagttagaaggggtgatagtactgaaatggcgatcttacagctgatagatgctaccaactaacgatgtacgctgctctattagtacctttaaaaagatagttatgaaaaccaaaagtgctgcagtaaaacgctttaaactcaccaaatcaggacaaattaagcgcaaacacgcttatacttcccacctcgcgccccacaaatcaaccaaacaaaagcgccatttgcgcaagcaagctactgtgagcaacagtgaattgaaaagaattggtattttaatttagttatgcgtgttaagggaacaaatacaaccaggattagaagaaaaaaatggttaaaacaagctagtggtagctttgggacaagaaaagcttcttttaaggcagctaaacaaactgttatccaagcaagcaagtatgcttaccgtgataggagacagaaaaaacgtgagtttcgttcgttgtggatcttaaggttaaatgctgcactgcgtgcacaagggatgacttattcagtgtttatcaatgaattgaaaaaagccaagatagtcattaacagaaaggtactttctgaactagcaattaaagaacctaataagttaaatctgattatcaataccatcaaaaaaccaactaataaaccaactgttgcaaaaacttagatgaccttttgcaactagaaagttttgggagatattaatcttaatcatggataaagcgtaccacttttagtttggtgtagggttgacggtggccataacgcttgaggtggtgtttttgggagatgtgtttaattagtttaattttcgattttaaaccgtgtttttcaatcacacaaacaaccttagctttttcaaggtaaggtttgcctatcttttcatcaagcatcaataccttatcaagttggatctcctgaccaactttaccagctaatttttcaacaaaaatagactcgttttcatggactaaatactgcttagcaccacaaaccacaatagcatgcatttaggcatttagttggtgtttgatgatgttaacccgggttttgctttgtttactaccaaacttttgaaagcggacaatgccatcgctcaaagcaaagagggtgttatcactacccattgcaacattttgtcctgcaaagatcttagttcccctttgtctatagataatctgaccaactctaatcatctgaccatctgccttcttagcgcccaagcgcttagaatgtgaatcacgtccattcttagtggaaccaaccccttttttggaagcgaaaaactgtaagttaatttggtagcagtaactgtttttactcattgttgttaattttaaataagtttaagataagctggagataatgcttataatcttcaaggacttaaatttagtgaaaacaaacaattgccaaattattggctgatgaaaacaactttccttaccacaaaagacattcttaagtttcattcccttatttttggtcactagtgcttttgttttaactggaattgttgaaagtcttttaacatttggaactattattgaacaaattgataaattcactgatcagactaatgtgatgttattaatttatgcagttatctacacttttaatccaaaaagttgattgttaaaaaaccaacaattctttttaagtgcattagcttatatattatttacttttattggctataacctaattttgtcaatagctggtatagcttataaatcaacaaatccatataagttaacaagtagtatttttctccatgtaattgcaccaatagcattcttcatagcaagttttatcaaaataaaacatgagaaagatgtcaatattaacatgttctttaaaagcctattattattcatgatctatcctttaatatatgggctttatttagtaactattccatatgtaaggcattatctttttaatggtaggccatctacttataccatttatggcagcattacaaatactaaaaataatccttttgcttgattagttgtatttgcagttttatttatctatttccccttgagttacttagctatatatctattacaacttaagttaataaaaaaagccatacaaccgcaatttaatttgccttttacattaaataaatgaaaacaaaaataatgaatatattaatatttattggtctttgtcaaaaaataagatgaaaaaggcaatccactttcagagtcaaccagttgtttttaactgtgcttcatgcaatagcaactttaccattgactccactgccaaacaaaaggatcttgccattgacatttgtggaaaatgtcatcctttttacatagggcaattaaccaaacaaaccgtgcatggacgggctgaaaaactttctcaaaagttcaacgctggaaaggcttttttagaaaataaaactaaaaagagtaaccaagctaaagttgaaaaacaaactaggcaccgttctattaacgagctttagtttagccttgattattaagataatttaaaatcgaaaacacatgaaatatactggtagtattttcaaacgatcaagacgtttaggtttttctttacttgaaaacaacaaagagttttccaaaggaaaaaaacgtaaatctattcccggtcaacatggaaataggtttcgttcctcaactttatcaggttatgcccaacaactccaagaaaaacaaaggatgcaatacatgtatggtattactgataaacaatttcgtaggttatttcgctttgttttaaaacaaaaaggtaacttaacagttaatttatttagagttttagaatcacgtttggataacatagtttacagaatgggttttgcaccaacaagaaagagtgcaagacaaatggtaaaccacggtcatgtgattttaaatgatcaaactgttgacaccccttcaatcatcattaatcccggggataaagtccgtttaaaagcaagaataactaaatccccattagttaaaaattttattgaaaacagtgttatctcatcatttgtggaaaccaacaaaaaggcatttgaaggtacttatataagattcccagagcgtagcgaactacctgctggaataaatgaatcttacgttgttgagtgatacaagcgtttagttaaataatgtcgtaaggtagtattgcacaaagaggtaaaacgtaaatattatttacgttttacctctttgtgcaatactaccttacgacaacgtgaacaaaacttattaagtgctagtttttctggatttttcttgacgtttttaaaggttaaataattaatctcagaacattcattacaacctagtcgtgtgcttcttttaacagccatggatttatcatcgtttgtgctattgcgaatattgtagttaatggtagatagtaagaaaaataaaaaacagcaggttacggatttttctaatttactctctcaaagtaaaggatttgttatttttgactattcaggaatgtctgctgttgatgcaactttaatgagaaaaaagttgtttaataagggtagtaagataaaaattgttaaaaacaatatcttaagacgtgctttaaaaactagtaattttgaaggtgttgatgaatcggtcatcaaaggaaaaattgcagttgctgttggtattaacgagatcttagaaaccttaaaagttgttgatagtgtagttaaagaaaaagagttaatgaaatttgtttgtggtcattttgataaccgtatttttaatagtgatgacttacaaaaaatagcaaaactccctggtagaaatgaactttatggaatgtttctttcagttctacaagcaccattacgaaaatttctctatgctcttcaggcagtaaggaatgctaagtaaattaaataaatagaaaaatattatgggaaaactagataaaaaacaattaattgaatctctaaaagagatgactatagttgaaattgatgaaataatcaaggctgttgaagaagcatttggtgtaactgcaactccaatagtagctgctggcgcagctggtgctacacaagaagctgctagcgaagttagtgtaaaggtaacaggatatgctgataatgctaagttagctgttttaaaactttatcgtgaaattactggagttggtttaatggaagctaaaactgcagttgaaaaattaccttgcgttgtaaaacaagatattaaaccagaagaagcagaagaacttaaaaagcgttttgttgaagttggtgcaactgttgaagttaaataaagatggcagtacaacaacggcgttctagtaaacaccgtcgtgataaaagacgttctcacgatgcacttactctacaaactttaagtgtttgtaagaaatgtggtaagaagaagttatcacatcgtgtgtgctcttgtggtatgtacggtgaactaagagttaaaaaagctcactaatcaagataataatatactctaaaactaattaataacctaaatggctaatattaaatctaacgaaaaacgattacgtcaagacattaagagaaatttaaataataaaggacaaaaaactaaactaaaaactaatgttaaaaaatttaataaagagattaatttagataatctcagttctgtttattctcaagcagatcgtttagccagaaaagggattatttctttaaacagagctaagcgtttaaaatcaaaaaatgctgttattttgcataaaagtaatacaaattcaactgcaaaaaaacaataataataatattaaaatagaatgcggttttgtatctaaatatatgcaaaaaacatcgatgcttacaaaggaagaagccattaaaaacaggaagtggtatcttgttgacgctagtggtttggttttaggcaaattagcagttaaagctgcaaatttaattagaggaaaaaataaagctaattttactcctaatcaagattgtggagatcatctaataattattaacagtgatcaagtggttttaactggaaataaaaaagacaatgagttttgatatcatcactctcaatacatgggtggaattaaaaaaactagtggaagggatatgataaacaaaaattcagataaacttgttttcaatgctgttaagggaatgttacctgataatcgtttaagcagaagattaataactaaagtacatgtttttaagaatgataagcacaacatggaagcacaaaaaccaacattacttaattgaagttaaaagatatggataaaaaatctttttatggacttggtcgtcgtaaatcttctactgctaaagtttatttatatcaaagcaaagataagggtaaaataaccattaatcatcgtaatcctagcgattattttccaaataaattggtgattcaagatatggaacaacccttagagttaaccaaacttaaggataactttgatatcaatgttgttgttaaaggtggaggatttactgggcaggcaggagccattagattaggtattgttcgtgctttaataaaatttaatccagatcttaaaaagttattaaaaaccaaaaaattaacaacacgtgataagcgtgctaaggaacgcaaaaaatttggtttatatggtgctagacgtgcaccacaatttactaagcgttaggttttctaattttgttataattcaacgtgtttattaattaatgaaaattgataaagaacaaatcattaaggctcatcaacttcacaaaaacgatgttggcagtgtgcaagtacaaatctctatattaacagatcagattaaaaaattaacagaccacctgttagcaaacaaaaaggattttatttctaagcgtggtttatatacaaaggtatcaaaaagaaaacggctacttaaatatctaaaagaacgtaatattgaaacctaccgtgatttgattaaaaatttaaacctccggggttaatccatatatgcttatggattaagtttaacttaaatttaaattagaaacgattttgttttttaagggtacgaattgttttagttgaaactagaatccgtgttactttaccattagtatctttaattttgcatgactgaaggtttacattccattttcttcttgtaatagttttggaatgagaacgattattgccatacaaaggccctcttaaggtaagttggtctttttttgccattacagacctgatttatacaaaaaatacttagaaaataaaaatgaaaaaaataaataaacaagcattaattgatgcagtagaacaaaaacagttaaaggaatatgttcctgaatttggagcaggagatgaagttaatgttgctattaagttacgtgaaaaagaaaaagttcgagttcaaaacttcactggaactgttttaagaagaaggggaagagggattagcgaaactttcatggtaagaaaaaccactgatggaattcctattgaaaaaaactttcaaatccacaaccctaatatagacatagaagtaaaacgcaggggtaaagtaagaagagcatacatctcttatatgcgtgaaagatctggtaagtcagcaaaaattaaagaaaaaaagtcttaaatgcggatgggaagagtacactatccgctttatagaatagtagcggttgattcgcgagtaaagcgtaatggaaagtatatcgctttaattggacatctaaatccagctttaaaggaaaataagtgtaaattagatgaaactgttgccttagattgacttaataaaggggcaattccaactgatacagtccgttctttatttagtgaatctggtttgtggaagaaatttattgaaagtaaaaataagaaagaaacaagtcctaaaaagtaggataaagaaagtgcaataaacttaattaagcaatttattaatgaaacgaacataccaaccaagcaaattaaagcgtgctaaaacccatggttttatggctaggatggcaactgcacaaggacgtaaagttttaaggcaaagacgttttaaaaatcgtgctcaactcacggtttccagtgagcgttaa

SEQ ID NO: 7 - DNA, 10809 bp, M. genitalium
tcgttttcaaaatcaatttaaaaaaggagcaaaaccttagatgcttaaagtgaatgctgattttttaactaaagatcaagttatctatgatttagtgatagtaggtgctggccctgctgggattgctagtgccatttatggtaaacgtgctaacttaaatttagcaattattgaaggaaacactccaggagggaagatagtaaaaactaacattgtggaaaactatcctggttttaaaaccataactggtcctgaattaggtcttgagatgtacaaccacttgttagcatttgaaccagttgttttttataacaacttaatcaaaattgatcatcttaacgatacattcatcttgtatttagataacaaaacgacagtttttagcaaaactgttatctatgcaacagggatggaagagagaaaacttggcattgaaaaggaagattatttttatggtaaagggattagttattgtgctatttgtgatgcggctctttacaaaggtaaaacagttggtgttgtaggaggaggtaattctgcaatacaggaagcaatttatctttcaagtattgctaaaacagttcaccttattcacagacgtgaagtgtttagaagtgatgcattactagttgaaaaattaaaaaaaattagtaatgtagtttttcatttaaatgctactgtaaaacagttaataggtcaagaaaagctccaaactgttaaattggcaagcacagttgataaatcagaaagtgaaattgcaattgattgtctctttccttacataggctttgaaagtaataacaagccagttttagatcttaagcttaatttagatcaaaatggttttattttaggagatgaaaatatgcaaactaacattaagggtttttatgttgctggggattgtagaagtaaatcattccggcaaattgccactgcaattagtgatggggtaacagctgttttaaaggttagggatgacatttagtagattttattagaattgtttcaactaataaattggccttatggtaacagaaattagaagtcttaaacaacttgaagagatcttttcagctaagaaaaatgttattgttgacttttgagcagcatgatgtggtccttgtaaactaaccagccctgagtttcaaaaagcagcagatgaatttagtgatgctcagtttgttaaggttaatgttgatgatcatactgatatagcagcagcttataacattacctctttaccaactattgttgtttttgaaaacggggttgaaaaaaagagagccattggctttatgccaaaaaccaaaattattgatcttttcaataactaactctttgaaaaactaacagcttgaagtaaaattaatcctaatgaaatcactctttattggttattttgatggattacatcaaggtcatctatttttaaagcagaacagtaagtttgaaccaatggtgttattaattgataacccacctttaaaacaaaccaactggctttatgatttacaacaacgggttgcacaaataaaaacttacttgaaagcaactgtagaagtatttgatgttgccaaacataacatgaatgcacttagtttttttgaacaacagattaaaagattgaattgtgatgaaattattgttggtacagattggcattttggtaatgatcataaggatgggatctggttaaagaaactgtttaaaaatactgttattgttaataaaacaaacctatcaagtagtgttatccgtaactatctaactaataatgaacttgaaaaagctaaccaacttttagtggaaccttattatagagtgggcacagtagtacatggtttaaaaaaggcaaggttgcttggttttccaactgctaacattgttatggataaccacttattgactttaaataaggggagttatatagtaagagttttattaaataaccaaactttttatgggattggttttattagccaaaaggatcaggatttggtgtgtgaaacccatatctttaactttaataatgagatttatggttcactggtcaaatttacactgttaaagttcattagaacaattagtaagttttccagtcaagcagctttgcaaaaagcaattcaaagtgatgctaactttgctttaaagtggttggaaaaccaaaatttagataaaatttaatatccttaaatagcttaaaaaattactgcagctaagatatatgaaaaaagtgattgtgattggaataaatcacgctggtactagttttattagaactttactttcaaaaagtaaggactttaaggttaacgcttatgatagaaacacaaacatctcgtttctggggtgtggaattgcacttgctgttagtggtgttgttaaaaacactgatgatcttttctattccaaccctgaggagttgaaacagatgggcgctaacatctttatgagtcatgatgttactaacattgatctaatcaaaaaacaggtaacagttagagatttaacatcaaataaagagttcactgatcagtttgatcaactagtaatcgcttcaggagcatgacctatatgtatgaatgttgaaaacaaggtgacacacaagcctttggagtttaactacactgacaaatattgtggtaatgttaagaacttaattagctgtaagttataccaacatgcacttaccttaatcgatagttttcgtaaagataaaaccattaaatcagttgctattgttggttctggttacattggcttggaacttgctgaagcagcttggttatgcaaaaagcaagtaacagtaattgacttacttgataagcctgctggtaataactttgatcatgagtttactgatgaacttgaaaaagtgatgcaaaaagatgggttaaaactaatgatgggttgcagtgttaagggctttgttgttgatagtacaaacaacgttgtcaaaggagttgaaactgataagggaatagtaaatgcagaccttgtgaaccaatcaattgggtttagacctagcactaagtttgttcctaaagatcaaaactttgagtttattcacaacggttcaattaaagttaatgaatttctccaagcactaaatcataaggatgtttatgtcattgggggttgtgctgctatttacaatgctgctagtgaacagtatgaaaacatcgatcttgctaccaatgcagtaaagagtggattagtagctgcgatgcatatcattggtagtaaccaagttaaactccaatctatcgttggcaccaatgcactccatatctttggtttaaatttagcagcatgtggattaactgaacagcgtgctaagaagttaggttttgatgttggcatatcagttgttgatgacaatgatcgtcctgagtttatgggcagttatgacaaggtgcgttttaaacttgtatatgacaagaaaaccctaagaattttaggagcacaactcctttcttggaataccaatcacagtgagattattttctatattgcacttgcaatccaaaagcagatgttactaactgaactgggtttagtggatgtttattttcttccacattacaacaaaccgtttaactttgtgttagcaactgttttacaagcacttggttttagctattacattcctaaaaaatagtatttttttatcaatttaatatctaaatcgaaaagaaaacatgtcgccacgggagatagttttaaaagaaactaatcaaatagatttcatttccaatcaaagtatttttgatatctcaccaattagcggttgaaaaccatttgcccctactgatcaaattcttggtatttttattgtttttgtactgcttctaactttttttattttttataagcttaagttaaaaaaagcagattctttaaaaaataattcatattttttgcttttatttcaaatgttgtttgtttgggtacaagatacaacagcagatcttttaggagaggaaaataagaaatttgctccctactttttaatgttgcttctgtacatagtatcaagcaacttagttagcttgcttggtggtatttcaccaccaacatcatctttaacatttactttttctttaggacttgcaacttttattgggattgttgttatggggattagataccaaagatgaaatttttttaaagagtttgcctttggaattactgttaaaggaaaaaagtattctactttcattccaaatccttttagtatattgagtggatttgcaccgcttttttctatttcattaaggttatggggaaacatattagcgggcacagttattttggcgcttttttataacttttgaatttttattttttcaagtattaataaccaaccattagcacttagcttaggaacagtttttgcaggtttaataaccccagtattacacatctattttgatgtaattgcaggtgtattgcagggttatgtttttgtaatgttgacttataattattgggctaaaatgcgcaatcaaggtttggaaaataataatgcaagtgaattacactttaaaggcataaaggtaattcaagaaaatatttagttatggaacatgttaatgaaattttagctacagttggtgttatattacaacaaactcaaactacccaggatgttaacgctagtgctaagctaggtgcttatataggtgctggtgttactatgattgcaggttcaactgtagggattggacaaggttatatttttggtaaagctgttgaggcaatagcaagaaatcctgaagttgaaaaacaggtttttaaactaattttcattggttctgctgtttctgaatctacagcaatttatggacttttaatttcctttatcttaatttttgtagcaggagcttaaggatggtaaaggcaaaaaaacttgtctttaaatgaagcttattagtttttagcttttttacactcagcttatttttggtttcttgtactgagaatgttagagaaattaagagtagttcagtaataaatgaactttttcctaacttttgggtatttattactcatttactagcatttttcatcttactaacactgatgattttcttgttttgaaaaccaactcaaaggtttttaaataaccgtaaaaatttactagaagcacaaatcaaacaagctaatgaattagaaaaacaagcaagaaatctacttgaagaatctaatcaaaggcatgaaaaagcactaatagtttctaaagaaattgttgatcaagctaactatgaagctttgcaattaaaaagtgaaatagaaaaaacagcaaatcgccaagctaacttaatgatttttcaagctcgtcaggaaattgaaaaagaaagacgttctcttaaagaacaatctattaaagagagtgtggaattggctatgttggctgcacaagaactaattctcaagaaaatagatcaaaaatcagatagagaatttattgataagtttattagagatttagaagctaacgaaacagaagatgattaatgcacaagcatttggaactgcactttttcaattaagtgaagagcaaaaacaagtaaagaaaatttatgaagagtgccatttttttctgaaattaatgcgtaattttaaagatggttcattatcgttcttacttaattcttatacactaacaaaaccagataaaataagacttgttgataagttgtttaaaaatcatttttgtcaagtttttgttgattttttaaaagtaattattttaaagggttactttactttagttgaacaggcaattaagtatttttttgataatgttgaaagtcaaaaacacattcaatttatcaaaataattactgcttttgaattaagctcaaaacaacttaacaaaattattgcaataatggaaaaacgttttaaaacaaaggttgtttataaaactgagattgatcgcagtttaatttcaggaattaggatagaatcaagttcccatttatttgaaaaaaatgtgcgtgatgaattaaaacgcataatggcccattttatttaagttaattgagaagttatggcagataaactaaatgaatacgtagcattaatcaaaactgaaattaaaaagtattccaaaaaaatatttaacagtgaaattggtcaagtcattagtgttgctgatggaattgccaaggttagtggacttgaaaatgctttattaaatgagttaattcaatttgaaaataatattcaaggaatagtattaaaccttgaacaaaatacagtcggaatagcactttttggtgactattcttcgttacgagaaggcagtaccgctaaaagaacccacagtgtaatgaaaactcctgttggtgatgttatgcttggtagaatcgtcaatgcacttggtgaagcaattgatggtagaggtgatattaaagctactgaatatgatcaaatagaaaaaattgctccaggtgtaatgaaaaggaaaagtgttaaccaaccacttgaaactggaatcttaacaattgatgctttatttcctataggtaaaggacaacgtgaattaattgttggtgatagacaaacaggtaaaactgctattgcgattgacactatcattaatcaaaaagataaagatgtttattgtgtttatgtagcaattggtcaaaaaaattcatcagtagcacaaattgtacaccaacttgaagttaatgattcaatgaaatacactacagtggtttgtgctacagctagtgactctgattccatggtttatttaagtccttttacaggaataactattgctgaatattgacttaaaaaaggaaaggatgttttgattgtatttgatgacctttctaagcatgctgttgcttacagaactctttcactcttgttaaaaagaccacctggtagagaagcttttccaggagatgttttttatttacattcaagacttttggaacgtgcatgcaagttaaatgatgaaaatggtggtggctcaattacagctttaccaattatagaaactcaagctggtgatatctctgcatatattcctacaaatgttatttcaattactgatggccaactgtttatggttagtagtctatttaacgctggacaacgccctgcaattcaaattggtttatcagtttcaagggttggtagtgcagcacaaacaaaagcgattaaacagcaaactggcagtttaaaactagaacttgctcagtatagtgaacttgatagttttagtcaatttggtagtgatcttgatgaaaatacaaaaaaggttttagagcatggtaaaagagtaatggaaatgattaaacaaccaaatggtaaaccttactctcaagtccatgaagcattatttttatttgctattaacaaagctttcattaagtttattccagttgatgaaattgctaaatttaaacaaaggataacagaagaatttaatggttcccatcctctgtttaaagagttatctaacaaaaaagaatttactgaggatttagaaagtaaaactaaaaccgcttttaaaatgcttgtgaaacgttttatcagtacattaacagattatgatattaccaaatttggtagtattgaggaacttaattaatggcttttatacaagaaattaagcgcagaatgaatacagtaaaatccaccattaagataactaatgcaatgaaaatggtgtcacgcgctaagtttattaagttcaaaaaacagtttcaagaaattagtttgttttttaatgaattttataaagctgttggccaagtagttgtttctttaaaagaaccaaaaaagaaaccagataaccaaaaaactttatggataatgatgagttcttctttaggactttgtggacagcataattcgaacatgaataagttattaaaagctaattttaaagctgatgataaaatcttttttttaggtagaaaaaaccaatcattttgaaataaaaatagtcaatataatcctgctgttggatttattgatatccaagatcgtgatattaattttgattattgtcaaacgatatttgatcagattatggatgcatttaaagagtttaaacttgatcgaatttgtatggtttacactaaatttaaaaactcattaatccaacaatctcagctctttcaagtttttcctttcgatgttgaaacttttaaaactttaaatccggttgtaactgatcaacaacttgattttgagccagatcaagccacgataattaatttaattactccacagttttttgatgtggctctgtatggtggccttgttgaaactaagttatgtgaatcagcttctagacaaaatgcaatggaagctgctacaaagaatgctaaagatttacttgataaatacactttacaatttaacaagctaagacaaaactctattacagaagagattattgaagttataggaggtatgaattaaattgataaaaaaagaaaacctaacatatggtaaagttcaccaagtcattggtcctgtagttgatgttatcttttcagaaagtaaacaattacctagagtttatgattgtttgagtgtacaactaaaaaaaagtgagctttttttagaagcaacccaattaataggtgatgacattgttcgttgcattgcattaggtcctacagaaggattagcacgtaatgttaaagttactaactataaccatccaatagaggtacctgttggcaaaaatgtattgggaaggatgttcaatgttttaggtgaacccattgatggaaaagaaccattaccaaaaaaaccaaagctatcaatccatcgtaacccacctgcttttgatgaacaaccaaatactgttgatatttttgaaacaggaataaaagtaattgatcttttaactccttacgttagggggggtaaaattggtttatttggaggagctggtgttgggaaaactgttttggtgcaagaattaattcataacattgccaaagaacattctggtttaagtgtatttgctggagttggtgaaagaacaagagaaggtaatgatctttactatgaaatgattcaaggtggggtgattgataaaacagttttagtttttggccaaatgaatgaaccaccaggagctagaatgagagttgctttaactgctttaacaatggcagaatattttcgtgatcatgataatcagaatgtgctgttattcattgacaatatttttcgttttactcaagcaggtagtgaggtttcagcattacttggtagaatgccatctgctgttggctatcaaccaactttagctattgaaatgggtaagttacaagaaagaattgcttctaccaaaacaggttctattacatctgttcaagctatctatgttccagcagatgatctaacagacccagcacctgcaacaacatttacccatcttgatgctaaaacagtgttggatcgtaatattgcagcactaggtatttttccagcaattaatcctttagaatcaacaagtcgtttattagatcctagtgttgttggtatcaaccattataaagtcgctttaggagtgcaaaatatcttgcagcgttttgcagaattacaagatatcattgctatactagggattgatgaattgtctgatgaagataagattattgttgaaagagcaagaaggatacgtaactttttatcccaacctttttttgttgctgaaaagttttcaggtattgcaggtaaatatgtatctttaaatgatactgttcaatcttttaaagaaattttggaaggtaagcatgatcatttgcctgaacaagcattcttttatgttggaaccattcaagaggctgttgaaaaagcaaaaagattaaatcaagagtttgataaaactaaatagttttatgaagttattgcgctttttggtacttagtcctagtggcataaaactagataaaaccattattagtgcgcaagttaaaactactgaaggttacataggattaaattttaatcgcgctcctttgattgctgctattcaatcccatctgtgcaaaattatttttgctgatcaaacaaaaagagaagcaattattggtgctggtttaatgcttattaaaaaaacagaagctaagattttcacagaaaattttgtttttgctgatgaagttgatattaatgaaaccttaaaaagaaaaacagaacttgaaagaaaaattcaccatatcaaggatgctaagctaaacgttaaaattgaacaaaatttaatgtttgaactattaaaactttcaagtaagaaaaaataaaattattatatgttttaagatttctattaattcaagtaatatgaaagaaatttattttggtggtggttgtttttgaggaatagaaaaatattttcaacttattaagggtgttaaaaaaacatctgttggttatctcaactctaggattagaaatcctagttatgagcaggtttgttctggttatactaatgctgttgaagctgtaaaagttgaatacgaagaaaaagaaatttctctttcagaattaattgaagcactttttgaagttattgatccaactataagaaatagacaaggtaatgatattggaacacaatatcgtactggtatttattgaactgatagcagtgatgaaaaaataattaatgataagttcttaaaacttcaaaaaaactacagtaaaccaattgttacagaaaataaaaaagtagaaaattattatcttgctgaagaataccatcaggattatttaaaaaagaatccaaacggttattgccacatcaaatttgactaa

SEQ ID NO: 8 - DNA, 21247 bp, M. genitalium
taattaatgattcattcagtgaggaaaatcaatagtagatatgcttgttaactttaaattgatgcttcaaaaagcaaagctaggtaaatatgcaatccctcacattaacatcaataactatgaatgggccaaagctgttttaacagcagcaaatcaagctaatagcccaattattgtttcagtatctgaaggtgctttaaagtacatgtctggttatagtgttgttatcccgcttgttaagggtttaattgaatcactaagtgttaaagtaccagtgacattacatttagatcatggtagttatgatgcatgtatccaagcattacaggctggatttagttcagtaatgtttgatggttcacatttaccatttgaagaaaatttcaataaatctaaaaagttaatagagatagcacaaaaaacaaatgcttctgttgaacttgaagttggtactattggtggagaagaagatggtgttataggacaaggtgagttagctaatgttgatgaatgtaaacaaatcgctagtttaaaaccagatgctttagcagcaggaattggtaatatccatggtatctatcctaagaattgaaaaggattaaactttcctttgattgaaacaatatcaaaaattactaacttacccttagttttacatggtggctctggaatcttagaaaatgatgttaaaaaagcaattagtttagggatttgcaaactaaatattaatactgagtgtcaattagcatttgcacatgaaattagaaaatacattgaatcaaataaagacttggatcttaacaaaaaaggttatgatcctagaaaacttttaaaagaacctactcaagcaattgttgatacttgcttggaaaagattgatttgtgtggttctagaaataaagcatagtttaatacctggtggtaaggggattaatgttgctattgtaatgaaatcacttggttttgatccaactgtcattacttttttgggacaacccactaaaaacttatttttagagttggtaaaaccttatgatctaaatatagttagcttcatttctgaaactaaaacaagaattaaccttaagttattaaaagatgaaaaaactactgaaattaatgatttaagtcctttaataacagatgctaatctaactgaattgttaacttttttaaaagctaatgttaagaataatgatttggttatcatcaacggaagatttaaatttgaagctttagaaaaagttctaaacttggtctttacattaacagaaaatgtggttatagatgttgatgaaagcaaaatgttaacgcttttaaatcagtctaaaccactagttatgaaacctaacattgatgagtttcaaactatgattaatactttttttcacgatcaacaaagcttaatagcagcaattaaaaaatttcattactgtaagctcttattattatctgatggtgacaaaggagcttatctttttgatcagaataagttattgtttgtaagttctatcactcctaaacaagtagttagcaccacaggagcaggtgatactttgttggcagtttttttagcaaatttgattctaaaggtagatttaaaaactgctttgattaaagcaactaactatgcaagtgcaacaattagtaagttaggtgttgttgatagtaaagacaaaattagtgttataaccccaaaaagttactatttataaaaatcctaaacaggttgaagagatccattgaatttagattatgaaatacttatatgccactcaacaccttactttaaatgctattaagcatgctaagggaggacatgttggcatggccattggtgcaagtcctatcttatttagtttatttactaaacactttcactttgatcctgaccaaccaaagtggatcaacagagatcgctttgttttaagtgctggccatggtagcatggcattatattcaattttccattttgccggacttatttctaaacaagagatcttacagcataaacatggtcaaattaacacttcttcccatcctgaatatgctccaaataacttcatagatgcatcaacaggccctttaggtcaaggctttggcatggcagttggcatggtgttagcacaaaagttattagctaatgaatttaaagagctaagtgataaattgtttgaccattacacctatgtggttgttggggatggagatctacaggagggggttagttatgaagttagtcaaattgctgggttatataaattaaataaactaattgtgcttcatgattcaaatagagtgcaaatggatagtgaagtaaaaaaagttgctaatgaaaatctaaaggttaggtttgaaaacgttggttggaattacatccatactgatgatcaactagaaaatattgatcaagctattattaaagccaaacaatcagataagccaacttttattgaagtgagaacaactattgctaaaaacacccaccttgaagatcagtatggaggacattggtttattcccaatgaagtggactttcaactttttgagaaaagaacaaatactaactttaacttttttaattatccagatagtatttaccactgattcaaacaaactgttattgaaagacaaaaacaaattaaagaagattacaacaatttgctaatttctcttaaagacaaaccactttttaaaaaatttactaattggattgacagtgattttcaagccctttatcttaaccaactagatgaaaagaaagtagcaaaaaaagatagtgctactagaaactatttaaaagattttttaaaccaaattaataatcctaattccaacttgtattgcttaaatgctgatgtatcacgttcttgttttatcaagataggtgatgataatctccatgaaaatccttgttctagaaatatccaaataggaattagggagtttgcaatggcaacaataatgaatggtatggcacttcatggtggtattaaagtgatgggtggtacttttttagcatttgctgattattcaaagccagcaattcgcttaggtgcattaatgaacttaccagtattttatgtttatacccatgactcttatcaagtagggggtgatggtcctactcatcaaccctatgatcaactaccaatgttaagagcaattgaaaatgtttgtgtatttcgtccttgtgatgaaaaggaaacttgtgctggatttaactatggtcttttaagtcaagatcagacaactgttttggttttaacacgtcaacccttaaaatccattgataacactgatagtttaaaaacactgaagggtggttatatccttttggatagaaaacaacctgatttaattattgctgctagtggtagtgaagtgcaacttgcaatagagtttgaaaaagttttaactaaacaaaatgtaaaggtaagaattctgtcagttcccaatataactttacttttaaaacaagatgaaaaatatctaaagagtttatttgatgctaacagttcacttatcaccatagaagctagtagtagctatgagtggttttgctttaagaagtatgttaaaaaccatgctcatttaggagcttttagttttggtgaatctgatgatggagataaagtttatcagcaaaaagggtttaatctggaaaggttaatgaaaatatttacttccctaagaaattaattcctaagctgtttggttaataaaatttagtagttttaaaatgcagattagtttagttaaaatccgcaataagtttaaacaaagaaaccgtggttcttttcgtcagtgagttggtaagctttccaacggtttgatgatccctattgcagttttgcctttagcaggtatttttttaggaatcggtgatgccatttcttccaattcatctggcattgttggtgtgaaattttttggtgaatttattaaacaaggtggtaatgtagtttttgctaacttacctattttgtttgcagttgcaattgcgatcaccttttctcaagatgcaggggttgctggattttctgcttttgttttttgggccacaatgaacgcgtttatgagttcattaattattcctgttgatgcaaataatactgcttcaggttataacatcctttattgaaaagcagtacctcagtcagcaattgcttctactttaggattaaattcactttcaacttcagtttttggtgggattatagtaggggctttaactgcatatttatataacaagttttatgcaattagattgcctgatgtaattgggttttttagtggtactaggtttgttcctattatttgtatgactattgctattccagtagcattacttttattgatggtttgacctggtgtttctatcttattaaatttaataggaactgggcttggaatcttaggtggaagaggatatggtgctaacagtttaatctttggatatatagaaagagcactaattccttttggagtacatcatgccttttatgcaccattatgatatacaagtgcaggcggtagtttgcaagaaattgcaaatcaacaagtttggattagagctcctggtagtgattatgtaaccagagtgataggttgagaagattttaatactccaggaaaatgagttattcctgctgctttagctaatggaacaagtggaatgatgaatggagctactacaacaggacaagatagtacatctgcactttcaaaatacatgagtaaagaatcaacaaactttctaagttgaaaagaacttgttgatggtcttacacgtaaaggtaactttgatgaattggctaaaaacggtttattagatggttctaacaagatttgaattggtttaaaccagtcagggatcttaggtaaaaaagtactgttaagtgatggtaaggactacactattacctttaaaacttttgctaacaccacgccaacattctgaagccatggtgctcatgcacttttaccaattagtggaactccaagtgcaataactaatggagttactgttaatggtactgctaattctaaaacctataatgtcagtcagttcactgttgcagttccttctttaaacccagcacaatattcccaaggtaaattcccattcatgctaattggaattccagcagctggacttgcaatgatcttagctgctcctaagggtagaagaaaagaagctagttctattattggtagtgctgcattcactagttttctaacagggatcaccgaaccttttgaatttacctttcttttcttagcaccatggttattctatggtatccacgctgtattagctgcagtaagcttttgattaatgaacttattgagtgctaacgttggacaaaccttctcaggttctttcattgactttatcttgtatggggctttacctgatggtaggggttgattagcaaactcttacttagtacctattattggtatctttttagcattgatttatttccctaccttctatttcttgacaattcgctttaacttagcaactcctggtagaggtggtaagttaattactaaaaaggaatatttagcagcaaaagcagctcaaaaaactgatcaaactactaacactaactttaatcaaacccaaattgaagctggtatgttactaagagcttatggtggaagtgaaaacattgctgaattaggggcttgcattactaaattaagagtaacagttaaaaaccctgaacttgttaatgaaactattattaaagacttgggagcagctggggtaatgcgtaccactccaacattctttgtagcagtgtttggtactcgagctgctgtttataaatcagcaatgcaagatattatccaaggcaaagtaaattgaacagagttgcaaaaagtcttagataaaaatgatagtactgttgaaaaaccagaaataaaaccaaccccagttttaaaagttcaagatgaaattgtgatcctctcaccagttaatggcaccttaaaaccgctcacccaagttcctgatgataccttcaaaaatcgtttggtaggagatggaattgctatcttacctagcgatgggcacttcaaagcaccaggtgatgtgggtgtgaaaactgaacttgctttccctactggtcatgcctttatctttgatgttgatggtgtgaaagtaatgcttcacattgggattgatacagtaaaaattaatgctgataaaaaaccaggggaacaacttgaagtgtttgatgtaaaaacaaaacaaggagaatacactaaattaaagagtgaaagtgttgttgaagttgatttaaagaaacttaaacgaaagtatgatccaatcactcctttcattgtgatgcaagaatcacttgataacttcaagttggtgccaattcgccaacgtggtgaaattaaagttggccaacctttatttaaactaatttataaagataagaagagttaaagaagtatagaaaaatgattaattaaaatcaactgcaaaagtgtttatgagtgataaattattaacaattgacttaagtcatgtttatggatttgataaagaaattatttttaagaaataccaaaaaaaagtagatcaaattcaccaagattttctagctcataaacttgctgatggtcacatgactgggtggtatgaccaacctgatcaaaaccaccaattccttttaaaaaccattaatcaaattgacaaaaagtttaaaagtttaaaagtaactgacattgtttatgttggtattggtggttcttttactggtattaaaacagttttagatttcttaaaaccaaaacaaagaacaggattaaaaatccactttgtccctgacctttctgcttttcaagctgcaagtgttattaaggaaattaaaaataaatcatgggctctaattaccacttctaagtctggtagaaccctagaaccagcactgaatttccgcatttttagaaacttattaaacaagcgttatggcaacaaacactaccaaagagtagttgttattactgatgaaaaaaagggattactaaccaaaatggcatcaaatcatggttaccaaaagttagttattgattcaaatatcggtgggcgtttttcaactctatctcctgctggtttgttactagccaaactttttggtcatgatcctaaggccatcttaaaaggaacattacaagccaaaaaggatttgcaaacaacttcacttgaaaacaattctgcatacctttatgcagtagttagacattgactatacaccacaaaaaaattcaaaattgaagtttgcattgcttatcacagtttgtatgaatatttgttattacagcatcgacaactttttggtgaatcagaaggtaagaacgataaatctttatttcctactttttcgatttttactgttgacttacactcaatgggacaactctatcaagaaggggaaaaagtgttttttgaaacagtaattgatgttaaaaatccacttgttaatattaatttacctccatctgattttgacaatgatgatgaacttgatttcttgttagataaaagcttaaatgagatttcagatgttgcaattgattcagttattaaagcgcactaccaagcaaatgtaagcattattaaattaactttaaaagaacaatctgcatttatgtttggttatttttacttttgactctctgttgctacagtgatgagtggatcattattagggcataatgtctttaatcaacctggcgttgaagtttataaaaagttaatgtttgaaaaactaagaagtggccactaacaaccgctatggtgatgaataaccttatataatttacaacatggataaaatagctattttaacttcgggtggtgatgctagtgggatgaatgccaccatcgcttatctaaccaaatatgcaattgcaaagcaattggaagttttttatgtaaaaaacggttattatggcttgtatcacaaccattttatcaccagtaaggaacttgatttaactgactttttctttatggggggaacagtaataggatcaagtcgtttcaaacagtttcaagatcctagcttacgaaaacaagcagttttaaacctcaaaaaacgtggtattaacaaccttgttgttattggtggggatgggagttatatgggtgctaaagcactcagtgaattaggattaaactgcttttgtttacctggtacgatcgacaatgatgtcaattccagtgaatttaccattggtttttgaactgctttagaagcaattcgggttaatgttgaagcaatttatcacaccaccaaatcccataaccgcttagcaatcatagaagtgatggggcgtgattgtagtgatctgaccatctttggggggttagctactaatgctagttttgttgttactagcaaaaatagcttggatctcaatggctttgaaaaagcagtgagaaaggtgttgcaattccagaactattgtgttgttttggttagtgaaaacatctatggtaagaacggtttacctagtttagaaatggttaaagagcactttgaaaacaacgcaattaagtgtaacctagtttcactaggacacacccaaaggggctttagtcctaatagtatcgaactctttcagattagtttaatggctaaacacacgattgatctggttgtaaataatgccaacagtcaagtaatagggatgaaaaacaaccaagcagttaactatgattttaacactgcttttaatttaccaaaagctgatagaaccaagttacttaaccaagttaacactgcaattatttaacgatgattgaccatttaaaaagaacaaagataatcgctacctgtggcccagctttaacaaaaagcttggttagcttaaagatgcttgatgataatgagtatgcagctattaaaaaggttgcttatgccaacattgaagcaattattaaaagtggggttagtgtgattaggcttaacttctctcatggtacccatgaagaacaacaagtgaggatcaagatagtaagggatgtagcgaaagcaatgaacatccctgtttctattatgttagatacaaatggtcctgagatcaggatagtagaaactaaaaaagagggtttgaaaatcaccaaagatagtgaagtgattatcaacaccatgagtaaaatgatcgctagtgacaaccagtttgctgtcagtgatgctagtggcaaatacaacatggttaatgatgtgaatataggtcagaaaatccttgttgatgatggtaagttaaccctggttgtcacaagggttgacaaacaacataaccaggttatctgtgttgcaaaaaacgaccacacagttttcactaaaaaaagacttaacctacccaacgcacagtactctatcccttttctcagtgaaaaggatctgaaggatattgactttggtttaagccaaggtattgactatattgctgcctcttttgttaatactgttgcagatattaaacaactgagagattatctgaaattaaagaatgctagtggggtgaagatcatcgctaagattgaatctaatcatgctttaaataacattgataagatcattaaagctagcgatgggattatggttgctaggggtgatttgggccttgaaatcccttattaccaagtcccttactgacaaaggtacatgattaaagcttgtcgcttttttaacaagcgttctattactgcaacccaaatgcttgattcactagaaaaaaacatccaaccaacccgagctgaagtgactgatgtttactttgcagttgatcggggtaatgatgcaactatgttaagtggggaaactgctagtgggctttaccctttaaatgcagtagcggtgatgcaaaagattgataaacaatcagaaaccttctttgattaccagtataacgttaactattatttgaaaaactccacggcaaataaaagtaggttttgacacaacgttgttttacctttaacaaaaaagactgttcctaaaagaaaacttgttaacagtgcctttaagtatgactttattgtctatcctactaataacattaacaggatctatgcattatcaaacgcacgcttagcagcagcagttattattttaaccaacaacaaacgggtttacactggccatggtgttgattatgggatcttctgttatttaattgataaaaaccccaaccagctaaccaaagctgaactgattgaacttgcttgaaaagcaattaaccactatcaggcttatggtgatttagaaaaactcaaacagtgtttagctgtctataatgaaacaattatcaatctttagtcctaaaaaatagcttagttttaaattagcatagaaatatatggcaatcttgattaaaaataaagttccaactaccctttatcaggtttatgataatgagggtaaattaattgatcctaaccacaaaattaccctaactgatgaacagttaaaacacgcttattacttaatgaacttgagtagaatgatggacaaaaagatgttagtttgacagcgtgctggtaagatgttaaacttcgctcctaatttgggagaggaagctttacaggttggaatgggattaggtttaaatgaaaatgattgggtttgtcctacgtttcgtagtggggctttaatgttgtatcgtggggtaaaaccagaacaacttttactctactgaaatggtaatgaaaaaggtagtcagatagatgctaaatacaaaactttacctattaacatcaccattggtgctcagtattcccatgctgctggattaggttacatgttgcactataaaaagcaacctaatgttgctgttactatgattggtgatggaggtacagctgaaggggaattttatgaagcgatgaacattgcaagcatccacaagtgaaacactgttttttgtattaacaacaatcagtttgctatctcaacaagaactaaacttgaatctgctgttagtgatctaagcgttaaagcaatagcatgtgggatcccaagggtaagggttgatggtaatgatctaattgctagttatgaagcgatgcaagatgctgctaattacgctagaggtggtaatggaccagtcttaattgagttcttcagctaccggcaaggtcctcacaccacttcagatgacccttctatctacagaaccaaacaagaagaggaggagggaatgaagagtgatccagtgaagcggttgcgaaacttcttgtttgatagatcaattcttaaccaagctcaagaagaagagatgttcagcaaaattgaacaggaaatccaagctgcttatgaaaagatggtactagatactcctgtatcagtagatgaggtgtttgattacaactatcaagaattaacccctgaactagttgaacagaaacagattgcaaaaaaatactttaaagactaatttaaaaaaagataactatgtcaaaaatccaagtaaataacattgaagcgttaaacaacgcaatggatcttgcactggaaagagatcaaaacgttgtactctatggccaggacgctggttttgaagggggtgtgttccgtgcaactaaaggcttacaacaaaagtatgggagtgaaagggtatgggattgtcctatagcagaaaactctatggctggtattggggttggggctgctataggtggtcttaaacctattgtagagatccagttttcaggcttttcattcccagctatgtttcaaatctttgtccatgctgctaggattagaaaccgttctcgtggtgtatataccgctccactagtagtgaggatgccaatgggtggggggattaaagcattggaacaccacagtgaaacattggaagcaatttatgcacagattgctgggcttaaaacagtgatgccatcaaatccttatgataccaaaggactttttctagctgctattgaatcacctgatcctgttatcttttttgaaccaaagaagctttatcgtgcttttcgtcaggagattcctagtgattattacactgtccctattggtgaagccaacttgattagtgaaggtagtgaacttacaatagttagctatggtcctacaatgtttgatttaattaacttagtttacagcggggaattgaaagataagggaattgagttaattgacttgcgtactatctccccttgagataaacaaacagtatttaactcagtgaagaaaacaggaagactacttgtagtgactgaagcggtgaaaagtttcactacaagtgcagagattatcacttcagtaactgaagaactattcacttatctcaaaaaagccccacaacgggtaactgggtttgatattgttgtgcctttagctagaggtgaaaaataccagtttgaaattaatgcacgggttattgatgcagttaatcaacttttaaaataacacttttttaaaatatagttacctagctttattttttagagctaggtacctcatttcataaattaaagtgatggaagacaacaagaaatgctgccaatgcaagtgcgaatgcgctaagtgcaacagctgctgtaaaaagtaagacaaaatttttgtctaacaactaaaaagccagtaactaaactggctttttttatttgttatagatcactactattaaatttaaactttaagtactatcaatacgatatggcaaatgagtttaaattcactgatgttggtgagggtttacatgaaggaaaagtaactgaaatcttaaaacaagttggtgatcagatcaagatagatgaagctttatttgttgttgaaactgataaagttacaactgaactaccttctccttttgcaggtacaattagtgctattaatgttaaagttggtgatgttgttagcattggtcaggtgatggcagttattggtgaaaagactagtacaccacttgttgaaccaaaacctcaaccaactgaagaagtagctaaggtaaaagaagcgggggcttcagtagtaggggaaattaaggtttctgataacctctttcctatctttggagtaaaacctcatgcaactccagctgttaaagacactaaagttgcaagtagtactaacattactgtagaaacaacccaaaaaccagaaagtaaaactgaacagaaaaccattgctatctcaacaatgcgtaaagcgattgcagaagcaatgacaaagtcgcacgcaattatcccaaccactgtattaactttttatgttaatgcaaccaagttaaaacaatatcgtgaaagtgttaatggttatgctttaagtaagtattccatgaaaatttcttactttgctttctttgttaaagcaattgttaatgcgcttaagaagttccctgtttttaacgctagttatgatcctgatcaaaacgaaattgttttaaatgatgacattaatgtaggaattgctgttgatactgaagaaggtttaattgtccctaacattaagcaagcccaaaccaaatctgtggttgaaattgcccaagcaattgttgatttagctaacaaagctagaacaaaaaagattaagttgactgatttgaataaaggtactatttcagttactaacttcggttcattaggagcagctgtaggtacacctattattaagtaccctgagatgtgtattgttgctactggtaatttagaagaacgcattgttaaagtggaaaatggaattgcagttcataccatcttacctttaacaatagctgcagaccaccgctgggttgatggggcggatgttggtaggtttggtaaggagattgcaaaacaaattgaggaattaattgatcttacagtagcttaatttatggattatgatctaattattttgggtgctggccctgctggttatattgctgcggagtatgctggcaaacataaacttaaaaccctagtgattgaaaagcaatactttggtggggtgtgtttaaatgttgggtgtatcccaactaaaacgttgttaaaaagagcaaagattattgattatttagttcatgccaaagattatggtatcactattaatggtcaagctaaacttgattgaaaacaactgttaaaacaaaaacaggaagtagttgataaattagttgcaggggtaaaaacaattattaagggtgctaaggtagaaagtattgaaggtgaagctactgttatagataaaaacaaggtgcaagtaaacaacacaacttacaccactaacaacattattgttgcaaccggatcaagaccaagatacttaactttaccagggtttgaaaaagcacaacaagctgggtttatcattgactcaacccaagctttggctttagagggagtacctaagaagtttgttgtagttgggggaggtgtgattggggttgagtttgcttttttatttgcttcattagggagtgaagtgaccattatccaaggtgttgataggattttggaggtttgtgatagtgatgtttctgaactgataagtaaaaccttaaaaaacaaaggagttcagattattaccaatgctcatgttgttagagctgaaaacaaccaactgttttacacagttaatggagttgaacagtctgtaattggtgataaaatcttagtttctataggaagaattgctaacacagagtgtttagatcaacttgatttaaaacgtgaccataacaacaaaattgttttaaatgaaaaactacaaacatcaactacaaacatctatctaataggtgatgttaacacgcaaatgatgttggcacactacgcttaccaacagggcagatatgctgttgatcaaattttgaaccaaaaccaggtaaagcctgctgaaaaaaacaagtgtcctgcttgtatttacacaaatcctgaagttgcttttgtaggttatagtgagatggaattgcaaaaagaaaagattgattatgtcaaatcttccttgccatttatttatagtggtaaagcaattgcagatcatgaaaccaatgggtttgtcaagatgatgtttaatcctaaaactggtgctatcttaggtggatgtattattgctagcactgctagtgatattatcgctgagcttgctttggtgatggaaaacaacctcactgtgtttgatattgccaattctatctcaccccatcctaccatgaatgaaatggtaactgatgtttgtaaaaaagcgatctttgattactttagttaaaataggctaaagtattaaaatctaattattaaattaaagtatggcagcaaagaatagaaccattaaggttgcaatcaatggttttggaagaattggaagacttgtttttcgttctcttctcagtaaggcaaatgttgaagttgtagcaattaatgatttgacccaacctgaagttttagcgcacctgttgaaatatgattcagctcatggtgaattgaaaagaaagattactgttaaacaaaacatcttgcaaattgatagaaaaaaggtttatgtttttagtgaaaaagatccccaaaatttaccttgggatgaacatgatattgatgtagtaattgaatcaactggtaggtttgtaagtgaagagggtgcttctctccatttaaaagcaggtgctaaaagagtaattatttccgcacccgctaaagaaaaaactatcaggacagttgtttacaatgttaatcacaaaaccattagtagcgatgataagatcatctcagcagctagctgtactactaactgtttagcaccattagttcatgtacttgaaaagaactttgggattgtttatggaacgatgctaacagttcatgcatatactgcagatcaacgcttacaagatgctcctcataatgacttacgtcgtgctcgtgctgcagctgttaacattgtgccaacaacaacaggagcagctaaagcaattgggcttgttgttccagaagcaaatggcaaacttaatgggatgtcactccgtgttccagtgttaactggttctattgtagagttaagtgttgtacttgaaaaaagtccatctgttgaacaagtaaatcaagccatgaagcgatttgcttccgcttcttttaaatattgtgaagatcctattgtatctagcgatgtggtaagttctgaatatggttcaattttcgattctaaactaaccaatattgttgaagttgatggcatgaaactttataaggtgtatgcatggtatgataatgaatcttcctatgtacaccaactagtgagagtagttagctattgtgctaagctctaatatgcttaatttcaaaacactccaagcaattgattttcaaaacaaaaccgttgttttaagaagtgattttaatgtcccaatgatcaatggggttattagtgatagtgaaagaattttagctggtttggatactattaagttcttagttaaaaagaactgcaagatagtgctactatcacacctttcaaggattaagagtttagaagataaactaaacaacaaaaaatctttaaagccggttgctgaattactccaacaactcttaccaactgtaaaggttcaattttcttgtaaaaacactggtgctgaagttaaacaaaaagtgcaagcattagcattcggtgaaatccttctccttgaaaacactcgctattgtgatgtaaacgataaaggagaaattgttaaattagaaagtaaaaatgatcctgaactagcgaaattctgggctagtttaggggaaatttttgttaatgatgcatttggtactgcccatagaaaacatgcttctaatgcaggaattgcaaagtatgttgcaaaatcctgtattgggtttttaatggaaaaagaactaaagaacctctcttacctaattcaaagcccacaaaaaccctttgttgttgttttgggtggtgcgaaagtatcagataaactaaaggtagttgaaaacttactaaaacttgctgataatatcttaattggcggagggatggtaaatacctttcttaaagcaaaaggcaaagctactgctaattccctagttgaaaaagagttaattgatgttgctaagcaaatcttggataaagatactcataataagattgtgctggcaattgatcaggtaatgggttctgaatttaaagatcaaactggcattactttagatgttagtgacaaaattcaagaacaatatcaatcctatatgtctctagatgttggatctaaaacaattgctttatttgaaagttatttaaaaacagccaaaactatcttttgaaacggtccccttggagtttttgaatttactaactttgctaaaggaacttcaaaaatcggtgagattattgctaaaaataaaactgcttttagcgttattggtggtggggattcagctgcagcagttaagcaaatgcaactatctgatcagtttagttttatctccactggtggtggtgcttctttagcactaattggtggggaagagttagtaggtattagcgatattcaaaaaaattcttaaaacatataataattttattaacaatatttttctatttaatatgggaagttcaaatctaaacatcaattcaaaaataaccgatatttttgcttatcaagtttttgattctcggggtgttccaacagtagcttgtgttgttaaattggcatctggtcatgtaggtgaagcgatggttccatcaggtgcttctacaggtgagaaagaagcaattgaattacgtgataatgatccaaaaaattattttggtaaaggcgttaacgaagccgttgataacgttaataaagttattgcccctaagcttattggcttaaatgcatttgatcaattaacagtggatcaagcaatgattaaactagacaatactcccaacaaagcaaaattaggagcaaatgctatattatctgtttcacttgcagtatcaaaagcagcagcaaaagcacaaaacagctcattatttcaatacatttcaaataaattaattggattaaatacaacaaattttgttttacctgtgccaatgttaaatgtaattaatggtggtgctcatgctgataactatattgattttcaagagttcatgatcatgcctttaggtgctaaaaagatgcatgaagctttaaaaatggctagtgaaacttttcatgctttacaaaatcttttaaaaaagcgtggattaaacacaaataaaggagatgaaggtggatttgcgcctaacttaaaacttgcagaagatgcacttgacatcatggttgaagccattaaattagctggatataagccttgagatgatattgctattgccattgatgttgctgctagtgagttttatgacgaagataaaaaactttatgttttcaagaaaggaataaaagctaatatccttaatgcaaaggattggagtttaacaagcaaagaaatgattgcttacttagaaaaattaacaaaaaaatatccaattatttcaatagaagatggtttgagtgaaaatgattgagaagggatgaaccaattaactaaaaccataggtagccatattcaaattgttggtgatgacacttactgtactaatgcagaacttgctaaaaaaggtgttgcacaaaatacaacaaactcgatattgattaaattaaatcaaattggttctattagtgaaacgattcaaacaattgaagttgcaaaaaaagctaactggagtcaagtaatttcacatcgcagtggtgaaacagaagatacaactattgctgatttggcagttgctgcccaaactggtcaaattaaaaccggttcaatgtcacgctcagaaagaatagctaaatacaatcgtttgttgtacatagaaattgaacttggtgataaaggaaaatacttaggttgaaatacctttacaaacattaaacctaaaaactttaacatctaagaaaagaaaatggtttttgaaaacttataaaatctttcatatgcgcacaaggtatttaattggcaattggaaaacaaataaaaatttaaaagacgcagttagttttgttgaacaatttcaacaaaataaacttaattacaatgccaaaattgggatagcacctgtttatgttcatctcactgaaataaaaaaaataattagtgatagtctccttttatttgcccaagacgctaactttattgaaagtggttcatatactggaactgtaagctttactcaacttcaagacattggtgttaacaacagtattattggtcattctgaaagaagaaaatactataacgaaaccagtgcagttattaatcaaaagctctttgcttgtctaaaagcatccatgcaagtagttttatgtattggtgaggctttaggacaagagattagctttcttaaaactgatcttactaattgcttagatacgattgacaaaagcttaattaaaaatttagttattgcttatgaacctttgtgagcaattgggacaggtaaaacagcaactcctgaagttgcaaatcaaaccattaaaaccattagggaatatattaatgacttatatgatgaaaatgttgctaacaatatctcaattctatatggcggatcagttgatcataataatatccaaaaactagcaataatggaacaaattgatggatttttagttggtaaagcatctttagaaattaaaaactttttagaaatggctagggtatatgcataaaaaagttttattagcaatccttgatggttatgggatctcaaatgctatttatggtaatgcagtacaaaatgcaaataccccaatgctagatgaattaatcaattcatatccttgtgtacttttagatgcatctggggaagcagttggattgcctatgggtcaaataggtaactctgaggtaggtcatctaaatattggggcaggtcgagttgtttatactggactttctttgattaatcaacatattaaggatcgtagtttttttgcaaataaagcttttttaaaaaccatagaacatgtagaaaaaaaccattcaaaaatccatttaattgggttattttccaatggaggagtgcatagtcataatgaacatctattagcactcattgaattgttttcaaaacatgcaaaggtagtattacatttatttggtgatggtagagatgtagcaccttgtagcttaaaacaagatcttgagaaattaatgatatttctaaaaaactatcctaatgttgttattggaactattgggggaagatactatggaatggatcgtgatcaacgctgggatcgtgaaatgattgcttataaagctttattaggagtttcaaaaaataaattcaatgacccaattggttatattgaaacgcaatatcagaaccaaattactgatgaatttatttatcctgcaattaatgccaatttaaattctgatcagtttgcattaaacaataatgatggagttatttcctttaattttagacctgatagagcaagacaaatgtcccatttgatctttaacagcaattattacaactatcaacctgaattgaaacgaaaagaaaatttattttttgtaacaatgatgaattatgagggaattgtacctagcgaatttgcttttccacctcaaaccattaaaaatagtcttggtgaagtaattgctaataataatttgaagcaattgaggattgcagaaactgaaaagtatgctcacgttactttcttttttgatggtggttttgaagttaatctcagcaatgaaacaaagacattaattccttctttaaaagttgctacatatgatttagctcccgaaatgtcatgtaaagctattactgatgcactactagaaaagcttaataactttgattttactgttttaaattttgctaatcctgatatggtaggtcatactggtaactatcaagcttgcattaaagctcttgaagcactcgatgttcaaattaaacgaatagttgatttttgtaaagctaatcaaataactatgtttttaactgcagatcatgggaatgcagaagtgatgattgataataataacaatccagttactaaacacactattaatcctgtaccatttgtatgtactgacaaaaatgttaactttaatcaaactggaattttagctaatattgctcctactatcttggaatatcttaaccttagcaaaccaaaagagatgactgcaaaatccttattaaaaaataacaattaa

SEQ ID NO: 9 - DNA, 3075 bp, M. genitalium
tttattagcacttgaaatgactcaaaaactaatctaatctatgaaattagaatacaaccggattattgatagcaccttagtcaaagctgatacgcttccccatgaaatagatactttatgtgctgatgctcataaataccagttttttgcagtgtgtgttaatcctagttatgttagttatgctaaaaacatcttgaaaaatactgcagttcaactctgttgtgttgttggtttccccttaggacaaacaacccaaaaacagaaggtatatgaagctaagattgctattaaagagggagcggatgaaattgatatggtaatgaatattgctgagtttaaaaaacgttgtgcttgtgttattactgaaattagagctgttaaaaaagtgtgtggcaagcgtaaattaaaagtaattattgaaactgcacttttaacaaatgatgaaatcaaagatgcagttaatgtttgcattgatggcaatgcagattatgttaaaacttccactggtttttctttccgtggtgcatctttagaagatgttcagattatgaataatgctgcagcaaatttaattaaaatcaaagcttcaggtgggattaaaacagcaaagcaatttatagatttatttcaagctggagctagtagaattggaacttcaaatgcggtccaaataatgcaagaattaaaaaaaatgaaccatgaatatcattaactgctcaaaaaacaataattattaataaataaaaattcctatggataaacttagattagaagttgaaagatggttaaatcatcctaatgttaattgggagttaaaacaacaaattaaggagttgaatgaatcagaaattcaagaactttttagtttggaaaaacctttatttggcactgcaggtgtaagaaacaaaatggcaccaggttatcatggtatgaatgttttttcttatgcctatttgacccaaggttatgttaagtacattgaatccatcaatgaaccaaagcgtcaactacggtttttagtagcacgtgatacaagaaaaaatggtggtttatttttagaaacggtttgtgatgtaattacatctatgggtcatttggcttatgtgtttgatgataaccagccagtttcaacacctctagtgtcccatgtcatttttaaatatggttttagtggaggtattaatatcacagctagccataaccctaaagatgataatggttttaaggtttatgatcatactggtgcacagcttttagacacacaaacaaaccaattgttaagtgatttaccttgtgttacatctatgctagatttggaattacaaccaaatccaaagtttgtccatactcttgacaatgaaaaggtttataaaaactatttcagagagttgaaaaaggtgttggttattaacaacaacaatttcaaagacattaaggtagtttttagtgggcttaatgggacttcagtttgcttaatgcaacgctttttaaagtaccttggttatagcaatattatcagtgttgaggaacaaaattggtttgatgagaattttgaaaatgctcctaacttaaatccagagtataaagatacatggatattagcacaaaaatatgctaagaaaaataatgctaagttaattattatggcagaccctgatgctgatagatttgcaattgcagagttaaataataatcaatgacattatttttcaggtaatgaaacaggagcaattactgcttactataaacttaatcataaggtttttaaatcaccttacattgtctcaacttttgtctcaacttatttggtaaataagattgctaaaagatatggcgcttttgtgcatagaaccaatgttggttttaagtacattggtcaagcaattaatgagttatcacaaacaaacgaattagttgttggttttgaagaggcaattggtttaataactagtgataaattaaaccgcgagaaagatgcttatcaagctgctgcattattgcttgagattgctagacattgcaaagaacaaaacatcacgcttttagatttttataaaagaattctttctgagtttggtgaatatttcaatttaacaatatctcatccctttaaagctactgctactgattgaaaagaagagattaaagctttatttaatcaacttataaatgctaatttaactgaagtggctggttttaaagtagttaaagtccatcttgataaacaaacaaatatcttagagtttggttttgaaaatggctgggttaaattccgcttttcaggtactgaacctaaattgaaattttactttgacctaactaatggcactagagaggctctagaaaagcaagctaagaaaatttataaattctttgtaaatttactcaaactcaacaaagcttaaagaagtatagaaaaatgattaattaaaatcaactgcaaaagtggccactaaggttgttttttcactcttaccacttttaaataggtttgacaagtcacttttagaaagttactttcaagatggattgaggttaatccattatgatgtgatggaccaatttgttcataatactgcttttaaaggtgaatatttggatgaattgaaaacaataggttttgatgttaatgtccatttaatggtggaacagatcatccctcaaataaatttttatctttcacaacctaatgtgaaaaggatttcgtttcatgttgaaccatttagttttgcaaagattaaagaactaatccaactagttaaagaaaatggtaaagaagttggtcttgcttttaaatttacaaccaatttacaactataccaaccattttttacaaccatcgactttatcactttaatgagtgttcctcctggtaaaggtggtcaagcttttaacgaagctgtttttacaaatttaaagattgctaaccattacaacttgaaaattgagattgatggtgggattaaagttaataacattgatcaaattaaagcctttgttgatttcattgtaatgggaagtggctttataaaattagagcagtggcaacgtcaaaaattgttgcaaacaatctaa

SEQ ID NO: 10 - DNA, 11899 bp, M. genitalium
tttacagttttagcaataataaaaaatctttgaatattgcttgagacccttcatctccaccattaaagaattccaacgtctaacttacctttgcataaataagaagttagacgttttgttttatattaggcaaaataagatgtcatttgatggaaaactaaaagcgcaatcaatcttagaaacttacaagaattttgattgatcaaaatgtaagttagtgattattcaagctaatgatgatgattcatcagacagttttattaaacaaaaacttattgcttgtaacactgtaggagcaaaaagtgaattaattaaactatctaatcaaataactcaagcagagttaatagaaaaaataattagtttaaatcatgatgtaaatgttactggtatcattttgcaattgccagtttatccacacttagataaaaactcactactagaagcaattaatcctttaaaagatgttgatggtttaacaactaatcatttggctgaaattaaaccttgtatagttgaagctataataacactaaaagaactatttaaccttgaatttaataatcaaaaaattgttgtggtaggtttgggaataactggtggcaaacctatttatgaatttttaaaaactagtggttataaagttcaagcatgcgataaagatactccaaatacatttgaattgattaaaagtgctgatatagtttttactgctattggaaaatctcatttttttcaagctaaaaactttaaaaaaggagttattttatttgatataggtgtttcaagaaacaagcaaaataaactttgtggtgatataaatcctgaaggcattgaaaaaaaagctagatgatgaactaaaacgcctggcggtgttggcccttttacagttttagcaataataaaaaatctttgaatattgcatgaaaaaaataaacgttgtttacaatccagcatttaataaaaaagaagataaattgaaatcctaattacaacttatcatggatctaaaaaaacaatacattattgccttagatgaaggtactagttcttgtcgatcaattgtttttgatcacaatcttaaccaaatagcaatagcacaaaacgaatttaacactttttttcctaatagtggttgagttgaacaagatccactagaaatttgatcagcccaactagctaccatgcaaagtgctaaaaataaagcacaaatcaaatctcatgaagtgattgcagttggtattaccaatcaaagagaaacaatagttttatgaaataaagaaaatggtttgcctgtttataatgccatcgtttgacaggatcaaagaactgcagcactatgtcaaaaattcaatgaggataagttaatccaaaccaaagtaaaacaaaaaactggattacctattaacccctattttagtgctactaagatagcttgaatcttaaaaaatgttcctttagcaaagaaactaatggagcaaaaaaagttgttatttggcaccattgatagctgattaatctgaaaactaactaatggaaaaatgcatgttacagatgtttcaaatgcttcaagaactcttttatttgacattgtcaaaatggagtgatccaaagagttatgtgatttatttgaagtaccagtttcaatcttacctaaagttctgagttccaatgcttactttggtgatattgaaactaatcactgatctagtaatgctaaaggtattgtaccaattagagcagttttaggagaccagcaagcagctttgtttggtcaactctgtactgaacctggaatggtaaaaaatacctatggtactggatgttttgtactcatgaacattggtgataaaccaacactctcaaagcacaatctgctcacaacagtagcatggcaactagaaaatcatccacctgtatatgcattggaaggtagtgtgtttgtagcgggtgcggctataaaatggttaagggatgcattaaaaattatctattcagaaaaggaaagtgatttttatgcagaacttgcaaaagaaaatgaacaaaacctagtttttgtaccagctttcagtggacttggagctccttgatgagatgctagtgctaggggtattatcttaggaattgaagcaagcactaaaagagagcacatagtaaaagctagcttagagtcaattgcttttcaaactaatgatttattaaatgcaatggcaagtgatctaggctataagattactagcattaaagctgatggggggattgttaaatcaaactatttaatgcagtttcaagctgatattgcagatgtaattgtttctatccctaaaaataaagaaaccactgcagttggtgtttgttttttagctggacttgcttgtggattttgaaaagacattcatcaacttgaaaaactcactactcttgataaaaagttcaaaagcactatggacccaaacataagaaaaaccaaaattaacagttgacataaagcagttgaacgtgctttaaaatggaaagaaattgattaatcgttatcttgattagactttaaattacactggtgataatatggcgataagaattaaaagtacaagagttggtagatttgtttctgaatcagtgggattaggtcatcctgataaaatttgtgatcagattgcagatagtatcttagaccaatgtttactacagagtaaaactagtcatgtagcatgtgaagtctttgcttctaaaaaccttattttaataggtggtgagatttcaacaagtggctatgttgatgttgttcaaactgcttgaagaattttaagaaatttaggttacaacgagactgatttcagttttttaagctgtatcaacaaccaatcactagaaattaatcaagcagttttaaaaaataatgagattaatgcaggagatcaaggcattactgttggttatgcagtgaatgaaacaaagcaactaatgcctttaggagttttactagcacactcgtttttaaaacaagcagaaaaactaacaaaacaatttgattttttaaaaaatgatatgaaaagtcaagtggttttaaactacagtttaaaccaagttgaatgtgaagaagttttactatcaattcaacacactaatgctattagtttaacagaattgagaaaagtgattgaaaataatgtaattctacctgttttaaaccaatatggttttcaagataaaaagccaacttgtttagtgaatcctggtggttcttttgttttaggtggacctatggcagatactggactaactggtagaaaaatcattgttgacacctatggtccatatgctcaccatggtggtggtagctttagtggcaaagatcctagtaaggtggatagaacaggtgcttattttgcacgttttatcgcaaaacatattgtaagtttaggctgggccagtgagtgtgaagtcagtattagctgagtcttttcaaaacccaatccacaatctattactgttaagtgttttaacactaacatacagtatgatgaagtgttaattaatagagttgtaaataactatttcaactgatcgattactaaaattattgacaagctaaaattacttgattttgttaagtattctgattatgcagtttatggacattttggtaatgatctttcaccatgagaacagcccactgaattggataaattagaatgcttaatcaaaaatttccattagttcattttggacaaaaagaaatttttatgctaagataaaaatgctaaataaccaacagatccaccagagtgtactgatcaatgaagtgatccataacctcaatattaacccttgtggtaactatttagatctaactgcagggtttgcaggacacagtcaaaagatcttagaaaaactaacaacaggaactttaacaattaatgatgttgataaagaaagtattaatttttgccaaaagcttttttttaaaaacaacaacgttgttattattcacgataactttgctaacttcccagttcatcttaaacaactatcaataaccaagtttgatgggatcttaatggaccttggtgtatcaagccatcaactcaaccaacctaatcgcggttttagttttaagaatgatggaccgattgacatgcgtatggaccaatccaatcagaaaaataccgcactaacagttttaaaaaacttaactgaacaaaagttaagtctaatccttaaaaagtatggtgatattaaacaccctaaaccaattgctattggattgaaaaaagcagttcaaactgaaaaaaatcttaccacaactcaactagcaaaagtggtaaaagaatgtgctactggatttgaaaaataccaatcaagaaactatcttgccaaagtttttcaagcaattaggatctatcttaatgatgagattactaatctgaaaactgcgttaacttttatccctaatcttttaaaaaacaacagcaggtttcttgtgattgtttttcactccattgaagaaaaaattgtaaggaatttcattgcaaaactaaccagctttatccaacctgaagctctacccattaaactcactcctgcttaccagttaattacaaaaaaaccaatcctaccttcccaaaaagaacttgaattaaacccgcgttcgcgtagtgccaaactctttgttatccaaaaaaactagcggttttatacaatgtataacctgtctaaaagacaatttcatgctaattgctatctgagcgatgacacaagaaggactaataggtaataacaacactttaccttggatgattaaacaagagctagctcactttaaaaaaactacgttatttcaagctttgttaatggggagaaaaacttacgaatcactccccaaggtatttgaaaaaagaacaatatttctcctttcaaaagatcaaaactaccgttttgaagaaaagggaagtgaagtgaaagttattaatgatttttgaccactaattaaaagttaccaagcaaataaagaaaaggatttgtttatttgtggtggaaaaagtgtgtatgaacagaccattaatgaatgtgatcagttaattgtttcaatcattaaaaagaagtataagggtgatcagtttttgaaggttgatctcagtaaatttgtacttaatgaagttgtagagtttgaggaatttaatgttaattattatagaaagaaacaacaataataaaaaaccataaatcactaacaaaagctttatattaacaatggttgataaaaacagtttaagaaaattaatgcttctaaaaagagcagaactaaatgatcttgaaaaatcgcatttagatcaaaagattaaccaaaaattaatggcttttttaataacaagaccaacaattaaaaatttagcactttacattcccattaaaaacgaagtggcttttttagataactttctagattttcttaagttaaataaaattacaagctgttttcctagtattgttgatcaatttaacatgaagtttattgatcaaaataataatgaaattaaccctaatgatattgattgtttttttatccctttattagcttttaataaggcaaaccacaggattggttttggtaagggttattatgaccgttatttatcattaactagcaaaaaacaactaaaaatagggatagcatatgactttcaatatgcagaattcactaatgatccttgggattatcaattagatttaattatttgcaatggataacgattaaataaagcttcataccgttgaagagatcttgataatgcataacaagcaattgcttttagcacataggggttattcattcattgctccagaaaacaccaaactagcatttgatttagcttttgaatattgttttgatggaatagagcttgatgttcatttaactaaagatgaacagttagttatcattcatgatgagacaacattgagaaccgcattagttaataaggaggttgagtttgaatcattagttagtttaaaaagagatgatcatagtgctttttttcaccttaaaattcaatttcaatcgatcctaactttaaaagagttcttagatctttatttagataaatttaagttaatcaatattgagattaaaactgatcaaaaaccatatttaggaattgaaaagaagcttgttgacctagttaaaggttatggtaaaaaagcaatagataagatcttgttttcatcctttaactttgaatctttgcaaaaagtttatgatttagataatagttacaaaaaaggttttttattttggactaaaaaacagtttgaaacaattagtacagctagaatccaaaagatttgtcaattcctccacccatgaaccaaaatatatgaaaagtatccccaaatgatcaaaaaacttaacttacctttaaatttatgaacagtaaacagtcaaaataagtttcagcagttcttagctgataatcatgtttatgcacaaattgctaacaaaaagtttgaaataaaaataaattaggcgatattcaaaaaaattcttaaaccaaattaataaaacaatgagtgttattgatatttttaaaaaacgattacaagctgttagtaaaaaacctgtaattatctttccagaaggttgatcagcaagtgttttaaaagcagttgaaatgcttaatgaatctaagctgatccaacctgcagttatctttcataatcgtcaggaaatccctgcaaattttgataaaaaaataactcattatgtgattgatgagatggatttaactagctatgctaactttgtctatgaaaaacgtaagcataaggggatggatttaaaagaagcacaaaagtttgtacgtgatcctagttctttagctgctaccttagttgctctaaaggttgttgatggtgaggtttgtggtaaagaatatgctacaaaagatactttaagaccagctttacagttactagcaactggtaattttgtttctagtgttttcatcatggaaaaaggtgaagaacgtttgtacttcactgattgtgcttttgctgtttatcctaactcccaagagttagcaacaattgctgaaaacaccttcaattttgccaaaagtttaaatgaggatgagataaaaatggcttttttaagctattcaacgcttggcagtggtaagggtgaaatggtggataaagttgttttagcaactaaactatttttagaaaaacaccctgaattgcatcaaagtgtttgtggtgagctccagtttgatgctgcttttgttgaaaaggttaggttacaaaaagcacctcaactaacttgaaaaaatagtgctaatatctatgtttttcctaatttagatgctggtaacattgcttataaaatcgcccaaagacttggagggtatgatgcaattggtcctattgttcttggactttcaagtccagtgaatgatctttcaaggggagctagtgtcagtgacatttttaatgttggaattatcactgccgctcaagcaattaaataaatcagagtaattttattaatatttatctaacttacatctggtgcgcttaagaaaagttaaaaacgctcttttaaaaattaatcaaagtccttatttttattcaaaagataagtttgctaagtttactaaaaaacaattagtgctggaattgggttgtggtaagggtacttttttaatcaaagaagcacaaaaaaataacaattttctttttataggaattgaacgtgaacctacaattgttttaaaagcaattaacaaaattaacaagttggattttaatttggaaaatatcttattgttgtgtacagatgcaaaacaacttgatgattattttcaagctgaatctgttcaaaaaatctttattaatttccctgatccttgacctaaaaagcgtcatatacaaagacgtctaacaagtccagattttttgaaacttttttgaaatttactagtaaaaaatggcttaattgagtttaagactgataatgataagttatttgaatatactttaacaacattgcaagaaaatagtcaaatttttgaaattatccatcaaataactgatcttaacaattctgaattcagttttcaaaatagtatcactgaatatgaacagcgctttatggaattagaaattccaattaaaaaactagtgattaagaaaataatttaaaagactcttgaattattagttaataataatatttattgatatggacaaatttttaattgatgttattgtagaaatccctaaaaacagcaaaataaagtatgagtatgatcgtcaaactggtcaaattcgcgttgatagaatcctatttggaagtgaatcatatccacaaaactacggttttattaaaaatacattagattgagatggggatgaacttgattgttttatctttgcagatcaaccatttttgcctgcaacagttgtgcctacaagaattgtaggagcacttgagatgattgatgatggggaaattgatactaagttattaggagttattgattgtgaccctagatataaagaaattaatcaaattagtgatttacctaaacatagaatagaagaaattcttatctttttaaaaacttataaattacttcaaaaaaagactgtaattattaagggtttaaaagatgtttgttgagctaaaaaagaatatgaaatttgtttgcaattaatgaaagattatggtcatttatcaaaagatcaatttatccaaaaaatgcaaattcttcatccagaacattaccaaaagtaatattattttttaataaataaggtaaatattcttcggttaaatgcaaagtcacaaaatcttggttgttaatgcaggtagcagttcaattaaatttcaactttttaatgataaaaaacaagtactagctaaaggactttgtgaacgtattttcattgatggtttttttaagcttgaatttaatcaaaaaaagatagaagaaaaggttcaatttaatgatcataatcttgctgttaagcattttttaaatgcgcttaaaaaaaacaaaattattactgaactttcagaaattgggctaatagggcatagagtagtacaaggagcaaattattttacagatgcagttcttgttgatacacattcactagcaaaaataaaagaattcattaagttagcaccgcttcataataaaccagaagcagatgttattgaaatttttctaaaagagataaaaactgctaagaatgttgctgtatttgataccacttttcacactactattccaagggaaaattatctttatgcagttcctgaaaattgagagaaaaataacttagtaagaagatatggttttcatggaacttcttataaatacattaacgagtttttagaaaaaaagtttaataaaaaaccacttaatttaattgtttgtcatcttggtaatggtgcaagtgtttgtgcgattaaacaaggcaaatcactaaacacatcgatgggattcactccccttgaaggattaataatgggaacacgtagtggtgatattgatcctgccattgttagttacattgctgaacagcaaaagctttcatgtaatgatgttgtaaatgaattaaataaaaagagtggaatgtttgctataacaggtagttctgacatgcgtgatatttttgataaaccagaaattaatgatattgctataaaaatgtatgttaatcgtgttgctgactatattgctaaatacctaaatcaactttcaggtgaaattgatagcttggtatttactgggggagttggtgaaaatgctagttattgtgtgcaattaataattgaaaaagttgcttcacttggttttaaaactaacagtaatttatttggaaattatcaagatagttctctaatttcaacaaatgaaagcaagtatcaaatttttagagttcgtacaaatgaggaattgatgattgtagaagatgctttgagagtaagtacaaacattaaaaaataagataaaaaacattactttaaattatatttaatgatgcaaaatgaataatgctaattttgaaaaatatgttgatttagtttttgaagcaaacaaaaatttcaacttaacaggatttaaaacaaaagaagctatttatcagaatttagttatagaaatattgacattatttaaaggatatgaaaaattttttattgacaaaactgtagcagacttgggaagtggaaatggttcgcctgggataatattaaaactgttatttcaaaaaataaaaaagttagttttaattgatagtaaacacaaaaaaattagctttttaaataaattaactaagcaactaaatctggagaaaactgttgcaatttgtgaacgaattgaagtacataaaaatcactatgatgttatctgttctcgtggtctaagtacgattattaaagttaatgatttagcattttccttgcttaactcaaaaggtattatttttcatataaaacaaagcttagaccaatacattgaatttgaaaaatcaaatcagaagaatcaatttaacttgttatttataaagcactttactagtcagaataaaaaactaattttgatagctttacaaaaaaatgattaacaatcaaaaaacaccgtttttattacgttaattaagttgaatgttttcaaaggtaagacttttacttaataaagagttacaacgtcaaagagaaaacatttgtttaattgcttcagaaaattacgttagccaagacatattagctgtaactggttcagtattaacaaataaatatgcagaaggctatcccagtaaacgtttttatcaaggctgtgaagttgttgatgaatctgaaaacttagccattgaaagttgcaaaactttatttggagcacaatgggctaatgtccaacctcattctggatcatctgctaactatgcagtttacttagcattgttaaaaccaggagatactatcttaggattagatcttaattgtggtggtcatttaacccatggtagccccgttaatttttcaggtaagcaatatcaagcagtaacttattcgttagattttgaaacagaaactcttgattatgatgcaattcttcaaattgctctcgaacacaaaccaaagttaattatttgtggtttttctaactattctaggactgttgactttaaaaaatttagtgcaattgcaaaacaagttaatgcgtatcttttagctgatattgcccatattgctggtttcatcgctgcaggtttgcaccaaaaccctttgccttttgtggatgttgtcacttcaacaactcataaaactttgcgtggtcctagggggggtatcattatgtctaacaaccaagcaattatcaaaaagcttgatagtggagtatttcctggatgtcagggtggacctttacaacatgtgatagcagctaaatatgtttgttttaaagaagctttgaatccaaagtttaagcagtatatgcaacaagttaaagataatgctttagcaatggcaaattgatttttaaagcagggttatcgtgttgtgtcaaaaggtactgaaacccacttattttcattagtggttggtaatggtaaagatgttgcgttgtggttacaaaaagctaacattgttttgaatatgaatacaatcccttttgaaacaaaatctgcttttagtccttcaggtattagacttggaactcctgcaatgacaaccagaggttttaaaactaatgactttatttttgttgccagtttgattgataaggttattaaaagtaatggtaatcaaaaggtaattagtcaaacaaaaacagctgttttaaatctcttaaaacgctttccgctctataagggtttagcttattaa

SEQ ID NO: 11 - DNA, 15051 bp, M. genitalium
attaaaaaaataccttgattttgacacaatcaagtaatttatgaataaaggtgtttttgttgttattgaaggagttgatggagcgggcaaaactgctttaattgaaggttttaaaaaactttatccaactaagtttttgaactatcaacttacttatactagagaacctggtggtactttgttagctgaaaaaattcgtcaacttcttttaaatgaaacaatggaacctctaactgaagcttatttgtttgccgcagctagaactgaacatatcagtaagctaattaaaccagcaattgaaaaagaacaactagttatttcagatagatttgttttctctagttttgcataccaaggattaagcaaaaaaataggcattgatacagtaaaacagattaatcatcatgcgttaagaaatatgatgccaaactttacctttattttggattgcaattttaaagaagcattacaaaggatgcaaaagcgtggtaatgataatcttcttgatgaatttattaaaggaaagaatgattttgatacagttcgttcttattatttaagcttagttgataaaaaaaactgtttcttgattaatggtgataataaacaagaacacctagagaaatttattgaattgttaacaagatgcttacaacaacccacgcattactaacaactaatttttagtttaaacttatttaaagataactaacgtgataaaaaaagttcaacatgctttaatcttgaatgaattgacaaaactgcgtgataaaaatacaacaacctcccagtttcgcatggccttgaatcaaatcacttcattactcttttttgaagcaactaaacagctaccactagcaacagttgaagttgaaactccctttgctaaaacaaagggctacaaattaaaaaatgacattgttcttgtacctattatgcgtgctggacttggaatgattgatgctattgttcgctattcagataaaatcagagttggtcatttaggaatctatcgtcaaacccaaacaaccagtgtaatttcatactataaaaagatgcctgaaaacatctctgattcacatgttattattcttgatcctatgcttgctactggaactacattgttaactgctattaaatctattaaagaagataaacctatcaaaattagtgttattgctatagtagcagcacctgaaggaattaataaagtagaaaaaatgcatcctcatgttgatatatttcttgcagcaattgatgaaaagttaaatgacaatagatacataatccctggtcttggtgatgctggggaccgtttatttggtactaaataatgtttttaatagagacttttgcaaatcttgaccaggttcaatggtttgtcctactgttttatctctaattgctttgatgaaatttttaacaacaagcaggttaacatcagcatcaagcaatgcaattctaatctcttttagaactaactctacatctttctcagtgatcgtttgagcgttaatttttttttgcatcgtgcgcataacgatgcttgataacattgctttgaacatgatttttaattatttattattaaataatgttttaataaaacaatattgcaatatgaccccacatataagtgctaagaaagatgacattagcaaagttgttttaatgccaggtgatccattgagagctaaatggatagctgagcaattcttagatcaagctaaattagtcaatgaagtgaggggaatgtttgcttatactgggcagtataaatctaaaacagttacagtaatgggccatggaatggggatcccttctattggaatttattcatatgagttgatgaatttttatgaggttgaaactatcattagaatcggaagttgtggtgctttagcaccgcaattaaaattaaaagatcttgttattgcttcaaaagcatgaagtgagtctatttatgctaaagacatgggtgttgaaattccagaagataagatcttatttgcaacaagttctttagtggaattagcaaaagaaactgcgattaagaacaagcttgattttcatgaaggattagtattttgtgaggatgctttttatcaaactagaaaagatgtaattagtcttgctaaagaaaaaaatagtttagcagttgaaatggaagcacatgcactttatgctaatgcaatcctgttgaagaaaaaagcacttacactcttaacagtatctgattctctagtaactcatgaagcacttagttctgaattaagacaaaagtcatttaagcaaatggctttattagcacttgaaatgactcaaaaactaatctaactgctcaaaaaacaataattattaataaataaaaattcctatgaaggtgaatttagagtggataattaaacagttacaaatgatagttaaaagagcatatactcccttttctaactttaaagttgcatgtatgattattgctaacaaccaaactttttttggagttaacattgaaaattcttcctttccagtaactttgtgtgctgaaagaagcgccattgctagcatggttacaagtggtcataggaaaattgattatgtttttgtttacttcaatactaaaaataagagtaactcaccctgtggaatgtgcagacaaaacttactggaattttcccatcaaaaaacaaagcttttttgtattgataatgatagtagttataaacaattttccattgatgaattattaatgaatggttttaaaaagagctaaacagcttatcagtttctgcaatatatgaacgtcacattgaatggtttgcgaacactttgctgatggtgaaacttatatccgttttgatgaatcagttcgtaacaaagatatctatatttttcaatcaacctgtcctaatgttaacgatagcttaatggaacttttaattgctattgatgcattgaaaagaggtagtgctaaaagtattactgccattctaccctattatggatatgcaagacaagatagaaaaacaaaaggaagagaaccaattaccagtaaattgattgctgatatgttaacaaaagcaggtgctaacagggttgttctaactgacattcatagtgatcaaacccaaggtttttttgatattcccgttgattctttaagaacttatcacatctttctttttagagttatagaactacttggtaaaaaagacttggtggttgtttcccctgattatggtggggttaaaagagcaaggttaattgcaaatacactagaactaccattagccattattgataaaagaagaccatctcataatgttgctgaatcaattaatgttttaggtgaagtgaaaaataaaaactgtttaatagttgatgacatgatagatactggtggtacagtaattgcagcagccaagctattacaaaaagaacaagctaaaaaagtgtgtgtaatggcaactcatggtttgtttaacaatgatgcagaacaaaagtttatggaagcatttgatcaaaaactaattgatttcttgtttgtatcaaactctattcctcaatataagtttaaagctgtaaagcagtttgaagtagttgatctagcatctttatatgaagaggttgttctgtgttacgctaacagcttatcagtttctgcaatatatgaacgtcacattgaatggatcaaaaagcacgtataaatagccaatctggttagcagctatcccaattcctggaataatgtcatattcttgtgctttaccatcatatgaagcatcaacatatgcaatcatctttttaatacaggtttcaatttgctcatctattggaaaattaacagcttcagttggcttgttaatcaaagcattatcatcaaaaacaagccaagttttagttggttgaaaagtcattgataaaaacagtaaagttaaaattattctaacaattgaagtgaacaatcaggggagaatttttgtcattactggtcctagcggtgttggcaaaagcacccttgttaaagccttattagatcatttcaaagaacaactgttctacagtatctctgcaactacaagaaaaaagcgcattagtgaaaaagagggaattgattatttttttaaagataaagatgagtttgaaaacttaataaaacaagatgctttcattgaatgggcttgctataataaccattattatggaacgctcaagtctcaagctgaacaagcaattaaaagcggaattaatttaatgcttgaaattgagtatcaaggtgctttacaggttaaaagtaaatatcctcataacgttgttttaattttcattaaaccaccttcaatgcaagagttgttaaaacgtttaaaaaagcgtaatgatgaagatgaaaccacaattaaaaaacgtttagaacaagctaagatagagtttcaacagattgataattttaagtatgttgtcactaacaaagagtttgataaaacccttaatgagttgaaatcaatcttactatctgagtttatttaaaccaaccttgattttgaaattttattaggtattttaaaaaatgattggagcaaagactagggttgcaatagttggcgggattggttacataggtagttgttttgctagttttatcaaagaacaaaatgataagctaattgttactgttattgataacaacaaaaataaccatgtaattaaactcttaaaaaagattggaattgaattctattttgctgatttactagatagacataagctaactgaagtaattgcagcaattcaacctgatgtggtatttcactttgctgctaaaacaagtgtaagtgaatcagtacataatccattgaagtactttgattgcaatgtaattggtactttaaacctaattagtgcaattagtaacttacagaagccaattaaattatttttcgcttctagtgctgcagtgtatggtcaaacaactaatagttacattagtgaagagattgtaataactgaaacacaagcaaccaatccttatggattgagtaagtttttagatgaattaatcttaaatgcagttgccaaaaatagtcaactacaagttgtttgcttacgcttttttaatgtggcaggtgcaattctgccatttggtaattttaatggtaataccacgcttttaattcctaacttagtaaaagcctttttaaaacaaactcccttttttttatatggcaatgattatgcaactaaggatggtagttgcataagagattacatccatgtttatgatatatgtaatgctcatttcttattatgaaagtggttaaatgatcatcgccaaattaaatttgaaacctttaacttggggagtgggataggaacttctaatttagaagttattgatattgctaaaaaagtgttttatcctagtagattaaatttagaaattagaccaaaaagaagctgagatccagcaattttagtagcaaatgttgctaaagcaaaacaaacctttcaattcaaaataacgcgtaatttgaaagatatgataagtgatgagcgtaatttttatgagaatttttataatgacgcttattaacagtgcaaactagcaaaggtagtgtgaaaattatcacctaatggtagcacagtttaataagttcattatcttaggacccccaggggcaggaaaaggtacagtttgtaaactgcttagcaaaacaactaagttagtccatattgctagtggtgatctgtttagagaagccattaaaaaccagagtgttattggtagaaagattgcagcaattatcagtcagggtggttatgttgatgatgccactactaaccagcttgtttatgaatatatcactaccaatccattaccaaatggttttatcttagatggttatccaagaacagagaaccagcttgattttctaaatattaaactaaccattgacatggtctttgaactagttgttagtgatctgaataaactgattacacggattgataacagggttatttgtaacaactgtaacagtgtttataacttgctttttcaaaaaccactagttgaaaatagttgtgatcagtgttcagctaaactagtgaaaaggagtgatgataacaaagcagtggtcaaagcaagaatggagttatatcaacaaacaattcaaccaatccacacttactttttcaacaaacaacttttagtacaaattgattgctttttaccactagaagaacaactcaagacaatcaaacaatttattagataacggttttatacaatgtataacctgtctaaaagacaatttcatgaaacagtatttagatttagctagttatgttttagcaaatggtaaaaaaagaaaaaaccgtacagatacagatactttaagtgtctttggttaccagatgaaatttgaccttactaatagttttcctttattgacaactaaaaaggttaattggaaggcaattgtccatgaattgttgtgatttattaagggtgataccaacattaagtacttagttgataatggggtgaacatctgaaatgaatgaccatatgaaaactttaaaaaatcaccaagttttcaaaacgaaacactccaagaatttatcttaaaggttaaaactgataatgagtttgctaaacaatttgctgatttgggtcctgtttatggcaagcaatgacgtaattttaatggtgttgatcaactcaaaaaagtcatccaagagattaaagaaaatcccaactcaagaaggctaattgtctcaagctgaaaccctagtgaattggaaaaaatggcattggctccttgtcattcactctttcagttctatgttgaagaagataaactaagcttacagctttaccagcgcagcggtgatatctttcttggtgtcccatttaacattgcatcttacgccttacttgtgtatttagttgctcatgaaactaagttaaaacctggttattttatccatacactaggagatgcacatatctatgaaaaccacattgaacaaattaaattacaactaacaagaacaaccctagacccccctcaagtggttttgaaaagtgataaatcaatctttgcttatagttttgatgatattgagttagttggttataattaccatccatttatctatgggagggttgcagtttaattaatgttaattattatagaaagaaacaacaataagagatatggcagctaacaataaaaagtactttttagaatcattttccccacttgggtatgtaaagaataattttcagggcaacttacgttctgtaaactggaatttggttgatgatgagaaggatttggaagtgtgaaacaggattgttcagaacttttggttacctgaaaagatccctgtatccaatgacatcccctcatgaaagaaactctcaaaggattgacaggatctgatcactaagacctttactggtttaacactacttgatactatccaagctaccattggtgacatctgtcaaattgatcatgctctaactgatcatgagcaggttatttatgcaaactttgcttttatggtaggggtacatgcccgttcctatggaacgatcttctcaactttatgtacatcagaacagattaacgctgctcatgagtgggttgtaaacactgaaagtctccagaaaagagcaaaggcattaatcccttactatacgggcaatgacccgttaaaatcaaaggtagcagcagctttaatgcctgggtttttactgtatggtgggttttatttgcctttttacttgtcatcaagaaaacaactaccaaatacatctgatattatccgcttaatccttcgtgataaagtgatccataactattacagtggttataaataccaacgtaaactagaaaaactccctttagcaaaacaaaaggagatgaaagcatttgtttttgaactaatgtatcggttaattgaacttgaaaaggactatttaaaagagctttatgaagggtttggaattgttgatgatgccattaagttcagtgtttacaatgctggtaagtttttacagaacttaggttatgactccccgtttactgcagcagaaaccaggattaaaccagagatttttgcccaactatcagcacgtgctgatgaaaaccatgactttttctcaggaaacggttcgtcgtatgtgatgggagttagtgaagagacaaatgatgatgattggaacttttaatttctttcaaaacagcaactagtatttatagttatccactatgacatccaaagaaaaaatccctacttttaatactgaagaagatgttgaaagttacatttcttttaatgcccaagccaaaatctatgatgattttgcaatcgatttacaagcagttgaaagctatattcaagagcatgtaaaacccaaaactaaggtctttcattccaccaaagaacgccttgattttctgattaagaacgattattatgatgagaagatcatcaacatgtacagttttgaacagtttgaagagatcacccataaagcatattcataccgctttcgttatgctaacttcatgggagcatttaagttctataatgcctatgctttaaagacatttgatggtaagtactacttggaaaactatgaggatagggtggtgatgaatgtattgatgttagctaatggtaacttcaataaggcattaaaactcttaaaacagattatccttaaccgttttcaaccagcaacccctacctttcttaatgctggtagaaagaaacgtggtgaatttgtttcatgttacctgttaaggattgaagataacatggaatcaataggtagagcgataacaactacactacaactatcaaaacgtgatgggggagtagcacttttgctttccaacttacgtgaagcgggagcgcccatcaaaaagatagaaaaccaatcatcagggattatcccaattatgaaattgttagaggactctttttcctattccaaccaacttggacaaagacaaggagcgggagcggtgtatctccattgtcaccatcctgatgttatgcagtttttagatactaaaagggaaaatgctgatgagaagatcagaattaaatcactctccttaggacttgtgattccagatatcaccttccaattagcaaaaaataacgagatgatggcacttttcagtccatatgatatctatcaggagtatggtaaggctttatctgatatctcagtaactgagatgtattatgaattgcttgaaaaccaacgcattaaaaagacctttattagtgctagaaagttctttcaaacaattgctgaactccactttgaaagtggttatccctacatcttgtttgatgatacagttaacaggagaaatgcccacaaaaacaggatagtaatgtctaacctttgcagtgaaattgtccaaccatctttaccttctgaattctattcagaccttacttttaaaaaggtaggtagtgatattagctgtaacttggggagtttaaatattgctagagcaatggaaagtggtagtgagttagctgaattgattcaactagcaattgaatcactggatttagtgtcaaggatcagtagtttagaaaccgctccttccattaaaaaaggtaattcagaaaaccatgcgttgggattaggagcgatgaacttacatggatttttagcaacaaatgctatctattatgattcaaaggaagcggttgattttactaacatctttttttatacagtagcataccatgcgtttagtgcttccaataaattagcattggaactaggtaaatttaaagactttgaaaatactaaatttgctgatggtagttactttgataagtacactaaggtagctagtgacttttgaacatgtaaaacagaaaaagttcaagccctttttgataaataccaagtaaaaattccaactcaggaaaattggaagcaattggtagcaagtatccaaaaagatggacttgcaaactcccatttaatggctattgccccaactggatctatctcatatctctcttcatgtaccccttcacttcaaccagtagtatctcctgttgaagtgagaaaagaagggaagttaggacggatttatgtccctgcttataagcttgataatgataactatcagtactttaaagatggtgcttatgaactgggctttgaacctattattaacatagtagcagcagcccaacaacatgttgatcaagcaatctctttaaccttgtttatgactgataaagctaccaccagagatctcaataaagcttatatttatgcttttaaaaagggttgtagttctatctattatgtcagagtaagacaagatgttttaaaagatagtgaagatcacactattaaaatcaaggattgtgaggtttgttctatctaataaaaataaaccttaacccaacatattaaaaagtgtttatatgcaactaaaaaagccccattttcaaccaaataaaattgctaattgtattgtgatcgggggaatgattgctttaggaaaaaccaccattgctaatacattagctaaccacattcaagctgcaaaagttgtttgtgaattggaaactaatgaccagttggttgaacttttactagcaaagatgtatgaacgtagtgatgaattgctctattcacctttgtttcagctttattttacgcttaatcgctttggtaaataccagaacaattgcaacactatcaatccaaccatttttgatcgttctatctttgaagactggttgtttgctaagcacaacatcattcgtcctgcagtcttttcatactataaccaactgtgaaatagattagcaaaagaactagttaataagcatggggttcctaatttatatgtcattttggatggggattgaaaattatttgaaaaaagactatttatgcgtaaccgcaaagtagagattgataactttactaaaaatcaactttactttcaaaatttacacagggtttacactggatttatggaagcggtttgtaatgattttgggattaattactgtattatagatgcaaaactaccaatagtaactattattaaaatgatccttgaaaaattaaagttacaaaagttagattgaaaatttatctaattaattgcaggaattaacagtttttaaaaaaaagatatttatggatcaaaactttaagttgcttgatcaagcaatcaagcgctttgaaaattttcccaaccaaggtacattgttttatgacattaccccagtattttccaatccccaactatttaattttgtgctaacccaaatggcacagtttattaaagctattaatgcagaagcgatagtatgtcctgaagcgaggggttttatctttgggggagcattagcttctaaaacccaactcccgttagtattggttagaaaagccaataaactcccagggcaattaattagtgctagctatgatttggagtacagaaaacatgctgtattggagatgtcaaccacttcattaatccaagctaataatgctaaaaggtgtgttattgttgatgatgtacttgccactgctggaacagttgctgctattgaccaattacttaaacagttaaatggtgaaactgtgggatattgctttttaattgagctgaaaaaactcaatggtaaagctaagttacaaccaaatgtggttagcaagattttattacattactagttttgattgttagttttgtttcatttgtttaaatttagttatgaattgacaaatagcaattgatggtccaagtagttctggaaagtccagtgttgctaaaaaaatagctgaagaacttgattttttttatttttctagtggaaaaatgtatcgtgcctttgcctatgtaatgcaagtgaatagattaaatattgatctttttttaaaaatcattaatcaaattaactgacgctttgagaaagatgctgtgtattataacaatgctgatattacaacagttattacaacccaatcagttgctaacattgctagtaaaatagctgttgatcctaacattagaaaaattgcagttattaaacaacagaaactagcagaaaataaaaacatagtgatggatggtagagacataggaacagtagttttaaaaaatgctcaattgaagttttttttagatgctaaagttgaaattagagcgcagcgaagattacaagatatgggaatttctctatcaaatgaaaaaaaactaaaggaactaattcaagaattaaagcaacgtgatcaaattgatagttctagaactgcagacccattaaaaaaagcccaggacgctatttatcttgacacttctgaactaagttttgatgcagtagtaaaacaaaccctcaaagaagcaaagaaggtttttaaactttaataaaaactcaataataaacgctttaaaatatttcactttgatggatgaaaaagggattttagttgcaattagtggtggtagttgctcaggaaaaactactgttgctgaaatgatttatcaacttttaagtaaaaaattaaaagttgcgatcatctgtcaagataactattacaagtcctataaaaataagccattattaaaaagaaaaacaataaactttgatcatcctgatgcttttgattgaaaacttttaagatcacacattgaagatcttctaaacggtagtatagttaatgttcctttatatgactacattaactataccagagctaaaaaaacagcaaaaattggtccaattgatgttgttattctagagggtttaatgccatgatttgatgaaaaattatcaagactttctaagctaaaaatatttatagaaacaaatggggaagaacgtttaattagaagaatagaaagagactgacaaaggggaagaaatattgattctattattaaacagtgacgcgaaatagtagcaccaatgtatgaaatatttgtagaaaaaatgaagcgaaatgctgatttaattctgccttgaagtcaacgcagagaagtaagtacaagtgtattggatgtcgcaattgaacacttatttcacaaaactgttgaaaaaaataattagaagtgctttactaagtgcaattagttgtcctagtttagcagtgcaaattctttcacagcaaactattgataaagcttttgaagagaatgactttgtcattttttcaggtggcactggtaatccttatttttccactgacactgcattagctttaagagcagtgcaaacaaaagcagttgctattctgattggaaaaaatggtgttgatggtgtttatacagctgatcctaaaaaagataaaaatgcaacctttttaccaacactcaactatgaccatgccattaaaaatgatttgaaaattatggatattactgcttttactatgtgtaaggaaaataatctgaaaataattatttttaacattaatgctgagaatgcattattagatgcattaaacaaaaaaggtcgctttactataattgaaaataactaatgttgttaattttaaataagtttaagataagctggagataatgaaaacaaaaataagaaaagcagttattcctgctgctgggttgggtgttaggttactaccagcaacaaaagcaattcccaaagagatgttaccattggtaaataaacctactatccaatacatagtagaggaagcagttaaaagtggcattgaacagattcttgtcattgtttcatccaaaaaaacagctatattagatcattttgattatgatctgatcttagaaaatgccttaattcaaaaaaataaattgcaggagcataaagagattgaagatattgctaatttagcacatatcttttttgttagacaaaaaaatcaagatggtttgggagatgcaatcttgtttgctgaatcttttgttggtaatgaagactttgcagtattgttaggtgatgatgttgtttttagtaaagaacctgctttaaaacaatgcttggaagcttattatgaaactaattgtcaaacaatcggtgtacaagaagtagatccttgtcatgttgataagtatggaattatcacccctgaaggtgattacaaaaataaagatcttattaaggttttagcaatgactgaaaaacctaaaccaaaagatgctaaaagtaatttagcaatcttagggcgatatgtactcaaaccatctattttcaaagcacttagaagtgtaccttatggagttggtggtgagttgcaactaactgatggtttaaatttttgtttgaaaaatgaaaacttttatgcaagaaagtttactggtactaggtttgatgttggcacaaagagtggttttattaaagcaaatttatttactgctttaaacaataaagatattagtaaaaaagaagttttagaacttttaaatttagttaaagcttaattagctgttgttttagtagaacgtcaaaaaactaaataagatgggtattaaatctattgttattaatgaacaacagatagaagaaggctgtcaaaaagcagttaattggtgcaatgctaaatttaataataaaaaggtaattgttcttggcattctaaaaggttgcatccctttccttggcaaagtgataagtaaatttagttttgacctccaactagattttatggcagttgcttcttatcatggttcacatgtacaaaaacaaccacctaagattgtgcttgatatgtcccatgaccctaaagataaagacatccttttaatagaagatattgttgatagtggtagatctattaaattagttattgatcttctaaaaacaaggcatgctaaatcaataactttaattagcttaattgaaaagattaaacccaaagcctttgatattaatattgatttttcttgttttaaagtaaaagataattttttggttggctttggtcttgactatgatggtttttatcgtaacctaccttatgttggtgtgtttgaaccagacaatccctaa

SEQ ID NO: 12 - DNA, 4055 bp, M. genitalium
ggaaaagattgatttgtgtggttctagaaataaagcatagatgttaagtgcagggatagttggtttacctaatgttggtaagtcaactttatttagtgctattactaatttgcaagttgaaatagcaaactatccttttgcaactatagaacctaatactggcattgttaatgttagtgatgagagattagataaattagctagcttaattaatcctgaaaagatagtttatacaacctttcgttttgttgatatagcaggtcttgttaaaggcgccagtcaaggtcaaggattgggtaatcaattcttagcaaacatccgtgaagtggatttaatttgtcatgttgtaagatgttttcaagataaaaaaattgttcatgttaacaatacaatagatcctgtttttgattttgaaattattgttaatgaactaatccaagctgattttgaattaataacaaacagaatcggcaagcttaaaagaaaagctgaatcaggtgataaaatcgctaaagaagagtttgtattacttgaaattgttttaaatggattaaaacaaggtcaaatgcccattcagactctaagtgaaagtgaattgaaaacaattaaatcacttaatctattaacagctaaacctattctaatagtagccaatgtatctgagaatgacttattaaaccttgataataatgaagctttaaaaaaattgaatgcttttcttgatcaaaaaaagattcccaaggcaatcacagtttgttctttaattgaaaaagaattaagcggtttgaaattagaacaacgtcaatactttttggatgaacttggcttaaaaaattattcaggtttaaaccgagtaattcaagctgcatatcaaactttaaacctttggtctttttttacttttggtaaaaaagaagttagagcatgaacatttaaaaagggttgaaatgctcctcagtgtgctgggcaaattcattctgattttgaaaaaggatttattaaagttgaagtaattagttgagatcaattgtttgcaatgaaatctttacaagaagctaaaaaacaaggtttgataagattggaaggcaaaaattacttaataaaagatggtgatgtttgtaactttaaatttaacgtcacttaagctttagttcacaaaaaaattttcgaattattcgttgaagatggatgcacattttttgaagagtgcaagtgatttaaaagattgtcctcaagataatatcccagagatttgtttcatgggaagaagtaatgttggtaaatcaagtttgattaatgctttttttaaaaagaaactggctaaaacttcagcaacaccaggtcgaacacaattactaaattactttgaatataaagacaaaagatttgttgatttaccaggttatggctttgctaaaataaacaaaaataagaaagattttattactaatttacttacccaatttttaaattttcgtagtaatctagttggtgttgttttgatagtagatagtggagttgttactgttcaagatcaggaagtggttaaaattattttgcaaacaggattgaactttttgattgttgctaataaatttgacaaactaaatcaaagtgaaagatatttttcgcttaagaatattgcaaattttttaaaagttaattttgataaatgtgtttttgcaagtacaaaaacacatcataatcttgctttagttcacaaaaaaattttcgaattattcgttgaagatgaaagataaggtgatattattttctttgatggttgtgaatttgtaattacagaaatcgtttttaaaataaagaaaaagaaaccaaaataatggctattacagattattgtgagtgtcgctttactgctggaaatggtggtaatggaatcatagcatgaaaaagagaagcacattatgataaaggtggtcctggtggtggtaatggtggtaatggtggtaatgttattttgcaagctgatcataactgcgattcactttttttcttgaagaataaaaaacacctgtttgcagaagatggacaaaatggtaaaccagatttagcacatggtaaaaatggttcagatcttttaattaaagtgccaataggaacaacagttaaaaacttagaaaataatagtgtattggtggattttgttcatgataaacagagttttattttgtgttttggcggaaaaggcggaaaaggaaatgctgcttttaaaagcccaattatgcgtgctcctaatctatatgaaaatggtgataaaggagagattttaaacgttagtttggaagttaaatatttagctaatgtaggaattgttggttttccaaattcaggtaaatcaacactaatctccaaactttcaaatgccaaacctaaaattgcaaactatcgctttactactttaatacccgtacttggtgtagttaaatatcaaaataatagtttagtttttgctgatattcctggtttgattgaaaatgctagtgaaggaagtgggttaggacatgattttttacggcatatagagaggtgtgaaatattaattcatttaatttcacttgatcctgtagataatgatgatccttgcaaagcatatttacaaataatggatgaactttcaaaatattctccccttctagttaaaaaaaagatgcttgttgtagctaataaaattgatgttaatgaaggtgagaaacggtttaaaaaattagaaaaatttcttcaaaaaaaatctatttctgtattaaaaatttctgctttaaagaaagaattaggcaatttacttgatagagtttttgaactttacaataaaacaatttcccaatttggagctaataagtttagtttgccaatggaacttgagaagcattatgtttttcaaaataccaatgaaaataataacgaccctttaaatattgaaaaagatagtttaaacagatgaattgttaattgtaaacgtctaagatattgatttgataaaattccacaaactacgcttgataacattcgtcgcttaggaaataaaattaaagaaatcggtattgaagatcaattaaaatccgttggtgctaaaaaaggtgatattattttctttgatggttgtgaatttgtaattaatgactaatgttttttcaagatgcaattcatgaagagatcttatcagtatgaaagttttaaaagttggtgttttaggacctactaatgcaggaaaatcaacgctaattaattttttacataacgatgatagtctgatggtatcatccatgaataacacaacgcttctttctatttccactgaggtaattaaccaagctaataaaaatattgtttttattgatgtccctggttttactgagaaaaaacattcaaattatgaattaataactaaagaaattcgcaaagcattaagtggaattgatgtattacttttagttgttagaagtgatcagaataacaagatagaatttttaaaaactcaattacaacaattaaagcgctatcaaaatttaacaagaatttttttaattaataaatttcatcaaaaaagtttaagtgaagttaataaagcaattattttagaagagtttaaaccacaaaaaacaattgaaataaacttgcttaagtttgataaaaacctcttttgatcaatttttaaacaagttgaacttaggtataatatctttcgtaaagatatcaattttattgatgcaaacaacgatgactttaagattcttgaaggattacgtgaacaaataattttttattgcaaaaacgaaattccccatatagcaagaattgaaattattgaaaagagtttcaataaagaaaaaaacttgttaaaaattcacctagtaattagtgttccaaaattaagccaaaaaaagattatcattggaaaaaatgcagaaatgatcaaagcaattgggatagcaactcgcaaaaaactattaaatcattttgactgtgatatttttattgatatttttgtaaaaacagaaaaacaaaaattgcctgtttatagttttctttctaaataa

SEQ ID NO: 13 - DNA, 31241 bp, M. genitalium
accaagttcttgaaagattttaaagagattaattacttcaatggaaggaagttgaagttataaattactttatgtttttttgtgtatagttcttggtattttatatggaattgctaaccctatcttattagcacaaggtcttggttttatttttcctattactagtagtaatggtcgtgctgttgactcaatatattcattaatttacccaacaaatttaaatgtattcattaggctcacaattgtgagcgtaactgtttttgtagcttatgcattaatctttgtatttaatgtagcgcaaaactatgtagggattaaactttaccaacaaacatgtgctactttgcgttgaaaggcatatttaaaaatgcagagtatgtcaaccagcttttttgatacgcaaaataatggtgatcttatgagtaggttaactaatgatatgtataacattgataacctattcactcaagctggtggacaagctattcaaagtttgtttaatattttaacaacctcagtattaatatttttattaagcccagttattgcacttatttcactttcaattttagctacattaattactttttcttttgcctttctaaagaaatcaaaaacttcatatagtcaagtacaaaataatttgggtgatatgtctggttatattgaagaggttttaactaatcataaggttgttcatgtcttgaagttgcaagagataatgattaaggattttgatcaatacaacaaatcaatgatcaaaccaactgtaagagggaatacatattcgatctttcttttttcttggtttggttttatatcaaatattacttatctggtttctatatcaattgctactgcttttagtgttaattctattccttcatttggaattagtgttattaactattcattcatgttgtcttacattgcttctttaaggcaaataactttagcattagatcaaatctttaccctttgaaacttagttcaattaggggttgttagtgcagaaagagtatttaaggtattagatcttaatgtagagaaagatactgctactattgacaaattacctgatattaaaggtaatataaggtttgaaaatgtagcatttggttacaataaagataaacctactttaacaggaattaactttagtgttaaacatggagatgtagttgcaatagtaggtcctacaggagctggtaaatcaactattattaatctattgatgaaattctataaaccttttgaaggaaagatttatatggataactttgaaattagtgatgtaactaaaaaagcatgaagagaaaagatttctatagtattacaagattcattcttatttagcggcacaattaaagaaaatattcgtttaggcagacaggatgctactgatgatgagattatcgctgcatgtaaaactgctaatgctcatgatttcatcatgcgtttaccaaaaggatatgacacttatatttccaataaagcagattatctttctgttggtgaaaggcaattattaacaattgccagagcagtaatccgtaatgctccagttttgctcttagatgaagcaactagttcagttgatgtccattcagaaaaattaattcaagaatcaataggaaggttaatgaaaaataaaacttcttttataatttctcatcgtctttcaattattcgtgatgcaacattaataatggttattaatgatggtaaagtacttgaaatgggtaatcatgatcagctgatgaaacaaaatggattttatgcacgtttaaaacaatcttcggttcgttaactttggtaatggtgcagttgcccaagttaatttaaagaagatggctacaagtgaaacaaaagccaagtttttaacagttgcacttacttgaggaataggtgttttatttggtgttttaactgctaatgctatctttaagggtagtggtcatttaaaccctgctatatcattattttatgcaattaatggcagtatcaaatcacctactgcattaatatgacctggttttgtaattgggattttagctcaattcttaggtgcaatgatagctcaaacaacacttaactttttattttgaaaacaactatcatcaaccgatccacaaacagttctagcaatgcattgtacaagtcctagtgtatttaacattactaggaattttctaactgaatttattgcaactttaatattgataggtggagttgttgctgctagtcactttcttcataacaacccaaactctgttcctcctggatttatggggctttgattggttgctgggattattattgcttttggtggcgctacaggctccgcaattaatcctgcaagggatttgggaactagaattgtgtttcaattaactccaattaaaaataaggatgcgaattgaaagtacagctgaattccagtaattgctcctttatctgcaggattagttttatcaataattattgggttttcccctgcacctgttctttaaatactaattaacgttttttattgaaaattaagtatttaaattgaacgaacattctttaattgaaattgaaggtttgaacaagacctttgatgatggttatgtttctataagagacattagcctaaatattaaaaaaggcgaatttattactattttaggcccttctggttgtggtaaaactaccctgttgaggttattagctggatttgaagatcctacttatggcaagatcaaagttaatggtattgacattaaagacatggcaatccataagcgtccttttgcgacagtttttcaagactatgctttattttcccatctaactgtttataaaaacattgcttatggtctgaaggtaatgtgaacaaagttagatgaaattccaaaacttgtaagtgattatcaaaagcaacttgctcttaagcatttaaagctagaaagaaaaatagagcagttacaaaaaaacaattctaatgctcaaagaataaagaaattaaaggaaaaattacaaaaacttttagaaattaacaaacaaaaagttattgagtttgaaaataaagaaaaactacgtagagaagatatttacaagaatttagagcaattaacaaaagaatgggatctactttctcaaaagaaactaaaagaagttgaacaacaaaaacaagcaattgataaaagttttgaaaaagtagagaataaatacaaaaaagatccttggttttttcaacacagtgaaatacgtttaaaacaatatcagaagaaaaaaactgagttgaaagctgatattaaagcaacaaagaacaaagaacaaatccaaaaattaactaaagaacttcaaaccttaaaacaaaaatacgctaataaaaaagcaattgacaaagagtatgacaaattagttgtagcttacaataagaaagactattgaacttcttattgagaaacatacacacttcaacaaaaagaagcttttgaaaaacgttatctttcaagaaaactaactaaagctgaacaaaataaaaaagttagtgatgttattgaaatggttggtttaaaaggtaaagaagatcgtttgcctgatgaattatcagggggaatgaaacaaagagttgctttagcacgttctttagtagtagaacctgaaattcttttattagatgaaccattatctgcacttgatgcaaaggttagaaagaatttacaaaaagaattacaacagattcataaaaaaagtggattgacttttatcttagtaactcatgatcaagaagaggctttagttttatcagatcggatagtggttatgaatgagggaaacatcttacaagttggtaatcctgttgatatttatgactctcctaagactgaatgaattgctaatttcattggtcaagctaacatctttaaaggtacttatttaggagaaaaaaagattcagttacagagtggtgaaatcattcaaactgatgttgataataactatgttgtaggtaagcaatataagatcttaattcgtcctgaagactttgatcttgttcctgaaaataaaggtttttttaatgttcgtgttattgataaaaactacaaaggattgctttgaaagataaccacacaattaaaagataacactattgttgatttggagagtgttaatgaagttgatgtaaataagacctttggtgttttatttgatcctatagatgttcatttaatggaagtttaacaagatgcacattaagaaaaaatactgacttctgctccccttctttttattaatgacaatcttctttattattccaatggcatggattattgttagtggattacaaagtgaagatggggctagtattagtcaaaaatatgaaccacttgttagtggcttaggtttttttaacagtttctgaaccagtttgtggatctcaatagtgactgtaattgttgcattgttgttttcttttcctttttgttactttctctcccaatcaaaaaacaaaatttttaaagcgtttgttatttcaattgcaacagttcctatttgaagtagttttcttattaagttaattggattgaaaaccctacttgatttattaattggactttctttaaacagagttggtgataacaacttaacttttggttcaggatataccttacttggaacaatttatctgtttactccttttatgtttttaccactttataaccacttctgtgttttacctaaaaacttgttgttagctagtcaagatttgggttataactggatttacagctttgtgaaagtagtaattcctttttctaaaaccgcaatgttatcaggaattgctttaacttttttccctgctttaacttcagttgcaattgctcagtttttagataactctaaccaagccgaaacccttggtaactacatatttaccttgggtaataatggttatgatagtgcaattgaaagaggcagagctgctggagcaattattattgctgctttaattacttttgcaatttactttactgttgtttttttgcctaaaattgtccgtattgttcataacaaatgaaaacaacatgaaaaagcattttaagaatttaattaaaaacagttatttctttctgttaataactttaatctatttaccacttttaatagttgtacttgttagtttaaacggttcttcttcaagaggaaatatagtgcttgattttggtaatgttttaaatcctaatcctgattctaaatctgcttatttaagattaggtgaaactgattttgcaacaccactaataaattcaatcattataggtgtgatcactgttttagtgtctgttcctattgctgttatcagtgcgtttgcgcttttaagaacaaggaatgctttaaaaaagacaatctttggaattactaatttttctttagcaactcctgatattattactgctatctctttagtgttgttatttgctaacacttgattaagttttaaccagcagttaggtttttttaccattattacttcccatatctctttttcagtgccttatgcattgattttgatttaccctaaaattcaaaaattgaatcctaatttaattcttgcttctcaagatttaggctattcgcctttaaaaacttttttccatattactctaccttatctaatgccaagtattttttcagcagtactagtagtatttgcaactagttttgatgattatgtaattacctctttagtacaaggatcagtaaaaactatagcaactgaactctattcatttagaaaaggaattaaagcatgggcaatcgcctttgggtctattctcatattgattagtgtcttaggagtctgtttaataaccctgcaaaagtatttaagggaaaaaagaaaggaaataatcaaaataagacaatgaaaaaacagttaaaatattgctttttctcactttttgttagtctctcatcaatattgagtagttgtggttcaacaacatttgtactagctaactttgaatcttatatttcgcccttattgctagaaagagtacaagaaaaacatcccttaactttcttgacttatcctagtaatgaaaaactaattaatggttttgctaacaacacttattcagtagcagtagcatctacttatgcagttagtgaattgatagaaagggatctattatcaccaatagattgaagtcagtttaatctgaaaaaaagtagtagttcaagtgataaagtaaataatgccagtgatgcaaaggatttgtttattgattcaattaaagagatcagtcaacaaaccaaagatagtaaaaacaatgaattactgcattgagcagttccttattttcttcaaaacttagtgtttgtttatcgtggtgaaaaaattagtgaacttgaacaggaaaatgtttcatgaactgatgtaattaaagcaattgtgaaacacaaagatcgctttaatgacaataggttagttttcattgatgatgctagaacgatcttttcacttgctaacatcgttaatactaacaacaattcagctgatgttaatccaaaggaagatggaattggttatttcactaatgtctatgaaagctttcaaagacttggattaacaaaatctaatttagatagtatctttgttaattctgattccaatattgtgatcaatgaattggcaagtggtagaagacaaggaggaattgtttacaatggtgatgcagtgtatgctgcattgggcggtgatttacgtgatgaattgagtgaagaacagattcctgatgggaacaactttcacattgtgcaacccaaaatttccccagttgctttagatcttttggttatcaataaacaacaatctaattttcaaaaagaagcacatgagatcatttttgatcttgctttggatggtgctgatcaaactaaagaacagttaattaaaactgatgaagaattgggtactgatgatgaagacttttacttaaaaggagcgatgcaaaactttagttatgtgaactatgtttcaccattaaaagtaatatctgatccaagtactggaatagtcagttccaaaaagaataatgctgaaatgaaaagtaaacaaatgtcaactgatcaaatgactagtgaaaaagaatttgattattacactgaaacacttaaagcattattagagaaagaagatagtgcagaattaaatgaaaatgaaaaaaaactagttgaaaccattaagaaagcttacactattgaaaaagatagttcaattcggtgaaaccaattggtcgaaaaaccaatttctcccttacaacgtagtaatttatcgttatcttgattagactttaaattacactggtgataatgaacaaaattaagattgacaaggaaatcaaaaactcctaatggattttttctctttaaacaaaatcataaaacccaaccagaaattcactagtaatgaagctgaatttctacagatagctactgattatttggaggaaagtcaaaactatcttcaaaagggtttaaagcaattaaaaaaagaatataaaagatccattatttataaccctaaccttgaatataaacgctttgttaaatgaaaagaaaatttcactgaaacatttgaaagttattatgacaggttttttattaccaaatacaaccattattcactaagcttactttttagctttattaatgaacagattgaaacagttattgctagttacaactcatttctaaatgagcataataagttagcttttaataaagttagttttagttttgaaaagaaactttttgaagctacacaacagtttaataacttagaaaaaaacactgctattagtgatgatttaccgctccagtttaaagttagaacaactcaactaaaagcccaaagagaaagggaattgaagaacttgttgaataaaatcaagcttaaaaatttaagtgaaaaaaaacaagaaattttgttaaataactggtttaatagcaacgaacgtttgtttttaaaaaatgaagtgaaaaaggttaattgactaaactcgccaagacaaaaacaacaagcagctcaaattgatgatcaaaacattattgaattgaaaaatgtgtataaatacatcactaatggcattactacaaatgcagttcttaaaggagttgatcttgccattaaaagtcatgattttattgtgattttaggcccttcaggatctggtaaaaccacattactaaacattatttcagggatggatagagcttctagtggtagtgttattgtcaatggttataacatgatttgtttaaatgatagaaagctcactaaattccgtcaaaagtatgttggttacatctttcaacaatacggtttattacctaatttaacagttagagaaaacattgagataggagcaaatcttcaaccagatcctagtaaaaggatcagcattgatgcacttttagaagcggttgggatggatagtttgcaaaagaagcttcctaatgaattgagtggtgggcaacagcaacgtgtttccattgcaagagcttttgctaaaaaccccttattaatttttggtgatgaacctactggggcacttgatcttgagatgacccaaattgttttaaaacagtttttagcaattaaaaagcgttatcaaacgacaatgattattgttacccacaacaatttaattgctaacttagctgatttagttatctatgtagcagatggaaaaataaaatcactacacaggaacttaaatcctaaacaggttgaagagatccattgattaaacattaaccgtcaacaacaagaaactttatagagcagtgaacagttgaacaggacttagtgaacaagcggcaattaaaagtcgtcaagaacatggtgctaattttcttcctgagaaaaaagctacccctttttggttgttatttcttcaacaatttaaaagtttagttgttattcttttactgctagctagcttgttatcgtttgtagttgctattgtcagtggtttgagaagtaactgaaactttaaccatgatctgattattgaatgggttcaaccttttattatcttattaactgtttttgccaattcactaattggttctatccaggaatttaaagcccagaaatctgctagtgctttaaagtccttgacaaagtctttcacaagggtttttaggaatggtgaattaattagcattaatgttagtgaagttgttgtaggagatattatttttgttgatgcaggagatattatccctgctgatggcaaattactacaggttaataacttacgttgtttggaaagctttttaactggtgaatcaactccagttgataagactattgatagcaatgaaaaagctactattcttgaacagacaaacttagttttttcaggggcacaagtagtttatggtagtggcgtttttcaagtggaagcagttgggattaaaacccaagttggaaaaattgctaaaactgttgatgatagtgtaactaaactctcacccttacaacaaaaactagagaagataggaaagtgatttagttggtttgggcttggtctttttgctgtagtttttcttgtccaaactgctttattaggatttgataatttcactaataactgatcaatagctttaattggtgctattgcgcttgttgttgcaattatccctgaagggcttgttacttttattaatgtgatctttgcattaagtgtgcagaaactaactaagcaaaaagccattattaagtatttatcagtaattgaaacacttggatcagtacaaattatctgtactgataaaactggtactttaacccaaaaccagatgaaagttgtcgatcacttctgttttaattcaacaacccaaactgatctagcaagagcattgtgcttgtgtaataatgcttctatttccaaagatgctaataaaacaggtgatcctactgaaattgctctcttggaatgaaaagatcgcagtcaattagatttaaaaacctattacagggtttatgaaaaagcctttgattcaatcagaaaacttatgacagttgttgttcaaaaagacaaccgcttcattgtgattgttaaaggtgctcctgatgtgttattaccattatgtaataacgttcaaaatgaagtaaagaacattgaaaacttacttgatcaaagtgctggtcaaggcttgcgtaccttagcagttgctttaaaggttttatataagtttgatcaaaacgatcagaagcaaattgatgaacttgaaaacaaccttgaattccttgggtttgttagtttacaagacccaccaagaaaagaaagtaaggaagcgattttagcgtgcaagaaagctaatataaccccaataatgattacaggggatcatcttaaaactgcaactgtaattgctaaagagttaggcattttaactttagataatcaagcagttttaggtagcgaactagatgaaaagaagatcttggattacagggtatttgctagagtaactccccaacaaaaattagccattgttagtgcttgaaaagaagcgggatttacagttagtgttactggtgatggggtgaatgacgcacctgcattaatcaagagtgatgtagggtgttgtatggggattactggggttgatattgcaaaagatgctagtgatctgattattagtgatgataatttcgctactatagtaaatggtattgaggagggtagaaaaacttttttaacttgtaaacgagttttattaaacctgtttttaacttcaattgcaggaacagttgtagttttattaggactattcatcttaggacaagtttttaaaactaatttattacaacaaggtcatgactttcaggtgtttagtcctacccaactgctaattattaacttgtttgttcatggttttcctgctgttgcattagcagtacaacctgttaaagaaaaattgatggtaggtagtttttctactaaaaatctgttttacaaccgccagggatttgatttaatctgacaatcactattcttaagctttttaactttattgttctatagcttaggaattatatatgcaattaataaccgtgatttacaaactagcggggatctaattaatcgtgctggatcaacgtgcggtttttttattttgggtgctagtgctgctttaaactcattaaacctaatggtagataaaccattgcttatgacaaacccttggttttttaagttagtttgaataggttcacttgcttctatactggtatttttattgatcatctttatcaaccctttagggttagtgtttaatgtcttgcaagatttaactaatcacccagttttaataagctatagttttgggggagttattttgtatatggggatgaatgaagttgttaaacttattagattaggttatggcaatatttaacctgaaaaaaacaggttctttttttatttctatttagtaaatgttcaagtatattcttaaacgattaggactagcagtagttgcgatgtttatcgtaatgtctatagtcttctttttagtgaacgctactggtaatgttcccttgtcagccacttctgcaagagatattgctgcagtgcaagcacaactacaagagtttgggtttaatgaccctattatagttaggtattttcgctattgagctaagctattttcctttcaagctgatgctttaggaatttattatgcaaaccctaaccaaacaattggtgagattgtgtttgcaagagtaccaaataccttatatgtggttttaatctcttttttaattggttcattgctagggatctttttagggatggtttcaggattgaatagagggaagtttttagatgcagcaattaatgtgttggtagttttatttgtatctattccttcatttgtagtgggattagggttacttaaactagcaggatttttaaatctaccaccacggtttattaactttgatgatgctttttttagctttgatcgtttcttgcttgcatcaattatcccgatcctttcattggtcttctattcatcagctgcttttacatacaggattagaaatgaggtggtggaagtgatgaatcaagactatattaaaactgcaaaaagtaagggacttgggatgtttgctgtagctaggtatcatatctttagaaactcgattattccttctattcccttgtttgtatttggaatctcaggtgctttttcaggtggatttattattgagtctttgtttggagtacaaggggtatctaggatcttaattgattcagtgcaagttaatgaaactaacatggtaatgtttaatatcttgtttatccaagggattcccttattagcaagtgtctttattgaatttatctatgttttagttgatcctagaattaggattgcaaatagttctaatgttagcttattaactaagttaaagttcttaagttcaagacaccaatggttaatgaagtgaaacaagattaacagtgataatgcccaaaatattgtgtttaactcgccactgcaccaccagctattagaactcaatgcaattgattacaaaacaaaaacagttcaactaacaactgaacaaaaaactgctctcaatatcagtgcaactgctaactttatcttacttggtaacaagtgtttaaaactcaaaacaatccatggatagaaataaaagttttgaccctaacttatttaaaagggttgatatcaacttattaaagcgaaatgatcagcttattggtaaaccaactaccaattcaatagaaattatcaagcgcttgtttcaaaacaagtgggccatcttattttttcttttaatagttgttattgtgctattagcaattattgtgcctttaacttcccctttttcagcagtaactcctgtttcaaccaatgccttagcacaaaatctaccaccacggtacttatggcataaaccaggtgacattttagttcataagattacagcaagatcaattgctgaaatctctcaagctagtggagttttagtaggaacattacctagtgcaaatagtaatcccttagcaactaatgtccagtatgatattgctccttttcaactccaagaattgcgtaattatttccctttattggggactaatggacttgggattgatatttgaaccttgttgtgagcttctgttgccaagtcattgtgaattgcagtagtagtagcaattatagcaatggtgtttggaaccatttatggagcggttgctggaagctttgttggacatatggctgataacattatgagtaggatcattgagattattgatatagtcccttctattctttgaattattgtcttaggagctacattccgctttggtggggttaaacaatttgatgatagtgttgtaatctttactttaatctttgtgttttgaacatgacctgctactacaaccagaatttacattttgaaaaacaaagatacagagtacatccaagcagctaagaccctaggggcacaccaaatcagaattatctttgttcatatgttacctgttgtatttgggagattagctgttgtgtttgttagtttaatcccagcagttattggttatgaagcttccttagttttccttgggttaaaaccagctactgatattggcttaggggcacttttaaaccaagtaacttcaagtgataatgtagctttaatcttaagttcgattgttagctttgcagttttaacagtagcagctagaacatttgctaatgctttaaatgatgcaattgaccctagggttgtaaaacgataaaatggcacttaaaagaagtaatttctttgttgataaagaccaacaactaaaggataatttgatcttagacatcactgatttacatgttaactttaaggttaaagatgggatcttacatgctgttagagggattgatcttaaggtagagaggggtagtattgtagggattgtaggtgaatcaggcagcggtaaatcagtgagtgttaaatcaattattggttttaatgacaatgcacaaactaaagccaaactgatgaactttaaaaacgttgatattaccaaactaaagaaacaccagtggaagtattatagagggacatatgtctcttatatttcccaagacccattgttttctctaaacccaacaatgacgataggaaaacaagtaaaagaagcgatttatgtggcttcaaaaagaaggtatttccaagctaaatcagacttaaaatttgctttatcaaataaggagattgacaaaaaaacttataaaagtaaactaaaagagatcaaacaaacctaccaacaaaaaataaaacctatcaatgtagagaaaaaaaccttagagatcctgcagttcattggtattaatgatgccaagaaacgtttaaaggcattcccaagtgagttttcaggagggatgagacagagaattgtgattgctattgcagtagcaactgaacctgatttaattattgctgatgaacctactactgcacttgatgtaactattcaagctaaggtattaactttaattaaacaactccgtgatctacttaatatcactattatctttattagtcacaatatctctttaattgctaatttctgtgactttgtttatgttatgtatgcagggaaaattgtagaacagggtctggttgaagagatctttacaaatccactccatccctatacatgggcattgatttcttcaattcctgaacagaaagataaaaacaaaccactaacttctatccctggagttattcctaacatgttaaccccaccaaagggtgatgctttcgctagtagaaaccaatatgctctagcaattgactttgaataccatccacccttttttgaagttactaaaacccataaagcagcaacttgattgctgcatccccaagcccctaaagttgaaccacctcaagcggttattgataacattaccttaaccaaaaaagcactgcaatttaaagatcaataatggaaaaccaaaacacaaaaaaaccacttgttaatgttaaggctttgagcatgatgttcaaggtcagaggaactctttttaaagcccttgatgaaattggttttactgttaatgaaggggacttctttggggttattggtgagagtggtagtggtaaatcaaccacgggaaaatgtttgattagattaaacattcctagtggtggaaagattgagattgccaaccacttactctcaggaaaaaaacttactaaagagaataaccagtggttaaaacaaaacatccaaatggtgtttcaagacccttattcatctattaaccctactaaaaatgtgctaactgtgatttcagaaccgctggtaattagtaaaactgtttttggggaaacaaaacaatacttaaagagtttgcaaaagctctcttttaaagtaaagaaaacattgttaaggaatgatattgaacttgaaaccaagtttcacaataacttttttaaaaccgttattaagcaaattaatgaatcattgtttaactttgaagatcttgattacaaggatttaaaaccatcacatttaaggcaaagaatcataaatgaaacagataaattcattgaaaaaattagaagtgagtttgcccttttttatgatttttatgctaaccaatcagtacccttgcaaaaggcattagatgatgcgaattcctctttaacaccatctagtgttattgagttaaaaaaccagttaaaagcattacaaaaacaagcaaagatttcaaaggcagcatgggatattttacaagccctaaagcaaaaccaaaaggagttgaaagattatgaaaattatgtccattttgaactccaaaaaaagccacgaatctatcttaatacctgacttttaacaaccaaaagctacattaaagattccaagcaaaacatgcagcttactgatgatatctttgctttttcatataacagtatggttgacaagaaaagaaacttggttttaattctttctaaatactataagctgttaccttatttctatgaccaatcagtatttgataatgctgatcaatttgatgaaattgctaaccttatcttttttgatttagttgaaacattgcttggtgtaactagtttatttaatgatgcattagcagctgataaagtcccactaattaagtttgctaagttcttaaataagttatgtgacttgcgctttttaaccttaaaaaagagctttaaaaaaacaagagtaagttgtagctttagttttaacagtgaacctgaaatcttgtttgccaacagctgctatgatttgcaacaaatgcctcaaatcattaaacccttttgagagaagctttttaatgaacagaactaccaaaagattattgattcagtttcaagactgaatgtaatgattgcaaattacattaccaaagcttttgaaattaaaaaaactattgatgaaaaactaagggagtttaaacaacaaaatttagctttaaaaaaagcttattcagctaacaagaaaagtgaggcaaacaaagcttccattaatgagttaaaagtcaatttaaaaacacttaaaaaacagcttaaacaagagaaaaatactactaaaaaacaatcaaaaaaggaattaaaaccacttttaaaagaacaccatactgctttaaaactccatgatgagtttaaccatgatttacgcaagtggttcaaaaaacttaactttatggttaagaaatacaaccgactggaaaacagccagaaaaagttttgtttagttaaaaagttaaaagcgcttttcaaaaaacaggatgaaacactgcaaagtgaattaagaccaaaactaaaaacatttggtgtaattaactttgagtacaaacgtgcagtcaaagagtccaatgtctttcgattggtgcattttgctaaaaatatctttaaaccattcttgttttttaacctcaccaagatttttatgagaaataaggtctatgaagcacttgatagtgttggtttaaaaagagaacatgcttacagatacccccatgaattttcaggcggacaaagacaaagaattgctattgcccgtgctttaatcactaaacccaaactgattattgcagatgaattgattagtgcacttgatgtttctatccaagcccaagttattaacatcttgaaagacttggctaaaaaacacaacttaactgtgcttttcattgcccatgatttatcaatggtgcaaactgtttgtaaccgtttgatcattatgcataggggcaagattgttgaacggggcagtgtggatgagatcttttcaaatccagttcatccctacacccgttccctaataaaagcatctcctaagttaagcaaaatcaatgttgatctcgcttcttttgatgaaaacttcacttatgatagtgattattcactaaccaatatgcccttttatattaaagttcctaacagtgaagaacatgaactttactgtactcaaaagcaatttgatagttgaatcaaagaggctacgccgataaattaaagaatttttataatgacgcttattaagtggtgtttaattaatggaaaaagttgccttcaaaatggagcatatctccaaaagttttgacaatggcaaaattaaggctaatgttgatgttagcttagttgtttatgaaaatactgtccacaccattttgggggagaatggtgcaggaaaatcaaccctgacttcgattttatttggtttatataaacctgatagtggcaagatctttattggtgaaaagcaagtaaattttaaatcttctaaagatgcagtaaaacataaaatcggaatggtgcaccagcactttaagttaatagaaaactacacggttttagataacatcattctagggaatgaaagtaggtttgggtttttacctttaattaatcgtaaagtaagtgaagcaaagattaaaaccatcatggaaaaatatggaatctttgttgatcttaaacaaaaagttagtaacttaacagtaggtcagcaacaacgggttgagatcctaaaggttttatttcgtgatagtaatatccttatctttgatgaacccactgcagttttaagtgatcttgaaattcaaaactttctcaagattattgctaactttaaaaagctaggaaaaacaattgttttaatctctcataaattaaatgaaattaaacaagttgctgatacagctactgtcttaagacttggcaaggtagttggtagttttgatgttaaaacaacaccagttgataagattgcgcttttaatgatgggcaaagagttaaaacaaactaaaaacaccacagattttgttgctaaagatgaacctgttttaaaagttcaaaacctgaatttgtttctcaataaatctttagcatacaagttcttagtgaggtgcaataacatccataaagcccaacaaattaagaaaaataaaccattaaaagacttatggataattagttttttaaataaactaaccaccagtaacaaaacccctaaattagtaaaaggcttgattaataagttaggactttcctatcaagaaaatacagatgaaaccattagttttgctatccataagggagaaatttttgctattgctggggttgagggtaatggtcaaagtcagcttgttaatttaatttgtggaattgaaaaagctgctagtaataagttaatttttaacaatattgatatctcaagatgatcaattagaaaacggattaatgctgggattagttttgttttggaagatagacataaatatggcttgatcttagatcaaaccgtgaggtttaatacggttaataaccagattaataaccgtccttttagtagttgaaactttttaaaaccaatggagattgctctttatagcaacactattattaaaaagtttgatgttaggggcagtgctgagggtagtgctgttgtaagaagactttcaggtggtaatcaacagaaactaattattggtcgagaaatgaccaaacaaaatgaccttttggtgttagcacaagtaaccagaggccttgatattggtgctattgcttttatccatgaaaacatcttattagctaaagctaataataaagctatcttattggtttcatatgaacttgatgagatcttagcacttgctgatacagtggctgttatcaataaggggagaatagttggtatgggaaaaagagatttaatggatcgccaatcgataggtagattaataatgcaataaaagactatgacaatgtggcaatttaaaagttactttaaacaccacctggtgttttgaaaagaccgatttttacatagctctgagaaacaaatgcaaagaagaagtatcctctcttcagtggttttgataatcctctcttttcttatatcgtttttactgattatttcaattcctggaggtagaggtgcgagcttctttgcactgtttactaagttatttttagataacactaatactgaaaatttcttaagacagattgctatttatatcctagctggattagcatttagtttctgtatgagtgttggtattttcaacattggtatctcagggcagatgatggctggagccatctttgggtttttaatgattctcaaggtgtttccaagttcatttcgacctggttttggaggtcagattattactgtattattgatggtaataggtagtgttagtgtggcagttgttgttgcaactttaaagatttttttcaaggttaatgaagttgtaagtgcaattatgttgaactgaattgtagtgcttattagtgcttatttagtagagacttacattaaagataatagtgggggtacagcccaattcttttccttaccactccctgatgaatttgctttatataacttctctcctttaacaaaaaagtttggttgattagcttcacttattattgctttcattagtgttattattgtggcagtagtattaaaatacacagtttttggacacaaattaaagtcaattggcagtagtgtatttggttctcaggcaatgggttttaatgttagaaaataccagttcttatcgtttattatctcaggaattttatcaggactattagcaacggttgtttacactgcatcaactgaaaaagtattgacatttaacaatgttggggatagtgctatttcagcagtaccagctactggttttgatgggattgcgattggtttaattgctttaaataacccctttaggattgttattgtttctgttcttattgcttttgttaacattggggcaagacctgctaatttaaaccctaatactgctagtttagttttaggaatcatgatgtattttgctgcactttataacctaatggtttactttaaaccatgaagatacctagtgaagctgaacattggaaagataaatctcaccacatatgaaacatatgaaaacaaactagctgctaacctagagtgactaagtttccaacgcttcttgtcaaaacagaaaaaaaagaatgacaaaactaaatttaattggtttgatactagtttatttgaacaatatgcaaaaaacaaacaagaaattgttcaagaataccatcacaattgtgcaactaatttaattgcttggtgattgaatgcaatccaaagtggcaatattaaaccttcaactacttttaagttggaatttgttaattttaaacaccaacagaagtttgtattaaattggtttaaaaatgaaagtgaatcactgcgtgatttccaatcacagtttgagagaatcaataagttagtggaaagggagtttgttaagtaaaaactaggtaggataacccaaagaaaataattaaaatattgtgaaaaaaaagatagtcccaattaaccctttaaaagcagatgagattttagcagttagtcacttatcatgtgtttttaacagtaaaactaacaatcccattaaggtgattgatgatttttcctatacctttcaaaagaaccaaatttactgtattattggtgatagtggcagtggtaaatcaacccttgttaaccacttcaatgggttgataaaacccaaccaaggtgatatttgggttaaagatatctatattggtgctaaacaacgcaagattaagaactttaaaaaactgcgaaaaactatctcaattgttttccagtttcctgagtaccaattgtttaaagataccgtggaaaaagacattatgtttggtccagtagcattaggtcaatccaagtatgatgcgcgccaaaaagcggcttattatctggagatgatggggttaaaatacccttttttagaacgtaatccctttgaattgagtggggggcagaaaagaagggtagcgattgctggtatacttgcaattgaaccagaaattctaatctttgatgaaccaactgctgggcttgatcctgaaggggaaagggagatgatgcagttaattaaaactgccaaacaacaacaaagaacggtatttatgatcacccaccagatggaaaatgtccttgaggtggctgatgtggttttggttttagctaagggtaaactagtaaaagctgctagtccatatgaagtgtttatggaccaaactttccttgaaaaaacaacgattgttctcccccctgtgatccaagtgatcaaagatctaattgcgattaatgctcactttaataagttaattgagttgcaaccaaagaacctagaacagcttgcatcagcaattaacaagactatagcaaaccatggataaatgtaaatttagctaacaagcttaactggttgtttttgagatggctaacaataagagtgcaattgagttgaaaaacatcgttgttgattttggtgaatcagttgcgattgacaacattaaccttagtgttgaaaaacaccaactagttagcttacttggtcctagtggttgtggtaaaaccactacacttgcagttattgcaggacttattaaaccaactagtggtcaggtgttatttaatggttatgatgtcaccaaaaaaccaccccaagaacgtaaactagggctagtttttcaaaactatgcactttatccgcacatgaatgtgtttgaaaacattgttttccccctctacagtgataactcgtgaaaacaagcagttttggaaaaaaacagtgttgcaaaccatgagattaactgtttgttacttactagcaacggtgcatcagttcaagagattgatcagctcaataagttatttcatgatagtattgaaaaacccaaacagatccaataccaaattaatgaccttaatgttagtgtttttaaaaacttaaatgaactaactgcaaaccttaagttaataccaagtaagcaccagtttgctattaccaatctcaacaaacaaactctaaaacagattaatgaactggaagctgagtttaaaacaaagtgaaagttacaaaaacaaaccccaattaagagtggggttgaacacaatgccaaactccaagcaattaaacaacactttagttatgaaaaacaacggttaaaaaaacactatttcaaaactaaagtggaactaaaacaaacccttgttgaaaaccttaagttagttaaaaaagcgattagtgaacaaactaagttaattaaacagagtagtgattacactaagttaaagcaattaaaacggttaattaaagttgaacctaaccaactcaaaaaacaatataaggtttttctcaatcagttaattaaaaactattcacttaaaactgataagttaactgatactcaacttaatgaaattgaacagattaaaaccagaattgtttcaataaaacagtttatcaacaaaactgcacttgaagtagctaacaaactagcgattaccaagattttaaccaaacgccctgataagatttctggtggacaacaacaacgcgtagcaattgctagagcaattgtcagaagacctaaactattgttaatggatgaaccactctctaacttagatgcaaagctaagggtacagacaagacagtggatcagacagtttcaacaggagttacaaattaccactgtttttgtcacccatgaccaggaagaagcgatgagtattagtgatgtcattgtttgtatgtcaactggaaaagtgcagcaaatcggcacacccagtgaactttatttaaaacctgctaatgagtttgttgcgcgctttttaggcacccctgagatgaacatcattgaatgtagtgtcaaaaacaaccagttgttttgaaacaaccatctgttagttactgagagttttaagcttaatgtagagaaactcttagttgggtttaggtatgaacaactagtggtcactactaacaaaagtagtttgcaagctaaactaattaacattgaaaacttaggtaaacacttagttgctaccattagtttgtttgataccaccttatcaatgcgcttagaattgaatagccacttaaaagtaggtgatagtttaaatttcattattaaagctaacaacctccatttttttgatattgatacaaaacaacggattgagatttaacttaagataattgaaacattaaataagataattacaagctatgaatcaagctagtgcaattgccattttggtcatttttagcctagcttctggttatctgttaggttcaattatttttgctgatattttcagcaaaatactcaagaaaaacgtcagggaatttggttcaaaaaacccaggagctactaactcaatgcgtgtttttggcttaaaaattggttttttggtggctatttttgatgcatttaaaggtttttttgcttttttattaacctgaattttattccgttttggtttacaaggttatttaacagaaaaagtgtatcaaagcacctattttttaagttatttaagttgttttgcagctacaataggtcatatctttccgctgtattttaagtttaagggtggtaaggcaattgctactactggtggatctttacttgcaatatctttatgatgatttttaatctgtcttttaatttggataatgattactttaataactaagtatgtttctttagcaagtcttattacattctttgtgttagctgtaatcatcttaataccctgacttgattatttatacttctttaacagtgatcctctaaagtcgattacttatcaaaatgaatggtatatcattttatttttttgcttgtgatattgacctttaactgtggttgttttctgattacaccgtgcaaacataatcagaattttacatggtaaggaaagcaagattactcaactaaattaatgatgtttgtaaaaaagcgatctttgattactttagttaattggtagttgatgcaaactttcattattacttcccctgttttcaatccgtattttaatgcagctttagaggagtgattgctaactgaatttagaaaaaatgagttagttaaggtcatctacttttggcagaacgctaacactattgtggtgggaagaaaccaaaatacttatgctgaggttaacttaaaggagttggaaagtgataaggttaacttgtttagacgtttttcaggcgggggagcggtgtttcatgaccttggtaacatctgtttttctattattttgccaagaacaggtaaagtgatggaaaatgcttatgaacaaactacaagaaatgtggtgaagttcttaaatagcttaaatgtacctgctgtatttcatggtcgtaatgaccttgagattaataacaagaagttttctgggttagctgaatatatcgctaaagacaggttattagtccatggaacattattgtttgacactgacttttctaagttagcaaagtatttaaatgttgataagaccaagatagcaagtaagggtgttgacagtgttgctaagcgcgttgttaatgtaaaggagtatttaccaaattgaacaacagcaaaatttttagaagagatgattaattttttcactgttactgaaaaagcagaaacaattgttttaactaaagatgcactagcaaaggttgaaaaaagagcaaaagaacactttcaatcatgggagtgaaactttggtaaaacttatgaatacaactttaaaaacaagcgttattttaataatgctggtttatttgagtgcaatgttcaagtagagaaaggaacagttgttgatattaagttttatggggactttttaagtgttgttgatatcaccccagtaacaaaaaaactaattggtcagaagtacgattataaaacctttgaaaaactcttcaatgaacttgatcattttagtgattactttggcagtttaaaacctgagcaactcttaggagtaatatttgataacaagtaacttaaataataaatctttataatttttactagatttactaatgcaaacgcatgaaattcttttaaaaattaaagaaattgctaaatcaaaaaactttaatcttaatttagatgaaaagacaataaatcaaccacttcgtgagttgaaaatcgattcacttgatatgtttagtattgttgttagtctagaaaatgaatttgggattagttttgatgatgaaaagttaatgaatctaaaaaatcttgctgacttggttttagaagttaaaaaccttttagcaaaaaaaggggtatagatttgatagcgttcttttaatgacaagaagacaaaaccagatggtaaaactcaccacttggttaaaaaaaattggctgaggtgagactattacccaacgaattttttgtttttatatctattgcatcttgtttggaagtttgctgttatttttgccaattgcactccaagataactaccaaaaagtggttagttatggaattgattgacagggaaaaagatttgaacagaaaactgattacaactttttagatgcattatttttatcaaccagtgcttttagtgatacaggactttctactgttgttgtatcaaaaacatacagtatctttggccagatagttttagcagtattactccagttaggagggattggatttgttgttattgcttttttagcatgacgattgtttaactttcacaagaaggaacaatacagtttttatgaaaagttaatgttgcaatcagaacgaggtggttctaagctaggtaatactagtgagatgatcttagtatctatcatctttctttttatcgttgaactaatttatggatttttatatggtattttgttttatttcatcccaggctttgaacctgctaacttgtttgcagatcatgcaaaagtttcaactcaattaaaagctttagtagttgattcaaaccaaacaatagcagcttttaatgatattaataaggcttttcaagcaggttttttccattccttatcagcagttaataatgctgggatagatctgatagggggtagttcttttgttccttatagaaatggacttggtattattattcagtggttaactattagccaaattatctttgggggaattggttatccttgtttgtttgatggctttgaagccattaaaaaaaagattaagtatggtagacacacaaaacaccaatttagtctatttaccaagttgacagtaattactaatatcgttgtaatcctgctttttttcaccttacttttaatggtggaatttattgctagtgatagtttaactaacactattgttaattttagtgatgaaaaaaagagtttaataaatacccaattgcaatcacaatctaaccaagcaatccatgcgtcagtttttggtaataaccctaatgcaagtagggtaatgcagctcttttttatggttatttcatcccgttcagcaggttttagtgttttccctgttgctagtgagattcaaactacaaaaataattattgcattggcaatgtttattggtgctagtccctcttctactgctggggggattagaacaactacgctagcagtaatctttttagctctagttgctaagtttaaaggtcaaaaggaagtaaaagcatttaagcgttcaatcgatcaaactacagtaatagatgcttttttagtactaatcataagcttaattgcagttttactaacagctgttcttctacctttaagtatggaacaaccagttagtttcattgatgctttatttgaaacaactagtgcttttggaacagttggactttcaagtggagctactgttaacattgctttagatccaaatagaaatacctttaatttccttgctttatgtctattaatggttatgggacaggttggtgtgtccagttctgtgctaacttttgttagaaaacatcccaaagcaaatagttattcatatcctaaggaagctgttaaaattggctagacacaaagttgaccaagttgctaataatttttaatcttacatgccacgtaagcatctaattgctaatcaaactaataaaaaacaacaaacaagtgcaaaacaacttcaaaaactagcaaaaagaatagcttcagctgttaaaaaaggtggaactaatatccagtcaaatccacatctaaaagttgcagttgatcttgctttagctaagggtctaagcatggattcaattaaaagaaatatccatggtagtgaaaaagatacaactaaaattagcgagttttgttatgagatttttggaccaaatggtgttggaattattgtgtttggactaactgataatcctaaccgtttactcagtagtttaaacggttatttagctaaactaaaaggacaattagccaagccaaatagcgtcaagattaattttcaagaagaagggattatctttgttaataaaaataactatttgaaagatgatctaattgaattattaattttggacaatattaacttaattgatgttgattatgatgaagagtgttttgaaattagcttgcattcaaatagttattttcatgcaaaggagctgttgaaaaaaaacaatttttcaatagtagatagtgaaattaaattggtacctcttttaactgttgatttagatagaaatcagcaaactttattatcacgttttctcaatgcttgtgaggaagatgatgacattcagtttgttgttcataatgccaacccatgggaagagtagatgactttgcttttagtaatcctatctttggagctttttagtgcaaaagaagattaaaaaacgtttaaaaaaagagaatcttttaagaatcttttcaaagactttagcattcttgtttttagttttatttataagtttttttgtttttcttttaacagaagcaacaaaaattggacctgattttgcaaagtccttgtttaatcttgaatttaatttaggtaataaacaggcaggaatttgattccccttattggtaagttttattgtatcaataggagctttaattattgctagttatataggggttagaacttcatttttccttgtttatcgatgcaaaccaaaaataagaaaaaaactttcacttattattgatatcctttcaggaataccatctgtaatttttggattatttgcatcacaaatattaagcattttctttcgggatatcttgaaattaccgccgctttcacttttaaatgtgatagctatgctttcttttatgatcattcctattgttatttcattaacaacaaatacattaacttatgtaaataacgatctaattagtgttgttgtttccttaggggaaaataaaacaagtgcgatctacaaaattattaaaaaagaaattaaaccacaattaacagttattttgaccttagcctttgcgagagcaattagtgaaacaatggctgttaactttgttttgcagagtgttaactatcaagaggtaattaacaacaatcgtttttttacttctgatttaaaaacactgggatcagttatttccacttttattttttcagaaaatggagatgaacagattaatggtgttttatatatctttggaatcataattttgatattagtttcattgttaaatttctttgccatttgatcagctaatccaaaaacactggaacgctatccctttttaaaaaagattagtaattttatttatcaagttgtgtgattcattcccaataatattagtgcactttttgttgatttaacatcaacaagacaaagtgttaaaaaaataaaagtaaacaacatcaatgaacgttcacttttttttaaagaaaggcttcaaagtgttgtttgaataaaacttaattattttttaaaaatattccaggaattaatttgtacttttttagcttttggatttgtgttagcaattttgctgtttgtatttattaatggaagtgttgctattaataataatggttctactgttttttcatttgaagctgattcaactggcagagcactagtaaatactctagtaattattttgattactatcaccattacttttccactagcacttttaattgcaatttgacttaacgagtacaataattcaaaagtggttaaaaatgtttttaactttgtaattgattcactaagttcaatgccatctattatttatggattatttggactttctttctttttaagagtcttgcagttaagtgctggaggagctaatggtactagtttaatagcaggcattctaactattagtgttgttatattactcttccttataagaacttgtcaacaagcactaaataatgtcagttgggatttaagaattagtgcttttgctttaggtataagtaaacgtgaagttattttcaaaatagttttacctagtgctttgaaaggattaatagttgcattaattttgtcaatcaacagaattattgctgaaactgcacccttctttatcacttcagggttatcatctagtaatttatttcatttgtcattgccaggtcaaacactaacaacaaggatatatggacagttattttctattaatagcaatgcaataagtgttatgttagaaacatcattggtctctgttgttttcttaattcttttaatctttttcagttcttatttaatcccgagtttatttttgttaaataaacaaaaatggctagtaattaaaagtaaatttcagtcctttaaattatggaaaagaacataaaagcactttgaaaaaattttcaattgaagcttgaaaaaattaagcattaccgaaagctttatgaacaacaaatcaaagaatataaaaagaaaattactggtttaaataatgaaacagatgcaaatgaaatctcccgtattaagaatgaaattgaaattttaaaccgtctaataaagattaaaaacaccaaagataatgtcattaaaaaggattttgatgaaaaaaatgtatttgaaattcgaaatttcaacttctgatataacaaaaacaaacaagtattatttgatatcaatcttgacattaaacgcaataaaataactgctttaataggtaaatcaggatgcggtaaatccacctttattaggtgcttaaataaattaaatgatttaaatgaaaacacacgttgaacaggtgacatatattttcttggtaagaatatcaattcaggaattattaatgatttaacattgcgcactagtgttggcatggtttttcaaaaattaactccttttaatttttctatttttgaaaacattgcttatggcataagagcacatggtattcacaataaaaatgctatcaatgaaatagtaagacaggcattgatatcagcagcattgtgagatgaagtgaaagataatttacataggaatgcaaacaccctttctggtggacaacaacaacgcttgtgtattgcgcgtgctattgctttacaaccagatgttcttttgatggatgaacctaccagtgctttagactcaattgccacaaactctattgaacttctaattcaacaactaaaagaaaaattcacaattgttattgttactcactctatggctcaaacaattagaataactgatgaaacgattttttttgctgatggaagagtaattgaacaaggcactacaaaacagatatttacaaagcctaagcaaaaagcaacaaatagttatataagtgggaaaaattag

SEQ ID NO: 14 - DNA, 4750 bp, M. genitalium
aacacctaacaaagtttgtccaacgcttggttaaataactatggatgaaaatgaaactcaattcaacaagttaaaccaagttaaaaacaagctgaaaattggtgtttttgggattggaggtgctggtaataacattgttgatgcatcactttatcactatcctaatttagcaagtgaaaacatccacttttatgctataaattcagatttacaacaccttgcatttaaaacgaatgttaaaaataaactcttaattcaagaccatactaacaagggctttggagcggggggtgatccagctaaaggagctagtttagcaataagctttcaagaacagtttaatacacttacagatgggtatgatttttgtatcttagttgctggatttggtaagggtactggtacaggtgctaccccagtttttagcaagatcttaaaaactaagaagatcttaaatgttgctattgttacctatccatctttaaacgagggattaacagtgagaaacaaagccactaaggggcttgaaattctcaacaaagcaactgatagttacatgctattttgtaatgaaaaatgtacaaatggtatctaccaactagcaaacacagagatagtcagtgccattaaaaacctaatagaactaattactattcctttgcagcaaaacattgattttgaagatgtacgtgccttttttcaaaccaaaaaaactaaccaagatcaacagctttttactgttactcacccctttagttttagctttgatagtaaagatagtatagaacagtttgctaaacagtttaagaactttgaaaaagttagttattttgaccactctatagtaggagctaaaaaagtgttattgaaagctaacattaaccaaaagatagtcaagcttaacttcaagcagatccaagatattatctgaactaaaattgacaactaccaacttgagattaggttaggggttgattttgtgacaaccatccctaatatccaaatttttatcctcagtgaacacaaaaatccagtttcgcttcccattgataataaatcaactgaaaacaaccaaaataagttgaaacttttagatgagctgaaagaacttggcatgaaatatgttaagcaccaaaaccaaatctactaatgaaaaatatgtatctgaaaatgattctaattaactaataatgggctttttaagcaagttaattgccaaactaaaaccaaaaaaatcagttgctaaacagcttaaagaagaagttgaaaaacaaagcctttttcaaaccaataataaaacttactatcagggtttgaaaaaatctgctacaactttcgctaaaactattaatgaactgtcaaaacgatatgttaatgttgacgaacagtttaaagaaaatctatttgaagggctagttttgcttgatgttggttatcatgctgcaaacaaaatttgtgatgctattattgaacagatcaagctaaacagaattacagattttcagctcattaaagagctaattattgaccaaattattgtttattacatccaagataaactctttgatactgatttaatagttaaacctaactttacaaatgtttatctctttgttggtgttaatggagttggtaaaacaactactttagctaagatagcggattttttcataaaacaaaataaacgtgttctacttgttgcaggtgatacttttagagcaggagccattgaacaacttaatcagtgagcaaagctgttaaactgtgacattgtacttccaaaccctaaagaacaaactccagctgttatctttcgtggcgtaaagaaagggattgatgataaatatgactttgttttatgtgatacatcaggaagattgcaaaacaagcttaacttaatgaatgaattgcaaaaaatttatcaaattattcaaaaggtaagtggaagtgaacctagtgaaacacttttagttttagatggtacagtaggtcaaacaggactatcacaagcaaaggtatttaatgaattttccaaactaacagggattgttttaactaaaatggatggttctgctaagggtggaattattttagctattaaagatatgtttaacctgcctgttaaactgataggttttggtgaaaaaactagtgatttagctatctttgatctggaaaaatatgttttaggtttacttaataacttaaacttagataataaagaaaattagtagcaataacagagctaataaagttttaaaaaataattatatggaaaaaacatcaaatacaagtaagccactttctcgtagtgaaatcaataaaataattgcagttgctactggtataaaagaaaaaaaaattaaggaaatctttaaataccttaacacattgttactaaatgaattggtaagtagaagtgtttgtatattacctgaaaatttaggtaaattaaggattactattagaaatgcacgttatcagaaggatatgcaaacaggtgagattagacatatcccaccaaaaccattagtgcgctacagtccaagcaaaacgatcaaagaaactgcagctaaagtgcgttgaaagtacgcagactaatcaaaccaaaaaaacccaaatcggtcaaaaactaacatagatgaaaaaaagaaataagggtttagtagaacaaacaactactgaaaaaaataatttttcacgtaaaactgcttgaaaagtcttttgatgagtcatcattttagctgttgttattggtgttttagcttatattttcagtccaagagctgctactgcagtagttgaaagctgaaaattaaatggaggtagtaacagcactttaacagcaaaagtaagcggttttagtaatgaactgacatttaaacaaataaatggttcaacttatgttactgataccattctccaagtttccattacctttgatggtttaaatagtccattaactgttactgctcacaaaactgttaatagtaatggcaatgttatctttaatattgctaacttatcaattaaccaaagtaatggtcagattaccgttaatagtaatggaaccatgatgaatggtggttctagtaataacacaaagagtattgcaggttttgaaacccttggtactttcattgctcctgatactagagctagagatgtattaaatggtttgtttggcttgctaccaattattatctttgtagttttctttttactcttttgaagaagtgctaggggtatatctgcagggggcagagaagaagataatattttttctattggcaaaacccaagctaagttggctaagtcaactgtgaaatttaccaatattgctggacttcaagaggaaaagcatgagttgcttgagatagttgattatttaaaaaatccattgaaatatgcccagatgggagcaagatccccacgtggggtaattttatacggtccacctgggacaggtaaaacattattagctaaagcagtagctggtgaagctggtgttcctttctttcaatcaacgggttctggatttgaagatatgcttgttggtgttggtgctaaacgagttagagatcttttcaataaagctaaaaaggctgctccttgtattatttttattgatgaaattgattcagttggttctaaacggggtagagttgaactctcttcttattctgttgttgagcaaaccttaaaccaattgttagctgaaatggatggatttacaagcagaacaggtgttgttgtaatggcagctacaaataggttagatgtattagatgatgcattattaagacctggaagatttgatagacatattcaaatcaatctccctgatattaaagaaagggaagggattttaaaagttcatgctgaaaataaaaatctctcttctaagataagtcttttagatgttgctaagagaactcctgggttttcaggtgctcaattagaaaatgttatcaatgaagctacattgttagcagttagagacaaccgtaccacaattaacattaatgacattgatgaagcaattgatagagtaatagctggtcctgctaaaaagtcacgtgtaattagtgatgaagatagaaaactagttgcttatcatgaggctggtcatgccttggttggtttacatgtccacagtaatgatgaagtacaaaagattaccattattcctcgtggtcaagcagggggttacacactttcaacacctaagagtggtgatcttaacctaaaaagaaaatctgatttacttgcaatgatagcaactgctatgggcggtagagctgctgaagaggaaatctatggtaatttagaaattactactggcgcttctagcgatttttataaagcaactaatattgcaagagcaatggtaacccagcttgggatgtctaaattaggtcaagtgcaatatgtaccaagtcaagggacactcccttctaatgtaaaactttattcagaacaaactgctaaagatattgacaatgagattaatttcattattgaagaacagtataagaaagcaaaaacaatcattaagagtaaccgtaaggaactagaattgcttgtagaagcacttttaattgctgaaactattttgaaaagtgatattgacttcatccataaaaacactaaactaccaccagaaatcttattgcaaaagcaagaacaacaagcaaagcaaaaactaaataaatctgaagtaaaaccagaaagtgaaacaaacagttag

SEQ ID NO: 15 - DNA, 13894 bp, M. genitalium
gcaagaattataattaacactctaaggatgcaagtgataaatggctgctggtaaaagggattattatgaagttctagggatatctaaaaacgctagttctcaagacataaaaagagcttttagaaagcttgcaatgcaatatcaccccgatcgtcataaagcagaaaatgaaactactcaaaaacaaaatgaggaaaagtttaaagaggttaatgaagcatatgaagttctaagtgatgaagaaaaacgtaagctttatgaccagtttggtcatgaagggttaaatgcttctggttttcatgaagcagggtttaatccttttgacatctttaatagtgtttttggtgagggattttcctttggaatggatggtgattcaccatttgatttcatttttaatcgttctaaaaaacgtcaacaacaaattgttgttccctataaccttgatattgctttagtaattgaaattaacttttttgaaatgactaatggttgcaacaaaaccatcaaatatgaaagaaaagtttcatgtcatagttgtaatggttttggcgctgaaggcggggaaagtggattggatctttgtaaggattgtaatggcaatggttttgttattaaaaaccaacgttctatctttggaaccattcaatcccaagtcttgtgttcaacttgcaatggacaaggaaaacaaattaaagttaagtgcaaaacttgtcgttctaacaaatacactgttaccaatcaaattaaagagattaatattccagcaggaatgtatagtggtgaagctttagttgatgaaagtggtggtaatgaatttaaaggtcactatggaaaattaatcattcaagtgaatgtattggcaagtaagattttcaaacgtagtgataataatgttattgccaatgttttagtagatccaatggttgctatagttggtggggtaattgaactacctactcttgaagggattaaagaatttaatattagaccaggcactaagagtggcgaacagattgttattcctaacggtgggattaaattctcaaagagttttaaaagaaaagctggggacttaatcattattattagttatgcacgtccttgtgaatacactaacttagaattgaaaaaattacgtgagtttatcaaacctaatcaagaggttaaacaatatttaaatactttaaaaaatgaatacaaaacttaattttattaaaacattatttaataataaataattaaaaatcatgttcaaagcaatgttatcaagcatcgttatgcgcacgatgcaaaaaaaaattaacgctcaaacgatcactgagaaagatgtagagttagttctaaaagagattagaattgcattgcttgatgctgatgttaacctgcttgttgttaaaaatttcatcaaagcaattagagataaaacagtaggacaaaccattgaacctggtcaagatttgcaaaagtctctattaaaaacaatcaaaacagaactaattaatatcttaagccaacccaaccaagaactaaatgaaaaaagacctttaaaaataatgatggttggtttacaaggatcaggtaaaacaacaacttgtggcaaactagcttattgacttgaaaagaaatacaagcaaaaaacaatgttagtaggcttggacatctacagacccgctgccattgaacaacttgaaacgctttcacaacaaactaacagcgtattttttgcacaaggcactcaaccagttgctaaaacaacaaaagcagcactcagtgcttttaaaactgcaaaatgtcaaacaatcatttgtgataccgctggtagattacaaacaaatgaaacattaatggatgaattggtaagtgttaaaaatgaattaaatcctgatgaaattatcatggtagtagatggattaagcggtcaggaaattatcaatgttgctcaaacgttccacaaacgtttaaaactaactggatttattatcagtaaattagacagtgatgctagagcaggagctgcactttcattagcttcacttttacaagtacccattaaattaattggtgtttctgaaaaattagatggattggaacaatttcatcctgaaaggatagccaatcggatcttaggtttgggtgatgtaatgagtttagttgaaaaagctgaacaagtttttgataaaaaagatttaactaaaaccatcagcaagatgtttttgggaaaaatggatttagaagatcttttgatctacatgcaacaaatgcacaaaatgggaagtgtcagttcactgataaaaatgttgcctgctaacttttctgtatcagaagaaaatgctgaattaattgaaaacaaaattgaactatgaaaggttttaattaactctatgactagagaagaaaggagacatcccaaattaattaatcgtgatcctaatagaaaacagcgcatcataaaaggttcagggagaaaaatggatgagttaaacaaactgatgaaggaatgaaataagatgcaactaaaagcaacagaaatgggtaaactattaaaaacaggtagtaacccgtttggtggatttggacaattcttttaacaatcaaaaaactaagcatctagattctttttaaaaagccatggaaaaaaaactgccttttagctttaaaaagaaggaaaagctaactgcttatgatgatgcttcaattcatgagttacataaacagctcaaacttagaacagaagccaagaaaagtaaagataaggaaagaactaaagaaaaagaaaagcatgaaagtttagcaaaggaaaagaaacccaagcttccttttaaaaaacgaattgttaatttatgatttggagttgataaagagatcaacaaaattgtttgagtaaaaggtagacaacttatcataatttttcttttaattttgctagttagtggactgatggtaggaatcttttttggtatcaatcaattgttaattacgttgggaatatttaaaaattaattaaacattaaccgtcaacaacaagaaactttatagagcaatggcaatatttaacttccttaagttaatttcacccaaaaacagaattctcagtaaggcaaataggattgccagtgaggttgagagttataaaaactactaccgtaacttaactgatcaacagttatttgaagagtcaaataaactagttgatcttgtcactaagcaaaattacaccattctagatgtttgtgttgctgcacttgctttaattagagaagtggtttaccgtgagactggtgaatttgcatatagggtgcagatcataggagcttttattgttttaagtggtgattttgctgagatgatgactggtgaaggtaagaccttaaccattgttttagcagcatacgtttctgcacttgaaaagcgtggtgtgcatgttgttactgttaatgaatatctagctcaaagggatgctaataatgcaatgaagatcttaaaacgggttgggatgagtgtcggttgtaactttgctaatctctcccctcagctaaaacaagctgcatttaattgcgatgttacctacaccactaacagtgaactggggtttgattatcttagagataacatggtccacagttatcaagataagaagatcagagagttgcactttgcaatagttgatgaaggtgattcagttttaattgatgaggcgcgaacgcctttaattatttcaggtcctagtaaaaatgagtttgggttatatgttgcagttgatcgatttgttaaatcattaactgaacaggagtttaagattgaccctgaatcacgtgctgcttctttaactgaacttgggattaaaaaagcagagcaaacatttaaaaaagaaaacctttttgctttggaaaacagtgatctttttcacaagatcatgaatggtttgactgctgtgaaagtttttgaacagggcaaagagtacattgttcgtgatggcaaggttttaattgttgatcactttacaggtaggatattggaagggagaagttacagtaatggcttacaacaagctgtacaagccaaagaatatgttgagatagaacctgaaaatgtgatagtagctaccattacctaccaatccttctttaggctatacaaccgcttagcagcagtatcaggtactgctttaactgaatcagaggagtttctcaagatttataacatggttgtagtaccagtgccaactaaccgtcctaacatcagaaaagaccgttctgatagtgtatttggtaccccacaaattaagtgaatggcagttgttaaagagataaaaaagatccatgaaacttctcgacctattctgattggaactgctaacatagatgattctgaactcttacataatctgttactagaagctaatattccccatgaggttttaaatgctaaaaaccattcaagagaagcggagatagtaactaaagcaggacagaagaatgcagttactatttcaactaacatggctggaagaggaactgatatccgtttaggtgaaggggttgctgaaatgggtggtctttatgtattgggaactgaaagaaatgagtcaagaaggattgataaccaactaagagggagagctgctagacaaggtgataaaggggaaactaagttctttatctcactaggtgattcattgtttaaacgttttgctcatgacaagattgaaagagcgattagcaaattaggtaatgaaacatttgacagtgccttcttttccaaaatgttaagtagaacccaaaaacgggtggaagcaattaactttgacactagaaaaaacctgattgattatgaccatgttcttgcaagtcaaagggaattgatttacaaacaacgtgataagtttttattagcaaacgatttaagtgaaatgatcgacaaaatgctagaaaagtttgtacaacagttttgtgatcaatatagaaaccaaaagaaccaaaacttaattaatcacattgcactagcagaagctttaaatcttgagatgaacatgcaaaacaccattaatccaaaggtgtttgaaaacatgacttttgatgttgctgttgataaaacccgtaacttagtagctaaaaagattagtgataaagttaatgttttgaccaaaccaattgctttaaacaggtttcgtgacattatcataacttcgatggataaacattgaactgaacacttggatagtgtttttaagttaagagaaggggttgtacttcgttctatggaacatacgagtcctttaaatgtttacattaaagaaacagatatcctttttaaaacaatgttgcaaaagattgctcaagatgtcattgtgcaaattgctaacctcacaactccagatgaatttgatcatagcttaatgcaagccaatgctttaaagaaactagcagcaattaaagcagatgaaaaatcaaaccaagagtaagcttaaaattaagataaataattttccaattttgttttcaatggaacaaaaaaacattagaaatttttctattattgcccatattgatcatggtaaatctaccttatcagaccgcttgttagaacatagtttaggctttgaaaaaagactattacaagcgcaaatgcttgatactatggagattgaaagagaaaggggtattaccattaaattaaatgctgttgaattgaaaattaatgttgataacaacaactatctttttcatttaattgacacccctgggcatgttgattttacttatgaagtgtctcgttctttagcagcttgtgagggagttttattgttagtagatgcaacccaaggaattcaagcacaaacgatttccaatgcttatcttgcgttggaaaataacctggaaattatcccagttattaacaagatagatatggataatgctgatattgaaacaacaaaagattcactccataacttattaggagttgaaaagaacagtatctgtttagtatctgcaaaagctaacttagggattgatcagttaattcaaacaattatagctaagatccccccaccaaaaggagaaattaatagacctttaaaagcattactctttgatagttactatgatccttacaagggggttgtttgttttattagggtatttgatggttgtttaaaggttaatgataaggttcgttttattaaaagtaattctgtttaccaaattgtggaactaggggttaaaaccccattttttgaaaaaagagatcaattgcaagcaggagatgttggttggttttcagcagggataaaaaaacttcgtgatgttggggttggtgatactattgttagttttgatgatcaatttacaaaacccctagcaggttataaaaagatcttacccatgatctattgtggtttatatccagttgataacagtgattatcaaaacctcaagttagcgatggaaaagatcataatcagtgatgcagcattggaatatgaatatgaaacatcccaagcgttaggttttggggttaggtgtggttttctaggtcttttacatatggatgttattaaagaaagattggaaagagaatacaacctaaaactcatctcagctcccccttcagttgtatataaggtgttgttaacaaatggtaaagagattagtattgacaatccctctttgttaccagaacgctccaagattaaagcaatcagtgaaccatttgtaaaagtctttattgatttacctgatcaatatttgggcagtgttattgatttatgccaaaacttcaggggtcaatatgaaagtttaaatgagattgatatcaacagaaaaagaatctgttatctgatgcctttaggggaaattatctacagtttttttgataagttaaagtcgattagtaagggttatgcatcgttaaactatgagttttataactaccaacatagtcaactggaaaaagttgagatcatgttaaacaaacaaaagattgatgcattatcttttatcagtcataaagactttgcttttaagcgggcaaaaaagttttgcactaagctcaaagaattgattcccaagcatctgtttgagatccctatccaagcaacaatagggagtaaagtaatagcaagagagacaatcaaagcagttagaaaggatgtaatagctaaactttatggaggggatgttagtagaaaaaagaagttattagagaagcaaaaagagggtaaaaaacgcttgaaagcagttgggagtgttcaattaccccaagagctatttagtcatttgctgaaagatgaagattaacattattaagaaataaaccaattagtgatctataaaaacaatgcaaactgtttcttcacccaaacaaaaacttaactttggtcaaaggttactaactctattacagaaccgtgactttatggtgtcgctggttttaacagtggtacttttaatcttgtttagggtgttagcaattatccccttaccagggattaggattaatgagagtgtcttggatagaaattccaatgactttttttcactttttaacttacttgggggtgggggattaaaccagctatcgttgtttgcagttgggatcagtccttatatctcagcccaaatcatcatgcaactgctttcaactgatctaattcctccactttcaaagctagttaacagtggggaagtggggcgaagaaagattgagatgatcacaagaattatcaccttaccctttgctttagtgcaagcatttgctgtgatccaaattgctactaatgcaggcactggttcaagtccgattagtttagctaatagtggcagtgagtttattgctttttatattattgctatgactgcagggacttatatggcagtgtttttgggtgatactatctccaaaaaaggggttggtaatgggattactttgttaattctctcagggattttatcccaactcccccagggctttattgctgcttacaatgttttgagtgggatagtaattactctaaccccacagttaactgcagcaattagcttctttatctatttcttagcattcttagttttactgtttgccactacctttatcacccaagcgaccagaaagattcccatccaacaatcaggacaagggttggttagtgaagtcaaaaccttaccttatttgcctattaaggtgaatgctgctggggtgatccctgtcatctttgcatccagtattatgtctatccctgtgaccattgcccagtttcaaccccaaactgagtcacggtggtttgtggaggattacctatcactttcaacacccgtagggatctttttatatgcagttttggttatccttttttcctttttttacagttacatccagattaacccagaacggttagctaagaactttgaaaaatctggcagatttatcccagggattcgaccgggcaatgatacagagaaacacattgcgcgggtgttaataaggattaactttataggtgctccttttttaactgttattgctattatcccttacattgtttcttatttcattaggttacctaactccttgagtttaggggggacggggattattattattgttactgctgtagttgaatttatcagtgcactgcgttcagctgctactgctactaactaccaacaactaaggagaaacttagcaattgaagtgcaacaaacagctaaacaagatagtctagagcagcttcaaaaagaagcaccagggattggtaacctatggtagaatacctctcccaagaacccattagttagaatttgttaatatgtgtgaaaaatcacaaacaattaaagagcttttaaacgccattagaaccttagttgtcaagaacaataaagctaaggttagtatgattgaaaaggaactgttagcttttgttagtgaacttgacaaaaagttcaaacaacaactcaacaacttcaatgaactacaacaaaagatcccactactccaaaaagctaacgaagagtttgctttaaagtttgaaaggatgcaacgcgaagcacaaaaccagatccaagccaaactagatgagttgaatcttaaaaataaaaaggagttagaacaagccaagaaatatgcgattgccaaaacccttgaccaacccttaaacatcatcgatcagtttgaaatcgcgctttcatatgcccaaaaagaccctcaagtaaaaaactataccactggttttaccatggtacttgatgctttttcaaggtgattggaagcaaatggggttaccaagattaagattgaaccagggatggaatttgatgaaaagattatgtctgcattggaactagttgattctaaccttgctaaaaacaaggtagtaagagtctcaaaatctggctataaactctatgacaaagtgatccgctttgcatcagtatttgtcagcaaaggtaataaaaaatcataaaaacttaagagtttaaacttatctttaaccgatccactccatgaaattaagaaaaaccaagtttttttcacaacttaaacaccaggttttaactgcaaaccaaaaaccatttttattctataaactgacaatgattgggtttgttggctttattatcttactgcaagttttcatattaagaaatgcgttaaatggtgagatggataacaccatggtagcaaatagtggttttattaatatctatgtgattagaaacaaaggggtagggtttagcttattacaaaaccaaactggcttagtttactttctccagggattattatcagtaattgcgttagtttttcttgtttttatggtgaaatatagttacatcttttgaattacaactttagcatttggttcacttggaaacttctttgatcgtttaacttcagctaatgattcagtgttagattactttatctttcagaatggtagttcagtatttaactttgctgattgttgtattacctttggttttataggtttattcttttgttttttaatccagatgttcaaagagtttaaacattccaaaaaccagtaatataattactgagtaattgttattgatctaaaaaaaaagtatgagtgcagacaatggtttaattattggcattgaccttggaactaccaattcttgtgtttctgtaatggaaggtgggagacctgttgtattagaaaatcctgaaggtaaaagaacaacaccttccattgtttcctataaaaacaatgaaattatagtaggtgatgctgctaaaagacagatggttacaaacccaaataccattgtctccatcaagaggttgatgggtacctcaaataaagtaaaagtccaaaatgctgatggtacaactaaggaattaagtcctgaacaagtttcagcgcaaatccttagttatcttaaggactttgctgaaaaaaagattggtaaaaagatttcaagagcagttattactgttcctgcatactttaatgatgcggaaagaaacgctactaaaaccgcaggtaagattgctggtttaaatgttgaaaggatcattaacgaaccaactgctgctgctttagcttatgggattgataaagcatcaagagagatgaaagtcttggtttatgacttgggtggtggaacttttgatgtatctttacttgacattgcagaaggtacttttgaagtacttgcaactgctggggacaaccgtttgggaggtgatgattgggataacaagatcattgaatatatctcagcctacattgccaaagaacaccagggtttaaacttatcaaaagataagatggcaatgcaacggcttaaagaagcagctgaacgtgctaagattgaactttccgctcaacttgaaacgattatttctctaccatttttaactgttacccaaaaaggtcctgttaacgttgagttaaaactaacccgtgctaagtttgaggagttaacaaaaccactacttgaaagaacaagaaaccctatttcagatgttatcaaggaagctaagattaaacctgaagagattaatgaaattcttttagttggtggttctacaaggatgcctgcagttcaaaagctagttgaatcaatggtaccaggtaaaaaaccaaaccgttctattaatcctgatgaagttgttgctattggcgctgctattcaaggtggggttttacgtggtgatgttaaggatgttttacttttagatgtaactccattaaccctttctattgaaactttaggtggtgtggctactcctttaattaagagaaatacaactatcccagtaagtaaaagtcaaatcttttcaactgctcaagataaccaagaatcagttgatgtggttgtatgtcaaggggaaagaccaatgtctagagataataagtcattaggaagatttaacttaggtggtattcaaccagcacctaaaggtaaaccccaaattgagattacctttagtttggatgccaatgggatcttaaatgttaaagctaaggatttaaccacgcaaaaggaaaacagtattaccattagtgacaacggtaatctttctgaggaggagatccaaaagatgatccgtgatgctgaagctaacaaggaacgggataacatcatccgtgaacgtattgaattacgtaatgaaggggaaggtattgttaataccatcaaagagatattagcaagtcctgatgctaagaatttccctaaagaagaaaaagagaagttagaaaagctaacaggtaacattgatgctgctattaaagctaatgactatgccaaactcaaagtggaaattgaaaactttaagaagtgaagagaagagatggcaaaaaaatataacccaactggtgaacaaggtccacaagcaaaataattcttttaaattagtttttaattattaaaatatttttattatgaacataacgccaattcatgacaacgtcttggtttcacttgtggaatcaaacaaagaagaagtctcaaaaaaagggattattacctcattggcaagtaatgataaaagcgatgctaatgctaataaagggattgtaattgctcttggtgctggtcctgcatatggcaaaacagaaaaaccaaaatatgcttttggtgttggtgatattatttactttaaggagtatagtggtatctcttttgagaatgagggaaacaagtacaaaattattggatttgaggatgtacttgcctttgaaaaaccagaaagtggtaagcaaagaaaaagataaaattaaacaattatggcaaaggaattaatctttggtaaagatgcgagaacccgcttgttgcagggtattaataagatagcaaatgctgttaaagtaacagtaggtcctaaaggccaaaatgttattttagagagaaaatttgcaaacccattaattactaacgatggggttacaatcgcaaaagaaatagaacttagtgatccagttgaaaatattggtgctaaggttatttcagttgctgcagtgtcaactaatgacattgctggggatggtacaacaacagctaccatattagcacaagaaatgacaaaccgtggtattgaaattatcaataaaggtgctaatcctgttaacatccgcaggggtattgaagatgcaagcttacttattattaaagaacttgaaaagtactctaaaaaaattaatactaacgaagagatagaacaagttgcagctatctcttcaggttctaaagaaattggtaaactgatcgctcaagcaatggctttagttggtaaaaatggcgtgataacaactgatgatgcaaaaaccattaatacaacattagaaaccactgaaggaattgaatttaaaggaacatatgcatcaccttatatggttagtgatcaagaaaaaatggaagttgttttagaacaacctaaaatcttagtaagctctttaaaaattaacacaattaaagaaattcttccgcttttagaaggtagtgttgaaaatggtaatccattattaattgttgcacctgactttgcagaagaagttgttactactttagcagttaataaactcaggggcaccattaatgttgttgctgttaaatgtaatgaatatggtgaacgtcaaaaagcagctttagaagatttagcaattagtagtggaaccttagcatataataccgaaattaatagtggttttaaagatgttactgttgataatttaggtgatgctagaaaggttcaaatagctaaaggaaaaactactgttattggtggtaaaggcaataaggataaaatcaaaaagcatgttgaacttctaaacggaagattaaaacaaaccactgacaagtatgattctgatttaattaaagaaagaattgcttatttaagtcaaggtgttgctgttatccgtgttggtggtgcaactgaactcgcacaaaaagaattaaaactcagaatcgaagacgctttaaattccaccaaagctgcagttgaagaagggattatcgctggaggtggtgttggtttattaaatgcttcttgtgttttaactaacagtaaactaaaagaacgatatgaaaatgaaactagtgttgaaaacattaaagaaatcctacttggttttgaaattgtgcaaaagtctctagaagcaccagcgcgtcaaattattcaaaactcaggagttgacccagttaaaattctcagtgaattgaaaaatgaaaaaactggtgttggctttgatgctgagactaaaaagaaggttgatatgattgcaaatggaatcattgatcccaccaaagtaactaaaactgcacttgaaaaagctgcttctgtagctagttcattaattactactaatgttgctgtgtatgatgttaaagagagaaaagataactccttttcagaataa

SEQ ID NO: 16 - DNA, 2556 bp, M. genitalium
cgtcaaaaattgttgcaaacaatctaattaaactttattggtggattggaaaacatcagagatgctattccctttccccgtgtacatggcaccattaacttctaaattcgctgcttataaaaaaaagattgcaaactggttaacagtttacagaatttttattgctttacctactattatttttattgctttagataatcaactaggagttttagctaacttttctgttggtgcaattagcattagtttacagatcagtttattgattggaggatttttgtttttaactgcagttatatcagattatttagatggatatttagcaagaaaatggctagcagtttctaactttggtaaattatgagaccccattgctgataaagtgattatcaatggtgttcttattgcactagcgattaatggatattttcactttagcttattaattgtttttatagtccgtgatcttgtgttggatggaatgcggatttatgcttatgagaaaaaggtggttattgctgctaactgacttggaaaatgaaaaactatcatgcagatggttggtattgtttttagttgttttgtttggagttttaaacaaagtgaaatagcttctttgaatagtggactgttcttttgattactaactcaactgccatattatttagcagcagttttttcaatttggtctttcattgtttataacatccaaatatatcagcaactaaaggcttataactccaagttataaactaattgccatgttagtgctatctgtttagcacaacaaaatggataaactatttaaaacaagttttagattcataataaggtttttacaaatcctgagtttaccagttgtttttccttactttttattaagctttttagcttgtttaattactagtaaaaactatgaatcactcccttataactatccccctgaaatccgattcaaaaaggtgtatagattggtatcaatgtgactttacattaagggaattaaagtagtgacagtaaatgacaagattatccctaaaaaaccagttttagtggtagctaaccacaaatctaaccttgatcctttagtattaattaaggcctttggcaggttgaaaaatagtccaccattaacctttgttgctaagattgaactgaaagatacagtcctttttaaactgatgaaattaattgattgtgtttttattgatcgaaaaaacatcagacaaattgccaatgcattggaaacccaacaacaactaattcgccagggcactgctattgctgtttttgctgaagggactaggattttaagtaatgacattggggaatttaaaccaggagcactaaaggttgcttacaatgcttttgtacctatcttaccagttagtattgtgggtagcttaggaaagatggaatcaaacaaaaggctaaaagaacatggtgttaagaaaagttcaaactatgaggttaaagtaatctttaacaagctaattaacccaattagttttaaccagattgattctaataaccttgctaataacattagaagcattattagtgatgcatacactagtgaaaaaccaagcaatgattagcataatcattattttaattgttggggtaattggttctctgatgatttgagagttgttcacaaacatactaaaaaataaaccaaaactaagcttaagtttaacgttgttaaatgctggaataattatttttgggatgattggtacttttgttgttgtttatttttacaaatgaaatgcaactgttaatggtatttgaacattaagttttactctttctgtggttttactttgaataatttacattgcttgcatgagtaaaacaagaattaagtttagcttacaactttcatatagcttaggagctattgcttgctttattgctagcataggtactatttacttttctgttatcaggggttgaactacaatctttttattgatgagtttagcagtcagtgttgatacatttccttttctttttggaaagcgctttggtaaaaatcctttaattaaaatttcaccatcaaaaacatgagaaggagctttttttggcatcattagcaccattgttgttgttgctttactttgtgttttatattcaattcctttctttgtagcaaagcctacttttaatcaaacaaatggaatagcgctcaatacaccccaaaattatgatagccataatcttattaccaatatttttttaattgcctttatctctggaggaagtagtttttatatctactggtgggtaagcactttagctttaatttttacaggatctgtttttgcaataggcggtgatcttttttttagttatattaaacgcttaattagtatcaaagatttttctaaggttttaggtaaacatgggggagttttagatcgatttgattcaagttcttttttaattagtttcttctttgtttatcatttaatagcaggaaccatttccaaccaaaggttgttgatggaacctaatacttatttcagtgcaatcactagtattcaaagctag

SEQ ID NO: 17 - DNA, 2601 bp, M. genitalium
aattgtaagtaggtataattacagataatttcattgataaatgttaattcttgttaacaatcctaaggctaaatatgactatcatttaatggaatcttattgtgctggaatagttttaaaaggaagtgaagttaaagctttaagtttaggtcaaggtagtttaaaagaagcttatgtttttgttaaaaacaatgagctttttttagaacagttcactattccaccttatagttttgcaggtccattaaatcacgcttcagatagaattaagaaacttttattaaataaacatgaaattaaacaaattatcaataaaaaacaacaacaatctttatctgtaatcccaagtaaagtattctttagaaatggcaaaattaaagtggaaatttgattggcaaaacctaaaaagaaatttgacaaacgtgaaactattaaaaagaaaacaatccgacgcgagcttgaagctgagtatcgataaatttagccttaggattgttaacaagaattaacatttatcaatgaaattatctgtaattatacctacttacaattgtgcatcatttattgaaaaagcaattaattcaattgttaaaaatagacctaatgatttggaaatagaagttttaattattgatgatggatcaattgacaatactaacaaagttattaagaaaattcaagaccaaattaataatttaactttgcagtatttttacaaaagtaatggtaactggggtagtgttattaattatgttagaaacaataaactagcaaaaggggaatgagtaacagtattggatagtgatgacattttttcaaaaaaaacaatttctatttttcaaaaatatgcccaaaaacaaagatatgatgcgattatttttgactactataaatgctgaaaaaagtttttgtgaaaaattcctacctatgcaaggtttagaaaagaaattaaaggtgaattgaaaaaacaaacacctttttgtattcccttagctaagttttttaaaaatgaggttttctatcaacttcctaaactaagagaaaatgttggttttcaagacgctatttatacgatgcatgcattacaaattgcaaataatgttttccatgtttctaaagctggaggatattacttttttaaaagggtaggtaactctatgagtatcccttgacacagttctaggtttgatattgaagtacaaatctgcaaggatctgattgaaaataatgcgcaagagatcgctttagtgcatttacttcgtttaaaatttcgtaatttagttgatgataaaaagattaaatttacagttaaaagagacttttgttttagtggttttagttggtatagtaggttaattttatctctgatgtataacttctgattgaaacgttatttcaacagttctgaataaggtgaaaaaccagtgtttagcacttttatacaaaagctaaatgaatagaccaagttgatcaactgcatttaatattggtggtggatttcccatccagtggtatgggatcattgtctcaattggcattatttttgccattttaatgtttgtctttaaactgatttattgttacaaattacaagacaacagtttttatttttttatctttattgctgttttaacgatggttttaggcgctcgcctctggtcatttgtaattggtgattccaattttgctaacaacaatttctttgattttcgtaacggtggattggccattcagggtgggattttgttaaccagtattgtcggagtaatctatttcaacttctttttaaatagtaagaccaataaaaccaaaacgattgctgaattactgaataataagaatgaaataaaagctgtttatgttgaaagaaatatctctgttctagtgatgttagatctgattgctccttgtgttttaattggtcaagcaattggcagatggggtaattttttcaaccaagaagtttatgggtttgctttagctggaacaatgaatgatccccaagcattggctaatacccagtggggatttttaaagatcttaatgcctaaggtttgggatgggatgtggattgatggtcagtttcgcattccgctctttttaattgagtcattttttaacactattttctttgtgttaatttactttgtaatggattttattaggggagttaaaagtggcacaattggttttagttattttcttgctactggaatcattcgtttaatcttggaaaactttagagaccaaaccttttattttcaaacttcaataaccactagtattttgtttattgtcgttggtattttaggaattttttattgccagtttatccatgtcaaattaagaaattacttctgaacttatttctttctttatgccttttataaagtagctgcttttttcactacactttttttgaataacagaaagcaaatggcacaacagaagtttgctttttatgaaaaatcacttcccaataagaagcgttctttttttgaaatgaagtattacaatgatgtaacaacacccaaaatttatcgtttaactgatcaggaaatgaagttatttgataaattagaggcagttacaaccagctag

SEQ ID NO: 18 - DNA, 3706 bp, M. genitalium
caaaaaccaaaattattgatcttttcaataactaaagtccatgattgatctgcttggtttggatctggatggaacgttattatctaaaactaaaaaaattaacaatccatcaaaattagcattaactaatttaattgctaaaaaaccaagtttaaaggtgatgattttaactggtagatcagttttttctactctaaaacacgttgaaaagctgaacagtttgtttaaaaaaccaattgttgattatttttgttgttatgggggtgctaaactttatcaaattgaagcaaataagccacaagaaagatacaagttttgcttggaaaacagtgttgttgaaactacctttagtattatcaaaaaacaccgcggattatgtttagcttacttagatagttatgtctctccttacctttgtttagctggtaacaagctccttgggtggttcactaaatacttttggtatagaaaaaggtgtgtgttttttaaccagaaccatttaaaacaaggtattctaaagattagtgtttactttttaagtgcaaaaaggtgtaaaaaagtttatgaaatcttaaaaaatacctttcaagaaaaggttaatgttttaagtttttctaataatttaattgagataactcatcatgatgctaataagggttatgcaattgaatatatggccaaaagagaacaactttcacttaatagaatagcagttattggtgattcttgaaatgattatgcaatgttcaaaaaagctaaatattcctttgcaatgtcaaaatccccttcccagttaaaattaattgctaccaataccagtaacaaaaccaaccgttaccgctttagtaccttacttaatttaattagtgaaacaatcattaatcaaaaagctgattagtaatgttcttttaaaaaaatagataaaagtatatagctaaatggaactgaaaaacattatttttgaccttgatggtaccttgctttcaagcaaccaaattccattagaacaaacagttgagtttttaaaggatttacagaaaaaagggattagaatcacttttgctagtggtagaagccatattttaattagaaacacagctacctttattacaccaaatctacctgtaatttcttccaatggtgcacttgtttatgattttgctagtgaaaaaccagttcatatcaaacctattgataataaagtaatacctgcaattatgcaaatgttgttggaatttcaagaaacattttatttctatacagataaaaaggtttttgcttttacacatgagcttgattcagctaaaattctttcaactagaagtcaaatagtaggaattgatctcattgaaaataactacatagttaacaagtttgaaaaagctttggattttgattttaagcaacatactattacaaagatcttactggtaactaaaaacagagaaaaagttccttttctagcaaaacaactagatcaaattcaagatattaactatgtgagttcaatgacatttgctcttgatatcatgcaaaaagatgttaataaggcttatggattgaaagtattagttgataattataatcttgatcctgaaaagactatggtctttggtgatgctgataatgatgttgaaatatttcaaagtgttaaatggccagttgctttggttaatggcactgatttagctaaaaaaaatgctaagtttattactgaatatgacaacaatcacaacggcatttatttctttctaaaaaagtttctagcaacttaagattagtaaacaggtttgctattcacgctgtttattaaaaatgtcaattattgcaaaaacagtttttataggtttaagcggtggtgttgattctgctgttagtgctttacttttaaaaaagcaataccaagaagttattggtgtttttatggaatgttgggatgagacacttaataatgatttttatggtcataagaaaataaataataacaaatcaggttgttcatcttttcaagacttccaacaggctaaaaaaatcgctaattctttaggaattaagttaataaaaaaaaacttaattgaagcttattgaaacaaagtttttttacctatgattcaaagtttcaaaaaagggttaaccccaaatccagacatctggtgtaatcgttttattaagtttggtttattgcatgatttttgtaagcaaattaaccctaattctctttttgcaactggtcattatgccaaaataaacatgatagaaaatcagcctttgctttctattcctaaagataccaataaagatcaaacttattttttagcaaatgttaaaaaagaacaatttcagaatgttatttttcctttagcagatttaaaaaaaataacagtgagaaatattgctagagaaaataattgagaagttgcagataaaaaagattcaactggaatttgttttattggtgaaagacatttcagtgattttttaaaaaactatttacctgtaaaaaaaggattaattaaggattgaaaaaccaaacaaactattagtgaacatgatggtgtttggttttatacgattggtcaacgcagtggattaaatttaggggggttaaaacaacgtcattttgttgttgctaaggatattgaaactaatgaattatttgtttcttgtgacaaagaagaattattgaaaacaacaattttattggatcaatttaactggttgtatacaccaaagcaacttcctagtcaagttctggtaagaattagacatgctcaaaaaccagaaattgcaaagttgaaattattatcagataataaactggaaataacatttaaaaatcctgttataagtgttgcatctggacagtttggtgtattatatacacttgatcaaatttgtttaggagcaggattaatttaaggtgatattattttctttgatggttgtgaatttgtaattaatgactaatttaattaaatatttaaaagaactccaaaactggctgtttgattatgtaaaaaaatctaaagctaaaggtgttatttttggcttatctggaggaattgattcagcagttgttgctgctattgctaaagaaacttttggttttgaaaaccatttagctttaataatgcatattaataattcaaaacttgattttcaagcaactagtgaacttgttaaaaaaatgcaatttaatagtattaacattgaactggaagagagtttcaatctgttagtaaaaacccttggaatagatccaaaaaaagattttttaacagctggtaacattaaagcacgtttacggatgataactttatatgcttatgctcaaaaacacaacttcttagttttaggtactggtaattttgtagagtatacacttggttatttcacaaaatgaggagatggagcttgtgatattgctcctttagcatggcttttaaaagaggacgtttacaaattagctaagcattttaatattcctgaaattgtaatcacaagagcgccaactgctagtctttttgaagggcaaactgatgagacagagatgggcattacttataaggaacttgatcaatatttaaaaggtgatttaatacttagttcagaaaagcaaaaaattgttttagatttgaaagcaaaagcagagcataaacataattcacctttgaaatttaaacatctctataatttccagaactaa

SEQ ID NO: 19 - DNA, 232 bp, E. coli
gatctatttatttagagatctgttctattgtgatctcttattaggatcgcactgccctgtggataacaaggatccggcttttaagatcaacaacctggaaaggatcattaactgtgaatgatcggtgatcctggaccgtataagctgggatcagaatgaggggttatacacaactcaaaaactgaacaacagttgttctttggataactaccggttgatccaagcttcctga